(19) United States
(12) Patent Application Publication
(10) Pub. No.: US 2026/0078451 A1
(43) Pub. Date: Mar. 19, 2026

(54) Diagnosis and Treatment of Pediatric Malignancies

(71) Applicant: ST. Jude Children's Research Hospital, Inc., Memphis, TN (US)

(72) Inventors: Xiaotu Ma, Memphis, TN (US); Jeffery M. Klco, Memphis, TN (US)

(21) Appl. No.: 19/382,380

(22) Filed: Nov. 7, 2025

Related U.S. Application Data
(63) Continuation-in-part of application No. 18/855,744.
(60) Provisional application No. 63/330,902, filed on Apr. 14, 2022.

(51) Int. Cl. C12Q 1/6886

(52) U.S. Cl. CPC C12Q 1/6886; C12Q 2600/156

(57) ABSTRACT

Methods of genetic screening, classifying a pediatric malignancy, monitoring the progression of a pediatric malignancy or response of a pediatric malignancy to chemotherapy in a subject comprising selectively detecting in a nucleic acid sample from the subject the presence or absence of one or more mutations in non-coding regions of a plurality of genes and a plurality of single nucleotide polymorphisms.

### REFERENCE TO RELATED APPLICATIONS

This application is a Continuation in part of U.S. patent application Ser. No. 18/855,744, filed Oct. 10, 2024, which is a 371 of International Application PCT/US2023/065129, filed Mar. 30, 2023, which claims benefit of priority from U.S. Provisional Patent Application Ser. No. 63/330,902, filed Apr. 14, 2022, further this application also claims benefit of U.S. Provisional Patent Application Ser. No. 63/880,627, filed Sep. 12, 2025, the contents of which are incorporated herein by reference in their entireties.

### STATEMENT REGARDING ELECTRONIC FILING OF A SEQUENCE LISTING

A Sequence Listing in XML format, entitled SJ0104USP1 ST26.xml, 86, 411 bytes in size, generated on Oct. 13, 2025 and filed herewith, is hereby incorporated by reference into the specification for its disclosures.

### BACKGROUND

Extensive insights on the genetic underpinnings (i.e., driver alterations) of childhood cancers have been uncovered in the past decade using next generation sequencing. Diagnostic sequencing has become part of clinical service in some institutions (Rusch et al. (2018) Nat. Commun. 9:3962; Hiemenz et al. (2018) J. Mol. Diagn. 20:765-776; Cheng et al. (2015) J. Mol. Diagn. 17:251-264). Although whole genome, and to lesser extent whole exome sequencing, is preferable to maximize the detection of cancer-associated variants in the clinical setting, there are significant resource and infrastructure requirements for these modalities that are not amenable to the majority of clinical labs. Further, the broad coverage of whole genome and whole exome sequencing renders it challenging to achieve ultra-deep sequencing that is essential for the analysis of specimens with low tumor purity such as for detecting minimal residual disease and for disease monitoring. Targeted gene panel-based sequencing holds the promise to address these challenges.

Although there have been multiple gene panels designed for adult cancers, such as MSK-IMPACT (Cheng et al. (2015) J. Mol. Diagn. 17:251-264), a comprehensive gene panel for pediatric cancers has not been described. This is important considering the recent pan-cancer study of 1, 699 childhood cancers that demonstrated a dramatic difference between adult and childhood cancers, where 55% of the 142 driver genes in pediatric cancers are not found in adult pan-cancer studies (Ma et al. (2018) Nature 555:371-376). In this study, 62% of driver alterations in childhood cancers are copy number alterations (CNVs) or structural variations (SVs) whose boundaries typically do not fall into protein coding regions. Indeed, a recent study of oncogenic fusions has shown that 55.7%, 22.5%, and 18.5% of pediatric leukemia, brain, and solid tumors have subtypes defined by oncogenic fusions, for which the DNA breakpoints typically fall into intronic regions (Liu et al. (2023) Nature Commun. 14:1739). This renders base pair level ascertainment of driver alterations in childhood cancers challenging when using conventional capture sequencing kits such as exome sequencing. Needed in the art is a dedicated gene panel for pediatric cancers that includes both coding and non-coding targets to maximize the detection of key alterations in pediatric cancers. The present invention addresses this need in the art.

### SUMMARY OF THE INVENTION

The invention provides a platform and method for genetic screening and treating a subject for a pediatric malignancy. According to one aspect of the disclosure, a method for genetic screening a subject for a pediatric malignancy is provided comprising obtaining a nucleic acid sample from the subject and selectively detecting in sequences of the nucleic acid sample one or more mutations in an exonic, intronic, and/or promoter region of the genes of Table 1 and single nucleotide polymorphisms of Table 2 thereby genetic screening the subject for a pediatric malignancy.

In another aspect, a method of classifying a pediatric malignancy of a subject is provided comprising obtaining a nucleic acid sample from the subject and selectively detecting in sequences of the nucleic acid sample one or more mutations in an exonic, intronic, and/or promoter region of the genes of Table 1 and single nucleotide polymorphisms of Table 2 thereby classifying the pediatric malignancy of the subject.

The disclosure also provides a method for monitoring progression of a pediatric malignancy in a subject comprising obtaining a first nucleic acid sample from the subject at a first time point; obtaining a second nucleic acid sample from the subject at a second time point; selectively detecting the presence in sequences of the first and second nucleic acid samples one or more mutations in an exonic, intronic, and/or promoter region of the genes of Table 1 and single nucleotide polymorphisms (SNPs) of Table 2; comparing the presence of the one or more mutations in the first nucleic acid sample with presence of the one or more mutations and SNPs in the second nucleic acid sample thereby monitoring the progression of the pediatric malignancy.

Further provided, is a method for monitoring the response of a pediatric malignancy to a therapy in a subject comprising obtaining a first nucleic acid sample from the subject prior to treatment with the therapy; obtaining a second nucleic acid sample from the subject after treatment with the therapy; selectively detecting the presence in sequences of the first and second nucleic acid samples one or more mutations in an exonic, intronic, and/or promoter region of the genes of Table 1 and single nucleotide polymorphisms of Table 2; comparing the presence of the one or more mutations and SNPs in the first nucleic acid sample with presence of the one or more mutations in the second nucleic acid sample thereby monitoring the response of the pediatric malignancy to the therapy in the subject.

### BRIEF DESCRIPTION OF THE DRAWINGS

FIGS. 1A-1D show capture uniformity per base (FIGS. 1A-1B) and per region (FIGS. 1C-1D) of the panel. Uniformity of coverage across in the SJPedPanel for high depth samples sequenced on Illumina NOVASEQ® 6000 (C1) and NOVASEQ® 500 (C2) respectively; and low depth samples sequenced on Illumina NOVASEQ® 6000 (C3) and NOVASE Q® 500 (C4) respectively. The histograms are made at base pair level (2.82 Mbp) (FIGS. 1A-1B) and region level (n=5009) (FIGS. 1C-1D). The vertical dotted lines indicate (μ−2σ) of the respective distributions. The statistical parameters (μ: average depth, σ: standard deviation, Cᵥ: coefficient of variance) were calculated by trimming observations in upper and lower 2.5 percentiles.

FIG. 2 shows the design of the study including panel content, investigation of ultra-sensitive detection, capture performance, diagnostic yield, and clinical applications.

FIG. 3 shows in silico coverage comparison between SJPedPanel and WES using percent coverage of variants (SNVs, Indels, Fusion, SV and ITDs) reported in the “Genomes for Kids” study (Newman et al. (2021) Cancer Discov 11:3008-3027). The last pair of bars with gray background shows combined percent coverage over “All” 485 variants.

FIG. 4 shows diagnostic yield of SJPedPanel by sequencing of previously reported 113 cases. Y-axis shows percentage of covered and detected variants by SJPedPanel over each variant type. The last pair of bars with gray background for “All” variants show combined detection rate. Numbers of reported driver alterations are indicated at the bottom of bars for corresponding variant types.

FIG. 5 shows mechanisms of oncogenic gene fusion formation. Scenario 1: the DNA breakpoints (10) can lead to fusion of coding exons (20) from N′ gene to 5′ UTR of C′ gene and result in conversion of the untranslated regions (30) into coding region, hence “neo-translational”. Scenario 2: the DNA breakpoints can lead to fusion of a coding exon from N′ gene to multiple possible coding exons of C′ gene, hence “versioning”. Scenario 3: the DNA breakpoints falling into a coding exon may disrupt the normal splice sites, and the cancer cell may use a novel splice site to ensure inclusion of corresponding exon, hence “neo-splicing”. In this scenario, a novel cryptic exon (40) may be created. Scenario 4: the DNA breakpoints may directly fuse two coding exons, hence “chimeric exon”. Scenario 5: a well-known phenomenon is promoter/enhancer hijacking.

FIG. 6 shows that the neo splice donor is essential to HAL-01 by CRISPR targeting using guide g₂. The induced Indels that happened to fall into coding region and lead to frameshift of TCF3-HLF are categorized into “Coding” group. Indels that directly disrupt the splice donor site are called “Loss”. Many induced Indels still leave a residual GT that may still serve as splice donor. The binding affinity of the donors after these Indels are predicted using position weight matrix (PWM) approach and categorized into different bins. Frequency of NGS reads carrying induced Indels are calculated according to such bins from day 3 to day 19 post editing. Data for three replicates is shown.

FIG. 7 depicts the pattern of neo splicing events due to incompatible exon frames between TCF3 exon 16 and HLF exon 4 in B-ALL cell line HAL-01. Guide RNAs were designed to target the cryptic exon (g₁, ATCTCAGGCGTGCCCGACTCNGG; SEQ ID NO: 1) and the neo splice sites (g₂, CTGAGATTTCTGGTGCAGGINGG; SEQ ID NO:2, and g₃, GATTCTATCACTCCTAGGCCNGG; SEQ ID NO: 3) as well as negative control guides (g₄, CTGGGGCTGGGAACTCCGTANGG; SEQ ID NO: 4, 180 bps upstream of g₃; g₅, TGTATGACTGTATCATAACGNGG; SEQ ID NO: 5, 35 bps downstream of g₂). A non-template insertion sequence of 27 bp (70) was observed.

FIG. 8 depicts the pattern of neo splicing events due to incompatible exon frames between TCF3 exon 16 and HLF exon 4 in the UoC-81 cell line. Shown is the theoretic analysis of open reading frames a, B, and 8 upon CRISPR editing, using either single guide or double guides. In the single guide scenario, there is always in-frame TCF3-HLF transcripts regardless of the length of induced indel. In the double guide scenario, three out of eight scenarios are predicted to disrupt all three isoforms (a, B, and 8) and lead to lethal effect.

FIG. 9 shows an exemplary workflow of cfDNA Pecan-Seq and variant detection. Targeted variants in cfDNA and genome-wide variants in bulk tumor were analyzed independently. ALL acute lymphoblastic leukemia.

### DETAILED DESCRIPTION OF THE INVENTION

This invention provides a pan-cancer gene panel (referred to herein as “SJPedPanel”) for diagnosing, classifying, treating, and monitoring progression and treatment of childhood cancers. The SJPedPanel encompasses genes relevant to childhood cancers and is shown to detect rare variants using ultra-deep sequencing via serial dilution experiments, as well as a use in disease monitoring in remission samples from acute myeloid leukemia (AML) patients. The SJPedPanel may be used to detect the presence or absence of one or more mutations, such as a single nucleotide variant (SNV), small insertion and deletion (Indel), gene fusion, structural variant (SV), internal tandem duplication (ITD), single nucleotide polymorphism (SNP), copy number variation (CNV) and/or loss of heterozygosity (LOH) in tumor samples or liquid biopsy samples. Once the one or more mutations are detected, the pediatric malignancy may be diagnosed and/or classified and the subject may be treated with an appropriate treatment regime for the particular type of pediatric malignancy. By way of illustration, the detection of an oncogenic gene fusion may include a targeted therapeutic approach for eliminating cancer cells by targeting neo splice sites or the cryptic exons found in oncogenic fusion genes.

Accordingly, the invention provides methods of genetic screening, classifying, and treating pediatric malignancy, monitoring the progression of a pediatric malignancy or response of a pediatric malignancy to a therapy in a subject comprising obtaining at least one nucleic acid sample from the subject and selectively detecting in sequences of the nucleic acid sample the presence or absence of one or more mutations in a region, e.g., an exonic, intronic, and/or promoter region, of the genes of Table 1 and single nucleotide polymorphisms of Table 2.

TABLE 1

| Gene | Transcript Isoform | Covered Exons |
|---|---|---|
| GNB1 | NM_002074 | 3-11 |
| RPL22 | NM_000983 | 1-4 |
| PIK3CD | NM_005026 | 3-24 |
| MTOR | NM_004958 | 2-58 |
| SDHB | NM_003000 | 1-8 |
| PAX7 | NM_002584 | 1-8 (intron 7) |
| ID3 | NM_002167 | 1-2 |
| RPL11 | NM_000975 | 1-6 |
| ARID1A | NM_006015 | 1-20 |
| ZMYM4 | NM_005095 | 1-30 |
| CSF3R | NM_000760 | 3-17 |
| MPL | NM_005373 | 1-12 |
| MUTYH | NM_001128425 | 1-16 |
| TAL1 | NM_003189 | 1-6 (introns 1-4) |
| CDKN2C | NM_001262 | 2-3 |
| JAK1 | NM_002227 | 2-25 |
| FUBP1 | NM_003902 | 1-20 |
| RPL5 | NM_000969 | 1-8 |
| RBM15 | NM_022768 | 1-2 (intron 1) |
| NRAS | NM_002524 | 2-5 |
| NOTCH2 | NM_024408 | 1-34 |
| BCL9 | NM_004326 | 4-10 (introns 8-9) |
| TPM3 | NM_152263 | 1-10 (introns 5-8) |
| RIT1 | NM_006912 | 2-6 |
| MEF2D | NM_005920 | 2-12 (introns 5-9) |
| NTRK1 | NM_001012331 | 1-16 (introns 8-9) |
| PBX1 | NM_002585 | 1-9 |
| RCSD1 | NM_052862 | 1-7 |
| ABL2 | NM_007314 | 1-12 (intron 4) |
| CDC73 | NM_024529 | 1-17 |
| MDM4 | NM_002393 | 2-11 |
| H3F3A | NM_002107 | 2-4 |
| ITPKB | NM_002221 | 2-8 |
| AKT3 | NM_005465 | 2-14 |
| GATA3 | NM_001002295 | 2-6 (intron 3) |
| MLLT10 | NM_004641 | 2-23 |
| WAC | NM_016628 | 1-14 |
| MAP3K8 | NM_005204 | 3-9 (intron 8) |
| RET | NM_020630 | 1-19 |
| NUTM2B | NM_001278495 | 1-7 (intron 1, 3, 4) |
| NUTM2A | NM_001099338 | 1-7 (intron 1) |
| PTEN | NM_000314 | 1-9 |
| TLX1 | NM_005521 | 1-3 (intron 1, 2) |
| LDB1 | NM_001113407 | 1-11 |
| SUFU | NM_016169 | 1-12 |
| NT5C2 | NM_012229 | 3-19 |
| SMC3 | NM_005445 | 1-29 |
| FGFR2 | NM_000141 | 2-18 |
| DMBT1 | NM_004406 | 1-40 |
| HRAS | NM_005343 | 2-5 |
| CDKN1C | NM_000076 | 1-2 |
| NUP98 | NM_016320 | 2-33 (introns 8-16) |
| LMO1 | NM_002315 | 2-4 (introns 2-3) |
| MYOD1 | NM_002478 | 1-3 |
| PAX6 | NM_000280 | 4-13 |
| WT1 | NM_000378 | 1-9 |
| LMO2 | NM_005574 | 3-6 |
| RAG1 | NM_000448 | 2 |
| RAG2 | NM_000536 | 2 |
| KBTBD4 | NM_018095 | 1-4 |
| MS4A1 | NM_152866 | 3-8 |
| ZFTA | NM_001144936 | 1-5 (introns 2-4) |
| MEN1 | NM_130799 | 2-10 |
| RELA | NM_021975 | 1-11 (introns 1-2) |
| CCND1 | NM_053056 | 1-5 |
| SHANK2 | NM_012309 | 2-27 (intron 9) |
| PICALM | NM_007166 | 1-20 (introns 17-19) |
| EED | NM_003797 | 1-12 |
| YAP1 | NM_001130145 | 1-9 |
| ATM | NM_000051 | 2-63 |
| KMT2A | NM_005933 | 1-36 (introns 6-11) |
| CBL | NM_005188 | 1-16 (introns 7-10) |
| USP2 | NM_004205 | 2-13 |
| CHEK1 | NM_001114122 | 2-13 |
| FLI1 | NM_002017 | 1-9 (introns 4-8) |
| KDM5A | NM_001042603 | 1-28 |
| CCND2 | NM_001759 | 1-5 |
| CHD4 | NM_001273 | 2-40 |
| ZNF384 | NM_133476 | 1-10 (introns 1-6) |
| ETV6 | NM_001987 | 1-8 (introns 4-5) |
| CDKN1B | NM_004064 | 1-2 |
| ATF7IP | NM_018179 | 1-15 |
| PIK3C2G | NM_004570 | 2-32 |
| KRAS | NM_033360 | 2-5 |
| ARID2 | NM_152641 | 1-21 (introns 1-2) |
| HDAC7 | NM_015401 | 1-26 |
| KMT2D | NM_003482 | 1-54 |
| ERBB3 | NM_001982 | 1-28 |
| STAT6 | NM_003153 | 2-22 |
| GLI1 | NM_005269 | 2-12 (introns 4-6) |
| CDK4 | NM_000075 | 2-8 |
| MDM2 | NM_002392 | 1-11 |
| BTG1 | NM_001731 | 1-2 |
| SH2B3 | NM_005475 | 2-8 |
| PTPN11 | NM_002834 | 1-15 |
| HNF1A | NM_000545 | 1-10 |
| POLE | NM_006231 | 1-49 |
| FLT3 | NM_004119 | 1-24 (intron 14) |
| BRCA2 | NM_000059 | 2-27 |
| PDS5B | NM_015032 | 2-35 |
| FOXO1 | NM_002015 | 1-3 (introns 1-2) |
| ELF1 | NM_172373 | 2-9 (introns 2-8) |
| RB1 | NM_000321 | 1-27 |
| ACIN1 | NM_014977 | 1-19 (introns 3-4) |
| BAZ1A | NM_013448 | 2-27 |
| NKX2-1 | NM_001079668 | 1-3 (introns 1-2) |
| OTX2 | NM_021728 | 3-5 |
| SIX1 | NM_005982 | 1-2 |
| MAX | NM_002382 | 1-5 |
| DICER1 | NM_177438 | 2-27 |
| BCL11B | NM_138576 | 1-4 |
| AKT1 | NM_005163 | 2-14 |
| IGH | chr14: 106309991- | |
| | 106390010, 106789991- | |
| | 106795010, 107047991- | |
| | 107049010 | |
| NUTM1 | NM_175741 | 2-8 (introns 2, 4, 5) |
| INO80 | NM_017553 | 2-36 |
| MGA | NM_001164273 | 2-24 |
| MAP2K1 | NM_002755 | 1-11 |
| NTRK3 | NM_001012338 | 3-20 (intron 14) |
| IDH2 | NM_002168 | 1-11 |
| TSC2 | NM_000548 | 2-42 |
| TRAF7 | NM_032271 | 2-21 |
| CREBBP | NM_004380 | 1-31 (intron 2) |
| TFAP4 | NM_003223 | 1-7 |
| GLIS2 | NM_032575 | 1-6 (intron 2) |
| USP7 | NM_003470 | 1-31 |
| MYH11 | NM_002474 | 2-41 |
| PALB2 | NM_024675 | 1-13 |
| CD19 | NM_001770 | 1-14 |
| FUS | NM_004960 | 1-15 (introns 3-14) |
| CBFB | NM_022845 | 1-6 (intron 5) |
| CTCF | NM_006565 | 3-12 |
| CDH1 | NM_004360 | 1-16 |
| GSE1 | NM_014615 | 1-16 |
| CBFA2T3 | NM_005187 | 1-12 (intron 11) |
| PHF23 | NM_024297 | 1-5 (introns 3-4) |
| TP53 | NM_000546 | 1-11 (introns 1-10) |
| MAP2K4 | NM_003010 | 1-11 |
| NCOR1 | NM_006311 | 2-46 |
| NF1 | NM_000267 | 1-57 |
| SUZ12 | NM_015355 | 1-16 |
| TAF15 | NM_139215 | 1-16 (introns 6, 9) |
| ERBB2 | NM_004448 | 1-27 |
| IKZF3 | NM_012481 | 1-8 |
| STAT5B | NM_012448 | 2-19 |
| BRCA1 | NM_007294 | 2-23 |
| UBTF | NM_014233 | 2-21 (introns 9-13) |
| MAP3K14 | NM_003954 | 2-17 (intron 5) |
| HLF | NM_002126 | 1-4 (intron 3) |
| PPM1D | NM_003620 | 1-6 |
| GNA13 | NM_006572 | 1-4 |
| PRKAR1A | NM_002734 | 2-11 |
| H3F3B | NM_005324 | 2-4 |
| SEPTIN9 | NM_001113491 | 1-12 |
| PTPN2 | NM_002828 | 1-9 |
| SETBP1 | NM_015559 | 2-6 |
| SMAD4 | NM_005359 | 2-12 |
| TCF4 | NM_001083962 | 2-19 |
| BCL2 | NM_000633 | 2-3 |
| STK11 | NM_000455 | 1-9 |
| DAZAP1 | NM_170711 | 1-12 (intron 6) |
| TCF3 | NM_003200 | 2-19 (introns 11-18) |
| GNA11 | NM_002067 | 1-7 |
| ZBTB7A | NM_015898 | 2-3 |
| MAP2K2 | NM_030662 | 1-11 |
| MLLT1 | NM_005934 | 1-12 |
| DNM2 | NM_001005360 | 1-21 |
| SMARCA4 | NM_001128849 | 2-36 |
| EPOR | NM_000121 | 1-8 (intron 7) |
| LYL1 | NM_005583 | 2-4 |
| JAK3 | NM_000215 | 2-24 |
| PIK3R2 | NM_005027 | 2-16 |
| ELL | NM_006532 | 1-12 |
| CEBPA | NM_004364 | 1 |
| UBA2 | NM_005499 | 1-17 |
| CD22 | NM_001771 | 2-14 |
| KMT2B | NM_014727 | 1-37 |
| AKT2 | NM_001626 | 2-14 |
| HNRNPUL1 | NM_007040 | 1-15 |
| CIC | NM_015125 | 1-20 (introns 16-19) |
| POLD1 | NM_002691 | 2-27 |
| CD33 | NM_001772 | 1-7 |
| CNOT3 | NM_014516 | 2-18 |
| U2AF2 | NM_007279 | 1-12 |
| MYCN | NM_005378 | 2-3 |
| DNMT3A | NM_175629 | 2-23 |
| ASXL2 | NM_018263 | 1-13 |
| ALK | NM_004304 | 1-29 (intron 19) |
| SOS1 | NM_005633 | 1-23 |
| ZFP36L2 | NM_006887 | 1-2 |
| SIX2 | NM_016932 | 1-2 |
| MSH2 | NM_000251 | 1-16 |
| MSH6 | NM_000179 | 1-10 |
| FBXO11 | NM_025133 | 2-23 |
| XPO1 | NM_003400 | 2-25 |
| GLI2 | NM_005270 | 2-14 |
| ZEB2 | NM_014795 | 2-10 |
| ACVR1 | NM_001105 | 3-11 |
| TBR1 | NM_006593 | 1-6 |
| HOXD13 | NM_000523 | 1-2 (intron 1) |
| SF3B1 | NM_012433 | 1-25 |
| IDH1 | NM_005896 | 3-10 |
| ERBB4 | NM_005235 | 1-28 |
| IKZF2 | NM_016260 | 2-8 |
| PAX3 | NM_181457 | 1-8 (intron 7) |
| SEPTIN2 | NM_001008491 | 3-13 |
| ASXL1 | NM_015338 | 1-13 |
| GNAS | NM_000516 | 1-13 |
| RUNX1 | NM_001754 | 2-9 (intron 6) |
| ERG | NM_182918 | 1-10 (intron 6-9) |
| U2AF1 | NM_006758 | 1-8 |
| SEPTIN5 | NM_002688 | 1-12 (introns 1-3) |
| DGCR8 | NM_022720 | 2-14 |
| MAPK1 | NM_002745 | 1-8 |
| BCR | NM_004327 | 1-23 |
| SMARCB1 | NM_003073 | 1-9 (introns 1-8) |
| MN1 | NM_002430 | 1-2 |
| CHEK2 | NM_007194 | 2-15 |
| XBP1 | NM_005080 | 1-5 |
| EWSR1 | NM_013986 | 1-18 (introns 8-12) |
| NF2 | NM_000268 | 1-16 |
| EP300 | NM_001429 | 1-31 |
| BRPF1 | NM_001003694 | 2-14 |
| VHL | NM_000551 | 1-3 |
| RAF1 | NM_002880 | 2-17 (intron 12) |
| MLH1 | NM_000249 | 1-19 |
| CTNNB1 | NM_001904 | 2-15 |
| SETD2 | NM_014159 | 1-21 |
| RHOA | NM_001664 | 2-5 |
| BAP1 | NM_004656 | 1-17 |
| MITF | NM_198159 | 1-10 |
| CD200 | NM_005944 | 1-6 |
| GATA2 | NM_001145661 | 3-7 |
| STAG1 | NM_005862 | 2-34 |
| PIK3CB | NM_006219 | 3-24 |
| ZIC1 | NM_003412 | 1-3 |
| MLF1 | NM_022443 | 1-7 (intron 1) |
| TERC | NR 001566 | 1 |
| TBL1XR1 | NM_024665 | 3-16 |
| PIK3CA | NM_006218 | 2-21 |
| BCL6 | NM_001706 | 3-10 |
| FGFR3 | NM_000142 | 2-18 (introns 9-17) |
| WHSC1 | NM_133330 | 4-24 |
| DHX15 | NM_001358 | 1-14 |
| PDGFRA | NM_006206 | 2-23 |
| KIT | NM_000222 | 1-21 |
| KDR | NM_002253 | 1-30 |
| AFF1 | NM_001166693 | 2-21 |
| TET2 | NM_001127208 | 3-11 |
| LEF1 | NM_016269 | 1-11 |
| NR3C2 | NM_000901 | 2-9 |
| FBXW7 | NM_033632 | 2-12 |
| DUX4 | NM_001293798 | |
| SDHA | NM_004168 | 1-15 |
| TERT | NM_198253 | 1-16 (introns 9-15) |
| DROSHA | NM_013235 | 3-35 |
| IL7R | NM_002185 | 1-8 |
| NIPBL | NM_133433 | 2-47 |
| PIK3R1 | NM_181523 | 2-16 |
| SSBP2 | NM_012446 | 1-17 |
| APC | NM_000038 | 2-16 |
| RAD50 | NM_005732 | 1-25 |
| AFF4 | NM_014423 | 2-21 |
| TCF7 | NM_003202 | 1-10 |
| CXXC5 | NM_016463 | 2-3 (intron 2) |
| NR3C1 | NM_000176 | 2-9 |
| CSF1R | NM_005211 | 2-22 (introns 11-12) |
| PDGFRB | NM_002609 | 2-23 (introns 8-10) |
| EBF1 | NM_024007 | 1-16 |
| TLX3 | NM_021025 | 1-3 (introns 1-2) |
| NPM1 | NM_002520 | 1-11 (introns 5-6) |
| FGFR4 | NM_002011 | 2-18 |
| NSD1 | NM_172349 | 2-24 |
| IRF4 | NM_002460 | 2-9 |
| DEK | NM_003472 | 2-11 |
| HIST1H3A | NM_003529 | 1 |
| HIST1H3B | NM_003537 | 1 |
| HIST1H3C | NM_003531 | 1 |
| DAXX | NM_001141969 | 2-8 |
| CCND3 | NM_001760 | 1-5 |
| PHIP | NM_017934 | 1-40 |
| ROS1 | NM_002944 | 1-43 (introns 31-35) |
| MYB | NM_001130173 | 1-16 (intron 1) |
| TNFAIP3 | NM_006290 | 2-9 |
| ARID1B | NM_017519 | 1-19 |
| MAP3K4 | NM_005922 | 1-27 |
| QKI | NM_006775 | 1-8 (intron 4) |
| MLLT4 | NM_001040000 | 1-30 |
| ACTB | NM_001101 | 2-6 |
| PMS2 | NM_000535 | 1-15 |
| RAC1 | NM_006908 | 1-6 |
| HOXA10 | NM_018951 | 1-2 |
| IKZF1 | NM_006060 | 2-8 |
| EGFR | NM_005228 | 1-28 |
| CDK6 | NM_001259 | 2-8 |
| SAMD9 | NM_017654 | 3 |
| SAMD9L | NM_152703 | 5 |
| CUX1 | NM_181552 | 1-24 |
| KMT2E | NM_182931 | 3-27 |
| PIK3CG | NM_002649 | 2-11 |
| MET | NM_001127500 | 2-21 |
| SMO | NM_005631 | 1-12 |
| KIAA1549 | NM_020910 | 1-20 (introns 15-16) |
| ZC3HAV1 | NM_020119 | 1-13 |
| BRAF | NM_004333 | 1-18 (introns 8-9) |
| EZH2 | NM_001203247 | 2-20 |
| KMT2C | NM_170606 | 1-59 |
| SHH | NM_000193 | 1-3 |
| FGFR1 | NM_023110 | 2-18 (introns 9-17) |
| TACC1 | NM_006283 | 1-13 (intron 6) |
| PLAG1 | NM_002655 | 4-5 |
| CHD7 | NM_017780 | 2-38 |
| MYBL1 | NM_001080416 | 1-16 |
| RUNX1T1 | NM_004349 | 1-11 |
| RAD21 | NM_006265 | 2-14 |
| MYC | NM_002467 | 1-3 (introns 1-2) |
| SMARCA2 | NM_003070 | 2-34 |
| JAK2 | NM_004972 | 3-25 (introns 10-20) |
| PTPRD | NM_002839 | 12-46 |
| MLLT3 | NM_004529 | 1-11 |
| CDKN2A | NM_000077 | 1-3 (introns 1-2) |
| CDKN2B | NM_004936 | 1-2 |
| PAX5 | NM_016734 | 1-10 (introns 1-9) |
| GNAQ | NM_002072 | 1-7 |
| NTRK2 | NM_006180 | 4-21 (introns 10-11, |
| | | 14-15) |
| PTCH1 | NM_000264 | 1-23 |
| TAL2 | NM_005421 | 1 |
| KLF4 | NM_004235 | 1-5 |
| FPGS | NM_004957 | 1-15 (introns 1-14) |
| ABL1 | NM_005157 | 1-11 (introns 1-3) |
| NUP214 | NM_005085 | 1-36 (introns 17, 31-34) |
| TSC1 | NM_000368 | 3-23 |
| NOTCH1 | NM_017617 | 1-34 (introns 25-33) |
| CRLF2 | NM_022148 | 1-8 (introns 6-7) |
| IL3RA | NM_002183 | 2-12 |
| P2RY8 | NM_178129 | 1-2 |
| PRPS2 | NM_001039091 | 1-7 |
| BEND2 | NM_153346 | 1-14 |
| BCOR | NM_017745 | 2-15 (introns 12-14) |
| USP9X | NM_001039590 | 2-45 (intron 31) |
| DDX3X | NM_001356 | 1-17 (intron 1) |
| KDM6A | NM_021140 | 1-29 |
| GATA1 | NM_002049 | 2-6 (introns 2-5) |
| CXorf67 | NM_203407 | 1 |
| SMC1A | NM_006306 | 1-25 |
| FOXR2 | NM_198451 | 1 |
| AMER1 | NM_152424 | 2 |
| MED12 | NM_005120 | 1-45 |
| ZMYM3 | NM_201599 | 2-25 |
| NONO | NM_001145408 | 4-13 |
| ATRX | NM_000489 | 1-35 (intron 10) |
| PRPS1 | NM_002764 | 1-7 |
| SEPTIN6 | NM_145799 | 1-10 |
| STAG2 | NM_001042749 | 3-35 |
| BCORL1 | NM_021946 | 1-12 |
| PHF6 | NM_001015877 | 2-10 |
| FMR1 | NM_002024 | 1-17 |
| RPL10 | NM_006013 | 1-7 |

Table 1 includes a portion of Table 1 of U.S. Provisional Application Ser. No. 63/880,627, incorporated herein by reference in its entirety. Table 1 of US 63/880,627 provides a list of 5009 exonic/intronic/promoter regions from 357 genes of the human genome (GRCh37/hg19) included in the SJPedPanel, wherein “Chrom” refers to chromosome number, “Start” refers to start position of the region (GRCh37/hg19), “End” refers to end position of the region (GRCh37/hg19), “Gene” refers to gene name, “Transcript Isoform” refers to Accession number of the transcript isoform used for the gene, “Length” refers to length of the region, “Covered Exon(s)” refers to exons covered by the region, and “Covered Intron(s)” refers to intron covered by the region with at least 80% of the corresponding intron length (Note: The regions were padded with ˜10 bases flanking the mentioned exons).

TABLE 2

| SNP | SNP | SNP | SNP | SNP |
|---|---|---|---|---|
| rs3813194 | rs10783269 | rs11672361 | rs34231214 | rs1158633 |
| rs12135382 | rs4113945 | rs3786717 | rs1402214 | rs724634 |
| rs61776850 | rs2277366 | rs4804546 | rs974959 | rs7798437 |
| rs882786 | rs3209584 | rs11880865 | rs7620058 | rs6967510 |
| rs61763695 | rs10875870 | rs440677 | rs6784862 | rs34069660 |
| rs1891143 | rs4237853 | rs3745681 | rs6801204 | rs7455882 |
| rs3819954 | rs1054376 | rs3745682 | rs1862074 | rs1524526 |
| rs6665286 | rs1051886 | rs2116876 | rs4511906 | rs4722744 |
| rs242048 | rs2293446 | rs8113582 | rs2862413 | rs10247091 |
| rs2199215 | rs7969091 | rs12608933 | rs6443361 | rs4719963 |
| rs973978 | rs11168850 | rs35863302 | rs6443362 | rs4722960 |
| rs2783557 | rs3741621 | rs7256770 | rs4455325 | rs702829 |
| rs9661578 | rs9325145 | rs9973204 | rs7609719 | rs1049305 |
| rs56233416 | rs2334930 | rs4804635 | rs10936907 | rs6966782 |
| rs4623725 | rs12229524 | rs5851 | rs6443391 | rs2108858 |
| rs1287623 | rs2290442 | rs286216 | rs4488841 | rs6980186 |
| rs3747992 | rs6580738 | rs431393 | rs7629858 | rs17741563 |
| rs962662 | rs706805 | rs286243 | rs9840357 | rs1894776 |
| rs4908583 | rs4761973 | rs2459041 | rs9875622 | rs10257524 |
| rs806104 | rs303764 | rs12975560 | rs3979210 | rs12536475 |
| rs806111 | rs659695 | rs4143973 | rs1201289 | rs10270017 |
| rs12047044 | rs10747658 | rs2112465 | rs6443434 | rs3801309 |
| rs3827724 | rs4759245 | rs6511906 | rs6809739 | rs10807886 |
| rs11121191 | rs2365345 | rs11085895 | rs7433120 | rs6462758 |
| rs2843493 | rs10876565 | rs3746295 | rs6443449 | rs2598027 |
| rs4908542 | rs7963067 | rs4808031 | rs7427204 | rs2534559 |
| rs12740426 | rs10876741 | rs12461484 | rs1995243 | rs10237576 |
| rs3810985 | rs10783768 | rs8108174 | rs9810634 | rs4723839 |
| rs200441 | rs10783818 | rs4808662 | rs4857707 | rs10282121 |
| rs2281190 | rs7486863 | rs8108738 | rs9820071 | rs1257699 |
| rs406028 | rs7298418 | rs1858846 | rs6443572 | rs40814 |
| rs11802071 | rs4760228 | rs4621106 | rs56266920 | rs273116 |
| rs179265 | rs4108243 | rs11673240 | rs7636789 | rs498403 |
| rs225115 | rs10876980 | rs2336460 | rs3774239 | rs4724103 |
| rs301819 | rs11172203 | rs11085450 | rs6771602 | rs2583872 |
| rs770027 | rs3809113 | rs2666451 | rs6802529 | rs17545513 |
| rs7518602 | rs11544238 | rs2163846 | rs10937062 | rs2079417 |
| rs1292666 | rs11172254 | rs6511416 | rs7647348 | rs10951758 |
| rs11121678 | rs1545783 | rs10417800 | rs1546751 | rs217370 |
| rs3125813 | rs923828 | rs12981188 | rs11925419 | rs6963847 |
| rs816063 | rs2307095 | rs62108583 | rs2251248 | rs1830064 |
| rs3924048 | rs1689600 | rs12974171 | rs6782682 | rs10229794 |
| rs376251 | rs813854 | rs1363330 | rs9820970 | rs62452737 |
| rs1033454 | rs2640636 | rs7252442 | rs13087397 | rs34894454 |
| rs868804 | rs7952989 | rs1048123 | rs4608682 | rs2708870 |
| rs6693394 | rs17852479 | rs33435 | rs4012241 | rs4351374 |
| rs11588341 | rs7971877 | rs278183 | rs35846970 | rs7789657 |
| rs3003413 | rs6581159 | rs8104214 | rs9849004 | rs6959240 |
| rs653561 | rs3110448 | rs1991812 | rs1820433 | rs13235510 |
| rs6684062 | rs4760368 | rs2867743 | rs7625652 | rs4917009 |
| rs12127986 | rs2653865 | rs73038273 | rs3732920 | rs28508243 |
| rs12410157 | rs9738727 | rs12150904 | rs9867947 | rs11761913 |
| rs4920481 | rs10783878 | rs8111376 | rs1598245 | rs2168589 |
| rs55786092 | rs1502817 | rs6510332 | rs2886244 | rs6583441 |
| rs2745301 | rs7304216 | rs11084699 | rs9291009 | rs6969942 |
| rs685300 | rs3913082 | rs4805852 | rs9815069 | rs6945253 |
| rs479064 | rs10877162 | rs11084707 | rs4687367 | rs12719056 |
| rs992661 | rs2221158 | rs8113143 | rs56320859 | rs1451371 |
| rs6426637 | rs1389256 | rs11673115 | rs2367936 | rs12538938 |
| rs6679593 | rs7488668 | rs10423902 | rs11716336 | rs2529388 |
| rs3813983 | rs2956512 | rs10775578 | rs9855580 | rs4947757 |
| rs7525201 | rs6581429 | rs7248219 | rs7640738 | rs1019000 |
| rs10799715 | rs1988657 | rs10420551 | rs1862768 | rs11238325 |
| rs10917454 | rs163683 | rs55993669 | rs13059550 | rs34868692 |
| rs7523442 | rs10784345 | rs11084744 | rs9836035 | rs1024606 |
| rs1925668 | rs57477949 | rs10401769 | rs7622678 | rs1107144 |
| rs10218584 | rs11175457 | rs10417398 | rs7616279 | rs9691442 |
| rs12131384 | rs1147094 | rs4805974 | rs28605783 | rs11972572 |
| rs2807331 | rs7955395 | rs379571 | rs3816683 | rs10280610 |
| rs12024783 | rs1143833 | rs34044778 | rs12641735 | rs1526769 |
| rs6677427 | rs3741742 | rs11670828 | rs9884421 | rs1220767 |
| rs486575 | rs373632 | rs328404 | rs13147602 | rs595452 |
| rs2135755 | rs1373877 | rs10853935 | rs7690727 | rs6974230 |
| rs35109971 | rs10784432 | rs8103093 | rs3903127 | rs7782765 |
| rs3767876 | rs2336165 | rs11669233 | rs1317924 | rs2709317 |
| rs10751735 | rs10878228 | rs12972325 | rs7655119 | rs11773594 |
| rs10794528 | rs2336433 | rs400312 | rs7657357 | rs6960351 |
| rs59607484 | rs10784441 | rs6510439 | rs13135515 | rs2330762 |
| rs11247955 | rs10878243 | rs2290655 | rs11248002 | rs887810 |
| rs392814 | rs1566318 | rs8101810 | rs7655221 | rs10899767 |
| rs6666121 | rs1370940 | rs1816055 | rs56142235 | rs723527 |
| rs11247593 | rs4762081 | rs755733 | rs35254228 | rs10259320 |
| rs834201 | rs1587056 | rs2545974 | rs6855904 | rs2222626 |
| rs6598870 | rs6581641 | rs2651111 | rs11737105 | rs7809890 |
| rs12752945 | rs4762082 | rs916694 | rs4865423 | rs10282700 |
| rs6598874 | rs7308337 | rs2242523 | rs34428528 | rs6972560 |
| rs3813795 | rs10878313 | rs2912420 | rs6852931 | rs2692608 |
| rs3766400 | rs7296827 | rs8110796 | rs4865425 | rs11238367 |
| rs10794514 | rs2118137 | rs2927743 | rs11248067 | rs7807357 |
| rs419335 | rs10784558 | rs1725472 | rs4865447 | rs6593261 |
| rs4654335 | rs2199828 | rs4547457 | rs7376928 | rs10250208 |
| rs10914603 | rs2700104 | rs31725 | rs13117684 | rs1844162 |
| rs12122914 | rs2870951 | rs62106867 | rs6599398 | rs4128371 |
| rs1188447 | rs1026792 | rs166539 | rs8051 | rs62459530 |
| rs7512805 | rs436881 | rs396118 | rs2236787 | rs150311808 |
| rs422215 | rs10878823 | rs10411012 | rs2249143 | rs13226734 |
| rs6701932 | rs7974106 | rs2116943 | rs4558926 | rs1524618 |
| rs1325243 | rs7955976 | rs1206032 | rs2896518 | rs4717922 |
| rs1993911 | rs2468416 | rs2259013 | rs56337234 | rs10236233 |
| rs11264155 | rs2468418 | rs304725 | rs11248078 | rs2949690 |
| rs7530283 | rs2546524 | rs2571067 | rs3135846 | rs2016325 |
| rs2983117 | rs7301067 | rs17658470 | rs405669 | rs4433018 |
| rs61780480 | rs6581833 | rs915915 | rs1923775 | rs11972225 |
| rs10908362 | rs1690920 | rs10419176 | rs1183191 | rs60391001 |
| rs10789307 | rs3741598 | rs3745758 | rs2051561 | rs7810725 |
| rs1930567 | rs1195739 | rs10403090 | rs11248095 | rs1699448 |
| rs950805 | rs6581841 | rs838131 | rs7693437 | rs62457240 |
| rs3131700 | rs1908674 | rs7256629 | rs13126907 | rs12672930 |
| rs3013461 | rs10878908 | rs1273643 | rs4974649 | rs11771277 |
| rs566235 | rs7306347 | rs2085459 | rs2031726 | rs7797779 |
| rs10889870 | rs7306118 | rs952108 | rs3135097 | rs4318932 |
| rs1327386 | rs6581874 | rs11084104 | rs1203767 | rs11763011 |
| rs16829262 | rs7135981 | rs2560878 | rs846083 | rs10235086 |
| rs841571 | rs4274230 | rs1550577 | rs2699432 | rs6963070 |
| rs634387 | rs315131 | rs10412187 | rs177794 | rs4729343 |
| rs3828139 | rs1152973 | rs10411415 | rs4234727 | rs1181901 |
| rs561842 | rs10879108 | rs28373215 | rs2280041 | rs798324 |
| rs5027205 | rs17109528 | rs4801183 | rs1121533 | rs17349903 |
| rs781226 | rs7486748 | rs2370136 | rs765831 | rs12705955 |
| rs4660882 | rs1318686 | rs9749195 | rs12503199 | rs7791175 |
| rs785502 | rs1121488 | rs12611212 | rs7681193 | rs728460 |
| rs658210 | rs10879684 | rs55800631 | rs57831033 | rs1358340 |
| rs10890422 | rs10879785 | rs11688307 | rs3756186 | rs7803395 |
| rs1502934 | rs11180344 | rs4971463 | rs13104605 | rs10266038 |
| rs1117514 | rs2471647 | rs4853837 | rs34301346 | rs215310 |
| rs12046348 | rs10785200 | rs7588195 | rs6831580 | rs6952691 |
| rs7519456 | rs35590166 | rs11127344 | rs10938996 | rs4732576 |
| rs2008713 | rs10779153 | rs1609389 | rs6819890 | rs2732766 |
| rs3122615 | rs10506743 | rs9308792 | rs10018548 | rs2463670 |
| rs2622908 | rs10777395 | rs4849939 | rs35522517 | rs2708542 |
| rs6795 | rs2694837 | rs9989764 | rs977031 | rs1534756 |
| rs543640 | rs10861082 | rs10929490 | rs12500577 | rs11970980 |
| rs12143164 | rs1245811 | rs792048 | rs3846407 | rs7802773 |
| rs1911829 | rs2255747 | rs10178841 | rs10939557 | rs9641021 |
| rs12031428 | rs7294856 | rs1462059 | rs10024821 | rs6953374 |
| rs6587765 | rs7966929 | rs9287683 | rs7699101 | rs6958559 |
| rs7554550 | rs7979434 | rs7570561 | rs11723911 | rs10270950 |
| rs11205608 | rs10746287 | rs12989276 | rs157617 | rs10953006 |
| rs12030579 | rs7972528 | rs34011894 | rs12507442 | rs732956 |
| rs9660278 | rs986489 | rs13423661 | rs7683620 | rs13222775 |
| rs3850874 | rs7138143 | rs6715057 | rs4508879 | rs2027950 |
| rs12035331 | rs7313029 | rs1035119 | rs10805211 | rs13236026 |
| rs7520502 | rs2219708 | rs2969887 | rs6833720 | rs6965936 |
| rs835036 | rs1024076 | rs2011812 | rs7666014 | rs10488514 |
| rs6662414 | rs11104061 | rs12479436 | rs3816874 | rs2097810 |
| rs6673692 | rs9804849 | rs4668754 | rs4697207 | rs38794 |
| rs4927007 | rs12816164 | rs1519545 | rs10516405 | rs42235 |
| rs9651202 | rs2468229 | rs1400152 | rs12504455 | rs1005346 |
| rs1147984 | rs1879205 | rs12052352 | rs2250074 | rs28758759 |
| rs1392141 | rs7308258 | rs11682654 | rs215429 | rs6944020 |
| rs1165499 | rs10858948 | rs2571634 | rs2970870 | rs9886048 |
| rs706392 | rs10858976 | rs714554 | rs13109835 | rs7808280 |
| rs6671760 | rs7980349 | rs35237017 | rs11728255 | rs6953868 |
| rs17459976 | rs7962133 | rs2111451 | rs4697446 | rs2188177 |
| rs11207077 | rs1387761 | rs2276664 | rs1548171 | rs4729062 |
| rs6587804 | rs2131711 | rs10153729 | rs2007816 | rs10266958 |
| rs12562366 | rs10859190 | rs7575749 | rs6817993 | rs10215895 |
| rs674612 | rs7298522 | rs6720127 | rs61794566 | rs7776961 |
| rs893958 | rs11106300 | rs4668980 | rs13123464 | rs2157746 |
| rs686119 | rs11106309 | rs2380702 | rs55900493 | rs2106169 |
| rs472913 | rs11106372 | rs36044429 | rs11945648 | rs10277338 |
| rs5016273 | rs7133028 | rs13028732 | rs11736194 | rs6965201 |
| rs6587938 | rs7358548 | rs3755135 | rs1007991 | rs12333722 |
| rs12406266 | rs790458 | rs6751458 | rs13138049 | rs6964942 |
| rs12406541 | rs1579655 | rs41459549 | rs12648866 | rs4268 |
| rs855331 | rs2805890 | rs3924150 | rs6851275 | rs42499 |
| rs1333739 | rs381966 | rs667089 | rs3796780 | rs10953093 |
| rs6671428 | rs409591 | rs1583104 | rs10006741 | rs42502 |
| rs6659197 | rs7966071 | rs7598974 | rs6448435 | rs42506 |
| rs699255 | rs481627 | rs11679606 | rs12644574 | rs42512 |
| rs10889629 | rs7967300 | rs12986604 | rs7676866 | rs2374616 |
| rs4655706 | rs1515561 | rs1031768 | rs1449761 | rs6976526 |
| rs12036629 | rs7312919 | rs13416608 | rs12645618 | rs2188510 |
| rs1924542 | rs2385096 | rs4485566 | rs1510789 | rs2519570 |
| rs9730004 | rs11106795 | rs4666544 | rs13148329 | rs10279629 |
| rs1913273 | rs10777486 | rs35135293 | rs4588423 | rs1926095 |
| rs655126 | rs4761573 | rs343161 | rs6858230 | rs1234475 |
| rs6670034 | rs10777596 | rs437624 | rs4438724 | rs10256944 |
| rs7411 | rs7310973 | rs7571528 | rs10461087 | rs1404510 |
| rs1213558 | rs3596 | rs6739738 | rs2062990 | rs34336263 |
| rs1776012 | rs4762652 | rs4233695 | rs10025535 | rs6955226 |
| rs1525979 | rs7315748 | rs1709333 | rs10008623 | rs4729431 |
| rs9633293 | rs7307168 | rs1559472 | rs6824546 | rs869029 |
| rs6424564 | rs7132990 | rs6545776 | rs1113371 | rs12666377 |
| rs1984229 | rs7975941 | rs7586928 | rs4833125 | rs533486 |
| rs3113979 | rs10777932 | rs6546148 | rs4833143 | rs12666785 |
| rs11163074 | rs10860361 | rs10180663 | rs1648396 | rs6465807 |
| rs10782582 | rs10745860 | rs6740340 | rs4832956 | rs382270 |
| rs55813741 | rs10778029 | rs6756654 | rs1487635 | rs3800985 |
| rs11162297 | rs35712 | rs2602992 | rs56409419 | rs6963104 |
| rs12126319 | rs10860611 | rs12992952 | rs7655170 | rs2711879 |
| rs7538245 | rs10778112 | rs11689092 | rs3912391 | rs12154738 |
| rs11162648 | rs7131821 | rs10203731 | rs2639290 | rs10085749 |
| rs987961 | rs10860879 | rs6739177 | rs1567231 | rs66482905 |
| rs4323660 | rs1568794 | rs7609004 | rs10029283 | rs4730097 |
| rs12757602 | rs10507158 | rs11687340 | rs4591641 | rs887881 |
| rs12740874 | rs7963412 | rs6546467 | rs2345475 | rs849387 |
| rs10399830 | rs10861199 | rs13413825 | rs7696058 | rs7781603 |
| rs12121851 | rs2468338 | rs13000546 | rs7659996 | rs423686 |
| rs4443886 | rs1196761 | rs4063544 | rs13118199 | rs10272114 |
| rs323936 | rs7961513 | rs10208202 | rs6829386 | rs6466266 |
| rs6703493 | rs10778482 | rs12989452 | rs552538 | rs990943 |
| rs1521518 | rs4964554 | rs67127891 | rs13109080 | rs401228 |
| rs603746 | rs5009017 | rs6729127 | rs4279178 | rs17438711 |
| rs709759 | rs1946374 | rs7604968 | rs13150235 | rs6956114 |
| rs6665375 | rs2375013 | rs12713741 | rs11732338 | rs7805703 |
| rs478277 | rs2300459 | rs17047388 | rs7679210 | rs3095045 |
| rs6576880 | rs10850481 | rs3806516 | rs2768948 | rs10239282 |
| rs7549602 | rs17789061 | rs12999113 | rs225170 | rs2396655 |
| rs1336584 | rs2157873 | rs4450561 | rs12500525 | rs2690826 |
| rs6659626 | rs4766529 | rs11126836 | rs10003612 | rs1852470 |
| rs12729209 | rs1001484 | rs6761095 | rs10212730 | rs2520580 |
| rs1215649 | rs6489979 | rs60039280 | rs59038013 | rs56147824 |
| rs2772331 | rs59214806 | rs2338016 | rs12500026 | rs1524469 |
| rs10783084 | rs2339717 | rs2329696 | rs1391761 | rs7795510 |
| rs10875153 | rs7137093 | rs1881424 | rs1996755 | rs3735646 |
| rs11164288 | rs933399 | rs1358516 | rs6857511 | rs725188 |
| rs7539757 | rs3184504 | rs7421353 | rs6817541 | rs2215628 |
| rs7417452 | rs4766578 | rs7592915 | rs12501810 | rs304512 |
| rs6603997 | rs653178 | rs72794477 | rs4864504 | rs4730915 |
| rs10782937 | rs11065976 | rs6547944 | rs6554170 | rs10464520 |
| rs7530780 | rs608848 | rs9789489 | rs2412563 | rs10233545 |
| rs6604013 | rs11066028 | rs7600906 | rs13114943 | rs13231854 |
| rs6675753 | rs4346023 | rs13003648 | rs985567 | rs2536189 |
| rs11164830 | rs741334 | rs2631971 | rs61219582 | rs2402585 |
| rs10493863 | rs6489858 | rs2339476 | rs6554190 | rs1916889 |
| rs619784 | rs2731303 | rs4666276 | rs218264 | rs2471211 |
| rs682967 | rs2434072 | rs4074252 | rs6554192 | rs10258817 |
| rs2785597 | rs1366029 | rs3922535 | rs7684939 | rs6966714 |
| rs566843 | rs7304462 | rs11685881 | rs3756197 | rs10954002 |
| rs1057257 | rs11067828 | rs35773364 | rs3796772 | rs66461031 |
| rs7517363 | rs1241219 | rs34168983 | rs4495106 | rs13233278 |
| rs622608 | rs2088236 | rs1562450 | rs4596303 | rs1719115 |
| rs2296558 | rs12423796 | rs13391574 | rs6854287 | rs7792723 |
| rs1000607 | rs1151901 | rs10865087 | rs13147017 | rs2535933 |
| rs6658257 | rs1499910 | rs223630 | rs2030364 | rs806180 |
| rs55943015 | rs11064682 | rs223647 | rs11133482 | rs322768 |
| rs3789688 | rs6490269 | rs3769597 | rs10028413 | rs10276417 |
| rs4847341 | rs11065085 | rs1454376 | rs269847 | rs339077 |
| rs9324353 | rs3914 | rs1597205 | rs2611163 | rs12534064 |
| rs1509171 | rs7135617 | rs7587055 | rs2166994 | rs58044348 |
| rs186502 | rs7484733 | rs1560882 | rs10017743 | rs7786074 |
| rs480547 | rs7305511 | rs2438428 | rs4860236 | rs3807306 |
| rs6663628 | rs4553407 | rs11883858 | rs6829981 | rs3778750 |
| rs2786521 | rs786425 | rs12478715 | rs13122165 | rs2305325 |
| rs6700680 | rs10846611 | rs7604508 | rs1900960 | rs9649520 |
| rs1434367 | rs4765615 | rs2372914 | rs2122643 | rs6959820 |
| rs4908303 | rs7134782 | rs11890606 | rs1455221 | rs3928439 |
| rs2392077 | rs12304983 | rs969876 | rs4467602 | rs68184415 |
| rs558035 | rs11058647 | rs1377862 | rs7662456 | rs6953598 |
| rs9434126 | rs1317982 | rs10211306 | rs10009664 | rs822040 |
| rs9434172 | rs2541986 | rs2192705 | rs7686405 | rs691555 |
| rs6702995 | rs12581795 | rs7562117 | rs13129711 | rs11761878 |
| rs12563004 | rs7966433 | rs2374141 | rs36115749 | rs56129173 |
| rs1517423 | rs7974992 | rs4493315 | rs1433630 | rs10279858 |
| rs4907990 | rs1562940 | rs11694429 | rs971712 | rs12666331 |
| rs1182598 | rs7956408 | rs7600439 | rs11561693 | rs1984823 |
| rs500497 | rs1236 | rs13012439 | rs13134748 | rs2727455 |
| rs6670085 | rs1527021 | rs7609363 | rs2711138 | rs10277874 |
| rs12757770 | rs10444602 | rs12712870 | rs1730873 | rs1126899 |
| rs12735409 | rs36123777 | rs12991132 | rs1902023 | rs12533615 |
| rs6582959 | rs7973326 | rs4952964 | rs13117003 | rs2334129 |
| rs4607912 | rs905226 | rs1864435 | rs11249494 | rs6951966 |
| rs11185102 | rs7993043 | rs12614953 | rs12640848 | rs12707053 |
| rs4914956 | rs9508917 | rs35266644 | rs2019873 | rs929714 |
| rs7555404 | rs7331288 | rs6749927 | rs6834847 | rs1946302 |
| rs12125002 | rs9509427 | rs11897732 | rs6835387 | rs2077671 |
| rs518650 | rs4770113 | rs3901678 | rs4018 | rs35106682 |
| rs6675402 | rs646343 | rs6544654 | rs10938106 | rs11970825 |
| rs4838890 | rs9509981 | rs6544660 | rs1797590 | rs2349462 |
| rs672992 | rs2335354 | rs12478601 | rs10050227 | rs7800170 |
| rs4839165 | rs584742 | rs6708660 | rs2175766 | rs3823552 |
| rs2761445 | rs4769288 | rs10182489 | rs1348204 | rs9757 |
| rs11810910 | rs9511098 | rs12992926 | rs4276307 | rs6946420 |
| rs2198612 | rs2050881 | rs7574610 | rs2866295 | rs2279860 |
| rs15651 | rs1924778 | rs7592384 | rs28433362 | rs9719149 |
| rs12564248 | rs4438154 | rs4148177 | rs55763205 | rs193948 |
| rs7534238 | rs4770957 | rs2166453 | rs4560439 | rs3924962 |
| rs12731815 | rs4769518 | rs4393800 | rs7663426 | rs6963099 |
| rs2640487 | rs1927681 | rs4953245 | rs11098983 | rs4342501 |
| rs1342855 | rs9512752 | rs13024886 | rs7664600 | rs518206 |
| rs1160413 | rs2491219 | rs2084345 | rs1012617 | rs7787017 |
| rs17026517 | rs9508166 | rs1865262 | rs11736583 | rs269266 |
| rs10857835 | rs3936102 | rs4953449 | rs2165942 | rs1267673 |
| rs10776726 | rs2263207 | rs2570516 | rs1389041 | rs9640168 |
| rs11102349 | rs9314991 | rs7586186 | rs3214025 | rs4726020 |
| rs463869 | rs912391 | rs6544972 | rs1980271 | rs801093 |
| rs6537743 | rs9532405 | rs4290703 | rs2868817 | rs7810541 |
| rs11102620 | rs2806632 | rs62139705 | rs28481766 | rs11767331 |
| rs1217419 | rs564701 | rs3771273 | rs6853486 | rs4726432 |
| rs668800 | rs9538608 | rs6544990 | rs1466488 | rs6978140 |
| rs926938 | rs6562331 | rs6720555 | rs346497 | rs981449 |
| rs2300694 | rs9542497 | rs10495945 | rs2904094 | rs10485835 |
| rs2797166 | rs2068462 | rs12476727 | rs12641570 | rs583684 |
| rs12726299 | rs9544512 | rs7609322 | rs2627737 | rs6952847 |
| rs517857 | rs1891671 | rs3844582 | rs3114018 | rs13223791 |
| rs1707112 | rs1517882 | rs61637835 | rs10428504 | rs2699476 |
| rs6698697 | rs883514 | rs2710163 | rs1431550 | rs5015740 |
| rs10923523 | rs6563570 | rs6742037 | rs17192189 | rs1404810 |
| rs10754384 | rs9532160 | rs4583515 | rs7664833 | rs7810281 |
| rs2246948 | rs2122211 | rs4952902 | rs11932865 | rs10270806 |
| rs662924 | rs9989076 | rs6545014 | rs10032172 | rs10273288 |
| rs12062618 | rs7338724 | rs6718037 | rs2089820 | rs851666 |
| rs10797649 | rs7994668 | rs9677729 | rs1433674 | rs2717821 |
| rs9660042 | rs9549229 | rs4953559 | rs6818880 | rs2160007 |
| rs6680845 | rs2701873 | rs6742830 | rs2452586 | rs11767479 |
| rs990034 | rs2721072 | rs2471403 | rs11722010 | rs6959357 |
| rs543163 | rs12585870 | rs7340322 | rs11944846 | rs2190640 |
| rs828508 | rs7993233 | rs11678857 | rs12507691 | rs7790647 |
| rs4970926 | rs4941989 | rs817009 | rs6834178 | rs243516 |
| rs2864700 | rs4943812 | rs17493439 | rs9997830 | rs11768249 |
| rs12095851 | rs11619117 | rs939425 | rs35369981 | rs6464926 |
| rs4845743 | rs9525434 | rs7559936 | rs1869351 | rs12534179 |
| rs4452994 | rs9532689 | rs12470929 | rs10023577 | rs716817 |
| rs406350 | rs9594473 | rs11683015 | rs10030511 | rs7778766 |
| rs11264573 | rs9532718 | rs4606968 | rs3775511 | rs6944096 |
| rs7521047 | rs9590576 | rs12053296 | rs7689825 | rs7784122 |
| rs7532286 | rs1332939 | rs7557898 | rs2732507 | rs34264850 |
| rs12047986 | rs7328265 | rs6545423 | rs10018021 | rs13236978 |
| rs4661079 | rs4941403 | rs1133182 | rs6810937 | rs740113 |
| rs3762281 | rs7995222 | rs2627765 | rs1609992 | rs10215009 |
| rs11264483 | rs9567049 | rs12476224 | rs4698892 | rs2229549 |
| rs10908505 | rs9562448 | rs2870065 | rs1501277 | rs11771445 |
| rs1609666 | rs9595037 | rs2870499 | rs17268078 | rs6972910 |
| rs11264498 | rs12867740 | rs848293 | rs4699133 | rs6464171 |
| rs11576266 | rs9533962 | rs6723710 | rs2522474 | rs6464178 |
| rs3806412 | rs7319966 | rs7586008 | rs2725741 | rs34552443 |
| rs12760406 | rs3759484 | rs155557 | rs4699141 | rs2374315 |
| rs10082042 | rs12427491 | rs2192512 | rs1490580 | rs10268412 |
| rs6337 | rs1469595 | rs7597268 | rs168989 | rs1544030 |
| rs865239 | rs2038789 | rs7562347 | rs56883672 | rs12671038 |
| rs4661077 | rs2406690 | rs2694619 | rs4698932 | rs10229388 |
| rs822579 | rs9534888 | rs10175114 | rs2189234 | rs6961745 |
| rs7544727 | rs9534900 | rs3935828 | rs7658539 | rs2098063 |
| rs10908536 | rs2406709 | rs12478556 | rs55838312 | rs12534818 |
| rs12755261 | rs9526447 | rs12713490 | rs2647266 | rs869337 |
| rs6427368 | rs607954 | rs13399701 | rs2726502 | rs12532839 |
| rs1953787 | rs1925747 | rs2422358 | rs2636713 | rs57395410 |
| rs1929843 | rs7984554 | rs1345351 | rs1994637 | rs1468187 |
| rs11584639 | rs9535002 | rs2194784 | rs4699185 | rs10257576 |
| rs12133354 | rs4151450 | rs3890755 | rs4699189 | rs2106776 |
| rs11265205 | rs9568036 | rs1110584 | rs7686722 | rs13234992 |
| rs6427488 | rs9535032 | rs10171480 | rs4699196 | rs34207852 |
| rs6700892 | rs9568042 | rs4671873 | rs994370 | rs9655619 |
| rs10752638 | rs9316394 | rs7589952 | rs6830544 | rs2535959 |
| rs2340723 | rs9526479 | rs58526351 | rs4295338 | rs2533164 |
| rs347310 | rs964413 | rs4852995 | rs1399405 | rs6944202 |
| rs6427702 | rs4942769 | rs3732266 | rs2074371 | rs1657292 |
| rs4291481 | rs4620866 | rs369994 | rs56373763 | rs12113230 |
| rs719285 | rs4942786 | rs10187472 | rs7678010 | rs288756 |
| rs1337373 | rs73184603 | rs7593986 | rs2131462 | rs2530760 |
| rs2348100 | rs9535112 | rs1459267 | rs10461142 | rs887607 |
| rs12131212 | rs942870 | rs2192014 | rs55664149 | rs12698056 |
| rs10753733 | rs1149833 | rs1430348 | rs11736916 | rs10229663 |
| rs503625 | rs1543525 | rs13008860 | rs7671885 | rs420450 |
| rs1723015 | rs9535882 | rs1618091 | rs1393824 | rs34876789 |
| rs4145461 | rs9536307 | rs673585 | rs2343138 | rs7013206 |
| rs2142744 | rs9536394 | rs7560262 | rs6837839 | rs11986778 |
| rs4142003 | rs2225203 | rs17702641 | rs219472 | rs13254474 |
| rs6670407 | rs2125316 | rs12475638 | rs7376484 | rs7817700 |
| rs6687517 | rs9569065 | rs10201530 | rs11723696 | rs2469403 |
| rs1537389 | rs1998695 | rs290024 | rs957623 | rs1714732 |
| rs10800551 | rs9563350 | rs13425291 | rs6837232 | rs10108689 |
| rs960845 | rs4116800 | rs17040590 | rs6843376 | rs4875560 |
| rs6698467 | rs6561900 | rs7563118 | rs10017233 | rs7460300 |
| rs9425568 | rs7988129 | rs557742 | rs900328 | rs2741105 |
| rs7517726 | rs2122273 | rs6754587 | rs3866832 | rs7845518 |
| rs1461024 | rs9537920 | rs6547364 | rs6838407 | rs7833246 |
| rs4447042 | rs9527822 | rs1432177 | rs326889 | rs2271342 |
| rs6688577 | rs9317048 | rs7581293 | rs4834261 | rs6601414 |
| rs10912963 | rs340216 | rs11126906 | rs6823012 | rs7011281 |
| rs4642850 | rs11843286 | rs2171273 | rs29367 | rs4841404 |
| rs1588047 | rs1601135 | rs4832067 | rs1978138 | rs2736306 |
| rs7520147 | rs9539222 | rs2365446 | rs2388999 | rs1497042 |
| rs1073598 | rs1354636 | rs1554109 | rs9684691 | rs2272623 |
| rs10798592 | rs9563988 | rs3755014 | rs35429096 | rs3843803 |
| rs2209169 | rs2992162 | rs7560886 | rs10025157 | rs12375315 |
| rs12034934 | rs6562321 | rs10208989 | rs1035718 | rs36077823 |
| rs1018669 | rs1508930 | rs4832316 | rs7678153 | rs4144411 |
| rs3766639 | rs359388 | rs66534649 | rs12512082 | rs187902 |
| rs4652329 | rs9571330 | rs13013591 | rs2114461 | rs965147 |
| rs4652331 | rs5020262 | rs6713961 | rs718281 | rs17485925 |
| rs6425517 | rs7984558 | rs842168 | rs56173225 | rs4621847 |
| rs9725887 | rs3843947 | rs3112992 | rs4431241 | rs2051660 |
| rs9727252 | rs1323913 | rs6712299 | rs11098598 | rs7837060 |
| rs2791936 | rs2325081 | rs2229169 | rs885103 | rs2410572 |
| rs2816201 | rs958120 | rs12997297 | rs10022380 | rs13276483 |
| rs2816167 | rs12323259 | rs34747246 | rs6853169 | rs7011083 |
| rs7522641 | rs1604613 | rs1546351 | rs34287772 | rs7838269 |
| rs2184575 | rs9317861 | rs12468113 | rs2663711 | rs4922184 |
| rs6698526 | rs973598 | rs6733285 | rs4833962 | rs580819 |
| rs7544494 | rs2325416 | rs7567996 | rs1390078 | rs10092469 |
| rs7548654 | rs1931472 | rs10183150 | rs4833994 | rs11993990 |
| rs55635521 | rs9542840 | rs6706943 | rs7696561 | rs62501068 |
| rs10494522 | rs11617201 | rs4850956 | rs6824020 | rs13264439 |
| rs3761903 | rs7982559 | rs2136435 | rs1355925 | rs4872181 |
| rs667586 | rs7330561 | rs7568913 | rs12648943 | rs4872232 |
| rs3766997 | rs7318088 | rs2192691 | rs11723731 | rs17752020 |
| rs10752868 | rs9318289 | rs2310349 | rs623393 | rs2979814 |
| rs10797807 | rs7318676 | rs6717408 | rs4864201 | rs4410916 |
| rs2027078 | rs7988372 | rs6543234 | rs869090 | rs4871971 |
| rs2767305 | rs4885370 | rs12712190 | rs425227 | rs4422781 |
| rs10797964 | rs283983 | rs2576790 | rs6849886 | rs6983452 |
| rs12127674 | rs9544409 | rs6738475 | rs12186299 | rs7842666 |
| rs10911741 | rs1759981 | rs4012760 | rs9998644 | rs7000894 |
| rs1322358 | rs2147486 | rs2169159 | rs7693443 | rs10112345 |
| rs6671112 | rs1041477 | rs10173377 | rs4864217 | rs62499159 |
| rs17526478 | rs767778 | rs4676121 | rs1386361 | rs10095128 |
| rs10798107 | rs4885712 | rs12712008 | rs2896821 | rs7000638 |
| rs12089759 | rs1176303 | rs1983063 | rs13126136 | rs6990131 |
| rs7410974 | rs9545595 | rs35154454 | rs66936369 | rs776370 |
| rs10801272 | rs12100409 | rs1588327 | rs11099403 | rs1383964 |
| rs544343 | rs1336742 | rs4849303 | rs1582745 | rs7820795 |
| rs860954 | rs9575137 | rs13005823 | rs6848489 | rs10097189 |
| rs1410249 | rs9546371 | rs67063370 | rs58381038 | rs7009022 |
| rs7531292 | rs4299040 | rs719667 | rs6536009 | rs28693643 |
| rs10801084 | rs2774076 | rs10206205 | rs769682 | rs4739395 |
| rs10921133 | rs1374427 | rs6720637 | rs11728088 | rs35439733 |
| rs6695451 | rs9547000 | rs62170143 | rs13126364 | rs2843917 |
| rs651691 | rs9575974 | rs10864931 | rs2635427 | rs933116 |
| rs1325429 | rs9602955 | rs13402497 | rs6831204 | rs10955014 |
| rs339651 | rs9556641 | rs11674259 | rs11100788 | rs28380463 |
| rs1576765 | rs2049200 | rs708663 | rs7664006 | rs2932005 |
| rs7551077 | rs1154933 | rs10199125 | rs13136472 | rs13248612 |
| rs1332667 | rs4594109 | rs332898 | rs6537301 | rs34036147 |
| rs4915311 | rs9515228 | rs13022184 | rs4305538 | rs4733951 |
| rs10922266 | rs9515514 | rs13010674 | rs2135963 | rs13276230 |
| rs1938378 | rs9522739 | rs332722 | rs13120390 | rs7818839 |
| rs28599571 | rs1491030 | rs13005938 | rs1437273 | rs9650336 |
| rs12750406 | rs828185 | rs6542527 | rs6852226 | rs895621 |
| rs6695659 | rs9523230 | rs4513300 | rs11734619 | rs7007981 |
| rs12048017 | rs1336220 | rs2580375 | rs906125 | rs10107406 |
| rs10800751 | rs6492584 | rs7603857 | rs13129809 | rs7828448 |
| rs927900 | rs1926609 | rs1371491 | rs13110548 | rs2037205 |
| rs10920530 | rs306670 | rs17242903 | rs1879828 | rs869471 |
| rs10920625 | rs3848066 | rs13033098 | rs7665528 | rs11990433 |
| rs4950998 | rs9561469 | rs13016000 | rs3846301 | rs2730166 |
| rs2366007 | rs7990565 | rs7564755 | rs13144030 | rs2589894 |
| rs10900551 | rs4148542 | rs2068359 | rs9997090 | rs1559739 |
| rs10159082 | rs2993561 | rs2193913 | rs13149615 | rs9886398 |
| rs10900562 | rs7995859 | rs6711745 | rs13127625 | rs1050262 |
| rs4287237 | rs1974086 | rs7586744 | rs6831212 | rs10087388 |
| rs4951366 | rs1771417 | rs4662726 | rs28374599 | rs1548104 |
| rs2055939 | rs6491404 | rs7599641 | rs1490467 | rs8185945 |
| rs4951077 | rs7997477 | rs734193 | rs970438 | rs11136249 |
| rs10458588 | rs9517514 | rs4662894 | rs10007581 | rs12680292 |
| rs4951397 | rs9517722 | rs10202906 | rs12645654 | rs11136248 |
| rs1999157 | rs17474996 | rs2464774 | rs2358463 | rs62539116 |
| rs11240277 | rs1998550 | rs7601626 | rs2270862 | rs4873619 |
| rs11240302 | rs479648 | rs36045169 | rs9683403 | rs4873272 |
| rs4950972 | rs1338049 | rs2622049 | rs28479793 | rs12545705 |
| rs2802847 | rs491234 | rs62162206 | rs2726778 | rs2385339 |
| rs6664706 | rs9518746 | rs4525746 | rs7685302 | rs3812424 |
| rs6702688 | rs7981306 | rs4368385 | rs10006803 | rs13277711 |
| rs28570494 | rs7328284 | rs7564332 | rs4696261 | rs1911836 |
| rs11579993 | rs938165 | rs1564935 | rs361166 | rs10958243 |
| rs2796259 | rs7321252 | rs10193171 | rs6845076 | rs62499448 |
| rs1621023 | rs6491949 | rs1375131 | rs361147 | rs11779921 |
| rs1417954 | rs3924165 | rs1446585 | rs13143024 | rs4873667 |
| rs10746419 | rs3918344 | rs4954552 | rs13130547 | rs2553881 |
| rs7523300 | rs1477921 | rs36098083 | rs7663766 | rs13281202 |
| rs10863811 | rs9888533 | rs12619319 | rs35752631 | rs2719372 |
| rs2092448 | rs9559035 | rs6728076 | rs13121739 | rs2929012 |
| rs6656336 | rs915046 | rs1037580 | rs2676329 | rs4737418 |
| rs784385 | rs719739 | rs10180719 | rs2255137 | rs2576601 |
| rs572235 | rs7333277 | rs3937999 | rs964475 | rs353726 |
| rs4951676 | rs34185463 | rs1473242 | rs1516820 | rs1451142 |
| rs11120121 | rs7321389 | rs10496848 | rs1516819 | rs13271149 |
| rs12727510 | rs867482 | rs13016322 | rs6810720 | rs35320500 |
| rs6667098 | rs4511429 | rs13418422 | rs4696138 | rs4738687 |
| rs7522552 | rs2094810 | rs1375612 | rs6841692 | rs3903445 |
| rs1319603 | rs11618227 | rs12691642 | rs6817172 | rs6987187 |
| rs12042718 | rs7399672 | rs4662304 | rs4276265 | rs2946578 |
| rs4846521 | rs944055 | rs12614491 | rs7660615 | rs28819228 |
| rs11117789 | rs9562187 | rs11898334 | rs62319916 | rs10109961 |
| rs6701537 | rs7493807 | rs7597006 | rs7662708 | rs7813413 |
| rs6675497 | rs8019785 | rs1106909 | rs4696369 | rs2634517 |
| rs11118179 | rs8013276 | rs2016340 | rs10018880 | rs13281543 |
| rs11118448 | rs10141533 | rs12617575 | rs992614 | rs7829715 |
| rs2378436 | rs8013567 | rs388614 | rs4696583 | rs10095333 |
| rs4255390 | rs61972197 | rs7563149 | rs6536051 | rs4738742 |
| rs4546929 | rs3794452 | rs1234416 | rs717695 | rs397467 |
| rs1890615 | rs6573084 | rs1404186 | rs2705455 | rs7842820 |
| rs11577934 | rs222686 | rs7559078 | rs28714301 | rs2956796 |
| rs298738 | rs12886684 | rs10181626 | rs2113992 | rs3110141 |
| rs1604778 | rs7143576 | rs10190013 | rs12645401 | rs7838118 |
| rs12729445 | rs1080815 | rs60076148 | rs1907093 | rs3110127 |
| rs4653718 | rs2105274 | rs10432476 | rs1350025 | rs10957081 |
| rs6667260 | rs13379237 | rs7584868 | rs2053346 | rs6999719 |
| rs10753430 | rs7154053 | rs6744391 | rs10012081 | rs4579516 |
| rs6679942 | rs11626997 | rs60497708 | rs2881907 | rs10110690 |
| rs10916245 | rs2877847 | rs949068 | rs12507639 | rs13280933 |
| rs241336 | rs2209402 | rs1822771 | rs58484527 | rs56970093 |
| rs600136 | rs12882003 | rs2113477 | rs35126302 | rs653657 |
| rs238763 | rs1191567 | rs3106658 | rs2712676 | rs948421 |
| rs10005 | rs1191600 | rs1524911 | rs13106852 | rs7842384 |
| rs1752177 | rs225951 | rs4572544 | rs7658409 | rs1946593 |
| rs9431651 | rs179648 | rs13425585 | rs2001227 | rs2350925 |
| rs1769795 | rs55675143 | rs13025721 | rs1394810 | rs13260909 |
| rs28759217 | rs4981905 | rs834129 | rs1406369 | rs10099741 |
| rs7354849 | rs35336096 | rs12467193 | rs4691215 | rs6472075 |
| rs6656327 | rs10146564 | rs917242 | rs6536967 | rs3850739 |
| rs10910157 | rs8018599 | rs2528611 | rs10018491 | rs298193 |
| rs1937258 | rs1268902 | rs13034428 | rs10033844 | rs4737692 |
| rs10924870 | rs8004518 | rs7590617 | rs7678260 | rs2001648 |
| rs7541057 | rs12432476 | rs6432616 | rs6811972 | rs13275168 |
| rs1445295 | rs378836 | rs13414363 | rs12647126 | rs4338090 |
| rs2275399 | rs55775461 | rs35505926 | rs6828910 | rs7004201 |
| rs12047281 | rs1169123 | rs2046490 | rs1596251 | rs9657034 |
| rs1842086 | rs1755770 | rs16847399 | rs13109542 | rs4737880 |
| rs578750 | rs2787429 | rs12692702 | rs7690716 | rs2591004 |
| rs2841408 | rs17648576 | rs2169219 | rs13124556 | rs7825569 |
| rs2689137 | rs3909613 | rs6707357 | rs17318489 | rs4623463 |
| rs6429169 | rs12590640 | rs355821 | rs13117656 | rs4434651 |
| rs10802932 | rs10047808 | rs1551337 | rs11133013 | rs17712160 |
| rs4660080 | rs3825635 | rs3791848 | rs12650554 | rs1593272 |
| rs1635518 | rs35495190 | rs6432896 | rs4695999 | rs12676480 |
| rs4276903 | rs5023326 | rs4233811 | rs6813831 | rs7008849 |
| rs3896337 | rs12883298 | rs2617367 | rs1390345 | rs60261585 |
| rs589091 | rs7147486 | rs13007801 | rs994252 | rs11997090 |
| rs6699271 | rs2064547 | rs2685804 | rs3775202 | rs11988654 |
| rs10927382 | rs2764956 | rs830957 | rs10020708 | rs2941477 |
| rs6664091 | rs10467709 | rs6729098 | rs9990533 | rs1449543 |
| rs12131884 | rs12896722 | rs2111461 | rs17709259 | rs12155523 |
| rs1151664 | rs1367031 | rs17581284 | rs6828067 | rs28515161 |
| rs12733732 | rs10129948 | rs6738839 | rs2605061 | rs6985088 |
| rs28481429 | rs12147950 | rs989530 | rs6826314 | rs748 |
| rs12027301 | rs966918 | rs588716 | rs1030186 | rs1564532 |
| rs4881256 | rs12589704 | rs10930640 | rs6840955 | rs7823953 |
| rs7095043 | rs4254231 | rs10930644 | rs12505558 | rs10504698 |
| rs10794737 | rs2415545 | rs2646167 | rs925669 | rs55982323 |
| rs4076747 | rs1570183 | rs1982235 | rs7659685 | rs890306 |
| rs1889644 | rs4899719 | rs1012357 | rs4862278 | rs7825743 |
| rs2068455 | rs7144539 | rs10930741 | rs1105207 | rs13274901 |
| rs10904028 | rs3910107 | rs4894233 | rs1405944 | rs12545172 |
| rs1872759 | rs11846556 | rs2364717 | rs13113913 | rs1382615 |
| rs12769562 | rs9323070 | rs2695746 | rs34559355 | rs1160419 |
| rs2123357 | rs10135639 | rs7589907 | rs10030678 | rs11778353 |
| rs2518039 | rs8008932 | rs13025070 | rs1040069 | rs317953 |
| rs10795281 | rs17278488 | rs4893868 | rs10866304 | rs13255168 |
| rs2692805 | rs12717357 | rs262266 | rs7378399 | rs1493302 |
| rs2275068 | rs8022465 | rs7419281 | rs6553246 | rs2930554 |
| rs1931902 | rs7152024 | rs4666979 | rs12506090 | rs6470858 |
| rs11255267 | rs4494463 | rs4667128 | rs890982 | rs10092073 |
| rs932971 | rs1999617 | rs6734501 | rs7702532 | rs6981706 |
| rs2802457 | rs8019784 | rs67791366 | rs4957112 | rs2664344 |
| rs1312781 | rs10148729 | rs56010743 | rs10065177 | rs13253448 |
| rs10905246 | rs8016892 | rs13026970 | rs35590277 | rs7824161 |
| rs11255455 | rs1959775 | rs949972 | rs4975536 | rs160423 |
| rs10752123 | rs2816702 | rs3953016 | rs4246739 | rs2107071 |
| rs10752124 | rs7160416 | rs1429421 | rs13159461 | rs4538831 |
| rs10905277 | rs7154992 | rs13391505 | rs11133678 | rs4620242 |
| rs7098620 | rs12432093 | rs1019429 | rs13173119 | rs1379252 |
| rs4747748 | rs34061700 | rs3112314 | rs6883980 | rs4425722 |
| rs12415654 | rs4444245 | rs3107390 | rs2075786 | rs35496046 |
| rs10795610 | rs10142065 | rs10931309 | rs7734992 | rs4734842 |
| rs7071055 | rs35828909 | rs13014693 | rs6866294 | rs10090370 |
| rs7082651 | rs7157154 | rs7603905 | rs10462720 | rs995024 |
| rs10905377 | rs7140673 | rs12623140 | rs4975557 | rs7842360 |
| rs10795649 | rs2181735 | rs13017460 | rs4975678 | rs1443560 |
| rs2031558 | rs7160007 | rs17439247 | rs62335954 | rs1443563 |
| rs11255890 | rs2295820 | rs6434504 | rs904736 | rs1552314 |
| rs10905549 | rs12588935 | rs952534 | rs66752559 | rs10956824 |
| rs7067974 | rs3783654 | rs1439651 | rs55905121 | rs12541257 |
| rs11256411 | rs1305025 | rs6733218 | rs16883484 | rs9657011 |
| rs2762628 | rs1814225 | rs7600163 | rs6554958 | rs11993469 |
| rs10905927 | rs1211190 | rs1527999 | rs35243540 | rs13264814 |
| rs7070468 | rs4898901 | rs1430701 | rs6554987 | rs7018420 |
| rs7081612 | rs3818777 | rs17177643 | rs7714838 | rs7828100 |
| rs2182700 | rs17714764 | rs12996633 | rs13173087 | rs13275569 |
| rs11258436 | rs12431592 | rs12999912 | rs12518533 | rs10096591 |
| rs4750440 | rs4901735 | rs7595528 | rs6859186 | rs10454352 |
| rs1955196 | rs12436123 | rs10931770 | rs4975805 | rs2445728 |
| rs4750608 | rs11621547 | rs4850759 | rs13168507 | rs7830278 |
| rs2354507 | rs4898921 | rs6736733 | rs459762 | rs34096856 |
| rs780840 | rs1189797 | rs7591636 | rs11739331 | rs6999279 |
| rs17347211 | rs17092873 | rs2712886 | rs491368 | rs10282981 |
| rs4747309 | rs7142046 | rs11693873 | rs11134070 | rs10955119 |
| rs10508556 | rs6573138 | rs12151767 | rs2964133 | rs2449517 |
| rs1750478 | rs6573142 | rs3792158 | rs10064239 | rs7016352 |
| rs7080850 | rs3759671 | rs17787829 | rs6898659 | rs10096329 |
| rs2026465 | rs7154383 | rs3828317 | rs7720723 | rs13253769 |
| rs11011685 | rs1998101 | rs10196961 | rs2924466 | rs9297291 |
| rs2681928 | rs10782438 | rs4850437 | rs1604977 | rs4377934 |
| rs12253846 | rs8011271 | rs700640 | rs886509 | rs2509760 |
| rs313793 | rs9323357 | rs700659 | rs10065959 | rs618854 |
| rs1675503 | rs33912345 | rs700689 | rs1978155 | rs7823289 |
| rs1979486 | rs10483727 | rs10048735 | rs1366405 | rs7001644 |
| rs7095713 | rs750685 | rs1455350 | rs6882812 | rs525817 |
| rs6482168 | rs217640 | rs994261 | rs1438299 | rs7828800 |
| rs7100759 | rs8013116 | rs993282 | rs6886752 | rs12541306 |
| rs7923552 | rs2355734 | rs10804097 | rs4701604 | rs10955338 |
| rs7922464 | rs2146940 | rs2270282 | rs835081 | rs6982816 |
| rs10828243 | rs1255865 | rs6712136 | rs304586 | rs2957477 |
| rs6482181 | rs28570137 | rs12693957 | rs257758 | rs302953 |
| rs7100317 | rs1542313 | rs6736794 | rs13176741 | rs1954749 |
| rs11012730 | rs1954052 | rs11680734 | rs3736148 | rs1784483 |
| rs12251016 | rs1953567 | rs6732162 | rs35489199 | rs2615081 |
| rs4553272 | rs12588780 | rs62182632 | rs692419 | rs4734992 |
| rs792456 | rs7153102 | rs13424874 | rs1609128 | rs1563571 |
| rs2807982 | rs4450323 | rs1355343 | rs1875502 | rs12678529 |
| rs2666756 | rs10134446 | rs969515 | rs4540180 | rs1783163 |
| rs2807971 | rs168961 | rs4147727 | rs10052023 | rs7813186 |
| rs11012847 | rs34450502 | rs34028795 | rs12522829 | rs7815044 |
| rs10828286 | rs4899317 | rs12466346 | rs10941831 | rs10283219 |
| rs1556010 | rs11626325 | rs6435414 | rs13178868 | rs28690656 |
| rs1922123 | rs12896829 | rs12694115 | rs1496454 | rs10098294 |
| rs4748846 | rs11158899 | rs1517344 | rs1261244 | rs1552785 |
| rs9418444 | rs1954693 | rs7561678 | rs1995264 | rs1599051 |
| rs2265268 | rs2239223 | rs7593548 | rs12652723 | rs1513521 |
| rs6482466 | rs10459542 | rs13028512 | rs1435814 | rs9942763 |
| rs1329257 | rs61987083 | rs10204060 | rs12520187 | rs10099381 |
| rs3006839 | rs17182680 | rs10932379 | rs62354596 | rs645385 |
| rs2505962 | rs10151964 | rs10168303 | rs7704148 | rs2178950 |
| rs2478251 | rs35591392 | rs12996008 | rs6860298 | rs10955778 |
| rs3824590 | rs10136948 | rs2178756 | rs6873856 | rs12542425 |
| rs927675 | rs11159161 | rs10192241 | rs62346464 | rs1793707 |
| rs2477345 | rs2360989 | rs9288501 | rs10067172 | rs13275942 |
| rs34772309 | rs10873298 | rs12468812 | rs2329736 | rs1209 |
| rs10740784 | rs12101153 | rs62178268 | rs1423286 | rs7813199 |
| rs1148184 | rs7153862 | rs1179672 | rs2217273 | rs13263296 |
| rs12265184 | rs8006086 | rs2162001 | rs4867223 | rs10100841 |
| rs11594878 | rs8009134 | rs4674240 | rs13177078 | rs13271651 |
| rs12246494 | rs2543581 | rs2556385 | rs4354056 | rs2656110 |
| rs2993984 | rs6574570 | rs6751108 | rs1318821 | rs279616 |
| rs2807761 | rs66523962 | rs12464951 | rs17506712 | rs4871252 |
| rs332133 | rs11159490 | rs6755653 | rs13355979 | rs7017662 |
| rs332179 | rs1957533 | rs6436254 | rs431572 | rs6470140 |
| rs1265845 | rs1954376 | rs1364386 | rs35414 | rs7000947 |
| rs4749345 | rs4899832 | rs34623567 | rs9765520 | rs9297689 |
| rs11007321 | rs1241927 | rs6436276 | rs6896757 | rs62530664 |
| rs7089414 | rs12886262 | rs4085400 | rs163841 | rs4466418 |
| rs7071146 | rs856678 | rs2894448 | rs286415 | rs6470399 |
| rs11007457 | rs4904247 | rs348979 | rs286388 | rs4870985 |
| rs4749417 | rs6574859 | rs348966 | rs12513652 | rs4871776 |
| rs4749511 | rs4445831 | rs13012148 | rs12153427 | rs400818 |
| rs7070569 | rs2254354 | rs6436300 | rs7448971 | rs10096351 |
| rs10826755 | rs10140965 | rs7588737 | rs6451213 | rs6985419 |
| rs3847404 | rs2896066 | rs6436304 | rs991570 | rs12547874 |
| rs2462021 | rs17774608 | rs34041628 | rs6451229 | rs35902790 |
| rs2505120 | rs2152978 | rs13020455 | rs984164 | rs10956381 |
| rs2795864 | rs35836300 | rs17563113 | rs700162 | rs4733665 |
| rs10740815 | rs1297789 | rs4592839 | rs1287278 | rs11782002 |
| rs11007990 | rs34876948 | rs12694581 | rs1287261 | rs4404878 |
| rs1042058 | rs10135502 | rs2290031 | rs11948259 | rs7841347 |
| rs17229476 | rs2295166 | rs7595001 | rs267754 | rs28459758 |
| rs8181386 | rs10135174 | rs10932970 | rs7731023 | rs7812386 |
| rs3124187 | rs2402170 | rs9678109 | rs12186384 | rs6986528 |
| rs1389626 | rs7158068 | rs4674870 | rs13153581 | rs4733828 |
| rs10826866 | rs4905324 | rs6708567 | rs2088732 | rs1499363 |
| rs7079229 | rs10140227 | rs639729 | rs210540 | rs3931280 |
| rs17229657 | rs4520781 | rs1522818 | rs7711452 | rs2608042 |
| rs4747740 | rs2145636 | rs1500999 | rs56114410 | rs2936594 |
| rs4749642 | rs28417537 | rs12465531 | rs270584 | rs7825258 |
| rs796083 | rs35833079 | rs34820446 | rs4635961 | rs1441993 |
| rs10827001 | rs7144145 | rs1996959 | rs10045655 | rs2579859 |
| rs4237395 | rs4133712 | rs6743294 | rs13177785 | rs394229 |
| rs988645 | rs72700712 | rs12694799 | rs12522588 | rs7008182 |
| rs10827252 | rs11848088 | rs1419960 | rs6861054 | rs6415521 |
| rs1213392 | rs2693703 | rs6746407 | rs318021 | rs4460349 |
| rs2474748 | rs944121 | rs9288662 | rs13167737 | rs2270876 |
| rs1451187 | rs2487507 | rs1509583 | rs2972396 | rs4736413 |
| rs1219484 | rs10136429 | rs10202701 | rs4866934 | rs4606040 |
| rs11010892 | rs2614485 | rs3107179 | rs1593971 | rs7825637 |
| rs1200816 | rs1257320 | rs6742242 | rs6891363 | rs6471161 |
| rs2504127 | rs11624357 | rs56265336 | rs10941667 | rs55732042 |
| rs2804654 | rs10136777 | rs683719 | rs2218080 | rs4400336 |
| rs11146783 | rs34699335 | rs11689017 | rs7717787 | rs7017531 |
| rs2466459 | rs12437096 | rs2163569 | rs2049656 | rs13267477 |
| rs1815715 | rs11160538 | rs7566042 | rs6414909 | rs11998343 |
| rs2435380 | rs11628052 | rs2317644 | rs8188289 | rs2245810 |
| rs11528455 | rs13065 | rs6743039 | rs1995795 | rs2088640 |
| rs9787634 | rs8004825 | rs13390880 | rs10471468 | rs10090796 |
| rs10793545 | rs10134016 | rs6431548 | rs35711462 | rs6577901 |
| rs2210497 | rs6575862 | rs925353 | rs12187185 | rs11780480 |
| rs10751387 | rs1044502 | rs13028091 | rs10059711 | rs3913642 |
| rs4128664 | rs6575935 | rs3828216 | rs1020510 | rs7835774 |
| rs10736370 | rs7156255 | rs10211588 | rs788592 | rs7821257 |
| rs4304703 | rs10138768 | rs10933515 | rs2910808 | rs13253978 |
| rs10857554 | rs35956368 | rs7581893 | rs10940447 | rs7014345 |
| rs7086070 | rs9671696 | rs34519321 | rs6879219 | rs11167104 |
| rs11101064 | rs7144412 | rs412889 | rs159574 | rs10095654 |
| rs10776563 | rs12913456 | rs3746801 | rs7734230 | rs3935087 |
| rs4838565 | rs11263680 | rs6033697 | rs7716644 | rs28523940 |
| rs2611512 | rs2344878 | rs202516 | rs32862 | rs4074834 |
| rs1436206 | rs2017497 | rs6082333 | rs1558575 | rs9650466 |
| rs2339711 | rs12908483 | rs6050260 | rs37530 | rs6989368 |
| rs10762809 | rs1045935 | rs2295553 | rs2675373 | rs28453985 |
| rs1992529 | rs973264 | rs241602 | rs364528 | rs600509 |
| rs1776614 | rs2573690 | rs6076614 | rs4700359 | rs2804264 |
| rs1343011 | rs4906679 | rs2326552 | rs5024245 | rs10809682 |
| rs10763122 | rs61999575 | rs6053285 | rs6887434 | rs4741619 |
| rs10740594 | rs17648892 | rs13038769 | rs190947 | rs7860700 |
| rs2461898 | rs2311468 | rs6053939 | rs12654132 | rs729365 |
| rs9416413 | rs138827794 | rs2876050 | rs6449593 | rs3847239 |
| rs10825806 | rs11072136 | rs2024720 | rs1514577 | rs7851261 |
| rs1568582 | rs2676015 | rs1028310 | rs12658862 | rs10814961 |
| rs7906714 | rs7179722 | rs6055494 | rs62380579 | rs10815011 |
| rs2537647 | rs12913404 | rs6055897 | rs1809291 | rs3780378 |
| rs11812567 | rs1895613 | rs6118454 | rs2278353 | rs10815292 |
| rs4578336 | rs588655 | rs6056871 | rs706672 | rs10976219 |
| rs7900212 | rs563663 | rs6039945 | rs13178999 | rs9408716 |
| rs1938542 | rs2632107 | rs1107576 | rs1703250 | rs10815713 |
| rs3887989 | rs12591859 | rs6040452 | rs2130335 | rs7028239 |
| rs718146 | rs8040732 | rs1513583 | rs35579160 | rs7039875 |
| rs2250177 | rs4780226 | rs2144645 | rs2302975 | rs7357637 |
| rs10995143 | rs11858408 | rs6033503 | rs651076 | rs10758932 |
| rs1896995 | rs440025 | rs243896 | rs281141 | rs12555635 |
| rs2256694 | rs347827 | rs6033804 | rs1217827 | rs1361319 |
| rs2660113 | rs347838 | rs1570485 | rs17312595 | rs4534178 |
| rs12355750 | rs180975 | rs6135220 | rs471349 | rs4742476 |
| rs1857608 | rs1820449 | rs461666 | rs4582254 | rs12235547 |
| rs7912878 | rs7175617 | rs2023386 | rs2840108 | rs1353983 |
| rs6480158 | rs6495689 | rs775144 | rs435212 | rs10815830 |
| rs10822984 | rs4924078 | rs1018425 | rs6453102 | rs2296101 |
| rs3096244 | rs11073056 | rs6044702 | rs2335418 | rs1998517 |
| rs717976 | rs524952 | rs6045359 | rs258509 | rs1415721 |
| rs7917200 | rs7172877 | rs6045730 | rs2243051 | rs1415723 |
| rs10823306 | rs499248 | rs6046100 | rs6886221 | rs10114687 |
| rs1119194 | rs12900194 | rs6136794 | rs7715204 | rs10124836 |
| rs10998973 | rs6495755 | rs6081858 | rs4267850 | rs2053126 |
| rs35253454 | rs1443815 | rs6082241 | rs2445887 | rs1368983 |
| rs1538677 | rs767784 | rs11696663 | rs3822568 | rs7870940 |
| rs10999801 | rs1435681 | rs6035995 | rs4639193 | rs2570301 |
| rs730334 | rs6495870 | rs1159466 | rs13160125 | rs10977344 |
| rs9415064 | rs4924180 | rs745659 | rs32950 | rs1897676 |
| rs7921419 | rs17627825 | rs6137821 | rs4703523 | rs1433552 |
| rs10824091 | rs13329072 | rs2983304 | rs12653788 | rs1331664 |
| rs11597563 | rs6492848 | rs6049612 | rs224905 | rs324533 |
| rs7908862 | rs10152999 | rs6050429 | rs2974441 | rs10977469 |
| rs10824305 | rs4924393 | rs4815448 | rs3777049 | rs560380 |
| rs2221169 | rs1017843 | rs4911204 | rs4703572 | rs10119225 |
| rs7900900 | rs2412525 | rs6060930 | rs1840843 | rs9644895 |
| rs1907719 | rs8041534 | rs6061036 | rs4131298 | rs62534654 |
| rs2670140 | rs2412544 | rs6058505 | rs12653861 | rs9299091 |
| rs7071351 | rs4924493 | rs6061080 | rs7708491 | rs7848297 |
| rs10824632 | rs6492961 | rs6058513 | rs4920769 | rs1467790 |
| rs10762831 | rs4924503 | rs4911543 | rs323776 | rs10816136 |
| rs1250567 | rs10152371 | rs6061206 | rs13168787 | rs844166 |
| rs6584760 | rs689594 | rs4911088 | rs7722396 | rs828832 |
| rs10788627 | rs4923888 | rs1022685 | rs13158247 | rs1326772 |
| rs3891440 | rs4923890 | rs2424884 | rs7732661 | rs10816159 |
| rs7902521 | rs3101436 | rs1737894 | rs1278696 | rs2821476 |
| rs2474270 | rs1659227 | rs293558 | rs4379236 | rs1408123 |
| rs4933836 | rs6492993 | rs6087399 | rs990124 | rs2476584 |
| rs12771439 | rs8036686 | rs2018002 | rs13177562 | rs66506857 |
| rs1336401 | rs7176588 | rs2424918 | rs6557066 | rs10978071 |
| rs10788316 | rs7178634 | rs2424936 | rs4242244 | rs10978099 |
| rs7903999 | rs8026197 | rs3746461 | rs981862 | rs10759117 |
| rs10887382 | rs7177641 | rs2268084 | rs11135420 | rs1343534 |
| rs11201538 | rs316613 | rs2424997 | rs2881773 | rs727756 |
| rs6585984 | rs1655557 | rs2425019 | rs10046014 | rs404317 |
| rs10887609 | rs2253936 | rs143383 | rs10074626 | rs291298 |
| rs2279602 | rs9672360 | rs13045050 | rs10071737 | rs291279 |
| rs11202426 | rs1107858 | rs2425236 | rs1919247 | rs4741037 |
| rs2311187 | rs2577960 | rs6130119 | rs6883365 | rs7031100 |
| rs10887729 | rs1197660 | rs2294439 | rs12716498 | rs2183412 |
| rs791888 | rs2305655 | rs2076381 | rs3850904 | rs62542866 |
| rs1358865 | rs1048166 | rs6128478 | rs1161239 | rs1889297 |
| rs7088594 | rs1704350 | rs4810261 | rs6865946 | rs17194667 |
| rs9651491 | rs2122674 | rs211892 | rs6595366 | rs7040930 |
| rs6586103 | rs11638531 | rs2065041 | rs390021 | rs7033951 |
| rs1236816 | rs4924629 | rs4812444 | rs1479063 | rs674657 |
| rs1234214 | rs9783683 | rs2223745 | rs10053573 | rs2891008 |
| rs2785070 | rs11632120 | rs2211355 | rs249496 | rs6475050 |
| rs2248456 | rs2615258 | rs6065418 | rs460651 | rs10756798 |
| rs1577661 | rs1706821 | rs6030227 | rs17399797 | rs263584 |
| rs7358173 | rs1346267 | rs4812698 | rs1217465 | rs4510955 |
| rs497099 | rs2947009 | rs6103489 | rs7723788 | rs10738495 |
| rs1426616 | rs8035322 | rs2179594 | rs10079531 | rs473817 |
| rs11202712 | rs7182457 | rs1111032 | rs591470 | rs4395970 |
| rs2488273 | rs11632739 | rs6124676 | rs13159387 | rs1556000 |
| rs792230 | rs2118918 | rs6130955 | rs6883082 | rs4977549 |
| rs2437874 | rs11635140 | rs3746497 | rs35842524 | rs7846924 |
| rs1926199 | rs55736202 | rs4810551 | rs2578483 | rs13289648 |
| rs303206 | rs11637582 | rs2868855 | rs6868564 | rs10448199 |
| rs7902781 | rs8041517 | rs2425934 | rs4957891 | rs10811229 |
| rs11186449 | rs3736485 | rs6019004 | rs10043631 | rs7864790 |
| rs2631685 | rs12396 | rs7260847 | rs247546 | rs4977337 |
| rs2259305 | rs4776037 | rs6125611 | rs166253 | rs7022483 |
| rs7911264 | rs1119894 | rs4810970 | rs6594583 | rs7045373 |
| rs12263369 | rs4774666 | rs6020572 | rs885137 | rs10811295 |
| rs11813225 | rs12438626 | rs2426226 | rs442947 | rs7028580 |
| rs2689691 | rs5027653 | rs4811177 | rs180473 | rs7027101 |
| rs7100415 | rs8024591 | rs2801008 | rs13154524 | rs10811316 |
| rs752416 | rs11635772 | rs4811421 | rs7380387 | rs10964443 |
| rs10882657 | rs8040189 | rs6022371 | rs6882543 | rs10738560 |
| rs3929900 | rs7178796 | rs200600 | rs6895205 | rs7867635 |
| rs756029 | rs12900045 | rs467670 | rs10213844 | rs10757126 |
| rs2817709 | rs4774931 | rs468211 | rs9326863 | rs7043657 |
| rs2211243 | rs12914388 | rs2781293 | rs12659119 | rs10757132 |
| rs1325494 | rs6493999 | rs2766661 | rs569940 | rs7030726 |
| rs2018085 | rs56403110 | rs17525247 | rs382427 | rs6475451 |
| rs11189915 | rs6151441 | rs6013763 | rs153561 | rs6475457 |
| rs2111326 | rs2899656 | rs6022713 | rs13445 | rs4422852 |
| rs7923726 | rs2062091 | rs2058248 | rs4705755 | rs6475464 |
| rs4919438 | rs28380001 | rs6064020 | rs13184187 | rs2780838 |
| rs1006544 | rs11631013 | rs1055253 | rs3922585 | rs2383142 |
| rs2077642 | rs2053154 | rs169639 | rs4705782 | rs2383147 |
| rs35911940 | rs12899093 | rs158542 | rs6876116 | rs10811394 |
| rs10883559 | rs8023852 | rs290465 | rs3846720 | rs4978017 |
| rs11190766 | rs9972569 | rs1150414 | rs4293904 | rs10811399 |
| rs752974 | rs112815338 | rs11697861 | rs337699 | rs10511684 |
| rs2275383 | rs11634017 | rs7273768 | rs285879 | rs1412443 |
| rs7092614 | rs4366668 | rs1041153 | rs712550 | rs7036993 |
| rs10883592 | rs28556680 | rs2426659 | rs2662453 | rs7854932 |
| rs11596561 | rs730708 | rs6025494 | rs254225 | rs10757169 |
| rs11190955 | rs7181798 | rs4810100 | rs153585 | rs1857649 |
| rs10883621 | rs11636980 | rs6070404 | rs267083 | rs10964817 |
| rs12414526 | rs405390 | rs2865469 | rs13171423 | rs4255228 |
| rs9420822 | rs11071996 | rs259960 | rs7444283 | rs10811483 |
| rs9420829 | rs12438040 | rs6123903 | rs9790885 | rs7038852 |
| rs3095804 | rs34433849 | rs1475028 | rs2036565 | rs632941 |
| rs3802682 | rs2679623 | rs1407146 | rs4131101 | rs10811554 |
| rs11191240 | rs60425277 | rs6071444 | rs4421134 | rs1545749 |
| rs9664049 | rs4776530 | rs2274934 | rs10054289 | rs1840051 |
| rs2485375 | rs12908697 | rs35927656 | rs2914598 | rs7875511 |
| rs7915589 | rs8042689 | rs6122371 | rs6595376 | rs2383198 |
| rs4917973 | rs7176235 | rs1757696 | rs337125 | rs6475551 |
| rs12779854 | rs28535571 | rs1148737 | rs57408535 | rs10757239 |
| rs10786695 | rs12148532 | rs2334463 | rs6874251 | rs1440993 |
| rs11191372 | rs4886844 | rs12482474 | rs10455013 | rs10965143 |
| rs284860 | rs413150 | rs2822650 | rs11241788 | rs1134871 |
| rs4919691 | rs11072508 | rs2178933 | rs12109128 | rs4607709 |
| rs1591915 | rs4414463 | rs439328 | rs329188 | rs12335941 |
| rs10748835 | rs28550045 | rs2822961 | rs6879081 | rs3814960 |
| rs17725614 | rs11072580 | rs2823054 | rs10463824 | rs2069418 |
| rs10883820 | rs8024737 | rs1736010 | rs61368741 | rs7028570 |
| rs11191513 | rs12438314 | rs9981901 | rs10039525 | rs1556516 |
| rs10786733 | rs8027520 | rs2242862 | rs1035405 | rs1333048 |
| rs2281859 | rs2056307 | rs2205454 | rs6876929 | rs7864275 |
| rs7897899 | rs1765250 | rs2823662 | rs1895279 | rs7035278 |
| rs34134675 | rs934134 | rs55957001 | rs10054163 | rs10965319 |
| rs1670008 | rs8025576 | rs2823809 | rs9918267 | rs1360136 |
| rs10884156 | rs7177957 | rs6517716 | rs2420148 | rs1327063 |
| rs11192865 | rs4778956 | rs4143392 | rs6890009 | rs527772 |
| rs7075966 | rs11639440 | rs8131134 | rs803220 | rs525185 |
| rs12761075 | rs11259948 | rs2824376 | rs154115 | rs586716 |
| rs10787055 | rs4778691 | rs243602 | rs6864166 | rs1112389 |
| rs1329480 | rs2642820 | rs197560 | rs7723128 | rs10811743 |
| rs10159686 | rs4842927 | rs1491767 | rs1860208 | rs62573495 |
| rs10509899 | rs7403597 | rs9979312 | rs6866160 | rs1448788 |
| rs10509901 | rs12907384 | rs13051177 | rs7703486 | rs2383242 |
| rs7899245 | rs891640 | rs2187166 | rs2867330 | rs7847958 |
| rs10787195 | rs28784669 | rs458715 | rs1109307 | rs4338198 |
| rs4918456 | rs9806348 | rs2825392 | rs2436445 | rs10965509 |
| rs2419305 | rs2679092 | rs34730803 | rs2436449 | rs13286845 |
| rs626198 | rs35177742 | rs2825608 | rs10479058 | rs1576676 |
| rs9420064 | rs8037820 | rs2407253 | rs2452857 | rs10965591 |
| rs2475277 | rs11073737 | rs2825914 | rs2431565 | rs12352937 |
| rs12258440 | rs12908985 | rs2825968 | rs6863158 | rs4977827 |
| rs10787224 | rs2196305 | rs1556307 | rs10036902 | rs1111819 |
| rs2501574 | rs3743164 | rs9984671 | rs329120 | rs10811872 |
| rs4918485 | rs11633200 | rs2155797 | rs474853 | rs4977611 |
| rs10884940 | rs7163925 | rs2826749 | rs4976247 | rs10811890 |
| rs2855469 | rs56332523 | rs1380591 | rs4976512 | rs1888540 |
| rs4918497 | rs8025146 | rs2826966 | rs7731706 | rs1330928 |
| rs11195096 | rs1899416 | rs13053027 | rs7737513 | rs10965929 |
| rs10884978 | rs2099842 | rs2827236 | rs7704886 | rs10125594 |
| rs11594629 | rs11073783 | rs2827459 | rs2349034 | rs7341738 |
| rs10885011 | rs1000587 | rs2827624 | rs3849046 | rs10966046 |
| rs1954179 | rs17196732 | rs11700744 | rs7705248 | rs7049077 |
| rs1853372 | rs7402591 | rs244275 | rs6596466 | rs10966136 |
| rs2181407 | rs8031405 | rs2827818 | rs11747498 | rs7033446 |
| rs1407696 | rs62023499 | rs2828073 | rs12518126 | rs7031303 |
| rs1327549 | rs150307 | rs2408741 | rs4835725 | rs829750 |
| rs4918614 | rs11639246 | rs6516579 | rs12515989 | rs7038811 |
| rs11195400 | rs1869179 | rs1910629 | rs4835729 | rs1158677 |
| rs638019 | rs2001217 | rs2260875 | rs55792153 | rs10812265 |
| rs2032023 | rs8037946 | rs28379174 | rs7378724 | rs9298877 |
| rs10749075 | rs7171664 | rs2829268 | rs261534 | rs6475912 |
| rs61873104 | rs1031900 | rs8131042 | rs261532 | rs655880 |
| rs4351741 | rs8035739 | rs6516659 | rs10040658 | rs10968221 |
| rs4918788 | rs4777647 | rs2829737 | rs12108986 | rs6476046 |
| rs10885441 | rs4238478 | rs1783011 | rs7727559 | rs824244 |
| rs2419859 | rs11856594 | rs11087971 | rs2060296 | rs7470546 |
| rs11196881 | rs6496024 | rs2829938 | rs155347 | rs2360194 |
| rs10885636 | rs1585235 | rs2830024 | rs2974422 | rs10813395 |
| rs2420110 | rs11638098 | rs1382179 | rs269767 | rs13301468 |
| rs12777802 | rs4984473 | rs222178 | rs4432943 | rs453019 |
| rs4751995 | rs11247084 | rs419895 | rs33998 | rs446885 |
| rs1638436 | rs11857691 | rs229117 | rs447923 | rs10971203 |
| rs363225 | rs12912927 | rs9980089 | rs712166 | rs697529 |
| rs4752137 | rs2045107 | rs363906 | rs1370982 | rs855508 |
| rs7070181 | rs8031024 | rs3906630 | rs12188243 | rs1928576 |
| rs4751677 | rs10902589 | rs2257008 | rs11740320 | rs565070 |
| rs11199019 | rs2881218 | rs2776155 | rs11167804 | rs4879915 |
| rs4237513 | rs2562189 | rs9978774 | rs258776 | rs7862798 |
| rs10886707 | rs9934799 | rs2250997 | rs33383 | rs10973041 |
| rs7901641 | rs9931183 | rs8132558 | rs34644584 | rs4255220 |
| rs11199908 | rs2235624 | rs2832137 | rs34876863 | rs4880007 |
| rs3962111 | rs26857 | rs2251130 | rs17401794 | rs10973066 |
| rs7383 | rs11076933 | rs2832411 | rs7712039 | rs7025042 |
| rs11248321 | rs11228 | rs7509953 | rs153517 | rs4878660 |
| rs7908901 | rs1149484 | rs2832516 | rs2910256 | rs4242705 |
| rs1955340 | rs4578649 | rs2065278 | rs1422875 | rs10758409 |
| rs7095944 | rs1129568 | rs1034127 | rs17413389 | rs34339593 |
| rs10901864 | rs13740 | rs2832875 | rs2546880 | rs10973149 |
| rs11244538 | rs758747 | rs10154124 | rs2130487 | rs10973156 |
| rs4962621 | rs9989424 | rs56343261 | rs467654 | rs9407059 |
| rs2184378 | rs1799892 | rs9305451 | rs10875612 | rs1021444 |
| rs61484573 | rs4786427 | rs9975969 | rs10454999 | rs7872067 |
| rs12764644 | rs129968 | rs4816408 | rs34434379 | rs7039784 |
| rs1475392 | rs7188906 | rs9979499 | rs13166300 | rs13285624 |
| rs563859 | rs8049367 | rs1892575 | rs11750453 | rs10283534 |
| rs9804321 | rs2238436 | rs2833799 | rs13354603 | rs12337316 |
| rs10829765 | rs11647828 | rs844990 | rs1153085 | rs7846747 |
| rs7906850 | rs405470 | rs844986 | rs6887366 | rs7026004 |
| rs7897094 | rs397666 | rs8130756 | rs352362 | rs12554976 |
| rs4075501 | rs35743442 | rs9653711 | rs4705074 | rs1339553 |
| rs4838707 | rs251732 | rs9984454 | rs10065816 | rs308495 |
| rs12419281 | rs3747571 | rs7281554 | rs10040689 | rs10814518 |
| rs34360522 | rs1045519 | rs2834248 | rs6892940 | rs6476622 |
| rs3793966 | rs8050388 | rs1109847 | rs152949 | rs10814519 |
| rs7933508 | rs12596055 | rs9980500 | rs30829 | rs834356 |
| rs4930102 | rs11077123 | rs8131131 | rs1541915 | rs308523 |
| rs1010173 | rs763649 | rs1557267 | rs3812008 | rs4472607 |
| rs10833322 | rs6501110 | rs2000436 | rs12517208 | rs309460 |
| rs276908 | rs13335217 | rs2186287 | rs882354 | rs2025440 |
| rs10742223 | rs9921894 | rs729871 | rs7711562 | rs7028020 |
| rs1368820 | rs66975383 | rs34835411 | rs6881743 | rs7027585 |
| rs2445304 | rs56993178 | rs2300395 | rs34335538 | rs28651528 |
| rs1994519 | rs1641007 | rs8134608 | rs2569093 | rs112855200 |
| rs2250886 | rs753071 | rs2834708 | rs2748244 | rs10868796 |
| rs3105718 | rs10852327 | rs73900767 | rs2273234 | rs2486452 |
| rs7112087 | rs4985061 | rs2834745 | rs7734132 | rs11138902 |
| rs10839861 | rs7189170 | rs2094869 | rs10044420 | rs7038061 |
| rs11041559 | rs12448702 | rs2242915 | rs1478392 | rs4744602 |
| rs371799 | rs4278749 | rs2242917 | rs253566 | rs1887971 |
| rs1842827 | rs4533284 | rs57820060 | rs1422345 | rs12378487 |
| rs11041715 | rs12596733 | rs11088316 | rs4130911 | rs6560284 |
| rs34812123 | rs2965906 | rs13048344 | rs6580024 | rs7027604 |
| rs10769865 | rs17568098 | rs8129061 | rs10477085 | rs10869264 |
| rs7932687 | rs11643736 | rs8132050 | rs17115870 | rs35560060 |
| rs7944370 | rs172729 | rs2835028 | rs286602 | rs4265248 |
| rs4758041 | rs6497662 | rs12482196 | rs7713997 | rs1537295 |
| rs1108277 | rs7193371 | rs438540 | rs244976 | rs744339 |
| rs1569128 | rs4300661 | rs11701331 | rs12652777 | rs12347453 |
| rs7937959 | rs350262 | rs2835338 | rs10076475 | rs629806 |
| rs6578931 | rs7198031 | rs2835382 | rs728693 | rs10781458 |
| rs6578934 | rs4780510 | rs9305604 | rs6883191 | rs357638 |
| rs10431029 | rs8055949 | rs2835517 | rs2964325 | rs10867255 |
| rs10734629 | rs66467937 | rs9977602 | rs12189195 | rs10117663 |
| rs12799778 | rs7201964 | rs1060577 | rs11135037 | rs1825279 |
| rs11041785 | rs11644210 | rs9982932 | rs2419921 | rs879043 |
| rs4539318 | rs4782185 | rs2835971 | rs2963469 | rs7019493 |
| rs10840002 | rs7196425 | rs2836054 | rs10075134 | rs7020372 |
| rs11041843 | rs11866321 | rs1121246 | rs12188736 | rs6559645 |
| rs10840010 | rs9646256 | rs2836151 | rs6556373 | rs514471 |
| rs10769907 | rs2341643 | rs4817916 | rs7705878 | rs1546316 |
| rs10840047 | rs3103820 | rs2836201 | rs6898769 | rs2811911 |
| rs7126013 | rs56036711 | rs2836257 | rs11745415 | rs4877814 |
| rs10743075 | rs208945 | rs9983358 | rs7707897 | rs566587 |
| rs9736791 | rs59618367 | rs4817943 | rs270648 | rs1332537 |
| rs6484485 | rs7198121 | rs2836442 | rs7732603 | rs1187325 |
| rs7113874 | rs7192301 | rs12482155 | rs194228 | rs1439048 |
| rs2036423 | rs13336995 | rs2244890 | rs6870828 | rs7023589 |
| rs7105248 | rs10400925 | rs460214 | rs4413517 | rs1443444 |
| rs10770074 | rs35454800 | rs4143428 | rs10077890 | rs11140776 |
| rs7938543 | rs11074759 | rs2410052 | rs12655506 | rs4310286 |
| rs10500733 | rs34652009 | rs1013777 | rs1949361 | rs11140831 |
| rs12801932 | rs4788000 | rs7276587 | rs252945 | rs4302927 |
| rs6485128 | rs7187776 | rs9680142 | rs13165451 | rs7872176 |
| rs4756830 | rs2252590 | rs12481753 | rs10056880 | rs477286 |
| rs12281511 | rs7187359 | rs2150411 | rs36126416 | rs7030902 |
| rs11522584 | rs4889489 | rs7278297 | rs1458357 | rs734761 |
| rs10832119 | rs9938088 | rs4816626 | rs7446327 | rs2841447 |
| rs11023088 | rs11150599 | rs980185 | rs4868746 | rs2841488 |
| rs1879888 | rs12443808 | rs2837282 | rs2195935 | rs7033930 |
| rs7106127 | rs12445650 | rs2837494 | rs10059669 | rs11141352 |
| rs4477405 | rs7199949 | rs2837529 | rs1799568 | rs10735577 |
| rs4237711 | rs56314408 | rs8134455 | rs7710179 | rs10868536 |
| rs12802656 | rs7190802 | rs9980324 | rs13174736 | rs3128510 |
| rs214106 | rs1052352 | rs9305733 | rs11951480 | rs34946096 |
| rs12295745 | rs2288005 | rs1638369 | rs1422423 | rs7855763 |
| rs10832913 | rs11641202 | rs9978931 | rs1875969 | rs3138488 |
| rs2553964 | rs11643752 | rs9976693 | rs2249184 | rs2380281 |
| rs4757854 | rs6565241 | rs170430 | rs2338832 | rs10739994 |
| rs4757026 | rs4519345 | rs11910048 | rs957998 | rs773514 |
| rs11025608 | rs4889548 | rs397322 | rs4868032 | rs1995933 |
| rs9804466 | rs12149872 | rs8131190 | rs1363281 | rs10761130 |
| rs11605156 | rs7204877 | rs11700464 | rs1345631 | rs4744134 |
| rs7127246 | rs12599106 | rs7275379 | rs11741392 | rs4744175 |
| rs11026620 | rs2163977 | rs2269162 | rs4867641 | rs4744243 |
| rs7937005 | rs12444879 | rs6586346 | rs4868052 | rs4477096 |
| rs10767130 | rs28407821 | rs66836472 | rs4302607 | rs7027708 |
| rs7126488 | rs17811434 | rs10154161 | rs779658 | rs4744446 |
| rs10767287 | rs4966454 | rs6586251 | rs2441017 | rs1979022 |
| rs11028393 | rs8053839 | rs9637301 | rs29647 | rs3852402 |
| rs11028932 | rs9933892 | rs9982232 | rs4867651 | rs1027710 |
| rs4923330 | rs194406 | rs9982622 | rs2043275 | rs7858493 |
| rs11029621 | rs9927895 | rs9978199 | rs6890307 | rs7389278 |
| rs10835138 | rs2270396 | rs11702457 | rs68063677 | rs7025117 |
| rs12223664 | rs7500375 | rs11911601 | rs29622 | rs354269 |
| rs1564745 | rs11076007 | rs500449 | rs29619 | rs1655829 |
| rs1914472 | rs1362557 | rs626045 | rs62643170 | rs6478965 |
| rs1502449 | rs11075668 | rs580007 | rs35278513 | rs4743317 |
| rs10742225 | rs4784312 | rs8131841 | rs33855 | rs2164157 |
| rs594982 | rs4783813 | rs631800 | rs6877005 | rs10818574 |
| rs10767879 | rs8051348 | rs1595167 | rs2089190 | rs2149564 |
| rs1848394 | rs12149854 | rs2838326 | rs3131913 | rs12000073 |
| rs2037613 | rs8055853 | rs8131005 | rs393952 | rs591486 |
| rs2745890 | rs11076104 | rs7282144 | rs9313669 | rs6479036 |
| rs224632 | rs35835960 | rs6518345 | rs1001675 | rs619497 |
| rs223054 | rs62035995 | rs11552067 | rs10057481 | rs7043341 |
| rs4922580 | rs1787784 | rs233273 | rs28857288 | rs1857083 |
| rs7119024 | rs2405011 | rs7275281 | rs4551090 | rs7853051 |
| rs1033629 | rs6499948 | rs11088977 | rs13177957 | rs4743239 |
| rs11031658 | rs8057892 | rs60321241 | rs11135001 | rs10116067 |
| rs6484567 | rs12599385 | rs7281500 | rs2962842 | rs1407905 |
| rs10835886 | rs1392501 | rs7276178 | rs353490 | rs2093696 |
| rs10742277 | rs2582596 | rs1005467 | rs446382 | rs1338121 |
| rs3858459 | rs1993020 | rs9982334 | rs7719125 | rs10760755 |
| rs10767949 | rs9931069 | rs9984708 | rs10077929 | rs7854555 |
| rs201834 | rs8043792 | rs2096507 | rs4976685 | rs7864269 |
| rs169326 | rs1967270 | rs4819272 | rs4976691 | rs1875039 |
| rs436895 | rs3011251 | rs6010323 | rs2545801 | rs1443837 |
| rs7116464 | rs1605962 | rs2241042 | rs1130857 | rs1523666 |
| rs10835958 | rs7191753 | rs4819654 | rs2544809 | rs2487054 |
| rs7927900 | rs9931228 | rs361534 | rs2545794 | rs2771041 |
| rs197707 | rs7191764 | rs2240110 | rs335429 | rs1360089 |
| rs1007270 | rs12918800 | rs5993761 | rs179810 | rs10820813 |
| rs12792396 | rs1095163 | rs419424 | rs335458 | rs4742955 |
| rs2476456 | rs8062611 | rs1558656 | rs335424 | rs3793640 |
| rs2615934 | rs363173 | rs240053 | rs335481 | rs6477451 |
| rs11032300 | rs7200919 | rs4822358 | rs435473 | rs10119254 |
| rs10742299 | rs7206718 | rs11912448 | rs186803 | rs9299110 |
| rs831596 | rs6979 | rs131421 | rs4458584 | rs2417650 |
| rs3817442 | rs255050 | rs5759963 | rs262012 | rs4598317 |
| rs3924417 | rs7200986 | rs139736 | rs6897783 | rs10759149 |
| rs7930960 | rs3743741 | rs546480 | rs464156 | rs2417660 |
| rs10836148 | rs2236393 | rs737817 | rs13164626 | rs4742659 |
| rs11032535 | rs8057271 | rs9613166 | rs399108 | rs10978357 |
| rs745281 | rs8049728 | rs79554 | rs9392504 | rs10816356 |
| rs1267475 | rs28733806 | rs5762393 | rs6923301 | rs10759156 |
| rs1116471 | rs3813904 | rs134176 | rs10793858 | rs10816388 |
| rs263099 | rs35593117 | rs5762553 | rs1543538 | rs4743030 |
| rs391277 | rs9940707 | rs5762670 | rs2479016 | rs2183360 |
| rs10836490 | rs12930285 | rs5762730 | rs1680856 | rs10512368 |
| rs1304854 | rs4788477 | rs5752772 | rs1040521 | rs6477643 |
| rs7125444 | rs4888286 | rs5752773 | rs9502099 | rs7027512 |
| rs10768201 | rs12446615 | rs2239815 | rs627358 | rs2210160 |
| rs2197600 | rs200283 | rs2269577 | rs2032987 | rs10759393 |
| rs4418777 | rs10871299 | rs6005936 | rs12202010 | rs10759450 |
| rs11033545 | rs8057128 | rs5997435 | rs7772158 | rs7857003 |
| rs10768207 | rs870890 | rs760628 | rs6939362 | rs2805485 |
| rs566797 | rs9922812 | rs134591 | rs267198 | rs7869209 |
| rs6484861 | rs12927360 | rs134602 | rs6917976 | rs4979223 |
| rs11033693 | rs2199533 | rs134662 | rs6597345 | rs56937115 |
| rs7104753 | rs7189855 | rs126077 | rs1155921 | rs9942975 |
| rs1105593 | rs1110137 | rs5752870 | rs9396715 | rs1249725 |
| rs6484869 | rs383673 | rs132277 | rs7763917 | rs4133032 |
| rs7102380 | rs7195655 | rs132282 | rs9283875 | rs2017976 |
| rs964524 | rs4545829 | rs132335 | rs1570377 | rs1405 |
| rs2139139 | rs11643733 | rs132375 | rs12194771 | rs756118 |
| rs7109868 | rs4888183 | rs132388 | rs4714115 | rs4837135 |
| rs10836619 | rs2303266 | rs12171266 | rs9296487 | rs10125591 |
| rs61877730 | rs11150481 | rs4820804 | rs821287 | rs10984112 |
| rs7120395 | rs1019567 | rs28755 | rs2841525 | rs10760025 |
| rs10836638 | rs2303234 | rs174764 | rs4715909 | rs10984663 |
| rs11033890 | rs9934611 | rs5763190 | rs233504 | rs9409227 |
| rs1871684 | rs13338394 | rs174709 | rs1739089 | rs10984879 |
| rs35533395 | rs7185290 | rs28380 | rs2619538 | rs871214 |
| rs1512359 | rs12931510 | rs737977 | rs9358081 | rs4620347 |
| rs11825996 | rs300010 | rs2857645 | rs11756744 | rs4837954 |
| rs7107489 | rs3843836 | rs9625870 | rs4716167 | rs10818768 |
| rs1606785 | rs8050209 | rs737939 | rs6899431 | rs1110320 |
| rs1897935 | rs9935713 | rs997913 | rs9477835 | rs10122047 |
| rs7110475 | rs3785415 | rs5763688 | rs9358223 | rs4838187 |
| rs7949532 | rs7185897 | rs5763751 | rs7753163 | rs360004 |
| rs1911630 | rs11649465 | rs1989870 | rs9358297 | rs512445 |
| rs11035789 | rs4968120 | rs4823075 | rs6917583 | rs4837041 |
| rs7120649 | rs866304 | rs757027 | rs10456012 | rs28641758 |
| rs1381582 | rs28769064 | rs7289941 | rs2025984 | rs1538503 |
| rs1484970 | rs10438742 | rs5998196 | rs196051 | rs2812287 |
| rs10837804 | rs2003968 | rs5754043 | rs2498230 | rs7041940 |
| rs12283338 | rs62089721 | rs929111 | rs12661269 | rs4837143 |
| rs7131270 | rs231690 | rs9607019 | rs6931151 | rs10819281 |
| rs7929418 | rs9899953 | rs2413211 | rs793832 | rs3802364 |
| rs3923808 | rs410520 | rs130900 | rs303895 | rs10819289 |
| rs10838336 | rs9914449 | rs2413365 | rs1206523 | rs7470971 |
| rs1000665 | rs2535590 | rs11089789 | rs198825 | rs2244624 |
| rs6485642 | rs11078638 | rs228965 | rs12526680 | rs2798430 |
| rs950105 | rs8072369 | rs2235573 | rs6930508 | rs1539567 |
| rs7947747 | rs17671352 | rs139312 | rs149976 | rs2245051 |
| rs11039097 | rs5417 | rs139322 | rs1005125 | rs504434 |
| rs10838660 | rs34280588 | rs5757764 | rs4713186 | rs2256139 |
| rs3740685 | rs55894190 | rs2143177 | rs2006770 | rs515182 |
| rs3740699 | rs3803798 | rs133072 | rs2734913 | rs514024 |
| rs3817334 | rs7640 | rs11703894 | rs3130359 | rs7033913 |
| rs11039345 | rs55809496 | rs5758064 | rs3130789 | rs4836591 |
| rs10838758 | rs12937245 | rs4821976 | rs2516456 | rs3739821 |
| rs3886262 | rs9899694 | rs4822002 | rs493515 | rs9792458 |
| rs10838769 | rs4792000 | rs4822006 | rs9394152 | rs2502730 |
| rs4752798 | rs11078724 | rs2038208 | rs9461933 | rs3003610 |
| rs4752801 | rs58412412 | rs5751061 | rs4711378 | rs7854612 |
| rs1228001 | rs2278635 | rs5758307 | rs9394248 | rs7023913 |
| rs6485802 | rs9913189 | rs5758311 | rs12525930 | rs10987969 |
| rs1113480 | rs2585399 | rs132906 | rs3176336 | rs7874044 |
| rs11039593 | rs55840146 | rs9607805 | rs1724092 | rs7855078 |
| rs66468703 | rs35212009 | rs2076199 | rs2814898 | rs7870070 |
| rs56080733 | rs11078853 | rs9611618 | rs7743381 | rs10113902 |
| rs10839342 | rs11869844 | rs2267437 | rs6935135 | rs9411343 |
| rs10794225 | rs28678685 | rs6002556 | rs3008798 | rs787878 |
| rs11245849 | rs8082548 | rs134870 | rs13198616 | rs13284231 |
| rs10902275 | rs12601853 | rs2858471 | rs2494945 | rs445690 |
| rs4362132 | rs12951787 | rs4823124 | rs2004197 | rs459983 |
| rs479013 | rs9894592 | rs2213373 | rs1015149 | rs1328402 |
| rs4300408 | rs9910879 | rs5764979 | rs2295278 | rs932887 |
| rs548321 | rs7217195 | rs8136562 | rs10948059 | rs215156 |
| rs10896596 | rs230936 | rs9330798 | rs2254303 | rs2231403 |
| rs10431128 | rs1476728 | rs2223245 | rs833066 | rs1964196 |
| rs3886146 | rs163376 | rs1548977 | rs1018125 | rs507586 |
| rs568468 | rs62071039 | rs5767677 | rs9472407 | rs3011274 |
| rs4939267 | rs578065 | rs5767835 | rs1406846 | rs526855 |
| rs1303615 | rs3760298 | rs9615322 | rs986737 | rs625186 |
| rs10897069 | rs12451075 | rs738553 | rs1527706 | rs35768185 |
| rs485639 | rs2285582 | rs9616290 | rs9381469 | rs6597600 |
| rs903778 | rs9896441 | rs739239 | rs1738829 | rs9411363 |
| rs1631096 | rs8080706 | rs13053855 | rs506816 | rs10751502 |
| rs7129775 | rs7207889 | rs13056386 | rs9463414 | rs514659 |
| rs4121883 | rs7222886 | rs7611849 | rs2490135 | rs8176649 |
| rs10897450 | rs9897388 | rs2196035 | rs990237 | rs176697 |
| rs661292 | rs2285579 | rs2063243 | rs1337823 | rs7041640 |
| rs548150 | rs178838 | rs6785718 | rs1492625 | rs59358281 |
| rs666194 | rs178833 | rs1617892 | rs2744494 | rs3124767 |
| rs669742 | rs9916220 | rs1533599 | rs9367470 | rs1858879 |
| rs4930387 | rs2109262 | rs34745764 | rs10948681 | rs2073935 |
| rs4930391 | rs4622550 | rs317555 | rs449690 | rs3124757 |
| rs2447595 | rs9889868 | rs4685802 | rs488161 | rs2789863 |
| rs308327 | rs34001712 | rs9819086 | rs1326553 | rs9410002 |
| rs312023 | rs11651620 | rs4686019 | rs4415156 | rs12377357 |
| rs10896401 | rs7501812 | rs2173516 | rs6899853 | rs10858216 |
| rs7109934 | rs3794782 | rs1385437 | rs9349781 | rs1183195 |
| rs11263587 | rs2076562 | rs9312020 | rs6915291 | rs10776888 |
| rs11235935 | rs12937053 | rs164467 | rs2098172 | rs7860634 |
| rs676889 | rs4925067 | rs2728936 | rs9396459 | rs67817384 |
| rs2096818 | rs8066143 | rs456520 | rs2676982 | rs28410669 |
| rs536637 | rs7504028 | rs6442166 | rs1851586 | rs7853207 |
| rs11237165 | rs11869425 | rs350663 | rs9344213 | rs3812573 |
| rs10899388 | rs11870550 | rs2606743 | rs4710672 | rs10735662 |
| rs478636 | rs11657456 | rs13060776 | rs6939191 | rs6563 |
| rs4980560 | rs4794872 | rs709161 | rs12195405 | rs7048814 |
| rs7102984 | rs9901372 | rs360741 | rs1884117 | rs710411 |
| rs4332544 | rs4483904 | rs4684999 | rs3013169 | rs4880106 |
| rs2919730 | rs2279959 | rs1902658 | rs7451329 | rs10735657 |
| rs10431150 | rs797974 | rs1038913 | rs1021832 | rs1332793 |
| rs1792313 | rs2628176 | rs6772489 | rs9363498 | rs4880132 |
| rs12806918 | rs6505171 | rs2077624 | rs1377696 | rs2784042 |
| rs651933 | rs55872374 | rs10440095 | rs9354622 | rs7852970 |
| rs947848 | rs28539246 | rs2733480 | rs9346072 | rs2271869 |
| rs10898935 | rs4541129 | rs12488591 | rs2802696 | rs2271862 |
| rs1670564 | rs2905788 | rs12714955 | rs9346322 | rs4962240 |
| rs4944888 | rs2905884 | rs336613 | rs495558 | rs10867070 |
| rs2851072 | rs9898664 | rs6551236 | rs782005 | rs766374 |
| rs604692 | rs12940303 | rs874984 | rs4706464 | rs6644389 |
| rs11236639 | rs7502556 | rs7609718 | rs717519 | rs5988544 |
| rs7939513 | rs2525570 | rs2133376 | rs1973843 | rs28395487 |
| rs34283727 | rs2854334 | rs1497507 | rs9446659 | rs9778721 |
| rs4945177 | rs7504054 | rs2054942 | rs6928101 | rs28692905 |
| rs10793285 | rs4794889 | rs1602575 | rs473675 | rs4589044 |
| rs478704 | rs178893 | rs1579072 | rs2065859 | rs28616689 |
| rs489724 | rs7216964 | rs1485385 | rs11751308 | rs28680859 |
| rs680895 | rs315429 | rs6807249 | rs7749761 | rs7877047 |
| rs7114833 | rs443259 | rs12495053 | rs9350589 | rs5948849 |
| rs7483712 | rs315450 | rs58136406 | rs1457960 | rs6588830 |
| rs10897847 | rs315498 | rs12497476 | rs9352433 | rs5948891 |
| rs4943853 | rs441695 | rs871963 | rs2504286 | rs7880408 |
| rs510621 | rs315502 | rs11716410 | rs818229 | rs6644913 |
| rs588888 | rs770512 | rs1386893 | rs235453 | rs6421529 |
| rs656537 | rs16966858 | rs9816609 | rs7753531 | rs67112992 |
| rs10792695 | rs220447 | rs6788643 | rs2655675 | rs34040133 |
| rs12270207 | rs11658318 | rs2126314 | rs465483 | rs3859965 |
| rs6592228 | rs3760454 | rs35585477 | rs1535075 | rs4460540 |
| rs286052 | rs9906766 | rs7630183 | rs310419 | rs5915797 |
| rs631375 | rs7217877 | rs1865301 | rs7741650 | rs2014145 |
| rs290193 | rs6505293 | rs6782211 | rs194633 | rs1552189 |
| rs609012 | rs763545 | rs7636377 | rs2983881 | rs12836764 |
| rs1445506 | rs8069673 | rs1902346 | rs1180243 | rs5989502 |
| rs1445496 | rs76541217 | rs1451523 | rs6930990 | rs5933854 |
| rs7107881 | rs9896320 | rs7429569 | rs1186082 | rs12014406 |
| rs671686 | rs728718 | rs6763773 | rs4434440 | rs739665 |
| rs541674 | rs62065463 | rs11705838 | rs1321741 | rs6640592 |
| rs545592 | rs4794923 | rs7618115 | rs12664099 | rs5933848 |
| rs583162 | rs8065328 | rs636164 | rs503887 | rs6640718 |
| rs3844143 | rs3859281 | rs6788583 | rs9351114 | rs7064813 |
| rs12271958 | rs4795854 | rs13077510 | rs9342120 | rs5935267 |
| rs3844145 | rs9903369 | rs56000017 | rs2787887 | rs4830863 |
| rs7120222 | rs2339213 | rs950146 | rs7760481 | rs4439289 |
| rs10898452 | rs2301730 | rs6550471 | rs9359815 | rs5936010 |
| rs291252 | rs226428 | rs4678570 | rs10502 | rs6527702 |
| rs2646727 | rs7217330 | rs169196 | rs10806424 | rs4986503 |
| rs614301 | rs7213342 | rs6599208 | rs4706362 | rs5909143 |
| rs4944573 | rs9303277 | rs2853712 | rs4707654 | rs5909268 |
| rs7926796 | rs3859189 | rs12715349 | rs2655620 | rs11795873 |
| rs7107136 | rs67138687 | rs4676572 | rs2452941 | rs7881721 |
| rs4144867 | rs7406717 | rs6803370 | rs641961 | rs5990811 |
| rs1540083 | rs3809876 | rs428903 | rs812479 | rs9306760 |
| rs10898524 | rs13412 | rs2700437 | rs9452479 | rs6418743 |
| rs4245426 | rs2079009 | rs13069836 | rs462880 | rs4824217 |
| rs7949665 | rs8072215 | rs6764909 | rs2248426 | rs11797675 |
| rs11235140 | rs1869365 | rs704918 | rs6902513 | rs12394178 |
| rs475030 | rs11079041 | rs61102363 | rs1028302 | rs5986510 |
| rs178244 | rs7218653 | rs4682949 | rs986103 | rs5944708 |
| rs594647 | rs8072391 | rs2059757 | rs2206094 | rs6526475 |
| rs319030 | rs957971 | rs9877499 | rs10457368 | rs6418572 |
| rs11018907 | rs4796791 | rs34478154 | rs2503776 | rs5943959 |
| rs575363 | rs2883456 | rs3796371 | rs1481444 | rs12861496 |
| rs7103276 | rs963987 | rs11710586 | rs6913836 | rs5926502 |
| rs10501760 | rs62075809 | rs13080080 | rs9321763 | rs4428795 |
| rs10830783 | rs2292755 | rs6777190 | rs36082012 | rs12007775 |
| rs8181573 | rs55985470 | rs7621046 | rs12530388 | rs5972128 |
| rs2605593 | rs4793056 | rs1108301 | rs9404105 | rs6526890 |
| rs562371 | rs7224577 | rs6808742 | rs1417178 | rs6631042 |
| rs12574162 | rs8067269 | rs34103310 | rs9322650 | rs4487947 |
| rs7131373 | rs9783806 | rs13094672 | rs1853411 | rs1482833 |
| rs7902 | rs12942842 | rs11130115 | rs9386413 | rs5926972 |
| rs559477 | rs6503415 | rs12629262 | rs6942307 | rs5927652 |
| rs1874059 | rs1052504 | rs73069104 | rs9372090 | rs1484851 |
| rs2555321 | rs34774205 | rs62246474 | rs9386476 | rs725979 |
| rs2597587 | rs2074186 | rs1531875 | rs2219666 | rs3361 |
| rs2155904 | rs2072441 | rs73081205 | rs11153045 | rs6527072 |
| rs6589648 | rs11079830 | rs2291298 | rs6942222 | rs16989486 |
| rs2726396 | rs4410130 | rs2255532 | rs7764597 | rs4642801 |
| rs808828 | rs575791 | rs9884022 | rs932934 | rs2733468 |
| rs978426 | rs1918733 | rs6781790 | rs508197 | rs2704904 |
| rs7940118 | rs9906242 | rs4955420 | rs3818933 | rs4829222 |
| rs10791492 | rs4793696 | rs12631989 | rs4945848 | rs6527094 |
| rs10895184 | rs2214016 | rs13078949 | rs9481066 | rs4829227 |
| rs1939045 | rs9906461 | rs6795772 | rs1022092 | rs7066656 |
| rs10895202 | rs8075417 | rs7621003 | rs463860 | rs5972430 |
| rs4429028 | rs9898710 | rs7637999 | rs9398298 | rs5927819 |
| rs2165588 | rs6504834 | rs6787417 | rs7744265 | rs6631405 |
| rs7107662 | rs1508010 | rs2352975 | rs7752798 | rs1458377 |
| rs880474 | rs7216847 | rs2301166 | rs10872099 | rs17317304 |
| rs2508314 | rs9899776 | rs13095652 | rs9400679 | rs3932944 |
| rs7930416 | rs11079212 | rs352139 | rs1407731 | rs5927924 |
| rs10791558 | rs1984543 | rs1010553 | rs238596 | rs4829248 |
| rs1426398 | rs4461140 | rs35209711 | rs12197412 | rs1795581 |
| rs10791566 | rs17762344 | rs9846874 | rs6912656 | rs8181247 |
| rs7104685 | rs2233911 | rs6799361 | rs6925564 | rs1456737 |
| rs1894115 | rs437732 | rs551799 | rs1117046 | rs874829 |
| rs938600 | rs8073731 | rs9872330 | rs1853080 | rs331347 |
| rs10791579 | rs1292055 | rs484247 | rs1413751 | rs3788899 |
| rs6590973 | rs1024637 | rs6776772 | rs1605598 | rs6527189 |
| rs4285839 | rs11657378 | rs11130550 | rs509067 | rs3788904 |
| rs2846342 | rs7210457 | rs9311659 | rs1931228 | rs5972560 |
| rs1606669 | rs8069587 | rs12486363 | rs390665 | rs7881832 |
| rs12275420 | rs8075950 | rs2174031 | rs803595 | rs228314 |
| rs2174158 | rs2052119 | rs1616465 | rs6942315 | rs6631608 |
| rs7124521 | rs10853044 | rs11721188 | rs9372480 | rs36122067 |
| rs4755107 | rs9902971 | rs2734365 | rs2180811 | rs808517 |
| rs2458331 | rs9889486 | rs9828175 | rs210653 | rs6527214 |
| rs1793060 | rs3859214 | rs1981561 | rs210637 | rs762 |
| rs673092 | rs1877848 | rs13063565 | rs3752848 | rs2855693 |
| rs503951 | rs11079681 | rs9311847 | rs9401023 | rs5972687 |
| rs949286 | rs7226140 | rs3856943 | rs12202619 | rs761732 |
| rs61912810 | rs4968829 | rs3210560 | rs658481 | rs5927141 |
| rs746592 | rs333922 | rs929555 | rs672311 | rs5971760 |
| rs2884331 | rs7211423 | rs28599817 | rs10872159 | rs2940040 |
| rs12278067 | rs180186 | rs723230 | rs12662262 | rs5973208 |
| rs604148 | rs7220635 | rs55651217 | rs283088 | rs1037970 |
| rs7947952 | rs7359556 | rs782729 | rs1342794 | rs12839169 |
| rs574568 | rs9911577 | rs34637289 | rs6901418 | rs17245760 |
| rs11214207 | rs11077630 | rs6767651 | rs6932452 | rs9284567 |
| rs4937765 | rs7207897 | rs6797121 | rs2245847 | rs28868958 |
| rs1800498 | rs323401 | rs6548980 | rs1324096 | rs5917541 |
| rs2735200 | rs2290955 | rs6806205 | rs9401442 | rs4827145 |
| rs2852444 | rs17557592 | rs2018997 | rs1267948 | rs1441482 |
| rs2513477 | rs4789144 | rs7647578 | rs4368828 | rs207036 |
| rs10891818 | rs9898904 | rs6797697 | rs12661887 | rs150220 |
| rs1698160 | rs4789241 | rs1603834 | rs2785713 | rs5917866 |
| rs10750086 | rs553305 | rs2315310 | rs1322634 | rs6520594 |
| rs10892059 | rs11077911 | rs4855416 | rs12202920 | rs3002414 |
| rs2622934 | rs2748429 | rs11128132 | rs1777224 | rs12008436 |
| rs2508450 | rs4789865 | rs4855317 | rs9321065 | rs4342761 |
| rs2512156 | rs9907544 | rs7643531 | rs13204656 | rs6520604 |
| rs4938469 | rs9909193 | rs6786226 | rs11556354 | rs5917921 |
| rs675479 | rs9907354 | rs11712880 | rs479187 | rs5963154 |
| rs1080620 | rs7225137 | rs11923382 | rs5008408 | rs5963158 |
| rs7927810 | rs8078253 | rs9310178 | rs6930710 | rs4281235 |
| rs4936423 | rs1106434 | rs1157456 | rs208869 | rs6609060 |
| rs1793185 | rs9905991 | rs1027233 | rs1203341 | rs7876589 |
| rs869638 | rs3803771 | rs35026817 | rs1408027 | rs10482552 |
| rs1629083 | rs9674881 | rs35457492 | rs2608923 | rs2961383 |
| rs12576100 | rs2621189 | rs13099927 | rs6918923 | rs952836 |
| rs11216845 | rs1225977 | rs7638933 | rs1406932 | rs4521899 |
| rs3825051 | rs4797925 | rs6792331 | rs4897614 | rs6417848 |
| rs11216853 | rs532950 | rs1996817 | rs56256859 | rs5963851 |
| rs517124 | rs1785282 | rs13080543 | rs9385674 | rs4827045 |
| rs12796373 | rs10468738 | rs12374061 | rs13199020 | rs5963199 |
| rs687664 | rs500214 | rs9861616 | rs10223556 | rs5918053 |
| rs1784300 | rs2658261 | rs2028240 | rs6905755 | rs12394954 |
| rs7127565 | rs11081160 | rs2322692 | rs2205843 | rs5918069 |
| rs1786684 | rs1062837 | rs7653851 | rs728030 | rs4827248 |
| rs589925 | rs9950715 | rs6794128 | rs7749106 | rs12845211 |
| rs1129013 | rs341213 | rs7428935 | rs4895438 | rs5918115 |
| rs7925499 | rs4605246 | rs35976325 | rs11755229 | rs887496 |
| rs2511854 | rs413139 | rs13094233 | rs4896131 | rs5918122 |
| rs6589729 | rs7230303 | rs7373838 | rs2050020 | rs4827050 |
| rs11217255 | rs9304008 | rs13068118 | rs210946 | rs2239492 |
| rs10750144 | rs635882 | rs1374851 | rs9494195 | rs5963951 |
| rs7945998 | rs8097838 | rs7622824 | rs2614277 | rs787094 |
| rs906823 | rs2215498 | rs6548520 | rs2244745 | rs3021319 |
| rs7928238 | rs10468615 | rs7433616 | rs2614265 | rs3013127 |
| rs4938719 | rs7227650 | rs9874934 | rs2614267 | rs2998257 |
| rs7478880 | rs1483126 | rs1563383 | rs4526212 | rs2998250 |
| rs1939665 | rs507790 | rs6809924 | rs9321508 | rs12008943 |
| rs1784931 | rs644004 | rs12494644 | rs9402724 | rs6610604 |
| rs677731 | rs531635 | rs276105 | rs7767066 | rs6609159 |
| rs614319 | rs45476491 | rs6785385 | rs7739635 | rs5917447 |
| rs7114175 | rs558128 | rs1375811 | rs2876317 | rs4827289 |
| rs1944607 | rs35963756 | rs979545 | rs6906871 | rs6609187 |
| rs4936876 | rs8095160 | rs11127808 | rs9494561 | rs5950943 |
| rs4322399 | rs1592643 | rs12494827 | rs3908195 | rs1799836 |
| rs2276084 | rs12960997 | rs9284802 | rs6912003 | rs2312466 |
| rs10893425 | rs2542177 | rs6785531 | rs4896369 | rs5953419 |
| rs493725 | rs12457255 | rs9876219 | rs6910623 | rs62591382 |
| rs494442 | rs2542157 | rs34506181 | rs7770507 | rs5905877 |
| rs1940365 | rs8090471 | rs12107900 | rs3015432 | rs6609338 |
| rs3802826 | rs9951698 | rs9860908 | rs9376510 | rs6611055 |
| rs548132 | rs12605006 | rs4859025 | rs12525884 | rs4824523 |
| rs7110832 | rs8096860 | rs13061110 | rs12192625 | rs4824393 |
| rs12416690 | rs1787013 | rs872976 | rs4896576 | rs6521004 |
| rs4937505 | rs476191 | rs66757381 | rs2175165 | rs5905934 |
| rs7122268 | rs498876 | rs1521806 | rs6570544 | rs4320664 |
| rs2324521 | rs55809306 | rs13082399 | rs9390084 | rs5905452 |
| rs10791179 | rs35664252 | rs4857135 | rs3736746 | rs12011108 |
| rs494050 | rs17681958 | rs7432402 | rs672468 | rs5906027 |
| rs3019856 | rs34176389 | rs9860147 | rs6927255 | rs2205717 |
| rs496848 | rs4797029 | rs1392695 | rs7744166 | rs851235 |
| rs10791302 | rs786000 | rs301921 | rs2144476 | rs4529579 |
| rs3019663 | rs62087736 | rs12492632 | rs6923492 | rs1207481 |
| rs4140962 | rs291795 | rs1520677 | rs6570789 | rs5953215 |
| rs10744725 | rs291781 | rs792831 | rs582437 | rs3014649 |
| rs12298813 | rs9949287 | rs7650727 | rs7758859 | rs4824716 |
| rs9805049 | rs518897 | rs9865411 | rs4895752 | rs7066028 |
| rs723672 | rs4302145 | rs13079711 | rs4897068 | rs6608814 |
| rs2074985 | rs1629847 | rs535173 | rs4609053 | rs2974420 |
| rs2191208 | rs56009341 | rs771785 | rs7762217 | rs5905712 |
| rs7973859 | rs2879223 | rs6441657 | rs655254 | rs2239453 |
| rs10774203 | rs12456739 | rs344670 | rs6901199 | rs28727466 |
| rs4766266 | rs273749 | rs6778525 | rs7349925 | rs4824458 |
| rs10744667 | rs12608116 | rs2398954 | rs2077647 | rs4824743 |
| rs1800830 | rs533813 | rs9837630 | rs214965 | rs2037973 |
| rs749928 | rs9952788 | rs9843391 | rs4145214 | rs179824 |
| rs2532520 | rs582813 | rs2399069 | rs2790092 | rs5915287 |
| rs7135073 | rs9960600 | rs2399083 | rs581564 | rs7472729 |
| rs9300125 | rs62086876 | rs13099754 | rs10782332 | rs1590666 |
| rs10842366 | rs12953390 | rs696377 | rs1385732 | rs11091784 |
| rs7310511 | rs1440824 | rs697951 | rs9371900 | rs6643518 |
| rs7973535 | rs11083386 | rs6786808 | rs4870428 | rs17276490 |
| rs2537759 | rs1035270 | rs12638235 | rs6926624 | rs9803358 |
| rs1316942 | rs276932 | rs2399272 | rs11753286 | rs982598 |
| rs7960118 | rs1026123 | rs1525874 | rs1270446 | rs4826370 |
| rs10772483 | rs1807376 | rs6802694 | rs9384488 | rs2516026 |
| rs12810992 | rs9949235 | rs1477840 | rs7751695 | rs5947383 |
| rs10845395 | rs4799711 | rs7610103 | rs287887 | rs4132452 |
| rs205539 | rs9961203 | rs1500045 | rs913971 | rs5989087 |
| rs2856344 | rs9807153 | rs3749297 | rs284426 | rs12393406 |
| rs2855743 | rs4291985 | rs1553222 | rs9968806 | rs5964746 |
| rs2710266 | rs7234973 | rs13064126 | rs7744146 | rs6624149 |
| rs4262801 | rs9964802 | rs12634339 | rs6909426 | rs4486345 |
| rs3020815 | rs12965996 | rs13066495 | rs9480440 | rs471205 |
| rs7308547 | rs6507197 | rs12053863 | rs7756488 | rs4827407 |
| rs2416988 | rs4799469 | rs67704344 | rs9397999 | rs5936890 |
| rs2160523 | rs1365467 | rs4682086 | rs142440005 | rs443731 |
| rs2302684 | rs7238205 | rs6766996 | rs9347744 | rs1938022 |
| rs10845494 | rs1681029 | rs4234415 | rs4612196 | rs5980712 |
| rs4763795 | rs6507347 | rs6798486 | rs9457773 | rs5936949 |
| rs11054848 | rs9965581 | rs13093633 | rs607256 | rs5936984 |
| rs10459098 | rs520522 | rs4682402 | rs262831 | rs5937005 |
| rs11054898 | rs1942075 | rs11712917 | rs3127221 | rs4320701 |
| rs10772570 | rs553470 | rs9845240 | rs2451253 | rs61175834 |
| rs10845582 | rs2156549 | rs2399456 | rs11754926 | rs3123571 |
| rs4555 | rs518023 | rs9875150 | rs641990 | rs3125945 |
| rs7330 | rs727055 | rs1114404 | rs1897106 | rs11796153 |
| rs2417232 | rs1460537 | rs9682734 | rs4142069 | rs6624539 |
| rs7964001 | rs582472 | rs2399496 | rs9456721 | rs2341629 |
| rs11055133 | rs2243803 | rs2654761 | rs7341370 | rs5937076 |
| rs1618317 | rs967137 | rs9842735 | rs4602703 | rs6525489 |
| rs867179 | rs718515 | rs4831113 | rs7751960 | rs3091305 |
| rs12815728 | rs4890662 | rs4429629 | rs563248 | rs941413 |
| rs6488576 | rs2684818 | rs6803436 | rs9456837 | rs6624621 |
| rs1872626 | rs11082646 | rs9811942 | rs6455872 | rs5937309 |
| rs11559837 | rs2291226 | rs4532128 | rs6455886 | rs6648379 |
| rs4763946 | rs357887 | rs35866 | rs10945889 | rs1620574 |
| rs10744039 | rs11082778 | rs6805251 | rs12195744 | rs5937796 |
| rs10845867 | rs10853592 | rs13075658 | rs1764020 | rs5938389 |
| rs7312794 | rs2850545 | rs679016 | rs2784867 | rs2097527 |
| rs11055774 | rs9304407 | rs2131142 | rs2759398 | rs5982362 |
| rs17340720 | rs2928937 | rs17282022 | rs2745365 | rs62612778 |
| rs7296115 | rs722758 | rs3732832 | rs6916725 | rs62613716 |
| rs6488679 | rs1502230 | rs9861904 | rs4709001 | rs28633285 |
| rs7302054 | rs1593199 | rs6778215 | rs9365596 | rs28593319 |
| rs7358578 | rs59572318 | rs12493045 | rs10046342 | rs28470890 |
| rs7298305 | rs63630165 | rs9809750 | rs9356148 | rs28602939 |
| rs2287538 | rs616580 | rs628716 | rs9365614 | rs6608433 |
| rs7977144 | rs1229591 | rs2922200 | rs9347796 | rs4826225 |
| rs10846054 | rs655784 | rs1839032 | rs9356165 | rs5959975 |
| rs2195199 | rs1657419 | rs16837548 | rs6907523 | rs4826262 |
| rs7294463 | rs62093611 | rs6775019 | rs473823 | rs5912177 |
| rs7310026 | rs158865 | rs2358566 | rs206696 | rs3088074 |
| rs7312494 | rs11873676 | rs10934834 | rs1325415 | rs5912359 |
| rs11056520 | rs11152108 | rs4927892 | rs6929642 | rs5912445 |
| rs1581606 | rs11152170 | rs34445363 | rs1413100 | rs35658443 |
| rs10846378 | rs2125020 | rs11713142 | rs6927989 | rs34609346 |
| rs10770199 | rs12963901 | rs1606489 | rs4710060 | rs2643580 |
| rs1163956 | rs36011282 | rs1393555 | rs1040445 | rs5959164 |
| rs4237972 | rs8094305 | rs4083132 | rs12153952 | rs12391144 |
| rs10459066 | rs9944883 | rs1869862 | rs3800521 | rs1606778 |
| rs10734683 | rs3017360 | rs9883661 | rs2342630 | rs5959021 |
| rs10770496 | rs1942317 | rs6786384 | rs7773460 | rs5959693 |
| rs12313000 | rs60940002 | rs4974504 | rs2982403 | rs1578480 |
| rs4762970 | rs2959485 | rs7637367 | rs9396995 | rs5912506 |
| rs4149030 | rs9950581 | rs9877457 | rs9348278 | rs473692 |
| rs3759229 | rs597949 | rs589092 | rs74200166 | rs825546 |
| rs2900501 | rs10163912 | rs28458819 | rs35986921 | rs2205922 |
| rs2466966 | rs4570974 | rs9847650 | rs4725261 | rs5923176 |
| rs1881904 | rs1073630 | rs774801 | rs12699839 | rs5968532 |
| rs2418110 | rs12961847 | rs537210 | rs1124581 | rs5923523 |
| rs7980156 | rs9949233 | rs9817734 | rs1915984 | rs2858685 |
| rs10842502 | rs8090697 | rs1580642 | rs10256761 | rs6524843 |
| rs11048073 | rs10871650 | rs11918457 | rs4720142 | rs5924213 |
| rs1064750 | rs7226654 | rs7613113 | rs13232425 | rs5941144 |
| rs7137176 | rs9945661 | rs6440006 | rs4585633 | rs1099988 |
| rs4964060 | rs1962549 | rs4234469 | rs1130329 | rs2987881 |
| rs10843117 | rs11663675 | rs7630115 | rs4320451 | rs62591006 |
| rs2086100 | rs12458789 | rs9869841 | rs6972587 | rs6619052 |
| rs11050088 | rs12607826 | rs7614192 | rs1105108 | rs4278299 |
| rs10843500 | rs9953495 | rs4431106 | rs34519436 | rs5983629 |
| rs1909182 | rs11151953 | rs9826226 | rs12671708 | rs2799932 |
| rs10771750 | rs7235594 | rs1373859 | rs13234328 | rs3130079 |
| rs35036 | rs12956201 | rs3804668 | rs6952703 | rs5949714 |
| rs73305977 | rs1460264 | rs35336826 | rs4320462 | rs5949957 |
| rs326638 | rs12458180 | rs13069376 | rs852526 | rs4828089 |
| rs1045751 | rs1944558 | rs1604499 | rs1997147 | rs5920957 |
| rs1486889 | rs2727054 | rs4274714 | rs852454 | rs2185737 |
| rs2389274 | rs4989135 | rs9809259 | rs2345937 | rs5921251 |
| rs11536200 | rs592993 | rs1502114 | rs13237614 | rs5967095 |
| rs10880642 | rs4799020 | rs1502111 | rs6944358 | rs17257181 |
| rs7137139 | rs2036892 | rs9855652 | rs2112009 | rs2858263 |
| rs12298504 | rs1317903 | rs2318780 | rs308094 | rs1046929 |
| rs550669 | rs758500 | rs66507831 | rs62456183 | rs5944886 |
| rs7977943 | rs2240149 | rs58262807 | rs1133132 | rs5987617 |
| rs10879360 | rs1610096 | rs56778250 | rs646156 | rs612440 |
| rs2730830 | rs35628322 | rs2089076 | rs67579350 | rs5917108 |
| rs7305104 | rs3783501 | rs4611784 | rs12667826 | rs62602928 |
| rs7137522 | rs8106438 | rs9874796 | rs889964 | rs5916736 |
| rs1517662 | rs1715092 | rs56329577 | rs6962646 | rs408928 |
| rs1501433 | rs4806946 | rs6440491 | rs836562 | rs1285715 |
| rs7953009 | rs7247419 | rs4632513 | rs9648100 | rs5962874 |
| rs583575 | rs4806949 | rs17511826 | rs6977663 | rs73255612 |
| rs7979280 | rs10416362 | rs9874843 | rs2042546 | rs5943427 |
| rs55669472 | rs10419790 | rs6804393 | rs3823839 | rs1548455 |
| rs7315711 | rs10401421 | rs1874921 | rs10252645 | rs5942935 |
| rs2408273 | rs12974218 | rs1498886 | rs12702887 | rs2404538 |
| rs2408269 | rs747833 | rs13079348 | rs1620397 | rs12846665 |
| rs7314254 | rs3745981 | rs958465 | rs6966636 | rs1024514 |
| rs11183146 | rs2379623 | rs1154910 | rs4725311 | rs2490125 |
| rs7974245 | rs2305106 | rs4681595 | rs4719260 | rs5974238 |
| rs6582568 | rs12978130 | rs12330655 | rs29 | rs1321391 |
| rs1468993 | rs350916 | rs358971 | rs35649285 | rs5905265 |
| rs2059404 | rs353694 | rs1042201 | rs2240574 | rs11260121 |
| rs247918 | rs2380179 | rs7640801 | rs17701711 | rs6645955 |
| rs11183228 | rs8111858 | rs6440833 | rs6959580 | rs12851089 |
| rs7298703 | rs105037 | rs1490544 | rs6968886 | rs5910465 |
| rs10785593 | rs919798 | rs7645542 | rs10278501 | rs5909664 |
| rs1896539 | rs4807602 | rs16824028 | rs1072291 | rs5909733 |
| rs1550736 | rs11085141 | rs2314612 | rs17169388 | rs5909804 |
| rs2099983 | rs778813 | rs173369 | rs6957731 | rs633959 |
| rs10083082 | rs874232 | rs7643130 | rs2537579 | rs6648722 |
| rs1492893 | rs1631032 | rs7625266 | rs17419024 | rs73227296 |
| rs4768109 | rs452767 | rs13080798 | rs1629017 | rs2473168 |
| rs12302984 | rs8103560 | rs1500921 | rs28693939 | rs7064260 |
| rs11183527 | rs8107977 | rs11712832 | rs28808719 | rs5956569 |
| rs7979810 | rs8104313 | rs12107104 | rs2529032 | rs3005872 |
| rs855136 | rs3787073 | rs340279 | rs6947890 | rs5956616 |
| rs215397 | rs3787062 | rs6441272 | rs10239423 | rs1279795 |
| rs7965582 | rs11085168 | rs525498 | rs1637097 | rs1292209 |
| rs6582709 | rs872664 | rs4091703 | rs4552807 | rs5911814 |
| rs11832679 | rs1051784 | rs1839019 | rs28406908 | rs2283769 |
| rs2408803 | rs7247153 | rs1843080 | rs16145 | rs6649243 |
| rs10875663 | rs11665713 | rs9871478 | rs876307 | rs616 |
| rs855272 | rs34039661 | rs206311 | rs10085787 | rs2097317 |
| rs1153978 | rs372959 | rs7614012 | rs6948634 | rs5930810 |
| rs7311663 | rs339395 | rs66469106 | rs2698721 | rs6636102 |
| rs1548518 | rs467439 | rs13073971 | rs7802771 | rs16309 |
| rs73107967 | rs11669654 | rs9842109 | rs6461961 | rs12558168 |
| rs2853559 | rs11878235 | rs9872042 | rs12700757 | rs512303 |
| rs7299460 | rs1593072 | rs9990094 | rs7795313 | rs6637497 |
| rs4547172 | rs889180 | rs4680600 | rs12700762 | rs2364565 |
| rs10875723 | rs10411146 | rs6444350 | rs7784257 | rs3115826 |
| rs10747529 | rs11879509 | rs35788852 | rs10256799 | rs3131269 |
| rs10875749 | rs6511685 | rs6787682 | rs2769365 | rs5932661 |
| rs10875754 | rs901886 | rs10936577 | rs3801835 | rs2050011 |
| rs4760697 | rs885743 | rs1705601 | rs213530 | rs3747340 |
| rs2450986 | rs34883264 | rs13070405 | rs2462901 | rs210005 |
| rs7312017 | rs10407947 | rs6799025 | rs3807592 | rs209231 |
| rs734684 | rs5977273 | rs5977302 | rs717972 | rs5933183 |
| rs5932758 | rs5977298 | rs4240127 | rs2144559 | rs7357884 |
| rs11835614 | rs10418278 | rs3980306 | rs1990123 | rs5930392 |
| rs10776467 | rs17324622 | rs5975507 | rs11795867 | rs1727456 |
| rs5930626 | rs5930641 | rs5975710 | rs5907890 | rs1556920 |
| rs2267527 | rs4830286 | rs5930988 | rs12855555 | rs5904938 |
| rs7883190 | rs5933429 | rs5931021 | rs875931 | rs5904735 |
| rs175327 | rs6529636 | rs4829896 | rs12845159 | rs2748479 |
| rs2312983 | rs5930659 | rs1935026 | rs17325065 | rs2719828 |
| rs5933390 | rs12858878 | rs1934137 | rs859931 | rs2742894 |
| rs5933393 | rs5978053 | rs5931627 | rs5919740 | rs5952028 |
| rs4448442 | rs35281265 | rs2223618 | rs2061420 | rs1861582 |
| rs5904762 | rs6525882 | rs5936474 | rs1970766 | rs4379586 |
| rs5952060 | rs5904672 | rs1582301 | rs9778461 | rs5924797 |
| rs2121749 | rs2392921 | rs5936206 | rs5924975 | rs4898415 |
| rs5904817 | rs9308372 | rs6540375 | rs6627842 | rs3761527 |
| rs215111 | rs59460742 | rs5980428 | rs5924641 | rs2266887 |
| rs17435 | rs56999285 | rs2004651 | rs11799036 | rs5945282 |
| rs7063300 | rs5945194 | rs5945224 | rs28810103 | rs3870343 |

Table 2 provides a list of 7590 SNPs from Table 2 of U.S. Provisional Application Ser. No. 63/880,627, incorporated herein by reference in its entirety.

In particular, in one aspect of the disclosure is provided a method for genetic screening a subject for a pediatric malignancy comprising obtaining a nucleic acid sample from the subject and selectively detecting in sequences of the nucleic acid sample the presence (or absence) of one or more mutations in an exonic, intronic, and/or promoter region of the genes of Table 1 and presence (or absence) of one or more single nucleotide polymorphisms of Table 2 thereby genetic screening the subject for a pediatric malignancy. As used herein, “genetic screening” refers to testing a biological sample from a subject to determine the genotype of the subject, where the genotype of the subject results in a specific phenotype or a specific phenotype. Thus, in accordance with this method, the genetic screen may be used to diagnose, prognose, determine the risk or predisposition of a subject for having or developing a pediatric malignancy. In some aspects, the method further comprises treating the subject for the pediatric malignancy based upon the one or more mutations and SNPs detected. By way of illustration, the detection of a RUNX1-RUNX1T1 gene fusion in a genetic screen may identify a subject as having or at risk of having AML such that the subject may be prophylactically or therapeutically treated with a chemotherapeutic agent such as cytarabine and/or an anthracycline.

In another aspect of the disclosure, a method of classifying a pediatric malignancy of a subject is provided, which comprises obtaining a nucleic acid sample from the subject and selectively detecting in sequences of the nucleic acid sample the presence (or absence) of one or more mutations in an exonic, intronic, and/or promoter region of the genes of Table 1 and the presence (or absence) of one or more single nucleotide polymorphisms of Table 2 thereby classifying the pediatric malignancy of the subject. As used herein, the term “classifying” refers to sorting individuals into different strata or classes based on the features of a pediatric malignancy. For example, classifying an individual with pediatric malignancy may involve assigning the individual on the basis of the type of the disease and/or phenotype. In some aspects, the presence of one or more of mutations in the genes of Table 1 and/or presence of one or more of the SNPS in Table 2 in a nucleic acid sample from a subject may classify the subject as having or at risk of having anaplastic large cell lymphoma, acute myeloid leukemia, acute megakaryoblastic leukemia, alveolar soft part sarcoma, atypical teratoid/rhabdoid tumor, B-lymphoblastic leukemia/lymphoma, B-lineage acute lymphoblastic leukemia, chronic myeloid leukemia, malignant tumor, ganglioglioma, adenocarcinoma, diffuse astrocytoma, germinoma, primitive neuroectodermal tumor, adamantinomatous craniopharyngioma, desmoplastic small round cell tumor, anaplastic ependymoma, ependymoma, Ewing sarcoma, gastrointestinal stromal tumor, glioblastoma, anaplastic astrocytoma, infantile fibrosarcoma high-grade glioma, low-grade glioma, pilocytic astrocytoma, desmoplastic/nodular medulloblastoma, medulloblastoma, large cell/anaplastic medulloblastoma, desmoplastic/nodular medulloblastoma, melanoma, malignant peripheral nerve sheath tumor, neuroblastoma, high-grade osteosarcoma, retinoblastoma, rhabdomyosarcoma, embryonal rhabdomyosarcoma, alveolar rhabdomyosarcoma, synovial sarcoma, desmoid/aggressive fibromatosis, dysgerminoma, sarcoma, hepatoblastoma, cutaneous squamous cell carcinoma, congenital mesoblastic nephroma, papillary renal cell carcinoma, high-grade undifferentiated pleomorphic sarcoma, poorly differentiated cutaneous (adnexal) carcinoma, desmoid/aggressive fibromatosis, T-cell acute lymphoblastic leukemia, papillary thyroid carcinoma, or Wilms tumor. In some aspects, the method further comprises treating the subject for the pediatric malignancy based upon the classification of the pediatric malignancy.

In another aspect, the disclosure provides a method for monitoring progression of a pediatric malignancy in a subject comprising obtaining a first nucleic acid sample from the subject at a first time point (e.g., at the first sign of symptoms of a pediatric malignancy or at the time of diagnosis of a pediatric malignancy); obtaining a second nucleic acid sample from the subject at a second time point (e.g., after therapeutic intervention or at regular intervals after remission); selectively detecting the presence (or absence) in sequences of the first and second nucleic acid samples one or more mutations in an exonic, intronic, and/or promoter region of the genes of Table 1 and the presence (or absence) of one or more single nucleotide polymorphisms of Table 2; comparing the presence (or absence) of the one or more mutations and one or more SNPs in the first nucleic acid sample with the presence (or absence) of the one or more mutations and one or more SNPs in the second nucleic acid sample thereby monitoring the progression of the pediatric malignancy. As an illustration of this method, the detection of a RUNX1-RUNX1T1 gene fusion in first nucleic acid sample from a subject exhibiting one or more symptoms associated with AML, e.g., fatigue, anemia, fever, bone pain, and/or weight loss may positively identify the subject as having AML. Upon remission of the AML, a second nucleic acid sample from the subject may show a loss or absence of the RUNX1-RUNX1T1 gene fusion in the second nucleic acid sample thereby indicating a reduced risk of cancer recurrence. In some embodiments, monitoring of the pediatric malignancy may identify the subject as being in need of treatment, e.g., the subject has not gone into remission. Accordingly, in some aspects, the method further comprises treating the subject for the pediatric malignancy based upon the progression of the pediatric malignancy.

In another aspect, a method for monitoring the response of a pediatric malignancy to a therapy in a subject is provided comprising obtaining a first nucleic acid sample from the subject prior to treatment with the therapy; obtaining a second nucleic acid sample from the subject after treatment with the therapy; selectively detecting in sequences of the first and second nucleic acid samples the presence (or absence) of one or more mutations in an exonic, intronic, and/or promoter region of the genes of Table 1 and the presence (or absence) of one or more single nucleotide polymorphisms of Table 2; comparing the presence (or absence) of the one or more mutations and one or more SNPs in the first nucleic acid sample with presence (or absence) of the one or more mutations and one or more SNPs in the second nucleic acid sample thereby monitoring the response of the pediatric malignancy to the therapy in the subject. As an illustration of this method, the detection of a gene fusion associated with a pediatric malignancy in a first nucleic acid sample from a subject and subsequent loss or absence of the gene fusion in a second nucleic acid sample obtained after treatment of the subject with a chemotherapeutic agent may be indicative of a positive response of the subject to the chemotherapeutic agent. In some embodiments, monitoring of the subject may identify the subject as not positively responding to a therapy such that the subject may require a higher dose of said therapy or a different therapy. Accordingly, in some aspects, the method further comprises treating the subject for the pediatric malignancy based upon the response of the pediatric malignancy to the therapy in the subject.

In some aspects, a biological sample may be plasma, cerebral spinal fluid, pleural fluid, ocular fluid, stool, urine, or a combination thereof. In some aspects, the biological sample may be a liquid biopsy sample (e.g., a cell-free DNA sample), tumor sample (e.g., solid tumor sample), or blood sample. In some embodiments, a biological sample may be obtained prior to the subject being diagnosed with a pediatric malignancy. In some embodiments, a biological sample may be obtained after cancer relapse. In some embodiments, a biological sample may be obtained upon remission. In some embodiments, a biological sample may be obtained at a first time point, e.g., at the time of diagnosis of the pediatric malignancy and at a second time point, e.g., after treatment and/or after remission. In some embodiments, a biological sample may be obtained from a subject prior to and after treatment with the therapy.

In some aspects of the methods herein, a nucleic acid sample is isolated, extracted, or otherwise obtained from a biological sample of a subject. In some aspects of the methods herein, a nucleic acid sample comprises the genomic DNA of a subject. In some aspects, a nucleic acid sample comprising the genomic DNA of a subject may comprise all or a portion of the subject's genomic DNA sequence, e.g., 50%, 55%, 60%, 65%, 70%, 75%, 80%, 85%, 90%, 95%, 96%, 97%, 98%, or 99% of the subject's genomic DNA sequence. In some embodiments, a nucleic acid sample comprising the genomic DNA of a subject may comprise intact genomic DNA, e.g., genomic DNA that has not been sheared or fragmented. In some embodiments, a nucleic acid sample comprising the genomic DNA of a subject may comprise sheared or fragmented genomic DNA.

In some aspects of the methods herein, the pediatric malignancy comprises anaplastic large cell lymphoma, acute myeloid leukemia, acute megakaryoblastic leukemia, alveolar soft part sarcoma, atypical teratoid/rhabdoid tumor, B-lymphoblastic leukemia/lymphoma, B-lineage acute lymphoblastic leukemia, chronic myeloid leukemia, malignant tumor, ganglioglioma, adenocarcinoma, diffuse astrocytoma, germinoma, primitive neuroectodermal tumor, adamantinomatous craniopharyngioma, desmoplastic small round cell tumor, anaplastic ependymoma, ependymoma, Ewing sarcoma, gastrointestinal stromal tumor, glioblastoma, anaplastic astrocytoma, infantile fibrosarcoma high-grade glioma, low-grade glioma, pilocytic astrocytoma, desmoplastic/nodular medulloblastoma, medulloblastoma, large cell/anaplastic medulloblastoma, desmoplastic/nodular medulloblastoma, melanoma, malignant peripheral nerve sheath tumor, neuroblastoma, high-grade osteosarcoma, retinoblastoma, rhabdomyosarcoma, embryonal rhabdomyosarcoma, alveolar rhabdomyosarcoma, synovial sarcoma, desmoid/aggressive fibromatosis, dysgerminoma, sarcoma, hepatoblastoma, cutaneous squamous cell carcinoma, congenital mesoblastic nephroma, papillary renal cell carcinoma, high-grade undifferentiated pleomorphic sarcoma, poorly differentiated cutaneous (adnexal) carcinoma, desmoid/aggressive fibromatosis, T-cell acute lymphoblastic leukemia, papillary thyroid carcinoma, or Wilms tumor.

In some aspects of the methods herein, the one or more mutations comprise a single nucleotide variant (SNV), small insertion and deletion (Indel), gene fusion, structural variant (SV), internal tandem duplication (ITD), single nucleotide polymorphism (SNP), copy number variation (CNV) and/or loss of heterozygosity (LOH). In some aspects, the one or more mutations comprise a SNV, Indel, gene fusion, SV, ITD, SNP, CNV and/or LOH in a gene provided in Table 1. In some embodiments, the one or more SNPs are provided in Table 2. In some embodiments, the presence of one or more mutations in a gene provided in Table 1 and/or the presence of one or more SNPs provided in Table 2 in a nucleic acid sample of a subject is indicative of a pediatric malignancy.

In accordance with any one of the methods herein, a subject with or at risk of having a pediatric malignancy as determined by a method herein, may be further treated for the pediatric malignancy. For example, a subject identified or classified as having ALL may be treated with a tyrosine kinase inhibitor (TKI) in combination with chemotherapy. Examples of suitable TKIs include, but are not limited to imatinib, dasatinib, nilotinib, bosutinib and ponatinib. A subject identified or classified as having relapsed or refractory acute leukemia may be treated with revumenib, whereas a subject identified or classified as having AML may receive treatment with a combination chemotherapies such as cytarabine and anthracyclines.

The administration of a therapeutic described herein may be, for example, by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.02 to about 100 mg/kg of body weight, alternatively dosages between 1 mg and 1000 mg/dose, every 4 to 120 hours, or according to the requirements of the particular drug. The methods herein contemplate administration of an effective amount of a therapeutic to achieve the desired or stated effect. Such administration may be used as a chronic or acute therapy. Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a subject's condition, a maintenance dose of a therapeutic agent may be administered, if necessary. the dosage or frequency of administration, or both, may be reduced, as a function of the symptoms, to a level at which the improved condition is retained when the symptoms have been alleviated to the desired level. Subjects may, however, require intermittent treatment on a long-term basis upon any recurrence of disease symptoms.

In some embodiments, the presence or absence of one or more of the mutations and SNPs described herein may be detected or determined by DNA sequence analysis using, e.g., Illumina NovaSeq and NextSeq platforms. In accordance with DNA sequence analysis, a DNA-seq library may be prepared from DNA isolated from a biological sample and subjected to target enrichment prior to sequence analysis. Alternatively, or in addition to, capture oligonucleotides may be used to detect putative SNVs, Indels, SVs, ITDs and CNVs. Single nucleotide polymorphisms may be detected by DNA sequencing analysis, high-throughput array-based genotyping, and/or PCR-based methods such as real-time PCR (e.g., TaqMan®) using probes for real-time fluorescent detection of SNPs during PCR, high-resolution melt analysis, kompetitive allele-specific PCR, amplification refractory mutation analysis using allele-specific primers to amplify different alleles in a single PCR reaction, loop-mediated isothermal amplification, and/or MassARRAY.

In some aspects, the pediatric malignancy results from or is associated with an oncogenic gene fusion. Accordingly, the present disclosure also provides a method for eliminating an oncogenic gene fusion-associated cancer cell by cleaving at least one neo splice site or cryptic exon of the oncogenic gene fusion. The term “eliminating,” “elimination,” or “eliminates” means to kill a cancer cell or otherwise diminish or reduce the number of cancer cells in a population of cells, e.g., by at least 10%, 20%, 30%, 40%, 50%, 60%, 70%, 80%, 90%, 95%, 98%, 99%, or even 100% compared to an untreated control population.

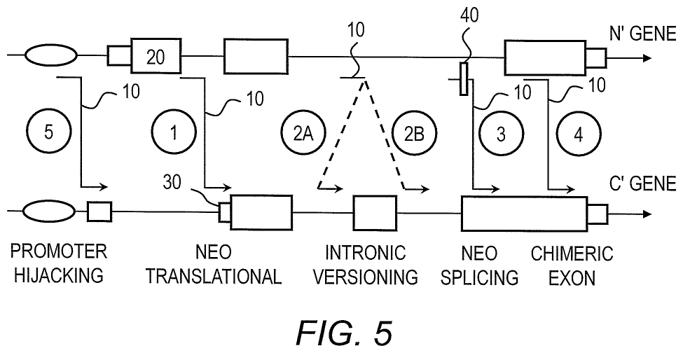

For the purposes of this invention, a neo splice site or cryptic exon is a genomic rearrangement which leads to a gene fusion that is not present in normal healthy cells (see FIG. 5). Thus, gene fusions in accordance with this invention are tumor-specific, cancer-inducing events and are therefore referred to as “oncogenic gene fusions.” In certain aspects, the gene fusion leads to the expression of a fusion product not present in normal healthy cells. In some aspects, the oncogenic gene fusion/gene fusion product is critical or essential to survival of the cancer cell such that cleaving or elimination of the gene fusion/gene fusion product is lethal to the cancer cell. In this respect, the “on-target/off-tumor” toxicity is extremely very low or absent.

The term “gene fusion” or “fusion gene” as used herein means the codifying region of a gene and also, the regulatory regions and other non codifying sequences such as promoters, enhancers and the like. In one aspect of this invention, the gene fusion includes at least one gene selected from MN1, PATZ1, CBFB, MYH11, C11orf95, NCOA2, TCF3, HLF, MAML2, BCOR, CCNB3, EWSR1, ATF1, CXXC5, TPM3, NTRK1, SPTBN1, ALK, FUS, FLI1, KAT6A, EP300, NUP98, BPTF, CBFA2T3, GLIS2, ATXN1, NUTM2B, MRC1, PDGFRB, YAP1, RELA, KDM5A, CIC or FOX04. In a preferred aspect of this invention, the oncogenic gene fusion is selected from MN1-PATZ1, CBFB-MYH11, C11orf95-NCOA2, TCF3-HLF, C11orf95-MAML2, BCOR-CCNB3, EWSR1-ATF1, MN1-CXXC5, TPM3-NTRK1, SPTBN1-ALK, FUS-FLI1, KAT6A-EP300, NUP98-BPTF, EP300-BCOR, CBFA2T3-GLIS2, C11orf95-MAML2, ATXN1-NUTM2B, MRC1-PDGFRB, C11orf95-YAP1, C11orf95-RELA, NUP98-DM5A or CIC-FOXO4.

As used herein, the term “cleaving”, “cleave” or “cleavage” means that one or both strands or chains of a DNA molecule (e.g., genomic DNA) are cut or one strand or chain of an RNA molecule (e.g., mRNA) is cut. Upon genome cleavage, when a double stranded molecule is cut, both sticky and blunt ends may be generated as a result of the cleavage. Ideally, cleavage of the oncogenic gene fusion in the genome leads to a deletion, an inversion, a frameshift or any combination thereof. In some aspects, cleavage does not result in the insertion of an exogenous gene, e.g., a suicide gene, as described in WO 2016/094888 A1. In some aspects, the method includes cleaving at least one, two, three, four, five or more sites of the gene fusion. Therefore, the method may include cleaving in at least one site to hundreds of sites, in cases where the genomic rearrangement includes hundreds of repetitions of a cancer-inducing oncogenic fusion gene.

In certain aspects of the invention, the cleavage is performed by at least one endonuclease. In one aspect, the endonuclease may be a CRISPR-related protein such as Cas protein, in particular a Cas9 protein, or a functional equivalent thereof, whose target site is driven by the sequence of a guide RNA. As used herein, the term “guide RNA” and “single guide RNA” are used interchangeably and are abbreviated as “gRNA” and “sgRNA.” As known in the art, ˜20 nucleotide spacer (or target domain or target sequence) of the gRNA defines the DNA or RNA target to be modified by the CRISPR-related protein. In particular, the target domain of the gRNA is designed to have complementarity, where hybridization between a target sequence and a guide sequence promotes the formation a CRISPR of complex. Full complementarity is not necessarily required, provided there is sufficient complementarity to cause hybridization and promote formation of a CRISPR complex. In certain aspects of this invention, a gRNA is provided, the target domain of which is complementary to at least one neo splice site or cryptic exon of an oncogenic gene fusion.

Exemplary Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c2, C2c3, homologs thereof, or modified versions thereof. These enzymes are known, for example, the amino acid sequence of S. pyogenes Cas9 protein may be found in the SwissProt database under accession number Q99ZW2. In some aspects, the unmodified CRISPR enzyme has DNA cleavage activity, such as Cas9. In some embodiments the CRISPR enzyme is Cas9 and may be Cas9 from S. pyogenes or S. pneumoniae.

In another aspect, the cleavage is done using endonuclease Cas13. Cleavage by Cas13 of RNA encoding a fusion gene is exclusive of the cancer cells and leads to the degradation of the RNA in the cell and eventually to its death. The Cas13 enzyme is a CRISPR RNA (crRNA)-guided RNA-targeting CRISPR effector. Under the guidance of a single crRNA, Cas13 can bind and cleave a target RNA carrying a complementary sequence. Through this mechanism, the CRISPR-Cas13 system can effectively knockdown mRNA expression in mammalian cells with an efficacy comparable with RNA interference technology and with improved specificity. Accordingly, in some aspects, Cas13 and crRNA are used in the methods of this invention to target a oncogenic gene fusion mRNA, in particular a cryptic exon of the mRNA.

Also, cleaving may be performed by endonucleases such as a zinc-finger nucleases (ZFN) or transcription activator-like effector nucleases (TALEN). Both of these approaches involve applying the principles of protein-DNA interactions of these domains to engineer new proteins with unique DNA-binding specificity. These methods have been widely successful for many applications.

In a preferred aspect of the method, cleavage is in a neo splice site of the oncogenic gene fusion. Splice sites are found at the 5′ and 3′ ends of introns. Most commonly, the RNA sequence that is removed begins with the dinucleotide GU at its 5′ end and ends with AG at its 3′ end. These consensus sequences are known to be critical, because changing one of the conserved nucleotides results in inhibition of splicing. Accordingly, in one aspect, a CRISPR-related protein such as Cas9 is used to cleave a neo splice site and the target domain of the gRNA (therefore the cleavage sequence) is specific for the neo splice site. As demonstrated herein, cleaving the genome of the cancer cells at two neo splice sites leads to the death of the cancer cell. Thus, in certain aspects, the methods of this invention provide for the use of at least two gRNA to cleave two neo splice sites. Preferred gRNAs are those codified by sequences (SEQ ID NOs: 1-7), useful for cleaving the TCF3-HLF fusion gene.

Another aspect of this invention provides for a kit for cleaving at least one neo splice site or cryptic exon of an oncogenic gene fusion. In one aspect, the kit includes (a) a CRISPR-associated endonuclease, preferably a Cas protein, more preferably Cas9 or a functional equivalent thereof; and (b) at least one or two gRNA to target the cleaving of the genome, preferably at a neo splice site or cryptic exon. In certain aspects, the kit includes one or more gRNAs as set forth in SEQ ID NOs: 1-7, which target neo splice sites of a TCF3-HLF fusion gene.

In a further aspect, the invention provides a kit including an endonuclease capable of cleaving a messenger RNA (mRNA), i.e., CRISPR associated protein Cas13 or another endonuclease derived from said Cas13 or a functional equivalent thereof (or a sequence coding said endonuclease); and at least one gRNA, i.e., crRNA, having a targeting domain specific for a cryptic exon of an oncogenic gene fusion.

Alternatively, a kit of the invention can include at least one of a zinc-finger nuclease (ZFN) or a transcription activator-like effector nuclease (TALEN), wherein said endonuclease specifically cleaves the genome at a neo splice site or cryptic exon of an oncogenic gene fusion. The kit may include the endonuclease or a sequence coding said endonuclease, preferably in an expression vector.

Another aspect of the present invention relates to the use of the methods or kits of the invention in the treatment of cancer. There are a number of cancers known in the art to be associated with or result from oncogenic gene fusions. Such cancers and their corresponding gene fusions are listed in Table 3.

TABLE 3

| | |
|---|---|
| Cancer | Oncogenic Gene Fusion |

Leukemias

| | |
|---|---|
| Acute myeloid leukemia | RUNX1-RUNX1T1, CBFB-MYH11, |
| (AML) | KMT2A-MLLT3, RPN1-MECOM, |
| | DEK-NUP214, PVT1-MECOM, |
| | RUNX1-MECOM, KMT2A-MLLT10, |
| | NUP98-NSD1, KMT2A-AFDN, |
| | CBFAT3-GLIS2, NUP98-KDM5A, |
| | FUS-ERG, HNRNPH1-ERG, |
| | KMT2A-SEPTIN6, KAT6A- |
| | CREBBP, RUNX1-CBFA2T3 |
| Acute promyelocytic leukemia | PML-RARA, ZBTB16-RARA |
| (APL) | |
| Acute lymphocytic leukemia | ETV6-RUNX1, BCR-ABL1, TCF3- |
| (ALL) | PBX1, KMT2A-AFF1, PICALM- |
| | MLLT10, IGH-CEBPA, TCF3-HLF, |
| | TRA-MYC, KMT2A-MLLT1, |
| | KMT2A-ELL, MEF2D-BCL9, |
| | EP300-ZNF384, TCF3-ZNF384, |
| | P2RY8-CRLF2, STIL-TAL1 |
| Chronic myeloid leukemia | BCR-ABL1 |
| (CML) | |
| Chronic lymphocytic | IGH-BCL1, IGH-BCL2, IGH-BCL3 |
| leukemia (CLL) | |

Sarcoma/Bone

| | |
|---|---|
| Ewing's sarcoma | EWSR1-FLI1, EWS-ERG, EWS- |
| | ETV1, EWS-FEV, EWS-E1AF |
| Alveolar rhabdomyosarcoma | PAX3/FOX01, PAX7-FOX01 |
| (RMS) | |
| Congenital spindle cell RMS | VGLL2-CITED2, VGLL2-NCOA2, |
| | TEAD1-NCOA2 |
| Alveolar soft-part sarcoma | ASPSCR1-TFE3 |
| Extraskeletal myxoid | EWS-TEC, TAF2N-TEC |
| chondrosarcoma | |
| Fibromyxoid sarcoma | FUS-CREB312 |
| Endometrial stromal sarcoma | JAZF1-JJAZ1 |
| Angiomatoid fibrous | EWSR1-CREB1, FUS-ATF1, |
| histiocytoma | EWSR1/ATF1 |
| Juvenile fibrosarcoma | ETV6-NTRK3 |
| Myxoid chondrosarcoma | EWS-NR4A3, TFC12-NR4A3, |
| | TAF2N-NR4A3, TAF15-NR4A3 |
| Synovial sarcoma | SYT-SSX1, SYT-SSX2, |
| | SYT-SSX4 |
| Mixoid liposarcoma | FUS-CHOP, EWS-CHOP |
| Spindle cell sarcoma | MLL4-GPS2 |
| Dermatofibrosarcoma | COL1A1-PDGFB |
| protuberans (DFSP) | |
| Clear cell sarcoma | EWS-ATF1 |
| Soft tissue angiofibroma | AHRR-NCOA2 |
| Undifferentiated round cell | BCOR-CCNB3, CIC-DUX4L10, |
| sarcoma (URCS) | CIC-DUX 4 |
| Chondroid lipoma | C11ORF95-MKL2 |
| Mesenchymal chondrosarcoma | HEY1-NCOA2 |
| Biphenotypic sinonasal | PAX3-M4ML3 |
| sarcoma | |
| Despoplastic small round | EWS-WT1 |
| cell tumor | |

Lymphomas

| | |
|---|---|
| Follicular lymphoma | BCL2-IGH |
| Mantle lymphoma | BCL1-IGH |
| Large cell lymphoma | NPM-ALK |
| Burkit lymphoma | MYC-IGH |

Brain Tumors

| | |
|---|---|
| Pilocytic astrocytoma | KTAA1549-BRAF |
| Glioblastoma | TPM3-NTRK1, FGFR3-TACC3 |
| Gliomas | MYB-QKI, PPP1CB-ALK, |
| | KIAA1549-BRAF |
| Sporadic pilocytic | KIAA1549-BRAF |
| astrocytomas/some pedriatic | |
| brain tumors | |
| Supratentorial ependymomas | C11orf95-RELA, YAP1-FAM118B |
| Meningioma | MN1-ETV6 |

Liver Tumors

| | |
|---|---|
| Fibrolamellar hepatocellular | DNAJB1-PRKACA, LRIG3/ROS1 |
| carcinoma | |

Kidney Tumors

| | |
|---|---|
| Clear renal cell carcinoma | SFPQ-TFE3, TFG-GPR1228 |
| Mesoblastic nephroma | ETV6-NTRK3 |
| Renal cell carcinoma | MALAT1-TFEB |

Lung Tumors

| | |
|---|---|
| Lung adenocarcinoma | EML4-ALK, LRIG3/ROS1 |
| Non-small cell carcinoma | EML4/ALF |

Prostate Tumors

| | |
|---|---|
| Prostate cancer | TMPRSS2-ERG |

Breast/Ovarian Tumors

| | |
|---|---|
| Breast cancer | BCAS4-BCAS3, TEL1XR1-RGS17, |
| | ODZ4-NRG1 |
| Secretory breast cancer | ETV6-NTRK3 |
| Serous ovarian carcinoma | ESRRA-C11orf20 |

Colon Tumors

| | |
|---|---|
| Colorectal Cancer | PTPRK-RSPO3, TPM3-NTRK1, |
| | EIF3E-RSPO2 |

Bladder Tumors

| | |
|---|---|
| Bladder cancer | FGFR3-TACC3 |

Salivary Gland Tumors

| | |
|---|---|
| Mucoepidermoid carcinomas | MECT1-MAML2 |
| Adenoid cystic carcinoma | MYC-NFIB |
| Pleomorphic adenoma | CTNNB1-PLAG1 |

Endocrine Cancers

| | |
|---|---|
| Papillary thyroid cancer | ETV6-NTRK3 |
| (PTC) | |
| follicular thyroid cancer | PAX8-PPARG |

Endocrine Cancers

| | |
|---|---|
| Aggressive midline carcinoma | BRD4-NUT |
| Melanoma of soft parts | EWSR1-ATF1 |
| Gastric cancer | CD33-SLC1A2 |

Accordingly, the present invention also provides a method for treating a subject with an oncogenic gene fusion-associated cancer by administering an effective amount of an exogenous endonuclease that cleaves at least one neo splice site or cryptic exon of the oncogenic gene fusion of the subject. The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, and the delivery system in which it is carried.

The exogenous endonuclease can be any one of a CRISPR-associated protein, a ZFN and/or TALEN described herein. As will be understood by disclosure elsewhere herein, when using a CRISPR-associated protein such as a Cas protein, in particular a Cas9 protein, one or more gRNAs are also administered to target the CRISPR-associated protein to the target neo splice site or cryptic exon.

Cancers that can be treated in accordance with the methods of this invention include, but are not limited to, leukemias, sarcomas, lymphomas, brain cancer, liver cancer, kidney cancer, lung cancer, prostate cancer, breast cancer, ovarian cancer, colon cancer, bladder cancer, salivary gland cancer, endocrine cancer, and gastric cancer. In certain aspects, the methods of this invention are used in the treatment of a leukemia. In particular aspects, the methods of this invention are used in the treatment of ALL, AML, APL, CML or CLL. Preferably, treatment is of cancers where there is a genomic rearrangement present in a cancer cell which leads to the expression a fusion gene not present in non-cancer cells. More preferably, treatment is of the cancers listed in Table 3. In some embodiments, a kit of the present invention is used. In this respect, the components of the kit are delivered to the patient in need of the treatment by specific delivery systems that are known to be useful in each particular cancer type.

The terms “subject” and “patient” are used interchangeably herein. The subject treated by the present methods is desirably a human subject, although it is to be understood that the methods described herein are effective with respect to all vertebrate species, which are intended to be included in the term “subject.” Accordingly, a “subject” can include a human subject or an animal subject. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some aspects, the subject is a human including, but not limited to, fetal, neonatal, infant, juvenile, and adult subjects.

Delivery systems include conventional viral and non-viral based gene transfer methods used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome, nanoparticle or macrocomplex. Viral vector delivery systems include DNA and RNA viruses, which have either episomal or integrated genomes after delivery to the cell. For a review of gene therapy procedures, see Anderson (1992) Science 256:808-813; Nabel & Felgner (1993) TIBTECH 11:211-217; Mitani & Caskey (1993) 11:162-166; Dillon (1993) TIBTECH 11:167-175; Miller (1992) Nature 357:455-460; Van Brunt (1998) Biotechnology 6 (10): 1149-1154; Vigne (1995) Restorative Neurology and Neuroscience 8:35-36; Kremer & Perricaudet (1995) British Medical Bulletin 51 (1): 31-44; Haddada et al. (1995) Current Topics in Microbiology and Immunology. Doerfler and Bohm (eds); and Yu et al. (1994) Gene Therapy 1:13-26.

Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in, e.g., U.S. Pat. Nos. 5,049,386, 4,946,787 and 4,897,355. Cationic and neutral lipids that are suitable for efficient receptor-recognition lipofection of polynucleotides include those described in WO 1991/17424 and WO 1991/16024. Delivery can be to cells (e.g., in vitro or ex vivo administration) or target tissues (e.g., in vivo administration).

Treatment according to the present methods can result in complete relief or cure from a cancer, or partial amelioration of one or more symptoms of the cancer, and can be temporary or permanent. The term “treatment” also is intended to encompass therapy and cure.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of the particular agent chosen, the dosing regimen to be followed, whether it is administered in combination with other compounds, timing of administration, the tissue to be imaged, and the physical delivery system in which it is carried.

The administration of kit components, i.e., endonuclease and optional gRNA, can be via different ways, depending on the target tissue or cancer cell in the patient. Thus, the administration may be oral or parenteral, subcutaneous, intramuscular or intravenous, as well as intrathecal, intracranial, etc., depending on the patient needs.

Also provided herein is a kit for detecting or determining the presence or absence of one or more mutations in non-coding regions (e.g., exonic, intronic, and/or promoter regions) of the genes of Table 1 and single nucleotide polymorphisms of Table 2. Such a kit may include, e.g., capture oligonucleotides, probes, and/or primers for PCR-based methods and/or sequence analysis. In some embodiments, a capture oligonucleotide, a primer and/or probe herein may include a label (e.g., fluorescent dye), an adaptor and/or barcode to facilitate detection and/or analysis.

The following non-limiting examples are provided to further illustrate the present invention.

### Example 1: Pan-Cancer Gene Panel for Childhood Malignancies

Panel design. Based on extensive research and literature review of pan-cancer genome profiling studies a list of exonic and/or intronic regions (n=5,009 regions, 2.82 genes that are frequently implicated in Mbp) from 357 pediatric cancers was compiled to detect single nucleotide variants (SNVs), small insertions & deletions (Indels), gene fusions, structural variants (SVs), and internal tandem duplications (ITDs). A list of 7,590 single nucleotide polymorphic markers (SNPs) evenly spread across human chromosomes was curated to detect large genomic structural rearrangements such as copy number variations (CNVs) and loss of heterozygosity (LOH). The details of all the genomics regions and SNPs used to assemble the pediatric pan cancer, termed as “SJPedPanel,” are included herein as Tables 1 and 2, respectively. In particular, Table 1 provides a list of 5009 exonic/intronic/promoter regions from 357 genes of the human genome (GRCh37/hg19) included in the SJPedPanel, wherein “Chrom” refers to chromosome number, “Start” refers to start position of the region (GRCh37/hg19), “End” refers to end position of the region (GRCh37/hg19), “Gene” refers to gene name, “Transcript Isoform” refers to Accession number of the transcript isoform used for the gene, “Length” refers to length of the region, “Covered Exon(s)” refers to exons covered by the region, and “Covered Intron(s)” refers to intron covered by the region with at least 80% of the corresponding intron length (Note: The regions were padded with ˜10 bases flanking the mentioned exons). Table 2 provides a list of 7590 SNPs from human genome included in the SJPedPanel, wherein the top level column header indicates the corresponding coordinate and allele frequencies between GRCh37/hg19 from gnomAD v2.1.1 and the GRCh38/hg38 from gnomAD v4.1.0, “Chrom” refers to chromosome number, “POS” refers to position in the genome, “ID” refers to rsID from dbSNP database at NCBI, “REF” refers to reference allele, “ALT” refers to alternate allele, “MAF” refers to minor allele frequency of the corresponding SNP as reported in corresponding version of the gnomAD database, and “Comment” provides a description for any discrepancies between a few number of SNP records from gnomAD v2.1.1 and v4.1 (Note: All the analyses were performed using SNP details mentioned for gnomAD v2.1.1; GRCh37/hg19).

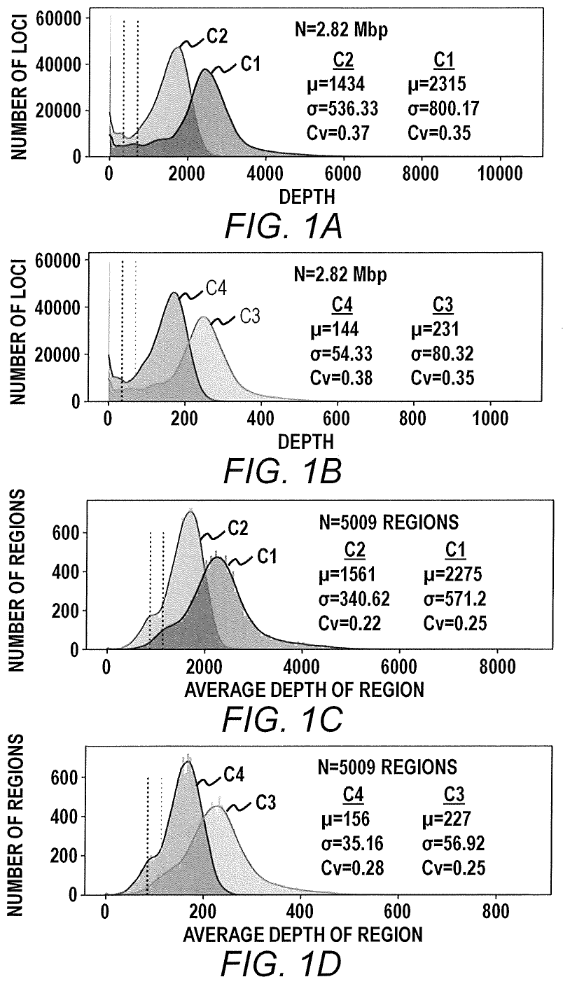

Capture efficiency of the SJPedPanel. One high-depth (˜2000×) and one low-depth (˜200×) targeted sequencing libraries were generated (two replicates) using SJPedPanel using COLO829BL cell line (a gold-standard cell line for clinical validation and other established studies (Craig et al. (2016) Sci Rep 6:24607; Pleasance et al. (2010) Nature 463:191-196). These libraries were sequenced on Illumina NovaSeq and NextSeq platforms, respectively. The data generated was used to evaluate the capture performance of the probes and uniformity of coverage across regions and loci of the SJPedPanel (FIGS. 1A-1D).

Dilution experiment. A dilution experiment using 6 cancer cell lines (ME-1, 697, Rh30, EW-8, K562, Molm13, courtesy of Elizabeth Stewart, Department of Oncology, St. Jude Children's Research Hospital) and a non-cancer cell line (GM12878) was designed to achieve seven tumor concentrations with two replicates each. The seven dilutions were divided in three groups (a) ultra-low (0.1%, 0.2%), (b) low (0.5%, 1%), and (c) medium (2.5%, 5% and 10%), which were sequenced at depths of 10,000×, 5000× and 2500× respectively. The cell lines were also sequenced independently in undiluted forms at 25,000× to estimate the original allele fractions of 26 cell-line-specific markers (14 SNVs, 4 Indels, 8 SVs; all these markers are confirmed to be exclusively detected from one of the six cell lines). Recall rate of these known markers across different dilutions was used to assess the limit of detection of the SJPedPanel.

The limit of detection (LoD) was determined by two critical factors: (1) the sequencing depth (also known as power) and (2) the noise level. For example, if the true allele fraction is 18, a sequencing depth of 913× will ensure 95% chance of detecting this variant with >=5 mutant alleles (Ma et al. (2020) Quantitative Biology 8:15-108). In consideration of the high range of dilution concentrations, the goal was 2, 500× depth for dilution ladders >18, 5,000× for ladders 0.5% and 18, and 10,000× for ladders 0.1% and 0.2%. On the other hand, the noise level is typically regarded as background error rate. Mutations with higher background error rates are more difficult to detect because the true signal can be overwhelmed by the background noises. Computational error suppression methods have been developed to achieve error rate of ˜Oct. 6, 2010-4 for substitutions (Ma et al. (2019) Genome Biol 20:50) and similar methods and results have been achieved for Indels and SVs.

In silico down sampling experiment. In silico down sampling of data from a set of cancer cell line dilution samples was performed to evaluate the trade-off between recall rate, down sampled depth of sequencing and associated cost estimates. The samples originally sequenced at 2, 500× were further down sampled to simulate depths of sequencing at 1,000×, 1,500× and 2,000×, whereas the samples which were sequenced at 5,000× and 10,000× were down sampled to simulate depths of sequencing at 1,000×, 1,500×, 2,000×, 3,000×. For each of the desired down sampled depth, 10 samples were simulated, each consisting of randomly sampled reads at loci of 14 SNVs. These simulated samples were used to determine the trade-off between recall rate and depth of sequencing.

Investigating diagnostic yield using clinical sample re-sequencing. Based on sample availability 113 specimens were selected from previously sequenced pediatric cancer cases treated at St. Jude Children's Research Hospital to represent a wide range of cancer subtypes common in pediatrics. Samples were chosen primarily from the pilot study cohort (n=40) (Rusch et al. (2018) Nat. Commun. 9:3962) and G4K studies (n=73) (Newman et al. (2021) Cancer Discov 11:3008-3027) and had previously reported clinically relevant markers identified by triple platform approach of whole genome, whole exome, and transcriptome sequencing. A list of cases and their cancer subtypes used for these purposes is provided in Table 3 of U.S. Provisional Application Ser. No. 63/880,627, incorporated herein by reference in its entirety. A demographic summary is provided herein in Table 4 below.

TABLE 4
Values/Categories Number a

| | |
|---|---|
| Demographic | |
| Factor | Values/Categories Number ^{a} % |

| | | | |
|---|---|---|---|
| Sex | Male | 66 | 58.41 |
| | Female | 47 | 41.59 |
| Age at initial | Median: 8 | | |
| diagnosis | Range: 1-20 | | |
| (year) | IQR: 3-12 | | |
| Pediatric sub- | Middle childhood (6 | 36 | 32.43 |
| groups | to <11 years) | | |
| (age range) | Early adolescence (11 | 26 | 23.42 |
| | to <16 years) | | |
| | Early childhood (2 | 17 | 15.32 |
| | to <6 years) | | |
| | Toddler (1 to <2 | 13 | 11.71 |
| | years) | | |
| | Late adolescence (16 | 12 | 10.81 |
| | to <21 years) | | |
| | Infant (1 month | 7 | 6.31 |
| | to <1year) | | |
| Ethnicity | Not Hispanic, Latino/a, | 97 | 85.84 |
| | or Spanish origin | | |
| | South or Central | 5 | 4.42 |
| | American | | |
| | Other Hispanic, | 4 | 3.54 |
| | Latino/a, or Spanish | | |
| | origin | | |
| | Mexican, Mexican | 3 | 2.65 |
| | American, or Chicano/a | | |
| | Puerto Rican | 2 | 1.77 |
| | Non-Hispanic | 1 | 0.88 |
| | Hispanic or Latino | 1 | 0.88 |
| Nationality | White, NOS | 42 | 38.89 |
| | North American | 39 | 36.11 |
| | Black, NOS | 9 | 8.33 |
| | South/Central American | 5 | 4.63 |
| | African American | 5 | 4.63 |
| | Asian Indian (India) | 3 | 2.78 |
| | Asian, NOS | 1 | 0.93 |
| | Vietnamese | 1 | 0.93 |
| | Pakistani | 1 | 0.93 |
| | Middle East/No. Coast | 1 | 0.93 |
| | of Africa | | |
| | Caribbean | 1 | 0.93 |
| Race | White | 83 | 74.77 |
| | Black | 17 | 15.32 |
| | Asian | 6 | 5.41 |
| | Black and White | 2 | 1.8 |
| | Other | 1 | 0.9 |
| | Multiple Race (NOS) | 1 | 0.9 |
| | Asian and White | 1 | 0.9 |

The recall rate of clinically relevant markers from these cases was used to establish the diagnostic yield of the SJPedPanel. Here seven markers from 3 hypermutator cases (SJHGG030335, SJHGG030336 and SJST030211) were downgraded to VUS (variant of unknown significance) for this analysis per communication with corresponding author of the G4K study, resulting in 140 of SNV/Indel, 55 of fusion/SVs, 184 CNV/LOH and 10 ITDs (total=389).

Library preparation, capture, and sequencing. DNA samples were obtained and subjected to DNA-seq library preparation and target enrichment followed by sequencing in the Clinical genomics laboratory as described below. An input of 100 ng of DNA was used to construct libraries using the Twist Library Preparation Enzymatic Fragmentation (EF) Kit 2.0 (Twist Biosciences, CA) following the manufacturer's instructions. Capture oligos were designed to detect putative SNVs, Indels, SVs, ITDs and CNVs in 357 genes of clinical interest. SJPedPanel was synthesized at Twist Biosciences and is described in detail in the section on panel design. Eight libraries were pooled at a time and target enrichment for the SJPedPanel baits was carried out using Twist hybrid capture protocol following manufacturer's instruction. Paired-end 150-cycle sequencing was performed on NovaSeq or NextSeq instruments (Illumina Inc, CA) as appropriate. Where necessary, additional sequencing (“top off”) was performed to ensure that a sequencing depth of at least 1000× was achieved in all cases.

Early detection of relapsed AML cases. To evaluate the panel's capability for disease monitoring, two pediatric AML cases (SJAML016582 and SJAML016551) with material available at diagnosis, relapse, and remission timepoints were analyzed. Both samples provide multiple trackable somatic markers, including structural variants and SNVs. Samples were subjected to deeper sequencing depths of >5000× after targeted capture to ensure detection of low-level variants at <18. Average of variant allele fractions (VAF) of detected somatic variants were used to estimate tumor burden at corresponding time points.

Coverage comparison between SJPedPanel and Whole exome sequencing. The content of SJPedPanel was compared with that of whole exome sequencing (WES) manifest to highlight the differences in coverage of hg19 genomic regions. An Illumina Exome 2.0 Plus hg19 BED file padded with 10 bp was used for region intersection analyses. Reported somatic variants from the Genomes for Kids (G4K) (Newman et al. (2021) Cancer Discov 11:3008-3027), a real-time three-platform sequencing study of 309 pediatric cancer patients to benchmark the coverage of reported pathogenic and likely pathogenic variants between SJPedPanel and WES was utilized.

Comparison of SJPedPanel with other panels. The content of the SJPedPanel was compared with DNA content of five other commercially available panels including FoundationOne Heme, FoundationOne CDx by Foundation Medicine Inc., MSK-IMPACT (Cheng et al. (2015) J. Mol. Diagn. 17:251-264) by Memorial Sloan Kettering Cancer Center, OncoKids (Hiemenz et al. (2018) J. Mol. Diagn. 20:765-776) by Children's Hospital Los Angeles and Oncomine Comprehensive Assay v3 by Thermo Fisher Scientific Inc. These panels collectively represent the breadth and diversity of clinical gene panels. Since most of the providers do not provide the exact coordinates of the regions in the panel, the content of these panels was compared using standardized gene names with the help of official gene symbols and synonyms from the NCBI gene database (Brown et al. (2015) Nucleic Acids Res 43: D36-42).

Bioinformatics analyses. The adapter trimmed paired end FASTQ files generated on the Illumina NovaSeq/NextSeq platforms were assessed for sequence and instrument quality using FastQC v0.11.9 and SequencErr v2.0.9. The reads were mapped against GRCh37 build using BWA aln 0.7.12-r1039. The utility commands in SAMtools v1.7 and BEDTools v2.25.0 were used perform simple operations using BAM and BED files. The count files obtained from BAM files using SequencErr were further passed as an input to DeepSeqCoverageQC v0.3.1 to compute depth of coverage QC metrics of the sequenced samples over loci/regions of the SJPedPanel. The genotyping of SVs and Indels to compute the allele fractions was carried out using SVIndelGenotyper. The CNVs were detected using CNVkit v0.9.10. The BAM files of 30 germline samples were used to create a pooled reference of per-bin copy number estimates. The segment & bin-level call files along with CNV diagrams generated by CNVkit batch command were used to review the CNV calls in tumor samples. To determine the LOH in sequenced samples, the minor allele frequencies of 7590 SNPs were computed using count files generated by SequencErr and subsequently used to generate allelic imbalance plots over chromosomes. The output files and diagrams generated by CNVkit v0.9.10 and the allelic imbalance figures used to review CNV and LOH events are available from Zenodo repository.

A “rotation control” method (Li et al. (2020) Blood 135:41-55) was used to obtain the background count of the variants for binomial testing and Q-values were used to assess the statistical significance of detection based on binomial testing. Briefly, frequency of any variant from the original mutation-positive sample was considered as foreground (V_{f}) and frequency of the same variant derived from the aggregated counts from rest of the samples, which were expected to be wild type in the original analysis, was considered as background or control (V_{b}) for the binomial testing. Thus, the background counts were rotated w.r.t. change in the foreground variant and sample of interest. A sample is called mutation-positive if the Q-value was found to be <0.05. All the statistical analyses were performed using R v4.0.3⁹⁸.

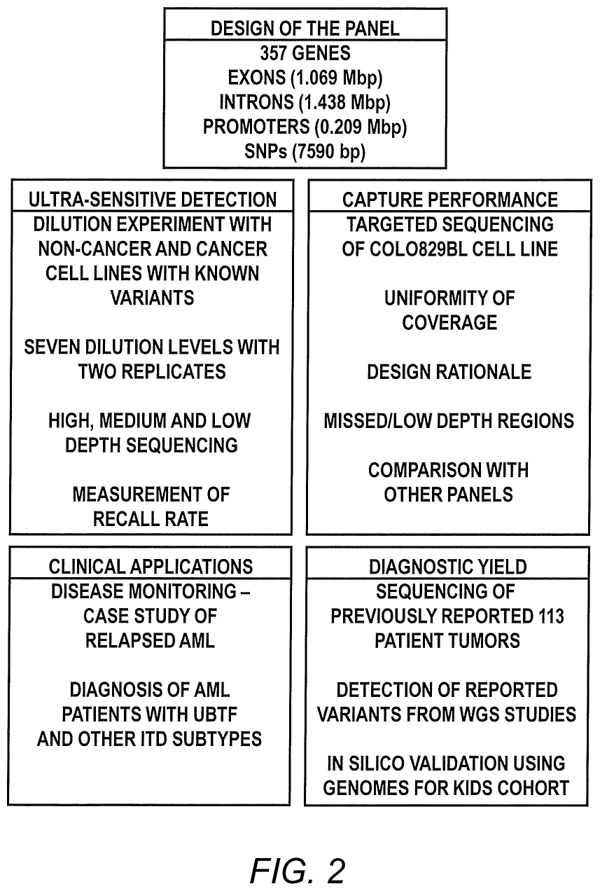

Panel Design. SJPedPanel was designed by integrating findings from 44 published tumor-normal paired genomics studies of childhood cancers that spans leukemia, brain, and solid tumor (Grobner et al. (2018) Nature 555:321-327; Ma et al. (2018) Nature 555:371-376; Rusch et al. (2018) Nat. Commun. 9:3962; Cheung et al. (2012) JAMA 307:1062-1071; Gruber et al. (2012) Cancer Cell 22:683-697; Roberts et al. (2012) Cancer Cell 22:153-166; Robinson et al. (2012) Nature 488:43-48; Wu et al. (2012) Nat Genet 44:251-253; Zhang et al. (2012) Nature 481:329-334; Zhang et al. (2012) Nature 481:157-163; Chen et al. (2013) Cancer Cell 24:710-724; Holmfeldt et al. (2013) Nat Genet 45:242-252; Jaffe et al. (2013) Nat Genet 45:1386-1391; Paugh et al. (2013) Cancer Res 73:6219-6229; Shah et al. (2013) Nat Genet 45:1226-1231; Zhang et al. (2013) Nat Genet 45:602-612; Chen et al. (2014) Cell Rep 7:104-112; Parker et al. (2014) Nature 506:451-455; Roberts et al. (2014) N Engl J Med 371:1005-1015; Tirode et al. (2014) Cancer Discov 4:1342-1353; Wu et al. (2014) Nat Genet 46:444-450; Andersson et al. (2015) Nat Genet 47:330-337; Li et al. (2015) Nat Med 21:563-571; Lu et al. (2015) J Invest Dermatol 135:816-823; Ma et al. (2015) Nature Commun 6:6604; Pinto et al. (2015) Nat Commun 6:6302; Tong et al. (2015) Cancer Cell 27:712-727; Zhang et al. (2015) N Engl J Med 373:2336-2346; Faber et al. (2016) Nat Genet 48:1551-1556; Iacobucci et al. (2016) Cancer Cell 29:186-200; Liu et al. (2016) EBioMedicine 8:173-183; Zhang et al. (2016) Nat Genet 48:1481-1489; de Rooij et al. (2017) Nat Genet 49:451-456; Liu et al. (2017) Nat Genet 49:1211-1218; Northcott et al. (2017) Nature 547:311-317; Alexander et al. (2018) Nature 562:373-379; Bolouri et al. (2018) Nat Med 24:103-112; Pajtler et al. (2018) Acta Neuropathol 136:211-226; Stewart et al. (2018) Cancer Cell 34:411-426; Brady et al. (2019) Mol Cancer Res 17:895-906; Newman et al. (2019) Nat Med 25:597-602; Brady et al. (2020) Nature Commun 11:5183; Brady et al. (2020) Blood 136:2235-2237; Waanders et al. (2020) Blood Cancer Discov 1:96-111). SJPedPanel includes 1.069 million exonic base pairs from 5,275 coding exons for detecting protein coding mutations in 357 known driver genes for childhood cancers (FIG. 2, Table 1). To account for the structural variations (SVs) that result in subtype-defining oncogenic fusions for which DNA breakpoints typically fall in intronic regions, 1.438 million base pairs from 297 introns of 94 genes (Table 1) were included. Moreover, 0.209 million bases from promoter regions were targeted for detecting promoter alterations including rearrangements and point mutations such as TALI in T-ALL (Mansour et al. (2014) Science 346:1373-1377). Highly recurrent oncogenes (MYCN in neuroblastoma) and tumor suppressor genes (such as CDKN2A, PAX5, and SMARCB1) were targeted by probes tiling the entire gene region for detecting CNVs. To account for the fact that breakpoints of structural alterations can fall outside gene regions, the target regions were extended to include frequent DNA breakpoints by using patient data from ProteinPaint and GenomePaint. Collectively, 2.82 million base pairs were designed for potential SNV, Indel, SV and CNV/LOH driver alterations. Notably, a few known childhood cancer drivers are intentionally excluded due to genomic space considerations. For example, MECOM and GFIIB are known to be involved in enhancer-hijacking alterations and were excluded due to the large space needed to cover the many possible breakpoints. In addition, 7,590 SNPs were selected for detecting copy number variations and loss of heterozygosity (CNV/LOH) across the genome (Table 2).

The median distance between these SNPs is 332 Kb, with 25^{th} and 75^{th} quantile being 60 Kb and 593 Kb, respectively. Notably, >80% of these SNPs have population frequency between 40% and 60%, which ensures that that nearly 50% of patients are heterozygous at each SNP site. Therefore, around 3,000 (=7590×0.5×0.8) heterozygous SNPs are expected for each patient, which leads to a theoretical resolution of ˜1 Mb for CNV/LOH detection. The number of SNPs chosen per chromosome is roughly proportional to the lengths of chromosomes. Considering the read length and the insert length (for target capture and sequencing), these 7, 590 SNPs actually occupy ˜250*7590=1.8975 million base pairs. Thus, the panel consisted of ˜4.7 million base pairs, or ˜0.15% of the human genome. The compact size of this panel enables us to reach 30,000× at the cost of a standard WGS (˜30×) per sample, thus enabling cost-effective cancer monitoring and/or early detection (FIG. 2).

Comparison of gene content with other panels. The gene content between the SJPedPanel and five other commonly used commercial panels for childhood cancers, including FoundationOne Heme, FoundationOne CDx, MSK-IMPACT, OncoKids, CHOP CHMP/CSTP and Oncomine Comprehensive assay v3 (OCAv3) were compared The list of 183 driver genes reported in two recent childhood pan-cancer studies (Grobner et al. (2018) Nature 555:321-327; Ma et al. (2018) Nature 555:371-376) involving 2,578 cases were used. SJPedPanel covers 159 (87%) genes whereas all other panels covered <60% of the reported pediatric cancer driver genes.

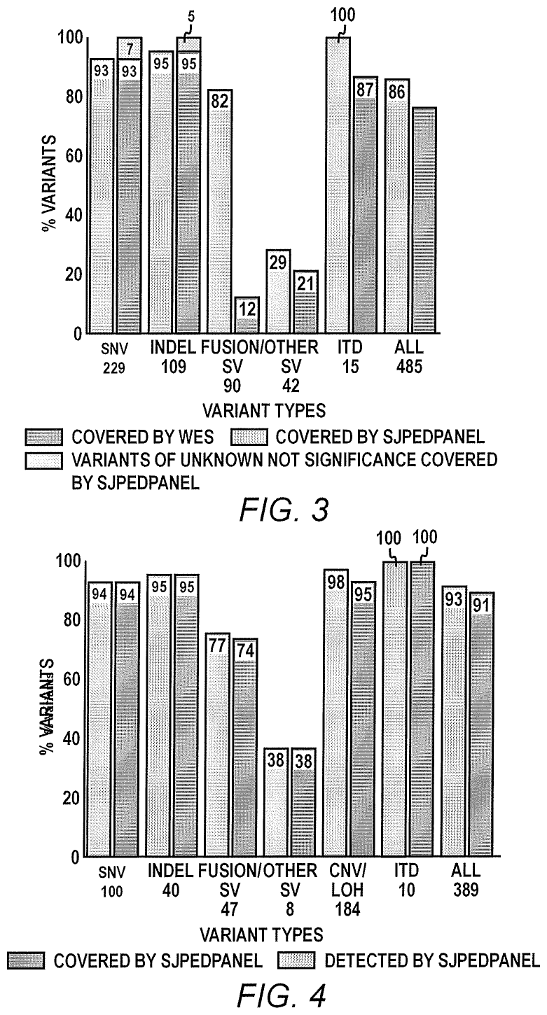

A comparison of gene names among the panels indicated that SJPedPanel has unique coverage of 105 genes (FIG. 3) when compared to the other panels combined, such as DGCR8 and SIX1 for Wilms tumor, SHH for medulloblastoma, ZFTA for ependymoma, UBTF-TD and PICALM for AML. Among all the panels, SJPedPanel provides the largest intronic regions (297 introns from 94 genes) responsible for rearrangements that generate fusion oncoproteins.

Capture performance of the panel. An important consideration in genomic sequencing (especially in panel sequencing) is the coverage uniformity. Four targeted sequencing libraries (C1-C4) prepared using COLO829BL (ATCC #CRL-1980), a non-cancer cell line that has been extensively used in literature for clinical proficiency testing or benchmarking were sequenced. To ensure reproducibility, technical replicates were generated to achieve high (C1, C2, ˜2000×) and low depth (C3, C4, ˜200×) of sequencing. Libraries in each set were sequenced with either the Illumina NovaSeq 6000 (C1, C3) or NextSeq 500 (C2, C4).

This analysis showed that the average depth was highly correlated (r²: 0.98) with the number of raw reads sequenced. With this data, the capture uniformity at base pair level (FIGS. 1A-1B) and at region level (FIGS. 1C-1D) was evaluated. Because highly uniform capture data would ensure most bases/regions to have similar depth (therefore a histogram with very small standard deviation), coefficient of variation (C_{V}, defined by σ/μ of the histogram) was used to measure sequencing uniformity. Here σ and μ are the estimate of standard deviation and mean, respectively, by trimming 2.5% of extreme values from both ends of the histograms (FIGS. 1A-1D). At base pair level, it was observed that C_{V} is close to 0.35 for libraries sequenced by NovaSeq, and to be between 0.37 and 0.38 for libraries sequenced by NextSeq. At region level, NovaSeq data has C_{V} close to 0.25, while NextSeq data has C_{V} range from 0.22 to 0.28.

Overall, the standard deviation is less than or around ⅓ of the mean, which ensures that most of the target bases/regions are sufficiently covered. Using the two-sigma rule (that approximates the 95% confidence interval), the percentage of bases/regions with depth higher than (μ−2σ) were measured. It was found that 97% and 95% of bases have depth higher than this threshold for NovaSeq data and NextSeq data, respectively. Similar trends were observed from the region level analyses (FIGS. 1C-1D). These data indicated that SJPedPanel has satisfactory capture efficiency that is reproducible over different sequencing platforms.

Regions that are consistently poorly covered (i.e., less than μ−3σ) were analyzed in the COLO829 data. Twenty-seven regions (26 regions are small exons) were identified, of which 10 regions consistently showed no coverage across all the four samples. These 27 regions occupy 8091 bp (˜0.3%) of the panel and more than half of these bases (4972 bp) belong to only two regions, NUTM2A (3152 bp including intron 1 with 2050 bp) and DUX4 (1820 bp including exon 1; Table 5). For STAG2 the coverage is slightly below the pre-defined cut-offs for 3 regions (351 bp) (e.g. ˜500× for C1 sample, Table 5).

TABLE 5

| | COLO829BL | COLO829BL | COLO829BL | COLO829BL |
|---|---|---|---|---|
| Region | C1 | C2 | C3 | C4 |
| PHIP | 183.82 | 28.44 | 24.46 | 2.92 |
| (chr6: 79787407- | | | | |
| 79787456) | | | | |
| EZH2 | 95.57 | 47.41 | 7.9 | 3 |
| (chr7: 148504728- | | | | |
| 148504808) | | | | |
| MED12 | 520.18 | 266.03 | 36.48 | 25.92 |
| (chrX: 70345503- | | | | |
| 70345573) | | | | |
| NUTM2A | 671.08 | 248.68 | 69.82 | 24.64 |
| (chr10: 88985578- | | | | |
| 88988729) | | | | |
| DMBT1 | 64.72 | 15.9 | 4.4 | 0.38 |
| (chr10: 124343828- | | | | |
| 124343877) | | | | |
| DMBT1 | 7.58 | 1.03 | 0.58 | 0.7 |
| (chr10: 124361303- | | | | |
| 124361646) | | | | |
| MED12 | 558.68 | 357.04 | 46.45 | 32.37 |
| (chrX: 70357398- | | | | |
| 70357495) | | | | |
| DMBT1 | 325.42 | 186.4 | 28.15 | 8.45 |
| (chr10: 124360497- | | | | |
| 124360549) | | | | |
| DMBT1 | 100.75 | 57.43 | 19.53 | 6.19 |
| (chr10: 124341299- | | | | |
| 124341351) | | | | |
| BEND2 | 13.61 | 9.19 | 0 | 0.63 |
| (chrX: 18231884- | | | | |
| 18232041) | | | | |
| NOTCH2 | 280.41 | 196.22 | 25.45 | 14.92 |
| (chr1: 120572519- | | | | |
| 120572620) | | | | |
| PMS2 | 286.43 | 138.04 | 22.12 | 15.04 |
| (chr7: 6013020- | | | | |
| 6013183) | | | | |
| PAX6 | 437.99 | 189.4 | 49.19 | 26.56 |
| (chr11: 31811472- | | | | |
| 31811577) | | | | |
| STAG2 | 584.4 | 384.74 | 52.85 | 33.19 |
| (chrX: 123195611- | | | | |
| 123195734) | | | | |
| STAG2 | 529.09 | 414.11 | 55.25 | 42.05 |
| (chrX: 123184961- | | | | |
| 123185079) | | | | |
| KDM6A | 669.28 | 450.54 | 63.19 | 47.67 |
| (chrX: 44918241- | | | | |
| 44918359) | | | | |
| STAG2 | 500.34 | 285.19 | 51.43 | 24.94 |
| (chrX: 123200015- | | | | |
| 123200122) | | | | |
| MLLT1 | 6.66 | 0 | 0 | 0 |
| (chr19: 6279774- | | | | |
| 6279805) | | | | |
| DUX4 | 0 | 0 | 0 | 0 |
| (chr4: 190998791- | | | | |
| 191000610) | | | | |
| NUTM2A | 0 | 0 | 0 | 0 |
| (chr10: 88991768- | | | | |
| 88991927) | | | | |
| NUTM2A | 1.32 | 0 | 0 | 0 |
| (chr10: 88993220- | | | | |
| 88993356) | | | | |
| NUTM2A | 0 | 0 | 0 | 0 |
| (chr10: 88990667- | | | | |
| 88990815) | | | | |
| DMBT1 | 0 | 0 | 0 | 0 |
| (chr10: 124342106- | | | | |
| 124342449) | | | | |
| DMBT1 | 0 | 0 | 0 | 0 |
| (chr10: 124362606- | | | | |
| 124362655) | | | | |
| DMBT1 | 0 | 0 | 0 | 0 |
| (chr10: 124366665- | | | | |
| 124366714) | | | | |
| DMBT1 | 0 | 0 | 0 | 0 |
| (chr10: 124365362- | | | | |
| 124365705) | | | | |
| DMBT1 | 0 | 0 | 0 | 0 |
| (chr10: 124364556- | | | | |
| 124364608) | | | | |

While looking for the potential reasons for poor coverage of these regions, it was observed that flanking regions (±50 bp) of most of these poorly covered regions comprise either high GC content, such as exon 1 of MLLT1 (94% GC), or homopolymer runs, such as T-runs around three regions of STAG2 gene.

Out of the five panels compared, only MSK-IMPACT panel was reported to have 31 consistently poorly covered regions, which all happened to be targeted by SJPedPanel as well. Interestingly, SJPedPanel demonstrated sufficient depth of coverage (>(μ−2σ) of respective COLO829BL sample level cut-offs) for 29 out of the 31 regions (Table 6). The remaining two regions, exon 2 of NOTCH2 and exon 15 of PMS2, consistently showed poor coverage as in MSK-IMPACT panel and were also part of the 27 poorly covered regions of SJPedPanel (Table 5).

TABLE 6

| | | Average depth of coverage in | | | | |
|---|---|---|---|---|---|---|
| | | four COLO829BL samples | | | | |
| | Chromosome | sequenced | | | | #PASS |
| Gene | Coordinates | C1 | C2 | C3 | C4 | Samples |
| CHEK2 | 22:29085123- | 2410.2 | 1953.9 | 263.93 | 194.02 | 4 |
| | 29085203 | | | | | |
| FLT3 | 13:28674605- | 2627.8 | 599.49 | 261.3 | 77.3 | 4 |
| | 28674647 | | | | | |
| H3F3A | 1:226259052- | 1678.3 | 1184.7 | 161.13 | 122.57 | 4 |
| | 226259180 | | | | | |
| KMT2C | 7:151970790- | 8854.3 | 7594.3 | 885.4 | 730.02 | 4 |
| | 151970952 | | | | | |
| KMT2C | 7:151962123- | 7894.7 | 6109.3 | 825.73 | 597.88 | 4 |
| | 151962294 | | | | | |
| KMT2C | 7:151935792- | 2673 | 2012.8 | 287.92 | 199.38 | 4 |
| | 151935911 | | | | | |
| KMT2C | 7:151932902- | 3532.8 | 2094.8 | 360.85 | 243.44 | 4 |
| | 151933018 | | | | | |
| KMT2C | 7:151927008- | 6972.1 | 6243 | 716.15 | 567.7 | 4 |
| | 151927112 | | | | | |
| KMT2C | 7:151921520- | 1699.4 | 1248.4 | 191.51 | 128.67 | 4 |
| | 151921701 | | | | | |
| KMT2C | 7:151921100- | 2092.8 | 1671.5 | 219.17 | 161.13 | 4 |
| | 151921264 | | | | | |
| KMT2C | 7:151919658- | 1924.6 | 1679.4 | 225.56 | 169.61 | 4 |
| | 151919767 | | | | | |
| KMT2C | 7:151904385- | 2340.2 | 2025.9 | 246.53 | 180.85 | 4 |
| | 151904513 | | | | | |
| NOTCH2 | 1:120611948- | 4477.3 | 1418.5 | 457.64 | 133.1 | 4 |
| | 120612020 | | | | | |
| NOTCH2 | 1:120572529- | 282.63 | 196.79 | 25.12 | 15.16 | 0 |
| | 120572610 | | | | | |
| NOTCH2 | 1:120547952- | 2103 | 1135.2 | 200.4 | 116.94 | 4 |
| | 120548211 | | | | | |
| NOTCH2 | 1:120539620- | 2189.9 | 1307.3 | 217.46 | 153.52 | 4 |
| | 120539955 | | | | | |
| PIK3CA | 3:178937737- | 1868.6 | 1469.7 | 195.48 | 151.13 | 4 |
| | 178937840 | | | | | |
| PIK3R2 | 19:18272089- | 1902.9 | 373.39 | 172.45 | 38.46 | 4 |
| | 18272305 | | | | | |
| PMS2 | 7:6022455- | 987.99 | 727.49 | 107.48 | 64.74 | 4 |
| | 6022622 | | | | | |
| PMS2 | 7:6018227- | 1954.5 | 1442.7 | 198.48 | 138.55 | 4 |
| | 6018327 | | | | | |
| PMS2 | 7:6017219- | 1256.9 | 886.91 | 101.76 | 90.61 | 4 |
| | 6017388 | | | | | |
| PMS2 | 7:6013030- | 291.58 | 140.35 | 23.02 | 15.07 | 0 |
| | 6013173 | | | | | |
| PTEN | 10; 89725004- | 1759.3 | 1478.4 | 196.96 | 118.2 | 4 |
| | 89725229 | | | | | |
| SDHA | 5:254508- | 1889.4 | 1192.7 | 181.05 | 116.19 | 4 |
| | 254621 | | | | | |
| STAT5B | 17:40371330- | 1224.5 | 388.51 | 120.8 | 36.49 | 4 |
| | 40371481 | | | | | |
| STAT5B | 17:40370741- | 2017.3 | 875.35 | 198.24 | 104.87 | 4 |
| | 40370896 | | | | | |
| SUZ12 | 17:30267305- | 1798.2 | 1735.6 | 202.57 | 182.38 | 4 |
| | 30267351 | | | | | |
| SUZ12 | 17:30267441- | 1041.5 | 942.12 | 121.54 | 81.86 | 4 |
| | 30267505 | | | | | |
| SUZ12 | 17:301274636- | 1645.6 | 1402.7 | 173.43 | 146.62 | 4 |
| | 30274704 | | | | | |
| SUZ12 | 17:30300165- | 1280.8 | 1079.5 | 124.65 | 101.05 | 4 |
| | 30300250 | | | | | |
| SUZ12 | 17:30310018- | 2013.9 | 1517.1 | 204.08 | 115.4 | 4 |
| | 30310123 | | | | | |

Similarly, the depth of coverage at designed SNPs was analyzed. Notably, 99.5% of all the 7590 SNPs have depth more than (μ−2σ) of the respective sample level cut-offs. This analysis showed that the variant allele frequencies (VAF) of all the SNPs in control COLO829BL samples were clustered around either 0, 0.5 or 1. A total of 3300 heterozygous SNPs (0.3≤VAF≤0.7) are observed in COLO829BL, supporting the informativeness of the designed SNPs as mentioned above (˜3000 heterozygous SNPs expected from any donors).

In silico comparison of SJPedPanel and WES in a real-time clinical genomics cohort. The coverage of reported pediatric cancer alterations using whole exome sequencing (WES) was compared, because WES is effectively a capture-sequencing kit that targets coding exons of all genes rather than a panel of genes (thus an upper bound of all coding-region-based gene panels). The SJPedPanel was evaluated to determine if t offered comparable coverage of driver alterations (with a focus on coding SNVs and Indels) in pediatric cancers to WES. The “Genome for Kids” (G4K) study reported pathogenic and likely pathogenic (P/LP) variants (called driver alterations hereafter) using three platform sequencing (WGS, WES and RNAseq) from 253 pediatric cases that encompassed 20 cancer subtypes in a real-time clinical genomics setting, thus enabling assessment of the potential of SJPedPanel to cover driver alterations from diverse childhood cancer types. In silico analysis was performed of regions targeted by SJPedPanel and WES using the curated positions of 485 driver alterations (including SNV/Indel/SV/ITD) from the G4K study. SJPedPanel covered 86% of the 485 reported driver alterations as compared to 76% by WES (FIG. 3, last pair of bars for “All” variants with gray background). The variants were classified into SNV, Indel, Fusion/SV (structural rearrangements that result in fusion oncoproteins), Other SV (structural rearrangements that do not result in fusion oncoproteins but affect cancer driver genes such as disrupting tumor suppressor genes), and ITD, by using the class labels in the G4K study. This analysis showed that WES only covered 12% of the Fusions/SV that have either of the breakpoints in exonic regions, while SJPedPanel covered 82% of these events. On the other hand, while WES covered all the reported driver SNVs and Indels, SJPedPanel did not cover 7% and 5% of SNVs and Indels, respectively. Interestingly, the host genes of these uncovered SNVs and Indels are rarely mutated (<0.1%) childhood cancers (FIG. 3). Of note, SJPedPanel covered 100% of the ITDs, while WES does not cover 13% of these. In fact, the ITDs missed by WES have DNA breakpoints that fall in introns and resulted in duplication of involved exons, such as tandem duplications in PAX5 and KMT2A, for which selected intronic regions were designed in SJPedPanel. Of note, SJPedPanel successfully captured UBTF exonic tandem duplications.

The percentage of patients who could benefit from SJPedPanel vs WES was also evaluated. At least one variant per case would have been covered by SJPedPanel in 93% of 210 cases (median: 1, range: 1 to 13), in contrast to 75% of the cases using WES (median: 2, range: 1 to 13). Most of this gain is due to the capture of intronic breakpoints that result in oncogenic fusions (FIG. 4). These data demonstrated that SJPedPanel has superior potential for detection and reporting of driver alterations in pediatric tumors than WES.

Comparison of diagnostic yield between SJPedPanel and WES. To validate the in silico findings, 113 clinical cases were re-sequenced with available specimens from previous clinical studies using SJPedPanel. These samples reflect the broad tumor types and subtypes common in childhood cancer, including 27 hematological malignancies, 43 solid tumors and 43 brain tumors. Common subtypes, such as AML (n=14), ALL (n=10), rhabdomyosarcoma (n=5), neuroblastoma (n=5), osteosarcoma (n=3), Wilms tumor (n=3), high-grade glioma (n=8) and medulloblastoma (n=14) are represented in addition to rare entities, such as melanoma (n=1) and desmoplastic small round cell tumor (n=2). Among these cases, 389 driver alterations (Methods) are reported via three-platform (WGS, WES, RNAseq) sequencing. These include 100 SNVs, 40 Indels, 55 SVs, 184 CNV/LOHs and 10 ITDs. Of these, 361 (92.8%) variants were targeted by the panel, including 94 SNVs (94%), 38 Indels (95%), 36 Fusion/SVs (76.59%), 3 other SVs (37.5%), 180 CNV/LOHs (97.82%), and 10 ITDs (100%) (FIG. 4). In total 28 P/LP variants were not covered by this panel. Of these, 6 were SNVs, 2 were Indels, 11 were Fusion/SVs (not designed), 5 were other SVs and 4 were focal CNVs. The uncovered variants belonged to 26 genes, which are not mutated in published pediatric pan-cancer cohorts except for COL1A1 that has a low mutation frequency of 0.2%, further supporting their omission from the SJPedPanel design.

A mean depth of ˜2500× was achieved for all the samples tested, which ensures 95% confidence of detection of variants with 218 allele fraction (AF). An average of 3300 heterozygous SNPs (median: 3405, range: 2227 to 3844) was found in germline samples (n=30). These samples were pooled as a reference to interrogate CNV/LOH in tumor samples. By using a “rotation control” method coupled with an Indel/SV genotyping tool, 98% (354 of the 361 covered variants) of reported driver alterations were detected. SNVs, Indels, ITDs showed detection rate of 100% (FIG. 4). The Fusion/SVs showed an overall recall rate of 97% (35 out of 36) of the covered alterations. One fusion/SV marker (RUNX1:RUNX1T1 from the case SJCBF100) that was covered in the panel design with single breakpoint in RUNX1 was missed due to insufficient depth (21×) of coverage, therefore poor capture efficiency in certain genomic regions should be further evaluated. By contrast, 38% (3 out of 8) of other SVs were detected, and the rest of the missed SVs had their breakpoints either in intergenic or non-covered regions, which is consistent with much larger genomic space for breakpoints in tumor suppressor genes. On the other hand, SJPedPanel detected 94.6% of the reported CNV/LOHs. Collectively, SJPedPanel detected 91% of the 389 reported driver alterations from these 113 cases (FIG. 4, pair of last bars with gray background]. Of note, at least one variant was detected for 96.5% cases (n=113), with a median of 3 variants per case (Range=0-16). Consistent with the in-silico analysis (FIG. 3), a comparison of SNV, Indel, SV and ITD variants (n=205 out of 389) discovered using three platform sequencing (WGS, WES, RNASeq) indicated that SJPedPanel covers 88% of these variants whereas WES covers 788. These data further confirm the superior performance achieved using SJPedPanel for childhood cancers with a panel size approximately 10% the size of WES.

The successes and challenges in panel design by using structural variants as examples should be considered. First, due to the large genomic size of intronic regions that can be involved in oncogenic fusions, inclusion/exclusion of intronic regions involves a difficult balance between panel size and effective coverage of patient population. In a prior study of fusion gene pairs involved in 5,190 childhood cancers, 72 representative genes were selected for 274 fusion gene pairs and SJPedPanel included 53 of these 72 genes. The maximum mutation frequency of the 19 genes not included in the SJPedPanel is 0.18. Furthermore, inclusion of all relevant introns of all genes involved in oncogenic fusions would need ˜8 Mbp. With the observation that some genes can have multiple fusion partners (e.g., 40 of the 53 included representative genes have between 2 to 32 partner genes), some partner genes were not included by relying on common representative genes, which reduced the space from ˜8 Mbp (280 introns) to ˜1 Mbp (156 introns). For example, including only the intronic regions of KMT2A was sufficient to detect KMT2A:MLLT1 fusion in pediatric T-ALL patient (SJMLL002) even though MLLT1 introns were not included in the panel content. Despite this success, SVs can be challenging to detect. For example, SJPedPanel missed RUNX1:RUNX1T1 in SJCBF100 because RUNX1T1 introns were not included (˜100K base pairs needed). The panel also missed an inversion involving RB1 gene in SURB0051 because the DNA breakpoints fall into an intronic region of RB1 and RB1 introns are not covered (˜180 kb are needed to cover all RB1 introns).

Apart from SV/Fusions, SJPedPanel covers well known ITDs such as FLT3, NOTCH1, BRAF, etc. SJPedPanel also provides exclusive coverage of UBTF gene in pediatric AML. Although in literature UBTF ITDs are typically mistakenly detected as small Indels, the SJPedPanel successfully detected UBTF TDS in two pediatric AML cases (SJAML015373 and SJAML016569).

Determining limit of detection. An important application of panel sequencing is disease monitoring, where the tumor burden is typically less than 18 and thus variants are rare and challenging to detect. To investigate the applicability of the panel, dilution experiments were performed (with 7 tumor concentrations of 10%, 5%, 2.5%, 1%, 0.5%, 0.2%, 0.1%, in addition to pure normal of 0% and pure cancer of 100%) using 6 pediatric cancer cell lines (697, EW-8, K562, ME-1, MOLM-13, and Rh30) and 1 non-cancer cell line (GM12878) as normal control. These 6 lines collectively contain 26 unique P/LP variants (14 SNVs, 4 Indels, and 8 SVs). The lack of shared driver alterations allowed for pooling of dilutions to reduce the experimental complexity while keeping the diversity of mutation types. For example, at ladder of 0.5%, 5:5:5:5:5:5:970 cell equivalents from the 6 cancer lines and the normal line, respectively were mixed. To ensure sufficient power of detecting variants with low allele fractions, an average depth of 5,000× for 0.5%, >7,000× for 0.2% and 0.1% dilution concentrations was achieved.

SequencErr (Davis et al. (2021) Genome Biol 22:37) and SVIndelGenotyper were used to perform allele counting followed by variant calling using binomial models with false discovery rate control, where the pure normal of 0% was used to estimate background error rates as no cancer-driving somatic alterations are expected in non-cancer cell line GM12878. The observed allele fractions closely represent corresponding dilution ladders, with R-squared values of 0.7 and 0.72, for biological replicates A and B, respectively. A greater than 90% detection rate was achieved when the dilution concentration is above 0.5%, it diminishes quickly at lower dilutions. At a dilution concentration of 0.18, recall rate was 69% and 42% in replicates A and B, respectively.

To investigate the effect of sequencing depth on detection rate, in silico down-sampling experiments were performed. For all the dilutions with concentrations ≥18, which were initially sequenced at 5000× and 2500×, recall rate is found to be close to 100% even after down sampling their depths to 1000×. However, recall rate declined with down sampled depths for dilution concentrations <1%. For samples with dilution concentration of 0.5%, recall rate dropped from 97% at 3000× to 75% at 1000×. Thus, markers with allele fraction of 0.5% can be reliably detected at 2500×˜3000×, which is concordant with the theoretical binomial calculation of 2,100×. However, for markers with allele fraction of 0.2% and 0.1% the recall rate was <50% even at initial 10,000× data. This finding suggests that the current LoD is between 0.1% and 0.5% and is consistent with a recent report using cfDNA data.

The above data suggests a depth of 2, 100× will ensure 95% chance of detecting >=5 mutant reads if the true allele fraction is >0.5%, However, considering the sequencing uniformity parameter, where 95% of targeted regions are covered at μ−2σ=0.33μ, 3×2,100=6,300× is recommended so that >95% of targeted regions will be covered >2, 100× and ensure the 95% chance of detecting variants with allele fraction of 0.5%. Since the total space of this panel is ˜0.15% of a human genome, 6,300× corresponds to whole genome sequencing at 9× coverage.

Case study: Real time tracking of relapsed AML using deep sequencing. To evaluate the suitability of the panel for disease monitoring in a real-world setting, two AML cases (SJAML016582 and SJAML016551) that had remission samples with low tumor purity were selected. These cases have multiple pathogenic and likely pathogenic variants (P/LP) reported by clinical sequencing of diagnosis and relapse samples and are ideal for panel sequencing. The diagnosis, remission, and relapse samples were sequenced to an average depth of 5,000× using SJPedPanel per above power calculations.

In case SJAML016582, apart from subtype defining NUP98:NSD1 fusion, 4 pathogenic variants were detected at diagnosis (day 0) and 6 pathogenic variants were detected at relapse (day 315), with 2 variants shared between diagnosis and relapse. With the ultra-deep panel sequencing data, all variants initially detected by whole genome sequencing, including 4 at diagnosis and 6 at relapse were recovered. Panel sequencing detected the SNV that encodes NRAS Q61R in diagnosis tumor with allelic fraction (AF) 0.12%, and the MNV that encodes NRAS Q61R in relapse tumor with AF 0.07%, both of which are beyond the detection power of whole genome sequencing. Further, in the day 26 remission data, a high tumor burden (6.56%) was detected. Notably, the tumor burden continued to decrease down to an allele fraction of 2.88% at day 97 as reflected by the SV responsible for NUP98:NSD1 fusion.

In addition to disease monitoring, ultra-deep sequencing was evaluated for detecting measurable residual disease (MRD) to investigate response patient to chemotherapy. Generally flow cytometry is a method of choice for such applications in addition to real time PCR and droplet digital PCR. However, these approaches are not as scalable as ultra-deep sequencing. In order to evaluate the efficacy of SJPedPanel to detect MRD, three samples from diagnosis (day 0), MRD (day 23) and relapse (day 344) were sequenced for AML patient SJAML016551. Here the diagnosis and relapse samples are used to define ancestral genetic alterations that are obligated to be present in the MRD sample. For this case, five pathogenic variants, including a KMT2A:MLLT10 structural variant, were identified to be shared between diagnosis and relapse and are expected to be detected in MRD sample. Although flow-based MRD detection was negative for this case, the deep sequencing detected all 5 pathogenic variants with allele fraction range between 0.7-18. Together, these data demonstrate the ability of SJPedPanel in measuring MRD and monitoring disease progression that could aid in early detection of relapse.

### Example 2: Etiology of Oncogenic Fusions in Pediatric Malignancies

Patient Cohort and RNAseq Data. Transcriptome sequencing (RNA-seq) data from 5,286 patients were collected from following public resources: (1) St. Jude cloud (McLeod et al. (2021) Cancer Discov. 11:1082-1099) that included the St. Jude/Washington University Pediatric Cancer Genome Project cohort (PCGP; n=777; Downing et al. (2012) Nat. Genet. 44:619-622), the St. Jude Genomes for Kids study (G4K; n=253; Newman et al. (2021) Cancer Discov. 10.1158/2159-8290.CD-20-1631) and the St. Jude Real-time Clinical Genomics initiative (RTCG; n=1006); (2) a collection of transcriptome study of childhood AML (n=314); (3) a genomics study of relapsed childhood ALL (n=101; Li et al. (2020) Blood 135:41-55); (4) NCI's Therapeutically Applicable Research to Generate Effective Treatments cohort (TARGET; n=759; Ma et al. (2015) Nat. Commun. 6:6604); AML transcriptome data from (5) Children's Oncology Group (n=1086); (6) Children's Brain Tumor Network (CBTN; n=820) downloaded from Kids First data portal; and (7) Childhood Rhabdomyosarcoma (RHB; n=84; study identifier phs000720) and Ewing Sarcoma (EWS; n=84; study identifiers phs000768 and phs000804) downloaded from dbGaP. In addition, 9525 transcriptome datasets from GTEx project were used as normal controls in relevant analysis.

Fusion Detection. Oncogenic fusions were detected by using state-of-the-art methods reported to have superior performance (Tian et al. (2020) Genome Biol. 21:126; Haas et al. (2019) Genome Biol. 20:213), including Cicero (Tian et al. (2020) Genome Biol. 21:126), Arriba (Uhrig et al. (2021) Genome Res. 31:448-460), STAR-fusion (Haas et al. (2017) bioRxiv 120295), and FusionCatcher (Nicorici et al. (2014) bioRxiv 011650). For potential discrepancies (detected by less than two tools), the findings were manually reviewed to determine the fusion status.

Neo-Versioner. An in-house python script (“Neo-Versioner”) was developed to determine the status of intronic versioning. For each gene pair (e.g., CBFB-MYH11), the translation frame was first checked for all possible exon-exon combinations of the two involving genes to build a database of in-frame exon-exon combinations. For each in-frame exon combination, a junction contig (60 nucleotides) was next constructed using 30 nucleotides from involving exons from the N′ gene and the C′ gene, respectively. A database of 20-mers was then constructed from these contig sequences to facilitate the efficient extraction of RNAseq reads containing one of such 20-mers. Each candidate read was compared to all junction contigs. A junction contig is determined to be supported once if it is a substring of a read. To account for partial matching, a read was allowed to contain a matching of as few as 10 nucleotides from either N′ or C′ side, provided that the other side of the junction contig was fully matched to the read. The above parameters assumed an error rate of <1% in short read Illumina sequencing that is justified by recent error profile studies on next generation sequencing (Ma et al. (2019) Genome Biol. 20:50; Davis et al. (2021) Genome Biol. 22:37).

Calculating Pseudo Binding Affinity for Splice Sites The binding affinity of candidate splice site to splicing machinery was calculated using the well-established Position Specific Weight Matrix (PWM) method. Human genes were downloaded from UCSC Genome Browser, protein coding genes (RefSeq ID starts with “NM”) and their exon boundaries were extracted and PWMs were constructed using 209, 192 donors and 205, 329 acceptors from these known protein coding genes. For donor, 3 base pairs 5′ to the GT and 10 base pairs 3′ to the GT were used, totaling a 15 base pair motif. For acceptor, 18 base pairs 5′ to the AG and 3 base pairs 3′ to the AG were used, totaling a 23 base pair motif. The motifs were denoted as Mᵢⱼ where i can be either of A, C, G or T and j=1, . . . , K where K is 15 for donor and 23 for acceptor. Mᵢⱼ represents the observed occurrences of known splice sites at position j for nucleotide i. Denote the candidate DNA sequence as Sⱼ, j=1, . . . , K, it can be scored by the PWM using a log-likelihood ratio score method:

$$LLR = {\sum }_{j} {\sum }_{i} log ( \frac{{M}_{ij}}{{B}_{i}} ) \times I ( i , {S}_{j} )$$

were Bᵢ is the genome-wide background frequency of nucleotides A, C, G and T. Here Bᵢ=0.3 when i is A or T and Bᵢ=0.2 when i is C or G to account for the A/T richness in the human genome. I(i,Sⱼ) is an indicator function that takes value of 1 when Sⱼ=i and 0 otherwise.

To ensure the quality of the constructed motifs, all splice sites of known human genes were scored and most of the splice sites received positive scores (>80% donors have score >4; >80% acceptors have score >4.3). As a negative control, 1.12 million potential donor (GT) sites and 1.76 million potential acceptor (AG) sites that do not belong to known human genes from forward strand of chr19 (one of the shortest chromosomes to save computation time) were extracted and scored. Notably, >90% of such false donors had a score <4 and >90% of such false acceptors had a score <4.3, validating the power of the PWM method in discriminating real splice sites from non-real sites.

Neo-Splicer. Cancer cells must create novel splice sites to allow production of functional oncogenic fusion proteins if the natural splice sites were disrupted by rearrangements. However, a novel splice may not necessarily lead to in-frame translation because multiple splice sites may be available for the cancer cells that will survive if there is one viable splicing isoform. To search for novel splice sites that can result in in-frame translation, an in-house script (“Neo-Splicer”) was developed. Given the ubiquitous nature of candidate splice sites (AG and GT; 1 in every 16 nucleotides expected by chance), the PWM described above was used to detect putative splice sites. Second, given the DNA breakpoints (42% (=834/2009) chance of detection in RNAseq data of an oncogenic fusion, all AG and GT dinucleotides were enumerated between intact exons of involving genes, hypothetical exons were generated, and corresponding translation frames were checked. RNAseq reads were then compared with above predictions to determine the neo splice sites and corresponding isoforms used by the cancer cells.

Expression Patterns of Oncogenic Fusions. Although N′ genes, which contributed enhancer and promoter regions for the oncogenic fusions, were expected to be constitutively expressed in the host lineage of corresponding tumor, the C′ gene may not be always expressed. An expression dominance score (EDS) was proposed to measure such expression patterns. For this, the expression level of the (fusion portion) C′ and N′ genes was first calculated as median sequencing depth (E_{c} and E_{N}) in corresponding RNAseq sample. The EDS score was then defined as EDS=E_{c}/E_{N} for each sample. For an index oncogenic fusion, the samples can be categories into (1) positive for the index fusion; (2) positive for other fusions; and (3) negative for fusions. Discrepancy in EDS scores between category and categories (2) and (3) would indicate potential dysregulation of the C′ gene. Because interest was in the relative expression ratio between C′ gene and N′ gene, the global RNAseq normalization procedures (Anders et al. (2010) Genome Biol. 11: R106; Robinson et al. (2010) Bioinformatics 26:139-140) were not needed which renders EDS analysis highly efficient. Such scores are similarly calculated in non-cancer samples from GTEx cohort.

Measuring Relative Selection Bias in Fusion Versioning. Alternative exon (and therefore protein domain) usage due to fusion versioning can potentially lead to differential oncogenicity and therefore selection bias (although it was expected that equal oncogenicity for the different DNA breakpoints would result in a particular fusion version where the same fusion protein is produced; indeed, the nearly uniform distribution of DNA breakpoints observed indicated a lack of additional selection force when conditional on a particular fusion version). Because patient prevalence was largely predicted by gene length (more precisely, length of introns), it was posited that discrepancy between intron length and corresponding patient prevalence can predict relative selection bias (RSB). For this, the observed patient prevalence (Nᵢ) was first calculated for all versions of a given fusion. Next, the patient prevalence was normalized by the length of corresponding intron (Lᵢ). The RSB score was then defined as RBSᵢⱼ=(Nᵢ× Lⱼ)/(Nⱼ× Lᵢ), where i and j indicated the two possible introns in evaluation, in either the N′ gene or the C′ gene. A similar score can be defined for exon-exon combinations. To evaluate statistical significance, chi-square tests were performed by comparing observed patient prevalence against expected patient prevalence under the null hypothesis that involving introns carry equal selection pressure.

Test of Uniformity of DNA Breakpoints in Intron Regions. The uniformity of distribution of DNA breakpoints in intron regions were assessed by using a two-dimensional extension of Kolmogorov-Smirnov test that has found application in astronomy to study the clustering of stars in a pseudo 2-dimensional space.

Splicing Dominance Score. To measure potential alternative splicing, a splicing dominance score (SDS) was introduced. For this, the read support (Xᵢ) was first calculated for all fusion versions i (with minimum of 3 supporting reads) detected in a sample with the index fusion. Next, the dominance score was defined as SDS=X₁/ΣXᵢ. A higher SDS score would indicate lack of alternative splicing.

To study whether alternative splicing in oncogenic fusions was an inherent property of host genes, SDS scores were defined for involving genes in samples without the index fusion (wild-type) in a similar fashion. For this, the fusion-target exon of N′ gene was first defined as the most downstream exon among these fusion versions, and the fusion-target exon of C′ gene as the most upstream exon among these fusion versions. The read supports (Yᵢ) were then calculated for splicing that spanned the target exon of N′ gene (or C′ gene). The dominance score was then defined as SDS=Yᵢ/ΣYᵢ.

Samples of a matched cancer type were categorized into (1) positive for the index fusion; (2) positive for another fusion; (3) negative for all fusions to study the extent of alternative fusions and whether such property was found in corresponding wild-type genes in samples without the index fusion. This method was also applied to GTEx samples as normal control.

Calculation of Hazard Ratio. Event-free survival (EFS) was defined as the time since end of induction I to relapse, death, or last follow-up. Cox proportional hazard regression models were employed to estimate hazard ratios for univariable analysis of EFS in the context of fusion breakpoint and other established prognostic covariates. A p-value <0.05 was considered statistically significant.

Cell Lines. Cell line HAL-01 (RRID:CVCL_1242) was purchased from DSMZ, and STR profiling were performed to confirm identity, followed by whole genome and transcriptome sequencing to confirm DNA and RNA breakpoints (Table 7). STR profiling, whole genome and transcriptome sequencing were also performed to confirm identity and DNA and RNA breakpoints of the cell line UoC-B1 (RRID:CVCL A296) (Table 7). Both cell lines are negative for mycoplasma contamination using MycoAlert Mycoplasma Detection Kit (Lonza).

TABLE 7

| | Wild-type DNA | Non-template | Wild-type DNA |
|---|---|---|---|
| Cell Line | (N′ gene; TCF3) | insertion | (C′ gene; HLF) |
| HAL-01 | GCCCTGTGCCTTCCACCA | AGGGACCGGAGT | TTTCTGGTGCAGGTGGG |
| | GCCCAGGAATCCTGCCTG | CGGGCACGCCTG | TCATTATTTTTAACAGC |
| | CTTTCCAGGCAGACTTTC | AGA (SEQ ID | TGCCAAGTATCCCTTTG |
| | CAAGTACCTTGATTCTAT | NO: 9) | TATGACTGTATCATAAC |
| | CACTCCTAGGCCAGGGCA | | GTGGTTGTTAAATCTCC |
| | TCTCACCGCAG (SEQ | | TATGCATAGTTTTTCC |
| | ID NO: 8) | | (SEQ ID NO: 10) |
| UoC-B1 | CCCTGTGCCTTCCACCAG | TTGGTCCCCTCT | CTTGCTCACCCAGGTAT |
| | CCCAGGAATCCTGCCTGC | CCACCTCGATCT | TCTTCAAAGAGCAGCCT |
| | TTTCCAGGCAGACTTTCC | A (SEQ ID | CCTCCCTCCTACCCAGA |
| | AAGTACCTTGATTCTATC | NO:12) | AGAATTCTGGTAACATC |
| | ACTCCTAGGCCAGGGCAT | | TATTTTGAAAATCGTTT |
| | CTCACCGCAGC (SEQ | | TTTTACCCTGTTGCAT |
| | ID NO: 11) | | (SEQ ID NO: 13) |

Cell Fitness/Dependency Assay. One million HAL-01 or UoC-B1 cells were transiently transfected with precomplexed ribonuclear proteins (RNPs) composed of 150 pmol of chemically modified sgRNA (Synthego) and 50 pmol of SpCas9 protein (St. Jude Protein Production Core) via nucleofection (Lonza, 4D-Nucleofector™ X-unit) using solution P3 and program CA-137 in a small (20 μl) cuvette according to the manufacturer's recommended protocol. For deletion samples, a bridging ssODN donor (3 μg; IDT) was also included in the nucleofection. A portion of cells (˜10% of well) was collected at the indicated day post-nucleofection. Genomic DNA was harvested, amplified, and sequenced via deep sequencing using a 2-step library generation method. Briefly, gene-specific primers with partial Illumina adapters were used to amplify the region of interest in step 1. Gene-specific amplicons were then indexed via nested PCR using primers that bind to the partial Illumina adapters in step 2.

NGS Analysis of Edited Cell Pools. Upon CRISPR editing, targeted amplicon sequencing (using Illumina MiSeq) was performed on the edited cell pools to quantify the induced indels across multiple observation timepoints. For exon targeting (g₁) in cell line HAL-01, the induced indels will lead to frameshift if the length is NOT 3n (3, 6, 9 etc.), which can be analyzed by CRIS.py that measures length of target amplicon reads (Connelly & Pruett-Miller (2019) Sci. Rep. 9:4194). However, the length measurement was not suitable for analyzing splice site disruption in the edited cell pools. Therefore, dedicated in-house methods were developed to analyze such data as below.

For guide g₂ (targeting neo donor in cell line HAL-01), it was expected that the neo donor site GT would be disrupted by the induced Indel. Because it is possible that the indel can happen slightly off the desired GT dinucleotide, the algorithm was designed to account for following three possible editing scenarios: (1) the indel falls into the 5′ coding exon of desired target GT, so that it is still exon targeting per se (“coding” category); (2) the indel falls into the 3′ side of desired target GT so that it that can affect the binding affinity between splicing machinery and the donor motif; and (3) the indel directly disrupts the GT dinucleotide (“loss” category). For scenario (1), the unedited donor motif (from GT to 10 bp downstream) must be intact, and the indel must locate to the 5′ end of this motif. The translation frame of resultant mRNA was subsequently checked by assuming this donor is utilized. To account for potential decrease of binding affinity, the PWM score was also calculated for this donor motif from the mutant read. For scenario (2), the exonic sequence must be intact, and the indel must locate to the 3′ end of the exon. The PWM score was also calculated as described above. For scenario (3), neither the exonic boundary nor the unedited acceptor motif can be found in the mutant sequence. The mutant sequence is scanned for all GT dinucleotides, their PWM scores are calculated, and their translation frame status is determined by assuming they can induce splicing.

This above procedure was similarly applied for guide g₃ (targeting neo acceptor) in cell line HAL-01, except dinucleotide AG was used for acceptor and the PWM was trained from known acceptors of all human genes.

For negative controls g₄ and g₅ (that targets upstream and downstream intronic regions in HAL-1), the percentage of edited reads for 3n and non-3n indels as a negative control for guide g₁ was counted because no functional consequences were expected. Indeed, the editing rate kept ˜95% for both guides from day 3 to day 19 post editing, indicating the high efficiency of nucleofecting approach for CRISPR editing and the non-lethality of g₄ and g₅.

A similar program was written for UoC-B1 editing, although in this cell line the reading frames of all three possible exons: α, β, and δ, were simultaneously considered.

The length of CRISPR-induced indels in the data were also investigated. To account for potential sequencing errors, the analysis was limited to indels with more than 3 read support. In HAL-01, >95% of induced indels had length between −9 and 9. Therefore, “On-Target” editing was defined as indels within 10 base pairs from the designed target position, so that indels with single read support could also be included. Notably, >80% of the induced indels were insertions. For UoC-B1 double targeting, both double focal indels and single large indels were studied. Notably, double focal indels demonstrated a similar pattern to that of single guide targeting. On the other hand, the single large deletions demonstrated lengths centered around −55.

Indel Calling. Considering the double focal indel and large deletion in UoC-B1 experiment, a dedicated script was developed to call indels. Briefly, the wild-type DNA was first prepared as a reference sequence for this locus for BLAST program. Each NGS read was then compared against this reference. Indels were then called by maximizing the perfect match from 5′ end and then from 3′ end. All remaining DNA segments were called as “reference allele” and “mutant allele”, respectively, for the indel, along with the position. Because this procedure generated the same representation for both the large deletions and double focal indels, a post-processing step was performed to further call double focal indels. For this procedure, because the splice site between exons α and β was the critical concern, the presence of a k-mer (CCCAG|GTATT, where the vertical line is the splice site between exons α and β) was confirmed in the mutant allele of each called indel. An indel containing this k-mer was then split to call focal indels by focusing on the DNA segments to the 5′ or to the 3′ of this k-mer, respectively.

Oncogenic fusions typically involve two genomic loci (genes) and said genes are denoted herein as the N′ gene (N-terminus) and C′ gene (N-terminus) for the fusion. All theoretically possible scenarios of gene fusion were enumerated (FIG. 5), where intron/exon structure and translation frame were the main constraints. This theoretical analysis revealed five fusion categories: (1) neo translational, where part of the untranslated region (5′ UTR) in C′ gene is converted into a coding region; (2) intronic versioning, where multiple introns are available to form slightly different fusion proteins; (3) neo splicing, where the DNA breakpoint disrupts natural intron/exon splicing structure so that novel splice sites are created and cryptic exons are formed; (4) chimeric exon, when DNA breakpoints fall into the coding regions of both N′ gene and C′ gene; (5) promoter/enhancer hijacking (e.g., IGH-CRLF2 in B-lineage acute lymphoblastic leukemia (B-ALL)). These five categories encompassed all possible combinations of promoter/enhancer, intron-intron, intron-exon, and exon-exon rearrangements. Because promoter/enhancer hijacking do not form a chimeric protein per se, categories (1)-(4) were focused on in this study using tumor RNAseq data from 5286 tumor samples from childhood cancer patients because promoter/enhancer hijacking does not create a chimeric protein per se and DNA sequence data are required for an unbiased study. Candidate oncogenic fusions were detected by using tools (Arriba, STAR-Fusion, Haas, CICERO, and FusionCatcher) reported to have superior performance (Tian et al. (2020) Genome Biol. 21:126; Haas et al. (2019) Genome Biol. 20:213). The detected candidate fusions were compared with previous genomics studies on childhood cancers (Ma et al. (2018) Nature 555:371-376; Roberts et al. (2014) N. Engl. J. Med. 371:1005-1015; Li et al. (2020) Blood 135:41-55; Chen et al. (2013) Cancer Cell 24:710-724; Zhang et al. (2013) Nat. Genet. 45:602-612; Crompton et al. (2014) Cancer Discov. 4:1326-1341; Parker et al. (2014) Nature 506:451-455; Shern et al. (2014) Cancer Discov. 4:216-231; Tirode et al. (2014) Cancer Discov. 4:1342-1353; Wu et al. (2014) Nat. Genet. 46:444-450; Andersson et al. (2015) Nat. Genet. 47:330-337; Lu et al. (2015) J. Invest. Dermatol. 135:816-823; Faber et al. (2016) Nat. Genet. 48:1551-1556; Bolouri et al. (2018) Nat. Med. 24:103-112; Hyrenius-Wittsten et al. (2018) Nat. Commun. 9:1770; Rusch et al. (2018) Nat. Commun. 9:3962) to establish the comprehensive list of oncogenic fusions. For detected oncogenic fusions, the fusion was classified into one of above four categories by using novel tools (Neo-Versioner and Neo-Splicer). If the fusion did not belong to either neo versioning or neo splicing categories by the automated analysis, the fusion was manually reviewed and classified into the categories of either chimeric exon or neo translational.

Landscape of Oncogenic Fusions in Childhood Cancers. Of the large cohort of 5286 childhood cancer patients, oncogenic fusions were identified for 55.7% of leukemia (1470/2642), 21.7% of brain tumor (337/1554) and 18.7% of solid tumor (204/1093) patients, respectively. Among the 2005 fusion positive patients, only 7 (0.35%) had two different fusions such as BCR-ABL1 and CBFB-MYH11 in patient PARBLV; FGFR1-TACC1 And FGFR3-TACC3 in patient PT 7DTGJYA7; KMT2A-MATR3 and MEF2D-DAZAP1 in SJBALL020141 patients and SJBALL020142; CBFB-MYH11 and RUNX1-RUNX1T1 in patients SJCBF124 and SJCBF149; and KLHL7-MET and VCL-NTRK2 in patient SJHGG009. Among the oncogenic fusions, 34 neo splicing (Table 8), 33 neo translational (Table 9) and 11 chimeric exon events were detected (Table 10).

TABLE 8

| | | | |
|---|---|---|---|
| | Wild-type DNA | Non-template | Wild-type DNA |
| Fusion | (N′ gene) | insertion | (C′ gene) |

B-Cell Acute Lymphoblastic Leukemia

| | | | |
|---|---|---|---|
| TCF3_HLF | TGCCTCTTCATTCGCCTG | ACTGAGAG | ACTTGCAGTTGAGGAAA |
| | CTCCCAGACGCTGTGTGC | | TGCAGAAAATGGAAAGC |
| | CTGGCAGCGCTCAGCACT | | TGAAGTCAGCGGATCAC |
| | GGGGAAGGGGCGAGGGGT | | TACCTGTTAGAGAAAGG |
| | GCAGCAGGATGCCTCTGC | | CTTAGCCTGGCTCCCAG |
| | CTCAGGGGAGA (SEQ | | GTTGTCTTGCTTCCCA |
| | ID NO: 14) | | (SEQ ID NO: 15) |
| TCF3_HLF | GGCTGCGGGGAGGACTTG | ATTAAAA | CACTGTTGTTAACTGGA |
| | GGATTTGGCCATGAGAAA | | GGCTTCACCACTTTGGG |
| | GGTGGCAGCCGTGGAGGG | | CCCCCTCACCACCATGA |
| | CTGAGGAGGGATGGGACC | | CGTCATTGACTCGCCTG |
| | TGACCCAGGTGCTCACAG | | ACTCCTCCCAGCCTCTC |
| | ATACCCTCTGG (SEQ | | CCCTGCCTCCAGCTCC |
| | ID NO: 16) | | (SEQ ID NO: 17) |
| TCF3_HLF | GGCCCTGTGCCTTCCACC | GTCTCCAAACCC | GCTTGCTCACCCAGGTA |
| | AGCCCAGGAATCCTGCCT | (SEQ ID | TTCTTCAAAGAGCAGCC |
| | GCTTTCCAGGCAGACTTT | NO: 19) | TCCTCCCTCCTACCCAG |
| | CCAAGTACCTTGATTCTA | | AAGAATTCTGGTAACAT |
| | TCACTCCTAGGCCAGGGC | | CTATTTTGAAAATCGTT |
| | ATCTCACCGCA (SEQ | | TTTTTACCCTGTTGCA |
| | ID NO: 18) | | (SEQ ID NO: 20) |
| TCF3_HLF | GCCCTGTGCCTTCCACCA | AGGGACCGGAGT | TTTCTGGTGCAGGTGGG |
| | GCCCAGGAATCCTGCCTG | CGGGCACGCCTG | TCATTATTTTTAACAGC |
| | CTTTCCAGGCAGACTTTC | AGA (SEQ ID | TGCCAAGTATCCCTTTG |
| | CAAGTACCTTGATTCTAT | NO:79) | TATGACTGTATCATAAC |
| | CACTCCTAGGCCAGGGCA | | GTGGTTGTTAAATCTCC |
| | TCTCACCGCAG (SEQ | | TATGCATAGTTTTTCC |
| | ID NO: 78) | | (SEQ ID NO: 80) |
| TCF3_HLF | CCCTGTGCCTTCCACCAG | TTGGTCCCCTCT | CTTGCTCACCCAGGTAT |
| | CCCAGGAATCCTGCCTGC | CCACCTCGATCT | TCTTCAAAGAGCAGCCT |
| | TTTCCAGGCAGACTTTCC | A (SEQ ID | CCTCCCTCCTACCCAGA |
| | AAGTACCTTGATTCTATC | NO:82) | AGAATTCTGGTAACATC |
| | ACTCCTAGGCCAGGGCAT | | TATTTTGAAAATCGTTT |
| | CTCACCGCAGC (SEQ | | TTTTACCCTGTTGCAT |
| | ID NO: 81) | | (SEQ ID NO: 83) |
| KMT2A-MLLT3 | CCGCCAAGAAAAGAAGTT | | AATACACAGAAAAAAAA |
| | CCCAAAACCACTCCTAGT | | TTCCTAGAAAATAGAGA |
| | GAGCCCAAGAAAAAGCA | | TTCTTTAATAAATA |
| | (SEQ ID NO: 84) | | (SEQ ID NO: 85) |

Acute Myeloid Leukemia

| | | | |
|---|---|---|---|
| CBFB_MYH11 | TTAGAACATTATTAAAAC | | GGCCCTGTCCCTGGCTC |
| | TCGAGTAATACTACTTTC | | GGGCCCTTGAAGAGGCC |
| | CATTTTTCTATGAATATT | | TTGGAAGCCAAAGAGGA |
| | TGTCTTGGTTTTATAACT | | ACTCGAGCGGACCAACA |
| | ATAATTGTCAGTCATTTG | | AAATGCTCAAAGCCGAA |
| | TGTGATTTTAA (SEQ | | ATGGAAGACCTGGTCA ( |
| | ID NO: 21) | | SEQ ID NO: 22) |
| CBFB_MYH11 | ATTACTTATTGTAACTGT | TC | GAGCTTCAGGCCGACTC |
| | ATTATTCCTAAAAGTATA | | TGCCATCAAGGGGAGGG |
| | AGGTCATGTTTAACTGAA | | AGGAAGCCATCAAGCAG |
| | ATTATATAATATTTTGAC | | CTACGCAAACTGCAGGT |
| | CCACAAATTGATTTTATT | | GGGTGACACTAGGAGCT |
| | ATATTGCTAGC (SEQ | | TGGGGCATGGGTGGAG |
| | ID NO: 23) | | (SEQ ID NO: 24) |
| CBFB_MYH11 | TATTTAGAAAAAAATAAA | | CCAAGCGGGCCCTGGAG |
| | ATTTGCTTTCAGTATTAC | | ACCCAGATGGAGGAGAT |
| | ACAGAATAATGAAAACAG | | GAAGACGCAGCTGGAAG |
| | AAGATTCTAGACCTTGCT | | AGCTGGAGGACGAGCTG |
| | ATCTCTATTCTCTGGCAT | | CAAGCCACGGAGGACGC |
| | ATAGGCTGTTT (SEQ | | CAAACTGCGGCTGGAA |
| | ID NO: 25) | | (SEQ ID NO: 26) |
| CBFB_MYH11 | TGTGATTCAGATTATCTT | | CTCAACGTGTCTACGAA |
| | TAGAGATTCAAACATCAT | | GCTGCGCCAGCTGGAGG |
| | CTAATCTAACATTATTAC | | AGGAGCGGAACAGCCTG |
| | AGTTGATAAAACTGAGAG | | CAAGACCAGCTGGACGA |
| | TCTCCAAAAAATTAATTG | | GGAGATGGAGGCCAAGC |
| | ACTTGCTCTCC (SEQ | | AGAACCTGGAGCGCCA |
| | ID NO: 27) | | (SEQ ID NO: 28) |
| FUS_FLI1 | AAAATTCCCAACTCCCAG | | GCTGGTCTTTCATTTGT |
| | CAATGCTTTGTCTGATTG | | CTTGTTTGTTTTTAAGC |
| | TTCATTTGCAGATGTCTT | | AAGAAGAATCCCTTTAG |
| | AGCGTGTTAATTTAAATG | | AGGAGGAATTAGGAAAG |
| | TCAAAGGTTTTGAGGTGT | | AAAAAAAAGTCAAACAG |
| | CCAGAACCACC (SEQ | | AAACAGAAGGAGTGGA |
| | ID NO: 29) | | (SEQ ID NO: 30) |
| KAT6A_EP300 | CCAGAATGACGACCACGA | TG | TTTTTTTTTGAGACGGA |
| | CGCTGATGATGAGGATGA | | GTTTAGCTCTTGTTGCC |
| | TGGCCACCTGGAGTCCAC | | CAGGCTGGAGTGCAGTG |
| | AAAGAAAAAGGAGCTAGA | | GTACGATCTCGGCTCAC |
| | GGAACAGCCCACGAGGGA | | TGCAACTTCTCCCTCCT |
| | AGATGTCAAGG (SEQ | | GGTTTCAAGCAACTCT |
| | ID NO: 31) | | (SEQ ID NO: 32) |
| NUP98_BPTF | AACTTTTTTGTATGGATA | | AAGTCCAAGAAAAAGAA |
| | TGTAGGGCTTGGCGAGTC | | AATGATCTCTACTACCT |
| | TAGGTCAAGCATTCCAGC | | CAAAGGAAACTAAGAAG |
| | CAAAGAATTGTGAAAGAT | | GACACAAAGCTTTACTG |
| | CACAACAATCTGGGAATA | | TATCTGTAAAACGCCTT |
| | ACAAAGATTCA (SEQ | | ATGATGAATCTAAGTG |
| | ID NO: 33) | | (SEQ ID NO: 34) |

Brain Tumor

| | | | |
|---|---|---|---|
| C11orf95_NCOA2 | GGGGCGCGCTGGCCACGC | | ATCCAGAAATGTAATTT |
| | TCAAGGTGAGCACCATCA | | ATTCTCAGTCTTCACTG |
| | AGCGCCACATCCTGCAGG | | AAGAGCATCTGGCTCTT |
| | TGCACCCCTTCTCCATGG | | GAGCTGGAAATATGGCT |
| | ACTTCACGCCTGAGGAGC | | CTATAAGCTTTATTGTA |
| | GCCAGACTATC (SEQ | | TAGCTGAGTTTCTCTG |
| | ID NO: 35) | | (SEQ ID NO: 36) |
| C11orf95_NCOA2 | CTACCAGCCGCGGTGGCG | | TGGTATGTAAATTCAAA |
| | GGGCGAGTACCTGATGGA | | ACTAGAATAATAGGCTA |
| | CTACGACGGCAGCCGGCG | | CATTATGTGCTCTCATT |
| | CGGCCTGGTGTGTATGGT | | GTCTGAAAAATAAGTTC |
| | GTGCGGGGGCGCGCTGGC | | CCTGAAAAAATCCAGGA |
| | CACGCTCAAGG (SEQ | | TACCTTAAGTGATATT |
| | ID NO: 37) | | (SEQ ID NO: 38) |
| C11orf95_NCOA2 | TGAGCACCATCAAGCGCC | GCGGCA | AGGTGTGGACTACCACA |
| | ACATCCTGCAGGTGCACC | | CCTAGCCTAAATCTAGA |
| | CCTTCTCCATGGACTTCA | | ACTTTCTATGTATATAT |
| | CGCCTGAGGAGCGCCAGA | | TTACAAATAATATTTTA |
| | CTATCCTGGAGGCCTACG | | GATTTTTGTTCTCTGGT |
| | AGGAGGCGGCG (SEQ | | TCAAATTAACTTCTCA |
| | ID NO: 39) | | (SEQ ID NO: 40) |
| C11orf95_MAML2 | CAGGTGCACCCCTTCTCC | | CGTCCGGCAACAAAGGA |
| | ATGGACTTCACGCCTGAG | | TGTTTTGTGCTACTACT |
| | GAGCGCCAGACTATCCTG | | GAGGTTTGTGTGTGTGA |
| | GAGGCCTACGAGGAGGCG | | CTTACTTTAGAACTCTT |
| | GCGCTGCGCTGCTACGGC | | TCTAGAAAATGCGATTA |
| | CACGAGGGCTT (SEQ | | CTATTTGCATAGGTCT |
| | ID NO: 41) | | (SEQ ID NO: 42) |
| C11orf95_MAML2 | GAGCACCATCAAGCGCCA | CGGGGCGGGCGG | AAAAAATCAGAATAAAC |
| | CATCCTGCAGGTGCACCC | CCCGAAGCCCTC | AATTTGGTCAAGTAAAA |
| | CTTCTCCATGGACTTCAC | GTGGGGTAATTA | TATTTCCCTCCAAGTAG |
| | GCCTGAGGAGCGCCAGAC | AAACGTTATTTT | TTAAGGCAAAGACTGAA |
| | TATCCTGGAGGCCTACGA | CTTTTCTTT | GGACCATTTGTAGGAAA |
| | GGAGGCGGCGC (SEQ | (SEQ ID | TGGAGAATCTTTCTAT |
| | ID NO: 43) | NO: 44) | (SEQ ID NO: 45) |
| MN1_PATZ1 | GGCAACTGAATCTAGCAG | | TCCATGCGGTCTATGTG |
| | TTTGGAGGTCTTAGAGCA | | GTAAGGTGTTCACTGAT |
| | TTTGTAATAACATGCTGG | | GCCAACCGGCTCCGGCA |
| | CTCTCTGTGAATGTCCCA | | GCACGAGGCCCAGCACG |
| | GAAGGAACATCTTCCATG | | GTGTCACCAGCCTCCAG |
| | GAATGGACTTG (SEQ | | CTGGGCTACATCGACC |
| | ID NO: 46) | | (SEQ ID NO: 47) |
| MN1_PATZ1 | ACTCTTCGTGTGTTCTTT | | GGCCTGAGGGAGGCAGG |
| | GATCAAGTCAGGACTATT | | CATCCTTCCATGCGGTC |
| | ACTTCCATTGCAGGGAGA | | TATGTGGTAAGGTGTTC |
| | CTGAGGCCCAGAGAGGGA | | ACTGATGCCAACCGGCT |
| | AAGTGCCTTGTCCAAAGT | | CCGGCAGCACGAGGCCC |
| | CACACAGCTGG (SEQ | | AGCACGGTGTCACCAG |
| | ID NO: 48) | | (SEQ ID NO: 49) |
| MN1_PATZ1 | GCTCAAGCATTGCTACGT | ACATT | CAAGCCTCTTTGCCTGT |
| | TCATTCCTTGAGATAATT | | GTTACCTGGGGTGGACC |
| | TGTGCAAAGTGGGGGAAA | | GCTTGCCCATGGTGGCT |
| | TAACCCCCTTTCAGATTT | | GGACCCCTATCCCCCCA |
| | TCTCTCTTTCTCTCTCTC | | ACTGCTGACTTCCCCAT |
| | TGTGGCAGGTA (SEQ | | TCCCCAGTGTGGCATC |
| | ID NO: 50) | | (SEQ ID NO: 51) |
| MN1_PATZ1 | CTGCTTTGCCCATCAGTC | ACGTCTCTGG | GCTGGGCTACATCGACC |
| | TGTCCTTTCAGAGTTGAA | (SEQ ID | TTCCTCCTCCGAGGCTG |
| | GCTGAGCTGCTGTTTGCT | NO:53) | GGTGAGAATGGGCTACC |
| | GGGCAGGCCATGCAGCCC | | CATCTCTGAAGACCCCG |
| | ACACGGGGGTCCTCAGAG | | ACGGCCCCCGAAAGAGG |
| | GCCTTGCAGGG (SEQ | | AGCCGGACCAGGAAGC |
| | ID NO: 52) | | (SEQ ID NO: 54) |
| MN1_CXXC5 | CCACTGTCCTACCCGAGT | | GCGCGTGGTGCAGGAGC |
| | GAGGCTTGTTACAGACAT | | ATCTCCCGCTGATGAGC |
| | CAGGGCCCACCTGACTGT | | GAGGCGGGTGCTGGCCT |
| | GGTGGGCTACACGAGGAT | | GCCTGACATGGAGGCTG |
| | GCTCACATTTCCTCCATT | | TGGCAGGTGCCGAAGCC |
| | AGTCACCTGAT (SEQ | | CTCAATGGCCAGTCCG |
| | ID NO: 55) | | (SEQ ID NO: 56) |
| TPM3_NTRK1 | CCAGGAAGGTCTAGCTCC | | TCTCGGTGGCTGTGGGC |
| | TGACACGTTCTATGGTAG | | CTGGCCGTCTTTGCCTG |
| | AGGGAGGAGGGTTGATGC | | CCTCTTCCTTTCTACGC |
| | TTGCTCAGGTTACTTGGG | | TGCTCCTTGTGCTCAAC |
| | AACATCTCTTCCCCAGTA | | AAATGTGGACGGAGAAA |
| | TGCCTTCCAAC (SEQ | | CAAGTTTGGGATCAAC |
| | ID NO: 57) | | (SEQ ID NO: 58) |
| SPTBN1_ALK | CCGCCACTTTGCTGGCAC | | ATGCACTAGCCCACTCT |
| | CTGCTCTAAACATCTGGT | | TCCCCAAACCAGCCCTC |
| | CTCTGCTGCTTGGCTCTC | | CACCACCCTCCAGGCAG |
| | AGGAGCAAAGGTATAAGG | | AGAGATAGGAAAATCGG |
| | ACGTGGCCAATGCTAGGT | | TTTCTGAGTATATTTCT |
| | TATTAGCTTAG (SEQ | | GTTCAGCCTGTGAGCC |
| | ID NO: 59) | | (SEQ ID NO: 60) |

Solid Tumor

| | | |
|---|---|---|
| BCOR_CCNB3 | CATATGAAAATATCTCTT | CTTTTAGTAATTCAGTA |
| | CTTTATATAAGAGAAATT | CCTGTTTGAGCTAGTCT |
| | ACTCCAGTCAGAAGGACT | GTGCTTTATAGTGTGGA |
| | TAGAAACATGTTTTTTTC | GACAACTTAACTTTCCA |
| | CTTTTAAACTTTTAAGTC | GGGATTCTCAGCAGCTG |
| | AGTTTTTATGA (SEQ | ACTGGTAGCTTGCCAG |
| | ID NO: 61) | (SEQ ID NO: 62) |
| BCOR_CCNB3 | CTTGGTGATATAACTTTG | GTGTGGCCACCACACCA |
| | TTTTGTTTACAGAGTACC | GCTTTTTTTTTTTTTTT |
| | TGCTCGGGCCAGGTAAAT | TTCGTATTTTTGTAGAG |
| | GCTATTGGATGTAATCCA | ACATGGTTTCACTATGT |
| | GTAGTGTGTAATATAAAT | TGGGCAGGCTGGTCTCG |
| | TCAAACCATAT (SEQ | AACTCCTGACCTGAAG |
| | ID NO: 63) | (SEQ ID NO: 64) |
| EWSR1_ATF1 | TAAATAGCATTTTTTAAA | CAAGGTACAACTATTCT |
| | AAACAGAATGAACTTCAA | TCAGTATGCACAGACCT |
| | AATTAAAGTTGATTTTTA | CTGATGGACAGCAGATA |
| | ACTTCCATATTAGCAAAT | CTTGTGCCCAGCAATCA |
| | ACTCTTCACTACTGAAAG | GGTGGTCGTACAAAGTA |
| | ACAGTACTATT (SEQ | AGTATGCTTTCTGTCT |
| | ID NO: 65) | (SEQ ID NO: 66) |

TABLE 9

| | No. of | Gene with Neo- | Exon being neo- |
|---|---|---|---|
| Fusion | cases | translation | translated |
| PPP1CB_ALK | 2 | ALK | E1 |
| C11orf95_RELA | 3 | RELA | Intron 1; also |
| | | | chimeric |
| | | | exon/intron |
| PAX5_NCOA5 | 1 | NCOA5 | E2 |
| CLIP1_ALK | 1 | ALK | E1 |
| MAP3K8_GNG2 | 2 | GNG2 | E3 |
| TCF3_ZNF384 | 1 | ZNF384 | E3 |
| EP300_ZNF384 | 8 | ZNF384 | E3 |
| ARID1B_ZNF384 | 1 | ZNF384 | E3 |
| TAF15_ZNF384 | 2 | ZNF384 | E3 |
| SMARCA2_ZNF384 | 1 | ZNF384 | E3 |
| TCF3_ZNF384 | 3 | ZNF384 | E2 |
| EP300_ZNF384 | 3 | ZNF384 | E2 |
| MAP3K8_SVIL | 1 | SVIL | E4 |
| YAP1_FAM118B | 4 | FAM118B | E3 |
| KMT2A_MLLT11 | 3 | MLLT11 | E2 |
| SFPQ_ZFP36L2 | 2 | ZFP36L2 | 5′ UTR |

TABLE 10

| | Gene Orientation | | |
|---|---|---|---|
| Fusioni | N′ | C′ | RNA contig |
| C11orf95_YAP1 | − | + | GACGAGGAGGAGGAGCCAGAGGAGGAGGAGGAG |
| | | | GAGTGGGGCGACGTTCCGCTGTCCCCTGGAGCT |
| | | | CCCTTGGAGCGGCCCGCCGAAGAAGAGGAGGAC |
| | | | GAAGAGGACGGCCAGGAGCCTGGGGGACTCGCC |
| | | | TTGCCGCCGCCGCCTCCTCCCCCGCCTCCGCCC |
| | | | CCGCCCCGCAGCCGGGAGCAGCGGCGGAACTAC |
| | | | CAGCCGCGGTGGCGGGGCGAGTACCTGATGGAC |
| | | | TACGACGGCAGCCGGCGCGGCCTGGTGTGTATG |
| | | | GTGTGCGGGGGCGCGCTGGCCACGCTCAAGGTG |
| | | | AGCACCATCAAGCGCCACATCCTGCAGGTGCAC |
| | | | CCCTTCTCCATGGACTTCACGCCTGAGGAGCGC |
| | | | CAGACTATCCTGGAGGCCTACGAGGAGGCGGCG |
| | | | CTGCGCTGCGACCTGGAGGCGCTCTTCAACGCC |
| | | | GTCATGAACCCCAAGACGGCCAACGTGCCCCAG |
| | | | ACCGTGCCCATGAGGCTCCGGAAGCTGCCCGAC |
| | | | TCCTTCTTCAAGCCGCCGGAGCCCAAATCCCAC |
| | | | TCCCGACAGGCCAGTTGTATAGTCTCCTGTCGG |
| | | | AGACCAAAGGGTTTTGGAACTCAGAAAAAAT |
| | | | (SEQ ID NO: 67) |
| C11orf95_MAML2 | − | − | CCGGGAGCAGCGGCGGAACTACCAGCCGCGGTG |
| | | | GCGGGGCGAGTACCTGATGGACTACGACGGCAG |
| | | | CCGGCGCGGCCTGGTGTGTATGGTGTGCGGGGG |
| | | | CGCGCTGGCCACGCTCAAGGTGAGCACCATCAA |
| | | | GCGCCACATCCTGCAGGTGCACCCCTTCTCCAT |
| | | | GGACTTCACGCCTGAGGAGCGCCAGACTATCCT |
| | | | GGAGGCCTACGAGGAGGCGGCAGGAGCTGGCAA |
| | | | ACACACCAAGGCCACCGCCACTGCTGCCACCAC |
| | | | TACAGCCCCTCCACCGCCCCCTGCTGCCCCTCC |
| | | | TGCGGCCTCCCAAGCAGCAGCAACAGCAGCCCC |
| | | | ACCGCCCCCACCAGACTATCACCATCACCACCA |
| | | | GCAGCACCTGCTGAACAGTAGCAATAATGGTGG |
| | | | CAGTGGTGGGATAAACGGAGAGCAGCAGCCGCC |
| | | | CGCTTCAACCCCAGGGGACCAGAGGAACTCAGC |
| | | | CCTGATTGCGGATATTCCTTAACTGATAAGAAG |
| | | | C (SEQ ID NO: 68) |
| ATXN1_NUTM2B | − | + | GATCGACTCCAGCACCGTAGAGAGGATTGAAGA |
| | | | CAGCCATAGCCCGGGCGTGGCCGTGATACAGTT |
| | | | CGCCGTCGGGGAGCACCGAGCCCAGGTCAGCGT |
| | | | TGAAGTTTTGGTAGAGTATCCTTTTTTTGTGTT |
| | | | TGGACAGGGCTGGTCATCCTGCTGTCCGGAGAG |
| | | | AACCAGCCAGCTCTTTGATTTGCCGTGTTCCAA |
| | | | ACTCTCAGTTGGGGATGTCTGCATCTCGCTTAC |
| | | | CCTCAAGAACCTGAAGAACGGCTCTGTTAAAAA |
| | | | GGGCCAGCCCGTGGATCCCAGCAAGGCCGGCCC |
| | | | CAAGGCCCCGACTGCCTGCCTGCCACCACCCAG |
| | | | GCCCCAGAGGCCAGTGACCAAGGCCCGCCGGCC |
| | | | ACCACCCCGGCCCCACCGGCGAGCAGAGACCAA |
| | | | GGCCCGCCTGCCACCACCCAGGCCCCAGAGACC |
| | | | AGCAGAGACCAAGGTCCCTGAGGAGATCCCCCC |
| | | | AGAAGTGGTGCAGGAGTATGTGGACATCATGGA |
| | | | GGAGCTGCTAGG (SEQ ID NO: 69) |
| MRC1_PDGFRB | + | − | CCTACAAAGGATATATTTGTAAAAGACCAAAAA |
| | | | TTATTGATGCTAAACCTACTCATGAATTACTTA |
| | | | CAACAAAAGCTGACACAAGGAAGATGGACCCTT |
| | | | CTAAACCGTCTTCCAACGTGGCCGGAGTAGTCA |
| | | | TCATTGTGATCCTCCTGATTTTAACGGGTGCTG |
| | | | GCCTTGCCGCCTATTTCTTTTATAAGAAAAGAC |
| | | | GTGTGCACCTACCTCAAGAGGGCGCCTTTGAAA |
| | | | ACACTCTGTATTTTGAGTCTGTGAGCTCTGACG |
| | | | GCCATGAGTACATCTACGTGGACCCCATGCAGC |
| | | | TGCCCTATGACTCCACGTGGGAGCTGCCGCGGG |
| | | | ACCAGCTTGTGCTGGGACGCACCCTCGGCTCTG |
| | | | GGGCCTTTGGGCAGGTGGTGGAGGCCACGGCTC |
| | | | ATGGCCTGAGCCATTCTCAGGCCACGATGAAAG |
| | | | TGGCCGTCAAGATGCTTAAATCCACAGCCCGCA |
| | | | GCAGTGAGAAGCAAGCCCTTATGTCGGAGCTGA |
| | | | AGATCATGAGTCACCTTGGGCCCCAC (SEQ |
| | | | ID NO: 70) |
| EP300_BCOR | + | − | GTGCGCTCTCCCCAGCCTGTCCCTTCTCCACGG |
| | | | CCACAGTCCCAGCCCCCCCACTCCAGTCCTTCC |
| | | | CCAAGGATGCAGCCTCAGCCTTCTCCACACCAC |
| | | | GTTTCCCCACAGACAAGTTCCCCACATCCTGGA |
| | | | CTGGTAGCTGCCCAGGCCAACCCCATGGAACAA |
| | | | GGGCATTTTGCCAGCCCGGACCAGAATTCAATG |
| | | | CTTTCTCAGCTTGCTAGCAATCCAGGCATGGCA |
| | | | AACCTCCATGGTGCAAGCGCCACGGACCTGGGA |
| | | | CTCAGCACCGATTTATGTCTACCCGCTGCTTAC |
| | | | TGTGAGCGTGCAATGATGCGCTTCTCAGAGTTG |
| | | | GAGATGAAAGAAAGAGAAGGTGGCCACCCAGCA |
| | | | ACCAAAGACTCCGAGATGTGCC (SEQ ID |
| | | | NO: 71) |
| CBFA2T3_GLIS2 | − | + | TGGAACTGCGGGCGGAAAGCCAGTGAGACGTGC |
| | | | AGCGGCTGCAACGCGGCACGCTACTGCGGGTCC |
| | | | TTCTGCCAGCATCGGGACTGGGAGAAGCATCAC |
| | | | CACGTGTGTGGCCAGAGCCTGCAGGGCCCCACA |
| | | | GCCGTGGTGGCCGACCCGGTGCCTGGACCGCCC |
| | | | GAAGCCGCCCACAGCCTGGGCCCCTCCCTGCCT |
| | | | GTGGGTGCTGCCAGCCTGGTGGATGACAGCCCC |
| | | | ACACCTGGCTCTCCAGGCTCCCCGCCCTCAGGC |
| | | | TTCCTGCTGAACTCCAAGTTCCCCGAGAAGGTG |
| | | | GAGGGACGCTTTTCAGCAGCCCCTCTCGTGGAC |
| | | | CTCAGCCTGTCACCACCATCTGGGCTGGACTCC |
| | | | CCCAATGGCAGCAGCTCGCTGTCCCCCGAGCGC |
| | | | CAGGGCAACGGGGACCTGCCTCCAGTG (SEQ |
| | | | ID NO: 72) |
| C11orf95_RELA | − | − | GGCGCCTGGAGAGGAGGCTGAAGGAGTCCCTGC |
| | | | AGAACTGGTTCCGGGCCGAGTGTCTCATGGACT |
| | | | ATGACCCGCGGGGGAACCGGCTGGTGTGCATGG |
| | | | CCTGTGGCCGGGCACTGCCCAGCCTGCACCTGG |
| | | | ACGACATCCGTGCCCACGTGCTGGAGGTGCACC |
| | | | CTGGCTCCCTGGGGCTCAGCGGCCCCCAGCGCA |
| | | | GTGCCCTGCTGCAGGCCTGGGGGGGCCAGCCCG |
| | | | AGGCGCTGTCTGAGCTCACCCAGTCCCCACCAG |
| | | | GCGATGACCTCGCCCCCCAGGACCTGACCGGAA |
| | | | AGAGCCGGGACTCGGCCTCCGCTGCTGGAGCCC |
| | | | CCTCCTCTCAGGATCCCTCTGGCCCCTATGTGG |
| | | | AGATCATTGAGCAGCCCAAGCAGCGGGGCATGC |
| | | | GCTTCCGCTACAAGTGCGAGGGGCGCTCCGCGG |
| | | | GCAGCATCCCAGGCGAGAGGAGCACAGATACCA |
| | | | CCAAGACCCACCCCACCATCAAGATCAATGGCT |
| | | | ACACAGGACCAGGGACAGTGCGCATCTCCCTGG |
| | | | TCACCAAGGACCCTCCTCACCGGCCTCACCCCC |
| | | | ACGAGCTTGTAGGAAAGGACTGCCGGGATGGCT |
| | | | TCTATGAGGCTGAGCTCTGCCCGGACCGCTGCA |
| | | | TCCACAGTTTCCAGAACCTGGGAATCCAGTGTG |
| | | | TGAAGAAGCGGGACCTGGAGCAGGCTATCAGTC |
| | | | AGCGCATCCAGACCAA (SEQ ID NO: 73) |
| EP300 BCOR | + | − | AAAACCTTTTGCGGACTCTCAGGTCTCCCAGCT |
| | | | CTCCCCTGCAGCAGCAACAGGTGCTTAGTATCC |
| | | | TTCACGCCAACCCCCAGCTGTTGGCTGCATTCA |
| | | | TCAAGCAGCGGGCTGCCAAGTATGCCAACTCTA |
| | | | ATCCACAACCCATCCCTGGGCAGCCTGGCATGC |
| | | | CCCAGGGGCAGCCAGGGCTACAGCCACCTACCA |
| | | | TGCCAGGTCAGCAGGGGGTCCACTCCAATCCAG |
| | | | CCATGCAGAACATGAATCCAATGCAGGCGGGCG |
| | | | TTCAGAGGGCTGGCCTGCCCCAGCAGCAACCAC |
| | | | AGCAGCAACTCCAGCCACCCATGGGAGGGATGA |
| | | | GCCCCCAGGCTCAGCAGATGAACATGAACCACA |
| | | | ACACCATGCCTTCACAATTCCGAGACATCTTGA |
| | | | GACCTGTGTTCTCCGGCTCTCCGCCCATGAAGA |
| | | | GTCTTTCATCCACCAGTGCAGGCGGCAAAAAGC |
| | | | AGGCTCAGCCAAGCTGCGCACCAGCCTCCAGGC |
| | | | CGCCTGCCAAACAGCAGAAAATTAAAGAAAACC |
| | | | AGAAGACAGATGTGCTGTGTGCAGACGAAGAAG |
| | | | AGGATTGCCAGGCTGCCTCCCTGCTGCAGAAAT |
| | | | ACACCGACAACAGCGAGAAGCCATCCGGGAAGA |
| | | | GACTGTGCAAAACCAAACACTTGATCCCTCAGG |
| | | | AGTCCAGGCGGGGATTGCCACTGACAGGGGAAT |
| | | | ACTACGTGGAGAATGCCGATGGCAAGGTGACTG |
| | | | TCCGGAGATTCAGAAAGCGGCCGGAGCCCAGTT |
| | | | CGGACTATGATCTGTCACCAGCCAAGCAGGAGC |
| | | | CAAAGCCCTTCGACCGCTTGCAGCAACTGCTAC |
| | | | CAGCCTCCCAGTCCACACAGCTGCCATGCTCAA |
| | | | GTTCCCCTCAGG (SEQ ID NO: 74) |
| CBFA2T3_GLIS2 | − | + | CGCGAGGAGCTCAACCACTGGGCGCGGCGCTAC |
| | | | AGCGACGCCGAGGACACAAAGAAGGGCCCCGCT |
| | | | CCCGCCGCGGCCCGGCCCCGCAGCAGCTCCGCC |
| | | | GGTCCCGAGTGCCTCTCGCCAGACCTGCCCCTG |
| | | | CCCAAGCAGCTGGTGTGTCGCTGGGCCAAGTGT |
| | | | AACCAGCTCT (SEQ ID NO: 75) |
| NUP98_KDM5A | − | − | ATTTGGAACAGCTCTTGGTGCTGGACAGGCATC |
| | | | TTTGTTTGGGAACAACCAACCTAAGATTGGAGG |
| | | | GCCTCTTGGTACAGGAGCCTTTGGGGCCCCTGG |
| | | | ATTTAATACTACGACAGCCACTTTGGGCTTTGG |
| | | | AGCCCCCCAGGCCCCAGTAGAAAAGGTAGAGCA |
| | | | ACTTTTTGGAGAAGGAAAACAGAAGTCCAAGGA |
| | | | GTTAAAGAAAATGGACAAACC (SEQ ID |
| | | | NO: 76) |
| CIC_FOX04 | + | + | CTGCGGCGCACCCTGGACCAGCGCCGGGCCCTG |
| | | | GTCATGCAGCTCTTTCAGGACCATGGCTTCTTC |
| | | | CCGTCAGCCCAGGCCACAGCCGCCTTCCAGGCC |
| | | | CGCTATGCAGACATCTTTCCCTCCAAGGTTTGT |
| | | | CTGCAGTTGAAGATCCGTGAGGTGCGCCAGAAG |
| | | | ATCATGCAGGCTGCCACTCCCACGGAGCAGCCC |
| | | | CCTGGAGCTGAGGCTCCTCTCCCTGTACCGCCC |
| | | | CCCACTGGCACCGCTGCTGCCCCTGCCCCCACT |
| | | | CCCAGCCCCGCAGGGGGCCCTGACCCCACCTCA |
| | | | CCCAGCTCGGACTCTGGCACGGCCCAGGCTGCC |
| | | | CCGCCACTGCCTCCACCCCCAGAGTCGGGGCCT |
| | | | GGACAGCCTGGCTGGGAGGTTACCGGCCCCTTA |
| | | | CACACCTACAGCAGCTCCCTTTTCAGCCCAGCA |
| | | | GAGGGGCCCCTGTCAGCAGGAGAAGG (SEQ |
| | | | ID NO: 77) |
| FGFR3_TACC3 | + | + | ACCTTCAAGCAGCTGGTGGAGGACCTGGACCGT |
| | | | GTCCTTACCGTGGCCAGCTCCTCGAGGAGCGGA |
| | | | CCTGTAAAACTAGAATTTGATGTATCTGATGGC |
| | | | GCCACCAGCAAAAGGG (SEQ ID NO: 86) |

The remaining 1,950 fusions belonged to the category of intronic versioning for leukemia (n=1,456), brain tumor (n=319), and solid tumor (n=198) (Table 11), from which recurrent fusions were illustrated for leukemia (>5 patients), brain tumor (>3 patients) and solid tumor (>3 patients). Leukemias had the most diverse recurrent oncogenic fusions (n=26), followed by brain tumor (n=9) and solid tumor (n=6).

TABLE 11

| Cancer | Fusion |
|---|---|
| Leukemia | RUNX1-RUNX1T1, CBFB-MYH11, |
| | KMT2A-MLLT3, ETV6-RUNX1, |
| | KMT2A-MLLT10, NUP98-NSD1, BCR- |
| | ABL1, KMT2A-AFDN, |
| | TCF3-PBX1, KMT2A-AFF1, |
| | CBFA2T3-GLIS2, KMT2A-MLLT1, |
| | NUP98-KDM5A, DEK-NUP214, |
| | KMT2A-ELL, PICALM-MLLT10, FUS- |
| | ERG, MEF2D-BCL9, |
| | RBM15-MRTFA, EP300-ZNF384, |
| | HNRNPH1-ERG, KMT2A-SEPTIN6, |
| | TCF3-ZNF384, |
| | KAT6A-CREBBP, RUNX1-CBFA2T3, |
| | NIPBL-HOXB9 |
| Brain | KIAA1549-BRAF, C11orf95-RELA, |
| | FGFR1-TACC1, EWSR1-FLI1, MYB- |
| | QKI, PPP1CB-ALK, |
| | TMP3-NTRK1, YAP1-FAM118B, |
| | CLIP1-ROS1 |
| Solid Tumor | EWSR1-FLI1, PAX3-FOXO1, PAX7- |
| | FOXO1, EWSR1-ERG, |
| | EWSR1-WT1, ETV6-NTRK3 |

A high dynamic range in patient prevalence of oncogenic fusions was observed. For example, in leukemia RUNX1-RUNX1T1 was observed in 227 patients, while KMT2A-ELL was observed in 26 patients. It was hypothesized that the length of involving genes may be a contributing factor to such prevalence discrepancy. Due to the relatively smaller cohort sizes of brain tumor and solid tumor, fusions in leukemias were first analyzed. Interestingly, a marginally significant linear association (R-squared=0.23; P=0.013) was observed between prevalence in patients and total gene length of the involved gene pairs. Considering the inherent sampling bias in the highly heterogeneous cohort from a diverse set of resources, the analysis was next limited to leukemia with rearrangement involving KMT2A, which has many known fusion partners (MLLT1, ELL, SEPTIN6, AFDN, AFF1, MLLT10, and MLLT3) with non-trivial patient prevalence (Marschalek (2016) Ann. Lab. Med. 36:85-100). Surprisingly, an excellent linear association (R-squared=0.79; P=0.008) was obtained between gene length and patient prevalence. These data indicated that among genes with oncogenic potential upon fusion, longer genes have more chance to be involved in DNA rearrangement and to generate tumors. This hypothesis implies that all eligible base pairs (under the constraints of splicing and translation frame; mostly intronic bases) in corresponding genes can contribute to functional gene fusion and therefore DNA breakpoints should be uniformly distributed along the gene. To test this hypothesis, DNA breakpoints were detected from the transcriptome data and it was found that 4 out of 5 oncogenic fusions (EWSR1-FLI1 and CBFB-MYH11) demonstrated a near-uniform distribution in their DNA breakpoints. However, an exception in TCF3-PBX1 fusion was also detected, where the DNA breakpoints tended to cluster in intron 16 of TCF3, which is consistent with previous observation (Wiemels et al. (2002) Proc. Natl. Acad. Sci. USA 99:15101-6). Together, these data indicate that random chance (or gene length), and, less frequently, local DNA properties can influence the formation of oncogenic fusions.

Extending gene length analysis to brain tumor and solid tumor did not yield statistical significance. These data may either reflect the diverse subtypes and corresponding smaller cohort sizes among brain tumor and solid tumor or indicate additional factors influencing the etiology of oncogenic fusions, as detailed in following sections.

Expression Patterns of Oncogenic Fusions. Inspired by the fusions formed by promoter/enhancer hijacking (e.g., IGH-CRLF2 or IGH-DUX4 fusion in B-ALL; Mullighan et al. (2009) Nat. Genet. 41:1243-46) that lead to aberrant activation of target genes that otherwise should be completely silenced in corresponding normal lineage of host tumor, the expression characteristics of the recurrent fusions (n≥10) was studied. This analysis was carried out by measuring the relative expression ratio between C′ gene and N′ gene using the fused portion with an expression dominance score (EDS), where a low EDS score indicated that the C′ gene was expressed at lower level than that of the N′ gene. To account for the effect of gene fusion on the EDS scores, samples of a particular tumor type were categorized (so that samples with matched tissue-of-origin were used) into three groups: (1) samples with the fusion-of-interest; (2) samples with fusions other than the fusion-of-interest; (3) samples without known fusions. In the first group, the fused portion of the C′ gene must be expressed because of the fusion, while in the second and third groups, the C′ gene may or may not be expressed, and these two groups can cross validate each other. As a result, the EDS score fluctuates between-3 and 3 among samples with the fusion-of-interest (i.e., RUNX1-RUNX1T1 (E6-2), TCF3-PBX1 (E16-3), CBFA2T3-GLIS2 (E11-3), CBFB-MYH11 (E5-33), KMT2A-AFDN (E8-2), EWSR1-FLI1 (E8-6), DEK-NUP214 (E9-18), KMT2a-MLLT3 (E8-9), BCR-ABL1 (E1-2), NUP98-NSD1 (E12-7), NUP98-KDM5A (E13-27), KMT2A-ELL (E9-2), KMT2A-MLLT1 (E9-2), ETV6-RUNX1 (E5-3), KMT2A-AFF1 (E8-5), KIAA1549-BRAF (E16-9), C11orf95-RELA (E3-2), PAX3-FOX01 (E7-2), and PAX7-FOX01 (E7-2)). On the other hand, the median EDS score in samples of group 2 and 3 can be as low as −10 in fusions including RUNX1-RUNX1T1, TCF3-PBX1, CBFB-MYH11, and CBFA2T3-GLIS2, indicating that corresponding C′ genes are typically not expressed in host lineages. For example, gene PBX1 (in fact, only the fusion portion) was expressed in B-ALL sample SJE2A059 that harbors the TCF3-PBX1 fusion but was not expressed in B-ALL sample SJBALL021772, which is TCF3-PBX1 negative. In contrast, NSD1 is constitutively expressed in AML samples both positive (SJAML064746) and negative (SJAML064774) for NUP98-NSD1 fusion. With this observation, four conventional oncogenic fusions, i.e., RUNX1-RUNX1T1, TCF3-PBX1, CBFB-MYH11, and CBFA2T3-GLIS2, with EDS scores less than-3 in group 2 and 3 samples (i.e., without the fusion of interest) were classified as “promoter hijacking-like fusions”, and the remaining fusions as conventional chimerism. Interestingly, several fusions were also observed, including KIAA1549-BRAF, C11orf95-RELA, and PAX3/7-FOX01, to have significantly higher EDS scores in group 2 and 3 samples, indicating a highly active role of the C′ genes (BRAF, RELA, FOX01) in corresponding normal lineages. Collectively, because the C′ gene is silenced or lowly expressed in corresponding normal lineage for promoter hijacking-like fusions, it is proposed that the corresponding C′ genes (RUNX1T1, PBX1, GLIS2, and MYH11) can serve as excellent drug targets because the expected “on-target, off-tumor” toxicity can be minimized. By comparison, the “on-target, off-tumor” toxicity can be much higher in the conventional oncogenic fusion group.

Alternative Splicing in Oncogenic Fusions. Since alternative splicing is a general phenomenon in normal physiological conditions (Baralle & Giudice (2017) Nat. Rev. Mol. Cell Biol. 18:437-451), it was next determined whether alternative splicing can play a role in oncogenic fusions. A gene fusion may or may not be subject to regulation by alternative splicing. Toward this possibility, a splicing dominance score (SDS) was designed to measure the percentage of junction reads that supports the canonical splicing over all junction reads spanning exons in N′ gene and exons in C′ gene. To determine whether alternative splicing is dependent on the rearrangement, tumor samples without the fusion of interest were also studied, wherein the SDS score was calculated as the percentage of canonical splicing over all junctions that encompass the involving intron of N′ gene and C′ genes, respectively. By applying the method to all recurrent (n>3) fusions, it was discovered that the majority (86%) of oncogenic fusions were not subject to regulation by alternative splicing. Interestingly, fusions involving KMT2A appeared to be strongly affected by alternative splicing. The detailed splicing patterns of three representative oncogenic fusions indicated that alternative usage of exon 10 in KMT2A was clearly observed in both KMT2A-rearranged AML tumors and AML tumors without KMT2A fusions. In contrast, fusion NUP98-KDM5A was not regulated by alternative splicing. On the other hand, CBFB-MYH11 appeared to have negligible (<1%) alternative splicing caused by weak exon 5 skipping that was observed in both fusion positive tumors and tumors without CBFB-MYH11. These data indicated that alternative splicing is likely a property of host gene that is not affected by somatic alterations for oncogenic fusions. To further study whether this is true in non-cancer tissues, analysis was performed in 9,525 RNAseq samples from healthy donors in GTEx (Consortium (2013) Genet. Nat. 45:580-585). Notably, alternative splicing in ETV6-RUNX1 (identified in B-cell leukemia) was recapitulated in RUNX1 gene in normal GTEx blood samples, and splicing alternative in C11orf95-RELA (identified in Ependymoma, a brain tumor) was recapitulated in C11orf95 in normal GTEx brain samples. Interestingly, alternative splicing in these genes was “averaged out” when all 9,525 GTEx samples were used indiscriminately. Indeed, alternative splicing involving KMT2A was not recapitulated in GTEx dataset by this analysis, which was reflected by the lack of myeloid specimens in GTEx samples. Together, these data indicated a clear role of regulation by alternative splicing in oncogenic fusions, although such regulation is not specific to tumors and therefore is likely an intrinsic property of the host gene.

Selection Bias in Fusion Versioning. Because intronic versioning can cause amino acid differences in the fusion protein which may in turn lead to potential functional difference, it was hypothesized that fusion versioning could confer differential fitness to the host cells in some oncogenic fusions. It was posited that a relative selection bias score (RBS) based on the observation that DNA breakpoints are generally distributed in introns in a near-uniform fashion and gene length can predict patient prevalence. In this model, the patient prevalence of a given intron should be proportional to its length if the resultant protein versions are functionally equivalent (i.e., confers the same positive selection pressure. However, because the involved exon may encode functionally important protein domains and thus lead to higher positive selection pressure, its corresponding t have disproportionately high patient prevalence. In the cohort studied, fusion versioning was observed in 19 fusions. Here, fusions subject to alternative splicing regulation were excluded from this analysis because tumors with such fusions cannot be unambiguously classified into a particular versioning category.

A critical constraint to gene fusion products is exerted by splicing and translation, which is clearly illustrated by CBFB-MYH11 fusion in childhood AML. Here, the translational frame was first defined for each coding exon by using the codon frame of its first base. Because all six coding exons of CBFB have length of 3n=0 (mod 3), CBFB has all exons in frame 0. On the other hand, MYH11 has exon frames encompassing all three possibilities of 0, 1, and 2. Although numerous exonic combinations can theoretically generate in-frame proteins, in patients only a limited variety of fusion versions were observed, including E5-33 (n=181), E5-28 (n=16), etc. These data also indicate a potential selection bias due to critical protein domains encoded by involved exons. To test this hypothesis, a circuit plot was generated, where the N′ gene is placed on y-axis and C′ gene is placed on x-axis, and the axes are proportional to gene length. Conditional on exon 5 of CBFB, a clear discrepancy between patient prevalence and intronic length is observed for different fusion versions: intron 32 of MYH11 (corresponding to fusion version E5-33, n=181 patients) has length of only 370 bps, while intron 27 (corresponding to fusion version E5-28, n=16 patients) has a longer length of 5509 bps. With these data, a RBS score of 168.4 was observed, indicating a strong positive selection pressure for version E5-33 relative to version E5-28 (chi-square P<2×10⁻¹⁶).

To validate the hypothesis that fusion versioning may influence clinical outcomes, hazard ratios were compared for event-free survival (EFS) across the CBFB-MYH11 AML cohort (n=164) as a function of fusion versions and several well-established prognostic variables, including exon 17 KIT mutation status, white blood cell (WBC) count at diagnosis, patient age at diagnosis, and initial response to therapy as measured by end of induction I (E011) minimal residual disease (MRD). Remarkably, the E5-33 version of fusion CBFB-MYH11 was the best prognostic variable in this analysis, followed by exon 17 KIT mutation status, confirming that positive selection bias in version E5-33 can predict clinical outcome.

By applying this analysis to 4 fusions with recurrence >60, it was discovered that additional fusions, including ETV6-RUNX1 (Q<10⁻¹⁵) and KIAA1549-BRAF (Q=8×10⁻¹⁰) demonstrated statistically significant selection bias. On the other hand, fusion EWSR1-FLI1 only demonstrated a marginally significant Q value of 0.02 (after Bonferroni correction for multiple testing). It was noted that the limited patient number in many other fusions may have prevented the detection of selection bias. However, collectively, intronic versioning analysis provided a novel tool to study potential functional importance of certain protein domains that can serve as therapeutic targets and prognostic biomarkers.

Neo splicing in oncogenic fusions. Oncogenic fusions harboring neo splicing were detected in 34 patient tumors (Table 3). For example, brain tumor PT E3ADF4ZB harbored oncogenic fusion MN1-PATZ1, where the DNA breakpoint resides in exon 1 of PATZ1 and disrupts the normal splicing acceptor. To compensate for this disruption, the cancer cell created a novel splice acceptor (AG) at 26 base pairs upstream of the DNA breakpoint in intron 1 of MN1 gene. This case clearly indicated the flexibility of splicing machinery in recognizing novel splice sites. Among the oncogenic fusions with neo splicing, it was discovered that all three tumors with TCF3-HLF fusion involved neo splicing between exon 16 of TCF3 and exon 4 of HLF, indicating a common mechanism governing expression this of fusion. Indeed, close examination indicated that exon 16 of TCF3 and exon 4 of HLF have incompatible translation frames. Therefore, the neo splice sites and corresponding cryptic exons are created by the host cancer cell to compensate for the translation problem. Although it has been suggested that the cryptic exons function to make up the translation frame problem by the cancer cells (Hunger (1996) Blood 87:1211-24; Hunger et al. (1992) Genes Dev. 6:1608-20), there is no functional evidence available to date. Therefore, the function of this cryptic exon and corresponding hypothetical neo splice sites were investigated through CRISPR-based genome editing.

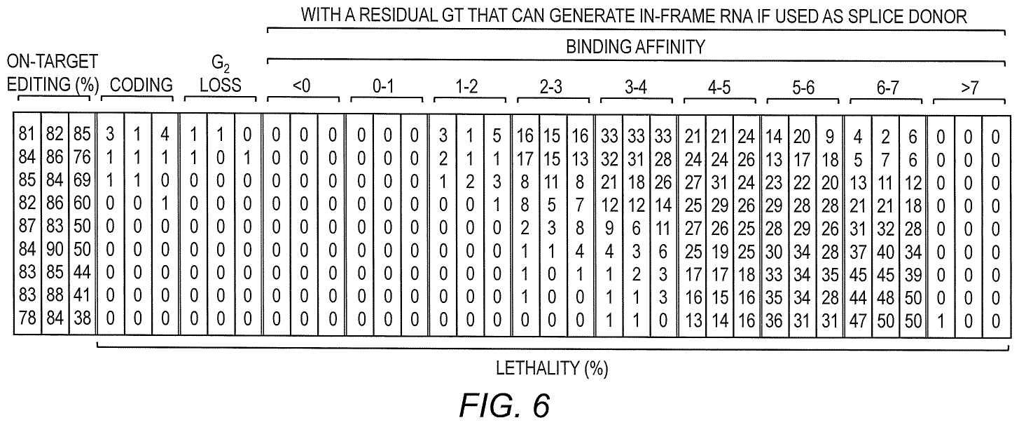

CRISPR Targeting of Neo Splicing. A TCF3-HLF positive cell line HAL-01 harbors a neo splicing pattern (FIG. 6) and provides an immediate in vitro model to validate the function of neo splice sites. Interestingly, this cell line harbored 27 base pairs of non-template insertion as part of the cryptic exon. Therefore, the essentiality of the cryptic exon was first tested by designing guide RNA (g₁, a ATCTCAGGCGTGCCCGACTCNGG; SEQ ID NO:1) targeting the non-template coding sequence by using the CRIPSR-Cas9 system with non-homologous end joining (NHEJ) mechanism that creates small insertion/deletions (indels). The effect of genome editing was measured using amplicon next generation sequencing (NGS) of targeted regions from day 3 through day 19 post editing. Because indels with lengths of 3n+1 and 3n+2 will cause frameshifts in this cryptic exon, it was expected that such editing would demonstrate stable reduction in abundance in NGS reads if the cryptic exon was functionally essential to the cancer cells. This analysis indicated a sharp decrease in NGS read abundance, from about 66% at day 3 to <1% by day 19 of out-of-frame indels (defined as indels with length 3n+1 and 3n+2 using CRIS.py), corresponding to >60-fold decrease with a T test P value of 0.0001. In contrast, putative non-lethal indels (defined as indels with length of 3n) demonstrated a stable increase in NGS read abundance from about 33% NGS reads at day 3 to 99% NGS reads at day 19 (Table 12). These data indicated that this cryptic exon is functionally essential for the HAL-01 cells. Indeed, RNA sequencing of CRISPR-edited HAL-01 cells confirmed a lack of alternative splicing in TCF3 (when exon 16 is used) so that the host cancer cells were completely dependent on cryptic exons via neo-splicing, which is in clear contrast with the weak E14-4 alternative splicing in B-ALL with E15-4 version of TCF3-HLF.

TABLE 12

| | OTE | | | % Lethal | | | % Non-Lethal | | |
|---|---|---|---|---|---|---|---|---|---|
| Day | 1 | 2 | 3 | 1 | 2 | 3 | 1 | 2 | 3 |
| 3 | 89 | 90 | 86 | 66 | 65 | 68 | 33 | 34 | 31 |
| 5 | 91 | 91 | 84 | 51 | 47 | 49 | 48 | 52 | 50 |
| 7 | 89 | 88 | 87 | 29 | 34 | 39 | 70 | 65 | 60 |
| 9 | 91 | 88 | 82 | 14 | 12 | 11 | 85 | 87 | 88 |
| 11 | 88 | 90 | 79 | 4 | 5 | 9 | 95 | 94 | 90 |
| 13 | 88 | 90 | 78 | 2 | 2 | 3 | 97 | 97 | 96 |
| 15 | 86 | 89 | 83 | 0 | 0 | 1 | 99 | 99 | 98 |
| 17 | 92 | 88 | 84 | 0 | 0 | 0 | 99 | 99 | 99 |
| 19 | 90 | 90 | 82 | 0 | 0 | 0 | 99 | 99 | 99 |

*Shown are % on-target editing (OTE; rate of induced Indels) rate, and % putative lethality (Indels causing frameshift of fusion transcripts are called lethal and other in-frame indels are called non-lethal) of NGS reads observed from day 3 to day 19 post editing for three replicates (1, 2 and 3).

The above data allowed for the investigation of the essential nature of neo splice sites in this locus. For this, a guide RNA g₂ (CTGAGATTTCTGGTGCAGGINGG; SEQ ID NO:2) was designed to target the splice donor. Because the actual (random) indel may or may not completely disrupt the splice donor, the binding affinity of residual donor site (if the GT still exists even though the indel has disrupted its context) was predicted (using a position specific weight matrix (PWM) method) and the translation frame status was simultaneously measured by assuming that such residual donor site can be used by the host cell. Only candidate donor sites with in-frame translations were evaluated for residual fitness as reflected by abundance of NGS reads from amplicon sequencing. To account for the fact that binding affinity is a continuous variable, the predicted binding affinity scores were divided into bins, and the change of NGS read abundance was studied for these score bins over time (from day 3 to day 19 post editing). Interestingly, a strong association between NGS read abundance and predicted binding affinity was observed (FIG. 6). For example, NGS reads from editing that resulted in residual donor site with binding affinity score between 2˜3 demonstrated a rapid decrease from >15% at day 3 to nearly 0% at day 19. In the next bin of binding affinity score between 3˜4, NGS read abundance decreased from 33% at day 3 to ˜18 at day 19. In comparison, NGS read abundance remained at a stable 15-20% abundance when the predicted binding affinity score was 4˜5. At bin 5˜6, the NGS read abundance increased from <20% at day 3 to >30% at day 19. Strikingly, when the predicted binding affinity was in bin 6˜7, the NGS read abundance increased from ˜5% at day 3 to ˜50% at day 19, indicating a strong gain of fitness of host cells. Collectively, by using binding affinity threshold of 4, the donor editing resulted in ˜60% putative lethal on-target editing rate that was comparable to that (65%) of coding exon targeting.

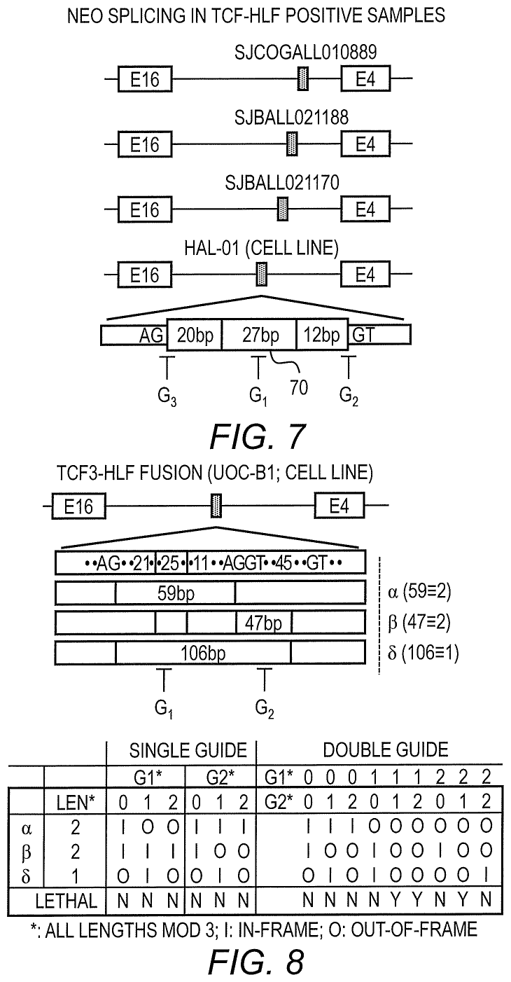

Subsequently, an attempt was made to target the neo acceptor AG by using a guide RNA g₃ (FIG. 7). The analytical procedure was similar as that of donor targeting. As it turned out, although a significant proportion (˜60%) of the editing fell into the coding region (and demonstrated expected lethal effect), ˜6% of induced indels resulted in a total loss of splice acceptor AG and demonstrated significant reduction in NGS read abundance to nearly 0% at day 19. Similar to the donor experiment, data these clearly indicated the essentiality of the neo splice acceptor which was also a therapeutic vulnerability for HAL-01 cell. Of note, targeting regions outside the cryptic exon and splice site regions had no impact on fitness, further demonstrating that the cryptic exon and its neo splice sites are a specific vulnerability to such cancer cells.

CRISPR Targeting in the Presence of Alternative Splicing. Although HAL-01 data indicated the feasibility of targeting the neo splice sites as well as the cryptic exon of oncogenic fusions, the potential effect of alternative splicing was not studied due to lack of natural alternative splicing in TCF3-HLF in HAL-01. For this purpose, another TCF3-HLF positive B-ALL cell line UoC-B1 was acquired, which harbors a DNA breakpoint more upstream to intron 3 of HLF than that in HAL-01, so that there are more splice site options for UoC-B1. In this line, parental UoC-B1 cells can theoretically generate three splicing isoforms by using the two candidate splicing acceptors AG and two candidate splicing donors GT (FIG. 8). Based upon published RNA sequencing for the UoC-B1 line (Accession No. SRR8816031), all three possible splicing isoforms were confirmed in parental cells: a (67% reads), B (12.5% reads), and 8 (20.5% reads). Although isoforms a and B can help the UoC-B1 cells to resolve the translation frame problem between TCF3 exon 16 and HLF exon 4, isoform δ cannot. Therefore, it was predicted that targeting isoforms a or β alone may not be effective due to compensatory splicing among them. To test this hypothesis, one guide (g₁, CAAGTAGATCGAGGTGGAGANGG; SEQ ID NO: 6) was designed to target isoform a and another guide (g₂, CCAGAATTCTTCTGGGTAGGNGG; SEQ ID NO: 7) to target isoform B. This analysis indicated that g₁ and g₂ alone lead to negligible reduction of putative “lethal” on-target editing that disrupted a and β, respectively (g₁, Table 13; g₂, Table 14). These data confirm the compensatory role of a and B exons when perturbed alone.

TABLE 13

| | OTE | | | % Lethal | | | % Non-Lethal | | |
|---|---|---|---|---|---|---|---|---|---|
| Day | 1 | 2 | 3 | 1 | 2 | 3 | 1 | 2 | 3 |
| 3 | 82 | 85 | 85 | 64 | 65 | 56 | 20 | 18 | 20 |
| 5 | 82 | 84 | 85 | 64 | 58 | 61 | 23 | 25 | 23 |
| 7 | 83 | 84 | 86 | 60 | 58 | 59 | 31 | 23 | 25 |
| 9 | 85 | 87 | 84 | 59 | 53 | 55 | 31 | 27 | 29 |
| 11 | 85 | 79 | 87 | 57 | 53 | 63 | 33 | 27 | 25 |
| 13 | 85 | 87 | 84 | 55 | 51 | 60 | 29 | 24 | 23 |
| 15 | 84 | 83 | 85 | 56 | 50 | 60 | 30 | 24 | 24 |
| 17 | 87 | 86 | 81 | 54 | 50 | 54 | 30 | 23 | 25 |
| 19 | 85 | 84 | 84 | 54 | 49 | 56 | 31 | 24 | 23 |

*Shown are OTE and % putative lethality of NGS reads observed from day 3 to day 19 post editing for three replicates (1, 2 and 3).

TABLE 14

| | OTE | | | % Lethal | | | % Non-Lethal | | |
|---|---|---|---|---|---|---|---|---|---|
| Day | 1 | 2 | 3 | 1 | 2 | 3 | 1 | 2 | 3 |
| 3 | 79 | 77 | 81 | 42 | 45 | 39 | 18 | 18 | 23 |
| 5 | 85 | 83 | 83 | 47 | 44 | 42 | 18 | 19 | 18 |
| 7 | 84 | 84 | 84 | 43 | 44 | 40 | 18 | 20 | 21 |
| 9 | 86 | 85 | 84 | 41 | 39 | 37 | 20 | 20 | 20 |
| 11 | 85 | 86 | ND | 39 | 35 | ND | 18 | 20 | ND |
| 13 | 85 | 87 | 84 | 32 | 29 | 30 | 19 | 21 | 19 |
| 15 | 84 | 86 | 87 | 32 | 29 | 27 | 18 | 22 | 18 |
| 17 | 85 | 86 | 85 | 32 | 32 | 26 | 18 | 20 | 21 |
| 19 | 86 | 87 | 85 | 27 | 27 | 25 | 20 | 18 | 23 |

*Shown are OTE and % putative lethality of NGS reads observed from day 3 to day 19 post editing for three replicates (1, 2 and 3).
ND, no data.

It was posited that double editing that simultaneously disrupts all possible isoforms may lead to synthetic lethality. For this, the theoretical possibilities of CRISPR targeting were analyzed using double guides g₁+g₂. By categorizing the effect of induced indels into two states (being in-frame (I) or being out-of-frame (O)) for each of the three isoforms α, β, and δ, it was predicted that only reads that lead to “O” state for all three isoforms can result in lethal effect, which comprises 37.5% (=3/8) of all on-target editing. This analysis (g₁+g₂, Table 15) indicated that the putative lethal editing demonstrated a sharp decrease of NGS read abundance from ˜37% at day 3 to nearly 0% at day 19.

TABLE 15

| | OTE | | | % Lethal | | | & Non-Lethal | | |
|---|---|---|---|---|---|---|---|---|---|
| Day | 1 | 2 | 3 | 1 | 2 | 3 | 1 | 2 | 3 |
| 3 | 78 | 83 | 83 | 40 | 35 | 39 | 19 | 20 | 22 |
| 5 | 83 | 84 | 81 | 31 | 35 | 30 | 22 | 22 | 25 |
| 7 | 85 | 83 | 83 | 18 | 17 | 13 | 29 | 22 | 25 |
| 9 | 81 | 86 | 85 | 7 | 6 | 6 | 26 | 20 | 23 |
| 11 | 84 | 84 | 84 | 5 | 5 | 6 | 26 | 21 | 25 |
| 13 | 83 | 85 | 83 | 4 | 2 | 1 | 22 | 16 | 18 |
| 15 | 85 | 86 | 85 | 2 | 0 | 0 | 23 | 15 | 20 |
| 17 | 86 | 86 | 85 | 1 | 0 | 1 | 20 | 13 | 15 |
| 19 | 83 | 85 | 86 | 0 | 0 | 0 | 23 | 13 | 19 |

*Shown are OTE and % putative lethality of NGS reads observed from day 3 to day 19 post editing for three replicates (1, 2 and 3).

In contrast, the putative non-lethal editing (that can keep at least one of α, β, and δ being in-frame) remained a stable NGS read abundance from day 3 to day 19. Because double guides theoretically can lead to double focal indel editing and single large deletion, the NGS reads of these two categories were also studied. Indeed, nearly 50% of lethal editing are large deletions, and both large deletions and double focal indels had comparable decreases in NGS read abundance. These data clearly demonstrated the functionally compensatory nature of alternative splicing in TCF3-HLF in UoC-B1 that posed a significant challenge in gene targeting using only single guide approach.

Together, these experiments indicated that neo splicing in corresponding oncogenic fusions are functionally essential for host cancer cells and offer novel therapeutic vulnerability. TO facilitate targeting, computational approaches are used to accurately predict outcomes of CRISPR editing to enable rationale design of CRISPR guides and minimize escaping effect.

### Example 3: Genomic and Global Gene Expression Profiling in Pediatric and Young Adult Acute Leukemia with PICALM:MLLT10 Fusion

Patients and samples. Twenty-one samples from twenty patients with acute leukemia with PICALM:MLLT10 fusion diagnosed by whole transcriptome sequencing (RNA-seq) clinical testing and/or conventional cytogenetics on patients' tumor samples at St Jude Children's Research Hospital (SJCRH) were included in this study. One patient, SJ061501, had both diagnostic (D) and relapse (R) samples available for the study. Conventional cytogenetics was performed on 8 of 20 patients. Molecular profiling was performed by whole genome sequencing and whole exome sequencing in 16 patients samples), whole exome (17 sequencing only in one, targeted panel NGS testing in two patients, and the remaining one had no DNA available for sequencing. Appropriate informed consent was obtained from patients. This study was reviewed and approved by the Institutional Review Board (IRB) at SJCRH.

Cytomorphology evaluation and Immunophenotyping studies. The diagnoses were made with the cytomorphologic evaluation and immunophenotypic data based on the 4^{th} edition/5^{th} edition WHO classification and International Consensus Classification (ICC). Cytomorphological assessments were performed on the available peripheral blood smear, bone marrow aspirate smears and/or core biopsy obtained at the time of diagnosis. Flow cytometry immunophenotyping was performed using comprehensive panels when material was available. Cases from collaborative institutions with established diagnoses underwent only limited flow cytometry immunophenotypic studies.

Clinical RNA-seq test. The clinical RNA-seq test at SJCRH was validated to identify recurrent gene fusions/rearrangements that have been recognized to be associated with human neoplasms. Total RNA was isolated from patients' tumor sample and RNA-Seq libraries were constructed using the TRUSEQ® Stranded Total RNA Kit (Illumina, CA). The RNA-seq analysis was performed as described elsewhere herein.

Conventional cytogenetic analysis. Conventional cytogenetic analysis was performed on 24-hour unstimulated bone marrow cultures with or without synchronization according to standard procedures. When available, 20 metaphases were analyzed, and karyotypes were interpreted according to the International System for Human Cytogenetic Nomenclature.

Whole genome sequencing and whole exome sequencing. Whole genome sequencing (WGS) and/or whole exome sequencing (WES) were performed on paired tumor-normal samples for patients indicated in Table 16.

TABLE 16

| | | PICALM- | |
|---|---|---|---|
| | | MLLT10 | |
| | | Exon- | |
| | | Exon | |
| Case ID | Diagnosis | Junction | Karyotype |
| SJ030459 | AML | E19-E4 | 46, X, −Y, t(10; 11) (p12; q14), |
| | | | i(17) (q10), + 19[16]//46, XX[4] |
| SJ030988 | AML | E19-E9 | 46, XY, t(10; 11) (p12; q14-22) |
| | | | [6]/46, XY[1] |
| SJ031965 | AUL | E19-E9 | 46, XY, del(6) (q13q25), add(7) |
| | | | (q36), t(10; 11) (p12; q14- |
| | | | 22) [3]//46, XX[17] |
| SJ033011 | AML | E19-E8 | 45, X, −X, add(5) (p13), t(10; 11) |
| | | | (p12; q14), add(17) (p11.2) [20] |
| SJ032188 | AML | E19-E10 | N/A |
| SJ061255 | AML | E19-E9 | 46, XY, del(2) (p21p25), inv(5) |
| | | | (p15.1q11.2), t(10; 11) (p12; q14), |
| | | | i(17) (q10) [19]/46, XY[1] |
| SJ062998 | AML | E19-E4 | 47, XY, +4, t(10; 11) (p12; q14) [2]/47, |
| | | | idem, i(17) (q10) [3]/46, XY[15] |
| SJ061501 | AML | E17-E4 | 46, XY, t(10; 11) (p12; q14), der(12) |
| | | | t(12; 13) (p13.1; q12.3), −13, +19 |
| | | | N/A |
| SJ074161 | AML | E19-E9 | 46, XY, del(9) (q13q22), t(10; 11) (p12; q14- |
| | | | 22), der(19) t(11; 19) (q14- |
| | | | 22; p13) [20]/46, XY[1] |
| SJ033167 | AML | E17-E6 | N/A |
| SJ033585 | AML | E17-E9 | N/A |
| SJ030880 | T-ALL | E19-E4 | N/A |
| SJ030907 | T-ALL | E19-E4 | N/A |
| SJ031662 | T-ALL/LLY | E18-E10 | N/A |
| SJ031798 | MPAL T/B | E19-E9 | N/A |
| SJ031904 | T-ALL | E19-E10 | N/A |
| SJ031944 | ETP-ALL | E19-E9 | N/A |
| SJ032955 | T-LLY | E19-E4 | N/A |
| SJ033522 | T-ALL | E19-E10 | N/A |
| SJ033817 | T-ALL/LLy | E19-E10 | N/A |

Transcript IDs: PICALM NM_007166.3, MLLT10_NM_004641.3
Abbreviations: AML, acute myeloid leukemia; AUL, acute undifferentiated leukemia; E, exon; ETP, Early T-cell precursor; MPAL, mixed phenotype acute leukemia; N/A, not available; T-ALL, T-lymphoblastic leukemia; T-ALLY, T-lymphoblastic lymphoma.

WGS libraries were constructed using the TRUSEQ® DNA PCR-Free sample preparation kit (Illumina, CA) and for WES using the Illumina TRUSEQ® Exome Library Prep Kit (Illumina, CA). Sequencing libraries were sequenced on HISEQ4000® or NOVASEQ® 6000. The average WGS depth of the samples studied was 64×; the average WES depth was 155×. Data process, analysis, variant calling and annotation were performed as described herein. In brief, tumor-acquired single nucleotide variations (SNVs) and small insertion-deletions (indels), structural variants as well as copy number variants and copy neutral loss of heterozygosity (LOH) were evaluated. The limit of detection of variant at low allele frequency in coding genes is estimated to be ˜5% for SNVs and indels with a positive predictive value of 97-99%.

Gene expression profiling by RNA-seq analysis. Whole transcriptome sequencing FASTQ files were processed and aligned to Human Reference genome hg19 using the Illumina DRAGEN RNA pipeline v.4.0.3 with “--enable-rna-quantification true”. The obtained quantification files were read into R v.4.3.0 and differential expression analysis was performed by DESeq2 using publicly available bulk RNA-seq data of a set of CD34+CD38-CD45RA-EPCR+ hematopoietic stem cells (HSC) derived from human cord blood samples as control. Principle component analysis (PCA) was generated by PlotPCA function within the DESeq2 package using the variance stabilizing transformed (VST) counts and without the constraint of priori group information. GO enrichment analyses were performed using the clusterProfiler package specifically the functional enrichment analysis was conducted using the enrichGO function with pvalueCutoff and qvalueCutoff of 0.01. The gene set enrichment analysis (GSEA) was performed using the gseGo function with p-value cutoff of 0.05. The p-value adjustment method in this study is Benjamini-Hochberg. As for the expression of HOX genes, TPM (transcripts per million) value of whole blood was retrieved from GTEx Analysis 2017-06-05_v8_RNASeQCv1.1.9_gene median tpm.gct.

Clinical, cytomorphologic and immunophenotypic characteristics. The 20 patients with PICALM:MLLT10-positive leukemia were predominantly male (18 male, 2 female) and teenagers at diagnosis (median age 14.5 years, range 5 to 18 years). The demographics and clinical characteristics of the twenty patients are summarized in Table 17.

TABLE 17

| Characteristic | AML | T-ALL | MPAL, T/B | AUL | TOTAL |
|---|---|---|---|---|---|
| Sex | 1F, 9M | 0F, 8M | 1F, 0M | 0F, 1M | 2F, 18M |
| Age (years) | | | | | |
| 1-9 | 0 | 3 | 1 | 0 | 4 |
| 10-20 | 10 | 5 | 0 | 1 | 16 |
| WBC Count | | | | | |
| >100000 | 4 | 1 | | | 5 |
| <100000 | 5 | 7 | | | 12 |
| N/A | 1 | | 1 | 1 | 3 |
| Extramedullary | | | | | |
| Disease | | | | | |
| Yes | 3 | 6 | | | 13 |
| No | 3 | 1 | 1 | 1 | 6 |
| N/A | | 1 | | | 1 |
| Refractory/ | | | | | |
| Relapse | | | | | |
| Yes | 10 | 1 | 1 | 1 | 13 |
| No | 0 | 7 | | | 7 |
| Survival | | | | | |
| Deceased | 8 | 1 | 1* | 1 | 11 |
| Alive | 2 | 6 | | | 8 |
| Lost @ follow-up | | 1 | | | 1 |
| Total # patients | 10 | 8 | 1 | 1 | 20 |

*The patient died of sepsis during induction therapy.
Abbreviations: AML, Acute myeloid leukemia; AUL, acute undifferentiated leukemia; F, female; M, male; MPAL, mixed-phenotype acute leukemia; N/A, not available; T-ALL/LLy, T-cell acute lymphoblastic leukemia/lymphoma; WBC, white blood cell.

Ten patients were diagnosed with AML, 1 with acute undifferentiated leukemia (AUL), 8 with T-ALL/LLy, and 1 with mixed phenotype acute leukemia (MPAL), T/B (T/B-MPAL). Among the 8 T-ALL/LLy cases, one fulfilled the diagnostic criteria of early T-cell precursor lymphoblastic leukemia (ETP-ALL), and 7 had extramedullary disease. In the T-ALL/LLy group, 6 of 8 attained complete remission and remain in remission; one patient relapsed and is alive, while the remaining one patient died of sepsis during induction therapy. The 11 non-ALL patients (10 AML and 1 AUL) were characterized by extramedullary disease in 7 cases, hyperleukocytosis in 4 cases, and poor outcomes with a median survival of 14 months. Furthermore, 6 of the AML cases had primary refractory leukemia, 2 had persistent minimal residual disease (MRD) and subsequently relapsed, and 2 achieved remission before relapse. Only 2 of the AML patients are alive. One patient (SJ62998) has remained in remission for more than seven years since receiving haploidentical transplant; the other patient (SJ033585) was treated at their local hospital after initial diagnosis and received hematopoietic stem cell transplant.

The PICALM:MLLT10 cohort included cases of several rare entities within the umbrella category of “Acute leukemia of ambiguous lineage” based on the ICC/WHO classifications. For example, case SJ031965 demonstrated no significant evidence of either myeloid or lymphoid lineage differentiation and is felt to be best classified as AUL; case SJ031798 demonstrated evidence of both B- and T-cell lineage differentiation (CD19+, cCD79a+, surface kappa light chain+, cCD3+) and is felt to be best classified as MPAL, T/B. Among the 8 T-ALL/LLY cases in our cohort of pediatric/young adult patients, 5 were gamma-delta T-ALL/LLy.

Conventional cytogenetic analysis. Conventional cytogenetics analysis was performed on 8/11 non-ALL cases as part of the routine diagnostic workup, and the chromosome translocation t (10; 11) (p12; q14) was detected in all 8 cases. Additional abnormalities were observed in all cases except one. The most frequent additional change was abnormalities affecting chromosome 17 with i (17q) found in 3 cases and add (p11.2) in one, all of which resulted in 17p deletion containing TP53 gene locus. The other recurrent secondary chromosome abnormality seen in this cohort is trisomy 19.

Transcriptomic profiling of PICALM:MLLT10 positive acute leukemia. The transcriptomic profile of the PICALM:MLLT10-positive acute leukemias was sought by evaluating the RNA-seq gene expression data of all 20 cases. Principle component analysis (PCA) stratified the cases into two groups, each containing 10 cases. Group 1 contained all PM-AML cases; and group 2 contained all PICALM:MLLT-T-ALL/LLy (PM-T-ALL/LLy) cases, one T/B-MPAL, and one AUL. Overall, the transcriptomic profiling indicated that PM-AML and PM-T-ALL/LLy belong to distinct transcriptomic groups. Despite the overall difference in gene expression profile, no HOXA cluster genes were differentially expressed between PM-AML and PM-T-ALL/LLy groups. Instead, elevated expression of HOXA5, HOXA6, HOXA9, and HOXA10 were observed in the vast majority of cases in both transcriptomic groups 1 and 2 compared to whole blood control

To examine in more detail the genes differentially expressed among subgroups, a differential gene expression (DGE) analysis was performed for each subgroup (PM-AML, PM-T-ALL/LLy, the AUL case and the T/B-MPAL case) compared to the control (CD34+CD38-CD45RA-EPCR+ human umbilical cord blood stem cells, “HSCs” for short). Significantly differentially expressed genes (fold change ≥4, adjusted p-value <0.05) for each subgroup were visualized across all samples in a hierarchically clustered heatmap. Hierarchical clustering produced a similar stratification as observed through PCA, largely separating PM-AML and PM-T-ALL/LLY patients in two groups. Although most of the PM-AML cases cluster separately from PM-T-ALL/LLy, SJ062998 clusters with PM-T-ALL/LLy cases, whereas the ETP-ALL case SJ031944 clusters with PM-AML cases. Gene expression profiling in these two cases that appear to be outliers is likely compromised by the low blast percentage (<10%) in the test samples; besides, only a subset of blasts (˜19% of blasts) in the ETP-ALL case expressing cytoplasmic CD3, a marker of T-lineage differentiation. The only case diagnosed with AUL (SJ031965) clustered within the AML group and the T/B-MPAL case (SJ031798) clustered within the T-ALL/LLy group. Although gene signatures could be derived to distinguish PICALM:MLLT10-positive acute leukemias from HSCs, unique signature could be hardly derived for subgroups, indicating genetic similarity across different immunophenotypic subgroups as well as heterogeneity within each subgroup.

Correlation between immunophenotypes and transcriptomic profiles. Immunophenotypic markers commonly used for diagnosis were integrated to assess the correlation with gene expression profiling. CD1A-positive cases appeared to group together both in the clustering heatmap and in the unsupervised principal component analysis (PCA). However, these CD1A-positive T-ALL/LLY cases demonstrated heterogeneity according to other immunophenotypic markers. Two out of the three CD1A-positive cases appeared double positive for CD4 and CD8 but lacked surface CD3, corresponding to the double positive thymocyte. The remaining CD1A-positive case appears to be CD4+, CD8−, with surface CD3 and TCRγδ expression. No apparent clustering was identified for other immunophenotypic markers, including TCRγ8. However, the evaluation of association is limited by the number of cases in this study cohort and limited workup in subset of cases.

Functional classification of differentially expressed genes in each subgroup. Functional classification of significantly upregulated genes compared to HSCs (fold change ≥4, adjusted p-value <0.05, TPM 21) in each subgroup by pathway enrichment analysis highlighted cell proliferation-related MSigDB Hallmark gene sets, G2M checkpoint and E2F targets, for all four subgroups. The enrichment of both gene sets in all four subgroups was further supported by gene set enrichment analysis (GSEA). GSEA analysis was extended to include the Gene Ontology Biological Function gene sets and demonstrated that genes associated with T cell receptor signaling and immune response process were highly enriched in PM-T-ALL/LLy and the T/B-MPAL case but had no significant correlation with PM-AML or the AUL case.

Subtype-specific molecular profiling in PM-AML and PM-T-ALL/LLy. Comprehensive genomic profiling was available for 19 out of 20 patients who had samples available for DNA sequencing. The remaining one AML case had no DNA for genomic profiling, and SNVs and small insertion-deletions (indels) were determined on selected genes (PHF6, TP53, NF1, SUZ12, EZH2 and PTPN11) based on RNA-seq data. Overall, PHF6 was the most frequently mutated gene in this study cohort, seen in 12 patients, including 7 transcriptomic group 1 (PM-AML) patients and 5 group 2 patients (3 PM-T-ALL/LLy, 1 T/B-MPAL and 1 AUL). PHF6 is located on the X-chromosome. Among the 12 patients with PHF6 alteration, 11 patients had a complete loss of PHF6, including hemizygous loss of function (LOF) alterations in 10 male patients and bi-allelic LOF in one of the remaining female patients diagnosed with AML, SJ033011.

Alterations affecting TP53 and NF1 were hallmarks of PM-AML, seen in 8/10 (80%) and 7/10 (70%) of PM-AML patients, respectively. Notably, in patient SJ061501 who had both diagnostic and relapsed samples available for testing, TP53 alteration as well as 17p deletion were only detected in the relapsed sample. Aside from TP53 and 17p deletion, all other abnormalities shared by both the diagnostic and relapsed samples were present at similar variant allele frequency in this patient. Among the eight PM-AML patients' samples with TP53 alterations, inactivation of both TP53 alleles by “two hits” (one mutation coupled with 17p deletion/copy neutral LOH or two in trans mutations) was observed in five. Similarly, among the seven PM-AML with NF1 alterations, bi-allelic LOF was identified in four cases. In addition to TP53 and NF1, deletion or LOF alterations of SUZ12 were also observed in 60% (6/10) of PM-AML cases, of which one case had biallelic LOF of SUZ12. SUZ12 and NF1 are located ˜600 Kb apart at 17q12 and these two genes are often affected by one focal segmental deletion in this cohort. Mutations in PTPN11 (3/10 patients, 30%) are also relatively common in PM-AML.

On the contrary, beyond PHF6 the most recurrent alterations in PM-AMLs were rarely seen in PM-T-ALL/LLY cases. Genetic alterations affecting TP53 were observed in only one case (SJ031904), which was a subclonal 17p deletion event. None of PM-T-ALL/LLy had bi-allelic LOF of NF1, and only one case (SJ031662) had a hemizygous deletion affecting NF1. Indeed, a completely different molecular signature was observed in PM-T-ALL/LLY. This group was specifically enriched for alterations in EZH2 (4/8, 50% versus 10% in PM-AMLs) as well as activating mutations in NOTCH1 signaling (4/8, 50%), followed by alterations activating JAK-STAT pathway (3/8, 37.5%) and biallelic LOF of CDKN2A (3/8, 37.5%).

Subtype-specific large-scale copy number variations in PM-AML and PM-T-ALL/LLy. In addition to gene-level alterations, large scale (>5 Mb) copy number variations (LS-CNVs) of 18 cases were evaluated, in which WGS and/or conventional cytogenetics were performed. Integrating the WGS and karyotype findings, it was demonstrated that the most frequent LS-CNV was 17p deletion and copy neutral loss of heterozygosity (CN-LOH), seen in 7 patients (6 PM-AMLs and one PM-T-ALL/LLy, SJ031904, as a subclonal event). More than half of the cases with 17p deletion were presented as isochromosome 17q, which also resulted in a gain of 17q. The next frequent CNV event was 12p deletion, in particular involving 12p13, seen in 5 cases (4 PM-AMLs and one PM-T-ALL/LLy, SJ031904). In contrast to 17p deletion/LOH and 12p deletion that were highly associated with PM-AML, 9p deletion/LOH were exclusively seen in PM-T-ALL/LLy.

Mutation and gene expression profiling of the AUL case. Global gene expression analysis clustered the AUL case SJ031956 between AML and T-ALL/LLy, but slightly toward the PM-T-ALL/LLy group, while genomic profiling revealed PHF6 inactivation as well as biallelic LOF of NF1 and SUZ12 in this case. As aforementioned, biallelic LOF of NF1 was exclusively seen in PM-AMLs in contrast to PM-T-ALL/LLy in this study. In the hierarchically clustered heatmap, this case clustered close to other cases of PM-AML that harbor alterations in NF1 and/or SUZ12. Together, the genomic and transcriptomic findings are compatible with the ambiguous immunophenotypic characteristic of this case. Functional classification of genes significantly upregulated in SJ031956 showed similar results as seen for other subgroups, i.e., enrichment in gene sets associated with cell proliferation (G2M checkpoint and E2F targets).

Mutation and gene expression profiling of the T/B-MPAL case. The T/B-MPAL case SJ031798 clustered within the PM-T-ALL/LLy group on PCA, but showed a greater variance according to PC2, suggesting that while similar to PM-T-ALL/LLy in terms of the dominant pattern represented by PC1, there are additional variances in gene expression profiling distinguished SJ031798 from PM-T-ALL/LLy. Nevertheless, functional classification of significantly upregulated genes in SJ031798 and GSEA demonstrated similar features between SJ031798 and PM-T-ALL/LLy cases. In addition, genomic profiling revealed heterozygous mutations in PHF6, EZH2, and JAK1, as well as a biallelic missense variant in STAT5B that is associated with a CN-LOH of chromosome 17q, all of which were recurrent seen alterations in PM-T-ALL/LLY. Interestingly, the T/B-MPAL case is distinguished from all other cases in the study cohort by the extremely low expression of HOXA9 and HOXA10; however, this observation is based solely on one T/B-MPAL case and remains to be evaluated in future studies.

### Example 4: Genomic Profiling of Circulating Tumor DNA for Childhood Cancers

Patients. Pediatric cancer patients treated at St. Jude Children's Research Hospital with plasma samples collected at diagnostic tumor biopsy or resection, prior to the initiation of treatment were identified for this study. Eleven patients with no somatic lesions detected in tumor and cfDNA sequencing data were excluded after initial analysis, resulting in 233 patients in the study cohort. All patients had tumor biomarkers identified by clinical genomics testing and other molecular pathology assays.

Ethics approval and consent to participate. The study was approved by the St. Jude Children's Research Hospital Institutional Review Board #00000029 (FWA00004775). Written informed consent was obtained d from patients, parents or guardians in accordance with the Declaration of Helsinki.

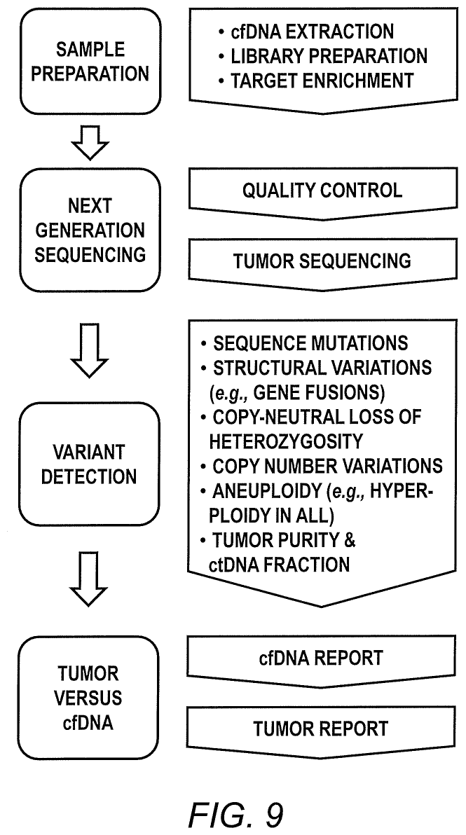

Plasma cfDNA preparation. Whole blood samples were collected in EDTA or cfDNA BCT tubes (Streck), double centrifuged to collect plasma, and stored at −80° C. The workflow was optimized to perform cfDNA extraction and library construction from 1 ml plasma (FIG. 9). cfDNA was extracted using the NUCLEOSNAP® cfDNA kit (Takara Bio) following the manufacturer's instructions, quantified with QUBIT™ dsDNA HS assay kit (Thermo Fisher Scientific), and assessed for quality and size distribution by TAPESTATION electrophoresis (Agilent). Libraries were prepared from up to 20 ng or 30 μL of cfDNA without fragmentation or size selection, using THRUPLEX® Tag-Seq HV kit (Takara Bio) with unique molecular identifiers (UMIs) for error suppression. All patient cfDNA samples underwent PeCan-Seq with target enrichment, where eight prepared libraries were pooled in an equimolar manner to achieve up to 4000 ng per reaction, and then hybridized with capture probes from the SJPedPanel described herein, using Twist Target Enrichment reagents and following the manufacturer's protocol. Twelve cfDNA samples also underwent whole genome sequencing (WGS) without target enrichment. In addition, Pecan-Seq was performed using cfDNA from serial plasma samples, collected on days 15, 22, and end of induction from 14 children with B-ALL.

SJPedPanel. As described herein, the SJPedPanel is a next-generation sequencing capture panel that covers common driver lesions in pediatric cancers. For this analysis, the SJPedPanel version 1.1 was used, which covers 357 pediatric cancer driver genes (Table 1). Covered alterations included somatic mutations, gene fusions, focal CNVs, and noncoding regions covering hotspots for SVs. The panel design also included 7590 common heterozygous single-nucleotide polymorphisms (SNPs; Table 2), which were evenly spaced ˜0.32 Mb apart across the human reference genome. These were included to facilitate the detection of chromosomal aberrations such as CNV and copy-neutral loss of heterozygosity (cnLOH) events. The SJPedPanel probe set (Twist Bioscience) covered 2.82 Mb of genomic content. An additional “add-on” panel was designed to include 11 additional pediatric cancer gene targets that were not included in SJPedPanel v1.1, and enrichment was performed using a mixture of probes from both panels for the re-sequencing of 9 samples.

Sequencing and mapping. A total of 228 patients had clinical WGS and/or whole exome sequencing (WES) of diagnostic tumor samples. The median genome-wide sequencing depth per tumor genome was 68-fold haploid coverage (range, 36 to 149-fold), and the median sequencing depth of exons per tumor exome was 337-fold (range, 71 to 781-fold). For cfDNA samples, the captured libraries were sequenced for 150 bp paired-end reads using the Illumina NOVASEQ® 6000 System at the Hartwell Center Genome Sequencing Facility. Sequencing requirements were 250 million reads per diagnosis sample or 1 billion reads per monitoring sample, additional top-off sequencing was performed for quality control purposes where necessary. The raw reads were trimmed using fastp v0.23.1 mapped to the human genome assembly GRCh38 using BWA-mem v0.7.17, grouped and filtered with UMIs following the manufacturer's instructions. Finally, the clean reads were mapped to GRCh38 without further deduplication.

SNV and indel mutation analysis. Plasma CfDNA sequencing data were paired with normal WGS and/or WES, and somatic SNVs and indels were called by Mutect2 v4.1.8.0 and LoFreq v2.1.3.1. For cfDNA samples without paired normal sequencing data, the variants were called in tumor-only mode. Starting in variant call format, SNVS and indels were annotated and ranked for potential pathogenicity using a web- and cloud-based platform PeCanPIE, with a maximum minor allele population frequency of 0.001 to remove germline mutations. Variants meeting the following criteria were considered as pathogenic or likely pathogenic: (1) non-synonymous variants, including missense, nonsense, in-frame indel, frameshift, and splice mutations; (2) Gold or Silver pathogenicity medal in PeCanPIE annotation; (3) at least two mutant allele counts in both orientations; (4) significant enrichment in cfDNA compared with normal WES (if available), using two-sided Fisher's exact test. Non-pathogenic variants that were covered by the panel were also curated, which included synonymous, intronic, and noncoding SNVs/indels, and these were used for the estimation of tumor purity and cfDNA fraction. Candidate variants were manually reviewed in Integrated Genome Viewer, and the unique mapping of variant supporting reads was verified by BLAT search.

Structural variation identification. SVs were identified using Manta v1.6.0. Manta was run using two configurations to improve sensitivity: (1) cfDNA data were paired with normal WGS; (2) cfDNA data in tumor-only mode. SV junctions were annotated with genes at breakpoints, as well as genes covered by deletions and duplications. For cfDNA only calls, polymorphic SVs were removed for consideration by comparing with gnomAD SV database. The coverage and genomic information of SV candidates were visualized and reviewed using GenomePaint, and images of sequence alignments among samples were generated and reviewed using Samplot. The evidence counts (paired reads and split reads) of SVs were used for the estimation of tumor purity and cfDNA fraction.

CNV and cnLOH detection by SubChrom. SubChrom is an analytical tool that interrogates the distributions of variant allele frequency (VAF) of heterozygous SNPs and genomic coverage of panel targets. Firstly, germline SNPs were called using GATK HaplotypeCaller v4.1.8.0, and those passing filters in gnomAD v3.1.2 and TOPMed Freeze 8 databases were used for downstream analysis. To screen for LOH events, each chromosome was segmented based on minor allele fractions (MAF) of heterozygous SNPs using circular binary segmentation (CBS), and then each segment was tested by fitting VAFs (v) of heterozygous SNPs into two models: unimodal distribution (u) and bimodal distribution (b):

$$u ( v \vert {v}_{u} , s ) = G ( v \vert {v}_{u} , s )$$

where G denotes a Gaussian distribution, Vᵤ is the mean, and s is the standard deviation.

$$b ( v \vert {v}_{b} , s , f ) = fG ( v \vert {v}_{b} , s ) + ( 1 - f ) G ( v \vert 1 - {v}_{b} , s )$$

where f is the fraction of SNPs on the minor allele modeled by the first Gaussian, (1−f) is the fraction of SNPs on the major allele modeled by the second Gaussian, v_{b} is the mean of the first Gaussian, 1−v_{b} is the mean of second Gaussian, and s is the standard deviation. A segment was considered as LOH if the probability of bimodal VAF distribution was higher than that of unimodal distribution. To characterize panel coverages, the coverage of panel targets was retrieved from BAM files using deeptools v3.5.0. Raw coverages were computed in a bin-based manner and normalized by a pooled reference. The bin coverages were then segmented by CBS and CNVevents were detected. Finally, LOH and CNV events were integrated to report copy number gain, copy number loss, and cnLOH. The fraction of cells harboring an event was calculated based on its VAF peaks and median coverages. The sensitivity of SubChrom was evaluated by in silico mixing of tumor-normal samples, indicating the limit of detection for CNV/cnLOH events to be at 0.1 ctDNA fraction, and a strong correlation between WGS and PeCan-Seq in estimating ctDNA fraction was observed. The visualization of CNV/cnLOH events for tumor and cfDNA samples from SJBALL031914 was a combination of outputs from SubChrom and ichorCNA.

Tumor purity and ctDNA fraction estimation. Events of CNV and cnLOH, as well as somatic SNVs (coding and noncoding) and SVs from diploid regions, were used to assess tumor purity and ctDNA fraction. For CNV and cnLOH, the purity was defined as the highest cell fraction among events from SubChrom. For SNVs, a previously described method was used (Rusch et al. (2018) Nat. Commun. 9:3962). For SVs, the purity was defined as two times the highest VAF value. The final fraction was the maximal of CNV/cnLOH/SNV/SV-based estimates, and the criteria were consistent between tumor purity and ctDNA fraction estimation.

Statistical analysis. Differences in cfDNA yield and sequencing depth between groups were examined using the Mann-Whitney test. Associations between cfDNA yield and sequencing depth, tumor purity and ctDNA fraction, and different estimates of ctDNA fraction were examined by Spearman rank correlation or Pearson correlation as indicated. Analysis was performed with the use of Prism v10 (GraphPad Software) and R software v3.5.1.

Study overview. For this study, 177 children with hematologic malignancies, 38 with solid tumors, and 18 with brain tumors (Table 18) were analyzed.

TABLE 18

| | |
|---|---|
| Characteristic | Patients (N = 233) |

| | | |
|---|---|---|
| Sex | | |
| Male, No. (%) | 138 | (59%) |
| Female No. (%) | 95 | (41%) |
| Age, median (range), Yr | 7 | (0.08-21.5) |
| White blood cell count, median (range), | 8.27 | (0.3-653.7) |
| 10³/μL | | |
| Peripheral blood blasts in leukemia | 40.6 | (0-95%) |
| patients, median (range), % | | |
| Diagnosis, No. (%) | | |
| Hematologic malignancy | 177 | (76%) |
| B-cell ALL | 114 | (49%) |
| T-cell ALL | 21 | (9%) |
| AML | 34 | (15%) |
| Lymphoma | 3 | (<3%) |
| Other Leukemia | 5 | (<3%) |
| Solid tumor | 38 | (16%) |
| Ewing Sarcoma | 6 | (<3%) |
| Papillary thyroid carcinoma | 6 | (<3%) |
| Osteosarcoma | 5 | (<3%) |
| Neuroblastoma | 3 | (<3%) |
| Wilms tumor | 3 | (<3%) |
| Others^{a} | 15 | (6%) |
| Brain Tumor | 18 | (8%) |
| Medulloblastoma | 7 | (3%) |
| Diffuse Intrinsic pontine glioma | 5 | (<3%) |
| Pilocytic astrocytoma | 2 | (<3%) |
| Others^{a} | 4 | (<3%) |

^{a}Diagnosis types with less than three samples available are grouped into “Others.”

The median age at diagnosis was 7 years. Plasma cfDNA yields at diagnosis were significantly higher for hematologic malignancies than for solid and brain tumors (median, 142.0 vs. 7.2 vs. 7.1 ng/ml, P<0.0001 for both comparisons), with the highest yields observed in TCF3:PBX1 B-ALL (median, 2376 ng/ml). The unique sequencing depth after error suppression was significantly higher for hematologic malignancies than for solid and brain tumors (median, 820 vs. 482 vs. 401-fold, P<0.0001 for both comparisons), primarily resulting from variations in cfDNA yields for library preparation. The cfDNA PeCan-Seq and analysis workflow was designed to capture diverse types of somatic variants. In addition to sequence mutations, PeCan-Seq was able to detect recurrent SVs in pediatric cancers, provided baits within the panel covered one of the two genomic breakpoints. For example, introns 4 and 5 of ETV6 are frequently involved in rearrangements that generate ETV6:RUNX1, and the panel coverage of breakpoints on ETV6 alone enabled the detection of ETV6:RUNX1 in cfDNA from B⋅ALL patients. The entire gene of PAXS was covered for the detection of focal copy gain/loss, and 27 out of 28 events were detected in cfDNA in this study. One large ˜1.1 Mb deletion spanning PAXS was not detected, because both breakpoints were outside regions covered by the panel. To detect subclonal chromosomal aberrations such as CNV and cnLOH events in cfDNA samples, we developed an analytical tool, SubChrom, to identify somatic events and quantify ctDNA fraction in PeCan⋅Seq data. SubChrom successfully detected hyperdiploidy, hypodiploidy, cnLOH, and focal copy number gains and losses.

Somatic variant detection and tumor burden quantification. All children with hematologic malignancies in this cohort had detectable somatic variants in plasma cfDNA at diagnosis The median ctDNA fraction in cfDNA was 0.77, comparable to primary tumor purity from diagnostic sequencing, and a correlation between primary tumor purity and ctDNA fraction was observed (Spearman p=0.617). For ALL, the most frequently mutated genes were ETV6 (26.7% of cases), PAXS (26.7%), and RUNXI (23.7%). For acute myeloid leukemia (AML), these were RUNXI (26.5%), NRAS (26.5%), and FLT3 (26.5%). Overall, the capture panel covered 81% of clinically important variants present in the tumor samples. Of 789 variants covered by the panel, PeCan⋅Seq detected 98% of SNVs and indels, 100% of ITDs, 95% of focal copy gains/losses, 99% of chimeric gene fusions, and 99% of chromosomal copy number aberrations in cfDNA, leading to 97% overall detection rate. These results demonstrate that mutational profiles CfDNA PeCan⋅Seq were highly from concordant with tumor sequencing. In addition, twenty one panel⋅covered tumor variants were not detected, the median VAF was 0.066. Fifty cfDNA-specific variants were identified that were not detected by sequencing of the primary tumor, with a median VAF of 0.028, most commonly NRAS and KRAS mutations. These tumor-specific and cfDNA-specific variants were subclonal, potentially resulting from spatial tumor heterogeneity and sampling bias, different sequencing depth, and/or different prevalence of tumor cells with these variants in bone marrow and peripheral blood.

Of 38 children with solid tumors, PeCan-Seq identified genomic aberrations in 19 cfDNA samples with varying ctDNA fractions. These included all six children with EWSR1 fusion positive Ewing sarcoma, four of five liver tumors (two hepatocellular carcinomas and two hepatoblastomas), four of five osteosarcomas, one of three neuroblastoma cases (MYCN amplification), two of three Wilms tumors, and one PAX3:FOX01 alveolar rhabdomyosarcoma. No somatic aberrations were detected in cfDNA from six papillary thyroid carcinomas, consistent with low levels of ctDNA previously described for this tumor type. Overall, the cfDNA yields were significantly higher in ctDNA positive patients than in ctDNA negative patients (median, 10.2 vs. 5.6 ng/ml, P<0.01). cfDNA only variants, including SMARCA4 p.R966W in a child with Ewing sarcoma, and CTNNB1 p.N387K in a child with Wilms tumor, were identified. Of 18 children with brain tumors, ctDNA was identified for one child with pineoblastoma. Somatic DICER1 variants were identified in the primary tumor, however the VAFs from cfDNA were low, and the estimated ctDNA fraction was 0.048.

Evaluation of the PeCan-Seq pipeline using the initial capture panel targeting 357 genes showed that several tumor drivers were not detected in cfDNA due to lack of coverage. Accordingly, the panel was expanded with capture baits targeting additional 11 genomic sites, including DNAJB1 and PRKACA for fibrolamellar hepatocellular carcinoma, and BCL11B for BCL11B-activated lineage-ambiguous leukemia. PeCanSeq using this expanded panel achieved expected coverage for all additional targets. Previously uncovered variants were detected in six ctDNA positive samples (previously determined using the initial panel), while variants in three ctDNA negative samples remained undetectable.

The performance of cfDNA WGS was evaluated for 12 samples, to recover variants not covered by the initial panel. Genome sequencing captured previously uncovered events, including sequence mutation TRRAP p.E3107K, focal copy loss of EZH2, focal copy gain of IKZF1, and chromosomal rearrangements involving ETV6, BCL718, LM02, and RUNX1 with breakpoints located in non-recurrent genomic regions. However, low VAF events in cfDNA such as EWSR1:FLI1 were missed, due to relatively low coverage of genome sequencing compared with Pecan-Seq (median, 64 vs. 794-fold).

Minimal residual disease monitoring by ctDNA in leukemia. Pecan-Seq was performed for 14 children with B-ALL, with serial samples collected on days 0, 15, 22, and at the end of induction chemotherapy. Plasma cfDNA yields were significantly higher at diagnosis (day 0) compared to later collections (median, 168 vs. 6.2 vs. 9.3 vs. 9.2, P<0.0001 for three comparisons). To maximize the recovery of cfDNA fragments, a higher number of sequencing reads were generated for these samples than for cfDNA at diagnosis (median, 656 vs. 255 million). Despite this, their unique sequencing depths after deduplication and error suppression were lower than those from day 0 samples, presumably resulting from the correlation between cfDNA input/yield and unique sequencing depth by Pecan-Seq, suggesting that regular sequencing depth (250 million reads) was adequate to achieve similar unique depth for these samples.

Minimal residual disease (MRD) levels as quantified by flow cytometry were compared to cfDNA Pecan-Seq data. Different types of variants were detected in cfDNA from patient SJBALL032304 (flow MRD>20% on day 15). These variants included sequence mutations on day 15 (involving CHD4, KRAS, PTPN11, and CREBBP), structural variants on days 15 and 22 (involving PAXS), CNVs and cnLOH on day 15. VAFs for these loci decreased over time, consistent with MRD levels by flow. In patients SJBALL032176 (flow MRD 8.44% on day 15) and SJBALL032931 (flow MRD 1.08% on day 15), ctDNA was positive on day 15 and negative thereafter. Similar results were observed in another 4 patients with flow MRD levels over 1%. In SJBALL032162 (flow MRD <0.1% on day 15), the ctDNA fraction on day 15 was 0.16 with ETV6:RUNX1, PAXS focal deletion, RPL22 mutation, frameshift and CNVs. In SJBALL031837 (flow MRD negative with levels <0.01%), ctDNA was positive on day 15 with NRAS p. G12D detected. However, ctDNA was not detected in additional patients with flow MRD less than 0.18. In summary, cfDNA analysis successfully detected somatic mutations in all seven B-ALL patients with flow MRD levels over 1%, one of three patients with flow MRD between 0.1% and 0.01%, and one of four patients with undetectable MRD by flow cytometry.

Multi-tumor detection in a single sample. One child was diagnosed concurrently with hyperdiploid B-ALL and neuroblastoma, followed by a relapse of neuroblastoma with distinct genomic features four months after diagnosis. PeCan-Seq and WGS of plasma cfDNA collected three days before the primary diagnosis were performed. Multiple chromosomal copy number aberrations concordant with B-ALL, the primary neuroblastoma diagnosis and/or neuroblastoma relapse were detected in cfDNA, indicating the presence of ctDNA derived from three distinct tumor entities in plasma at the time of primary diagnosis. Specifically, cnLOH events (e.g., chromosome 1p, 9, 13, and 15) and gains of 1q, 10, 21 were observed exclusively in the primary B-ALL tumor sample. The diagnosis and relapse neuroblastoma samples shared chromosomal gains (2p, 12, and 17q) and losses (4p, 11q, and 14q). Specific patterns of segmental chromosomal gain of chromosome 7 were only present at diagnosis, while 2q gain was only found at relapse; both findings were detected in cfDNA. Based on DNA fractions estimated by these events, it was found that 54% of total plasma cfDNA was derived from B-ALL, 8% from neuroblastoma at diagnosis, 23% from the relapse-fated subclone of neuroblastoma present at diagnosis, and the remainder from non-tumor cells.

What is claimed is:

1. A method for genetic screening a subject for a pediatric malignancy comprising obtaining a nucleic acid sample from the subject and selectively detecting in sequences of the nucleic acid sample one or more mutations in an exonic, intronic, and/or promoter region of the genes of Table 1 and single nucleotide polymorphisms of Table 2 thereby genetic screening the subject for a pediatric malignancy.

2. The method of claim 1, wherein the pediatric malignancy comprises anaplastic large cell lymphoma, acute myeloid leukemia, acute megakaryoblastic leukemia, alveolar soft part sarcoma, atypical teratoid/rhabdoid tumor, B-lymphoblastic leukemia/lymphoma, B-lineage acute lymphoblastic leukemia, chronic myeloid leukemia, malignant tumor, ganglioglioma, adenocarcinoma, diffuse astrocytoma, germinoma, primitive neuroectodermal tumor, adamantinomatous craniopharyngioma, desmoplastic small round cell tumor, anaplastic ependymoma, ependymoma, Ewing sarcoma, gastrointestinal stromal tumor, glioblastoma, anaplastic astrocytoma, infantile fibrosarcoma high-grade glioma, low-grade glioma, pilocytic astrocytoma, desmoplastic/nodular medulloblastoma, medulloblastoma, large cell/anaplastic medulloblastoma, desmoplastic/nodular medulloblastoma, melanoma, malignant peripheral nerve sheath tumor, neuroblastoma, high-grade osteosarcoma, retinoblastoma, rhabdomyosarcoma, embryonal rhabdomyosarcoma, alveolar rhabdomyosarcoma, synovial sarcoma, desmoid/aggressive fibromatosis, dysgerminoma, sarcoma, hepatoblastoma, cutaneous squamous cell carcinoma, congenital mesoblastic nephroma, papillary renal cell carcinoma, high-grade undifferentiated pleomorphic sarcoma, poorly differentiated cutaneous (adnexal) carcinoma, desmoid/aggressive fibromatosis, T-cell acute lymphoblastic leukemia, papillary thyroid carcinoma, or Wilms tumor.

3. The method of claim 1, wherein the one or more mutations comprise a single nucleotide variant (SNV), small insertion and deletion (Indel), gene fusion, structural variant (SV), internal tandem duplication (ITD), single nucleotide polymorphism (SNP), copy number variation (CNV) and/or loss of heterozygosity (LOH).

4. The method of claim 1, wherein the nucleic acid sample is from a liquid biopsy sample, tumor sample, or blood sample.

5. The method of claim 1, further comprising treating the subject for the pediatric malignancy based upon the one or more mutations detected.

6. A method of classifying a pediatric malignancy of a subject comprising obtaining a nucleic acid sample from the subject and selectively detecting in sequences of the nucleic acid sample one or more mutations in an exonic, intronic, and/or promoter region of the genes of Table 1 and single nucleotide polymorphisms of Table 2 thereby classifying the pediatric malignancy of the subject.

7. The method of claim 6, wherein the pediatric malignancy comprises anaplastic large cell lymphoma, acute myeloid leukemia, acute megakaryoblastic leukemia, alveolar soft part sarcoma, atypical teratoid/rhabdoid tumor, B-lymphoblastic leukemia/lymphoma, B-lineage acute lymphoblastic leukemia, chronic myeloid leukemia, malignant tumor, ganglioglioma, adenocarcinoma, diffuse astrocytoma, germinoma, primitive neuroectodermal tumor, adamantinomatous craniopharyngioma, desmoplastic small round cell tumor, anaplastic ependymoma, ependymoma, Ewing sarcoma, gastrointestinal stromal tumor, glioblastoma, anaplastic astrocytoma, infantile fibrosarcoma high-grade glioma, low-grade glioma, pilocytic astrocytoma, desmoplastic/nodular medulloblastoma, medulloblastoma, large cell/anaplastic medulloblastoma, desmoplastic/nodular medulloblastoma, melanoma, malignant peripheral nerve sheath tumor, neuroblastoma, high-grade osteosarcoma, retinoblastoma, rhabdomyosarcoma, embryonal rhabdomyosarcoma, alveolar rhabdomyosarcoma, synovial sarcoma, desmoid/aggressive fibromatosis, dysgerminoma, sarcoma, hepatoblastoma, cutaneous squamous cell carcinoma, congenital mesoblastic nephroma, papillary renal cell carcinoma, high-grade undifferentiated pleomorphic sarcoma, poorly differentiated cutaneous (adnexal) carcinoma, desmoid/aggressive fibromatosis, T-cell acute lymphoblastic leukemia, papillary thyroid carcinoma, or Wilms tumor.

8. The method of claim 6, wherein the one or more mutations comprise a single nucleotide variant (SNV), small insertion and deletion (Indel), gene fusion, structural variant (SV), internal tandem duplication (ITD), single nucleotide polymorphism (SNP), copy number variation (CNV) and/or loss of heterozygosity (LOH).

9. The method of claim 6, wherein the nucleic acid sample is from a liquid biopsy sample, tumor sample, or blood sample.

10. The method of claim 6, further comprising treating the subject for the pediatric malignancy based upon the classification of the pediatric malignancy.

11. A method for monitoring progression of a pediatric malignancy in a subject comprising obtaining a first nucleic acid sample from the subject at a first time point; obtaining a second nucleic acid sample from the subject at a second time point; selectively detecting the presence in sequences of the first and second nucleic acid samples one or more mutations in an exonic, intronic, and/or promoter region of the genes of Table 1 and single nucleotide polymorphisms of Table 2; comparing the presence of the one or more mutations and single nucleotide polymorphisms in the first nucleic acid sample with the presence of the one or more mutations and single nucleotide polymorphisms in the second nucleic acid sample thereby monitoring the progression of the pediatric malignancy.

12. The method of claim 11, wherein the pediatric malignancy comprises anaplastic large cell lymphoma, acute myeloid leukemia, acute megakaryoblastic leukemia, alveolar soft part sarcoma, atypical teratoid/rhabdoid tumor, B-lymphoblastic leukemia/lymphoma, B-lineage acute lymphoblastic leukemia, chronic myeloid leukemia, malignant tumor, ganglioglioma, adenocarcinoma, diffuse astrocytoma, germinoma, primitive neuroectodermal tumor, adamantinomatous craniopharyngioma, desmoplastic small round cell tumor, anaplastic ependymoma, ependymoma, Ewing sarcoma, gastrointestinal stromal tumor, glioblastoma, anaplastic astrocytoma, infantile fibrosarcoma high-grade glioma, low-grade glioma, pilocytic astrocytoma, desmoplastic/nodular medulloblastoma, medulloblastoma, large cell/anaplastic medulloblastoma, desmoplastic/nodular medulloblastoma, melanoma, malignant peripheral nerve sheath tumor, neuroblastoma, high-grade osteosarcoma, retinoblastoma, rhabdomyosarcoma, embryonal rhabdomyosarcoma, alveolar rhabdomyosarcoma, synovial sarcoma, desmoid/aggressive fibromatosis, dysgerminoma, sarcoma, hepatoblastoma, cutaneous squamous cell carcinoma, congenital mesoblastic nephroma, papillary renal cell carcinoma, high-grade undifferentiated pleomorphic sarcoma, poorly differentiated cutaneous (adnexal) carcinoma, desmoid/aggressive fibromatosis, T-cell acute lymphoblastic leukemia, papillary thyroid carcinoma, or Wilms tumor.

13. The method of claim 11, wherein the one or more mutations comprise a single nucleotide variant (SNV), small insertion and deletion (Indel), gene fusion, structural variant (SV), internal tandem duplication (ITD), single nucleotide polymorphism (SNP), copy number variation (CNV) and/or loss of heterozygosity (LOH).

14. The method of claim 11, wherein the nucleic acid sample is from a liquid biopsy sample, tumor sample, or blood sample.

15. The method of claim 11, further comprising treating the subject for the pediatric malignancy based upon the progression of the pediatric malignancy.

16. A method for monitoring the response of a pediatric malignancy to a therapy in a subject comprising obtaining a first nucleic acid sample from the subject prior to treatment with the therapy; obtaining a second nucleic acid sample from the subject after treatment with the therapy; selectively detecting the presence in sequences of the first and second nucleic acid samples one or more mutations in an exonic, intronic, and/or promoter region of the genes of Table 1 and single nucleotide polymorphisms of Table 2; comparing the presence of the one or more mutations and single nucleotide polymorphisms in the first nucleic acid sample with presence of the one or more mutations and single nucleotide polymorphisms in the second nucleic acid sample thereby monitoring the response of the pediatric malignancy to the therapy in the subject.

17. The method of claim 16, wherein the pediatric malignancy comprises anaplastic large cell lymphoma, acute myeloid leukemia, acute megakaryoblastic leukemia, alveolar soft part sarcoma, atypical teratoid/rhabdoid tumor, B-lymphoblastic leukemia/lymphoma, B-lineage acute lymphoblastic leukemia, chronic myeloid leukemia, malignant tumor, ganglioglioma, adenocarcinoma, diffuse astrocytoma, germinoma, primitive neuroectodermal tumor, adamantinomatous craniopharyngioma, desmoplastic small round cell tumor, anaplastic ependymoma, ependymoma, Ewing sarcoma, gastrointestinal stromal tumor, glioblastoma, anaplastic astrocytoma, infantile fibrosarcoma high-grade glioma, low-grade glioma, pilocytic astrocytoma, desmoplastic/nodular medulloblastoma, medulloblastoma, large cell/anaplastic medulloblastoma, desmoplastic/nodular medulloblastoma, melanoma, malignant peripheral nerve sheath tumor, neuroblastoma, high-grade osteosarcoma, retinoblastoma, rhabdomyosarcoma, embryonal rhabdomyosarcoma, alveolar rhabdomyosarcoma, synovial sarcoma, desmoid/aggressive fibromatosis, dysgerminoma, sarcoma, hepatoblastoma, cutaneous squamous cell carcinoma, congenital mesoblastic nephroma, papillary renal cell carcinoma, high-grade undifferentiated pleomorphic sarcoma, poorly differentiated cutaneous (adnexal) carcinoma, desmoid/aggressive fibromatosis, T-cell acute lymphoblastic leukemia, papillary thyroid carcinoma, or Wilms tumor.

18. The method of claim 16, wherein the one or more mutations comprise a single nucleotide variant (SNV), small insertion and deletion (Indel), gene fusion, structural variant (SV), internal tandem duplication (ITD), single nucleotide polymorphism (SNP), copy number variation (CNV) and/or loss of heterozygosity (LOH).

19. The method of claim 16, wherein the nucleic acid sample is from a liquid biopsy sample, tumor sample, or blood sample.

20. The method of claim 16, further comprising treating the subject for the pediatric malignancy based upon the response of the pediatric malignancy to the therapy in the subject.


SEQUENCE LISTING (86 sequences)

SEQ ID NO: 1 - DNA, 23 bp, synthetic construct
  source 1..23: mol_type = other DNA
atctcaggcgtgcccgactcngg

SEQ ID NO: 2 - DNA, 23 bp, synthetic construct
  source 1..23: mol_type = other DNA
ctgagatttctggtgcaggtngg

SEQ ID NO: 3 - DNA, 23 bp, synthetic construct
  source 1..23: mol_type = other DNA
gattctatcactcctaggccngg

SEQ ID NO: 4 - DNA, 23 bp, synthetic construct
  source 1..23: mol_type = other DNA
ctggggctgggaactccgtangg

SEQ ID NO: 5 - DNA, 23 bp, synthetic construct
  source 1..23: mol_type = other DNA
tgtatgactgtatcataacgngg

SEQ ID NO: 6 - DNA, 23 bp, synthetic construct
  source 1..23: mol_type = other DNA
caagtagatcgaggtggagangg

SEQ ID NO: 7 - DNA, 23 bp, synthetic construct
  source 1..23: mol_type = other DNA
ccagaattcttctgggtaggngg

SEQ ID NO: 8 - DNA, 101 bp, Homo sapiens
  source 1..101: mol_type = genomic DNA
gccctgtgccttccaccagcccaggaatcctgcctgctttccaggcagactttccaagtaccttgattctatcactcctaggccagggcatctcaccgcag

SEQ ID NO: 9 - DNA, 27 bp, Homo sapiens
  source 1..27: mol_type = genomic DNA
agggaccggagtcgggcacgcctgaga

SEQ ID NO: 10 - DNA, 101 bp, Homo sapiens
  source 1..101: mol_type = genomic DNA
tttctggtgcaggtgggtcattatttttaacagctgccaagtatccctttgtatgactgtatcataacgtggttgttaaatctcctatgcatagtttttcc

SEQ ID NO: 11 - DNA, 101 bp, Homo sapiens
  source 1..101: mol_type = genomic DNA
ccctgtgccttccaccagcccaggaatcctgcctgctttccaggcagactttccaagtaccttgattctatcactcctaggccagggcatctcaccgcagc

SEQ ID NO: 12 - DNA, 25 bp, Homo sapiens
  source 1..25: mol_type = genomic DNA
ttggtcccctctccacctcgatcta

SEQ ID NO: 13 - DNA, 101 bp, Homo sapiens
  source 1..101: mol_type = genomic DNA
cttgctcacccaggtattcttcaaagagcagcctcctccctcctacccagaagaattctggtaacatctattttgaaaatcgtttttttaccctgttgcat

SEQ ID NO: 14 - DNA, 101 bp, Homo sapiens
  source 1..101: mol_type = genomic DNA
tgcctcttcattcgcctgctcccagacgctgtgtgcctggcagcgctcagcactggggaaggggcgaggggtgcagcaggatgcctctgcctcaggggaga

SEQ ID NO: 15 - DNA, 101 bp, Homo sapiens
  source 1..101: mol_type = genomic DNA
acttgcagttgaggaaatgcagaaaatggaaagctgaagtcagcggatcactacctgttagagaaaggcttagcctggctcccaggttgtcttgcttccca

SEQ ID NO: 16 - DNA, 101 bp, Homo sapiens
  source 1..101: mol_type = genomic DNA
ggctgcggggaggacttgggatttggccatgagaaaggtggcagccgtggagggctgaggagggatgggacctgacccaggtgctcacagataccctctgg

SEQ ID NO: 17 - DNA, 101 bp, Homo sapiens
  source 1..101: mol_type = genomic DNA
cactgttgttaactggaggcttcaccactttgggccccctcaccaccatgacgtcattgactcgcctgactcctcccagcctctcccctgcctccagctcc

SEQ ID NO: 18 - DNA, 101 bp, Homo sapiens
  source 1..101: mol_type = genomic DNA
ggccctgtgccttccaccagcccaggaatcctgcctgctttccaggcagactttccaagtaccttgattctatcactcctaggccagggcatctcaccgca

SEQ ID NO: 19 - DNA, 12 bp, Homo sapiens
  source 1..12: mol_type = genomic DNA
gtctccaaaccc

SEQ ID NO: 20 - DNA, 101 bp, Homo sapiens
  source 1..101: mol_type = genomic DNA
gcttgctcacccaggtattcttcaaagagcagcctcctccctcctacccagaagaattctggtaacatctattttgaaaatcgtttttttaccctgttgca

SEQ ID NO: 21 - DNA, 101 bp, Homo sapiens
  source 1..101: mol_type = genomic DNA
ttagaacattattaaaactcgagtaatactactttccatttttctatgaatatttgtcttggttttataactataattgtcagtcatttgtgtgattttaa

SEQ ID NO: 22 - DNA, 101 bp, Homo sapiens
  source 1..101: mol_type = genomic DNA
ggccctgtccctggctcgggcccttgaagaggccttggaagccaaagaggaactcgagcggaccaacaaaatgctcaaagccgaaatggaagacctggtca

SEQ ID NO: 23 - DNA, 101 bp, Homo sapiens
  source 1..101: mol_type = genomic DNA
attacttattgtaactgtattattcctaaaagtataaggtcatgtttaactgaaattatataatattttgacccacaaattgattttattatattgctagc

SEQ ID NO: 24 - DNA, 101 bp, Homo sapiens
  source 1..101: mol_type = genomic DNA
gagcttcaggccgactctgccatcaaggggagggaggaagccatcaagcagctacgcaaactgcaggtgggtgacactaggagcttggggcatgggtggag

SEQ ID NO: 25 - DNA, 101 bp, Homo sapiens
  source 1..101: mol_type = genomic DNA
tatttagaaaaaaataaaatttgctttcagtattacacagaataatgaaaacagaagattctagaccttgctatctctattctctggcatataggctgttt

SEQ ID NO: 26 - DNA, 101 bp, Homo sapiens
  source 1..101: mol_type = genomic DNA
ccaagcgggccctggagacccagatggaggagatgaagacgcagctggaagagctggaggacgagctgcaagccacggaggacgccaaactgcggctggaa

SEQ ID NO: 27 - DNA, 101 bp, Homo sapiens
  source 1..101: mol_type = genomic DNA
tgtgattcagattatctttagagattcaaacatcatctaatctaacattattacagttgataaaactgagagtctccaaaaaattaattgacttgctctcc

SEQ ID NO: 28 - DNA, 101 bp, Homo sapiens
  source 1..101: mol_type = genomic DNA
ctcaacgtgtctacgaagctgcgccagctggaggaggagcggaacagcctgcaagaccagctggacgaggagatggaggccaagcagaacctggagcgcca

SEQ ID NO: 29 - DNA, 101 bp, Homo sapiens
  source 1..101: mol_type = genomic DNA
aaaattcccaactcccagcaatgctttgtctgattgttcatttgcagatgtcttagcgtgttaatttaaatgtcaaaggttttgaggtgtccagaaccacc

SEQ ID NO: 30 - DNA, 101 bp, Homo sapiens
  source 1..101: mol_type = genomic DNA
gctggtctttcatttgtcttgtttgtttttaagcaagaagaatccctttagaggaggaattaggaaagaaaaaaaagtcaaacagaaacagaaggagtgga

SEQ ID NO: 31 - DNA, 101 bp, Homo sapiens
  source 1..101: mol_type = genomic DNA
ccagaatgacgaccacgacgctgatgatgaggatgatggccacctggagtccacaaagaaaaaggagctagaggaacagcccacgagggaagatgtcaagg

SEQ ID NO: 32 - DNA, 101 bp, Homo sapiens
  source 1..101: mol_type = genomic DNA
tttttttttgagacggagtttagctcttgttgcccaggctggagtgcagtggtacgatctcggctcactgcaacttctccctcctggtttcaagcaactct

SEQ ID NO: 33 - DNA, 101 bp, Homo sapiens
  source 1..101: mol_type = genomic DNA
aacttttttgtatggatatgtagggcttggcgagtctaggtcaagcattccagccaaagaattgtgaaagatcacaacaatctgggaataacaaagattca

SEQ ID NO: 34 - DNA, 101 bp, Homo sapiens
  source 1..101: mol_type = genomic DNA
aagtccaagaaaaagaaaatgatctctactacctcaaaggaaactaagaaggacacaaagctttactgtatctgtaaaacgccttatgatgaatctaagtg

SEQ ID NO: 35 - DNA, 101 bp, Homo sapiens
  source 1..101: mol_type = genomic DNA
ggggcgcgctggccacgctcaaggtgagcaccatcaagcgccacatcctgcaggtgcaccccttctccatggacttcacgcctgaggagcgccagactatc

SEQ ID NO: 36 - DNA, 101 bp, Homo sapiens
  source 1..101: mol_type = genomic DNA
atccagaaatgtaatttattctcagtcttcactgaagagcatctggctcttgagctggaaatatggctctataagctttattgtatagctgagtttctctg

SEQ ID NO: 37 - DNA, 101 bp, Homo sapiens
  source 1..101: mol_type = genomic DNA
ctaccagccgcggtggcggggcgagtacctgatggactacgacggcagccggcgcggcctggtgtgtatggtgtgcgggggcgcgctggccacgctcaagg

SEQ ID NO: 38 - DNA, 101 bp, Homo sapiens
  source 1..101: mol_type = genomic DNA
tggtatgtaaattcaaaactagaataataggctacattatgtgctctcattgtctgaaaaataagttccctgaaaaaatccaggataccttaagtgatatt

SEQ ID NO: 39 - DNA, 101 bp, Homo sapiens
  source 1..101: mol_type = genomic DNA
tgagcaccatcaagcgccacatcctgcaggtgcaccccttctccatggacttcacgcctgaggagcgccagactatcctggaggcctacgaggaggcggcg

SEQ ID NO: 40 - DNA, 101 bp, Homo sapiens
  source 1..101: mol_type = genomic DNA
aggtgtggactaccacacctagcctaaatctagaactttctatgtatatatttacaaataatattttagatttttgttctctggttcaaattaacttctca

SEQ ID NO: 41 - DNA, 101 bp, Homo sapiens
  source 1..101: mol_type = genomic DNA
caggtgcaccccttctccatggacttcacgcctgaggagcgccagactatcctggaggcctacgaggaggcggcgctgcgctgctacggccacgagggctt

SEQ ID NO: 42 - DNA, 101 bp, Homo sapiens
  source 1..101: mol_type = genomic DNA
cgtccggcaacaaaggatgttttgtgctactactgaggtttgtgtgtgtgacttactttagaactctttctagaaaatgcgattactatttgcataggtct

SEQ ID NO: 43 - DNA, 101 bp, Homo sapiens
  source 1..101: mol_type = genomic DNA
gagcaccatcaagcgccacatcctgcaggtgcaccccttctccatggacttcacgcctgaggagcgccagactatcctggaggcctacgaggaggcggcgc

SEQ ID NO: 44 - DNA, 57 bp, Homo sapiens
  source 1..57: mol_type = genomic DNA
cggggcgggcggcccgaagccctcgtggggtaattaaaacgttattttcttttcttt

SEQ ID NO: 45 - DNA, 101 bp, Homo sapiens
  source 1..101: mol_type = genomic DNA
aaaaaatcagaataaacaatttggtcaagtaaaatatttccctccaagtagttaaggcaaagactgaaggaccatttgtaggaaatggagaatctttctat

SEQ ID NO: 46 - DNA, 101 bp, Homo sapiens
  source 1..101: mol_type = genomic DNA
ggcaactgaatctagcagtttggaggtcttagagcatttgtaataacatgctggctctctgtgaatgtcccagaaggaacatcttccatggaatggacttg

SEQ ID NO: 47 - DNA, 101 bp, Homo sapiens
  source 1..101: mol_type = genomic DNA
tccatgcggtctatgtggtaaggtgttcactgatgccaaccggctccggcagcacgaggcccagcacggtgtcaccagcctccagctgggctacatcgacc

SEQ ID NO: 48 - DNA, 101 bp, Homo sapiens
  source 1..101: mol_type = genomic DNA
actcttcgtgtgttctttgatcaagtcaggactattacttccattgcagggagactgaggcccagagagggaaagtgccttgtccaaagtcacacagctgg

SEQ ID NO: 49 - DNA, 101 bp, Homo sapiens
  source 1..101: mol_type = genomic DNA
ggcctgagggaggcaggcatccttccatgcggtctatgtggtaaggtgttcactgatgccaaccggctccggcagcacgaggcccagcacggtgtcaccag

SEQ ID NO: 50 - DNA, 101 bp, Homo sapiens
  source 1..101: mol_type = genomic DNA
gctcaagcattgctacgttcattccttgagataatttgtgcaaagtgggggaaataaccccctttcagattttctctctttctctctctctgtggcaggta

SEQ ID NO: 51 - DNA, 101 bp, Homo sapiens
  source 1..101: mol_type = genomic DNA
caagcctctttgcctgtgttacctggggtggaccgcttgcccatggtggctggacccctatccccccaactgctgacttccccattccccagtgtggcatc

SEQ ID NO: 52 - DNA, 101 bp, Homo sapiens
  source 1..101: mol_type = genomic DNA
ctgctttgcccatcagtctgtcctttcagagttgaagctgagctgctgtttgctgggcaggccatgcagcccacacgggggtcctcagaggccttgcaggg

SEQ ID NO: 53 - DNA, 10 bp, Homo sapiens
  source 1..10: mol_type = genomic DNA
acgtctctgg

SEQ ID NO: 54 - DNA, 101 bp, Homo sapiens
  source 1..101: mol_type = genomic DNA
gctgggctacatcgaccttcctcctccgaggctgggtgagaatgggctacccatctctgaagaccccgacggcccccgaaagaggagccggaccaggaagc

SEQ ID NO: 55 - DNA, 101 bp, Homo sapiens
  source 1..101: mol_type = genomic DNA
ccactgtcctacccgagtgaggcttgttacagacatcagggcccacctgactgtggtgggctacacgaggatgctcacatttcctccattagtcacctgat

SEQ ID NO: 56 - DNA, 101 bp, Homo sapiens
  source 1..101: mol_type = genomic DNA
gcgcgtggtgcaggagcatctcccgctgatgagcgaggcgggtgctggcctgcctgacatggaggctgtggcaggtgccgaagccctcaatggccagtccg

SEQ ID NO: 57 - DNA, 101 bp, Homo sapiens
  source 1..101: mol_type = genomic DNA
ccaggaaggtctagctcctgacacgttctatggtagagggaggagggttgatgcttgctcaggttacttgggaacatctcttccccagtatgccttccaac

SEQ ID NO: 58 - DNA, 101 bp, Homo sapiens
  source 1..101: mol_type = genomic DNA
tctcggtggctgtgggcctggccgtctttgcctgcctcttcctttctacgctgctccttgtgctcaacaaatgtggacggagaaacaagtttgggatcaac

SEQ ID NO: 59 - DNA, 101 bp, Homo sapiens
  source 1..101: mol_type = genomic DNA
ccgccactttgctggcacctgctctaaacatctggtctctgctgcttggctctcaggagcaaaggtataaggacgtggccaatgctaggttattagcttag

SEQ ID NO: 60 - DNA, 101 bp, Homo sapiens
  source 1..101: mol_type = genomic DNA
atgcactagcccactcttccccaaaccagccctccaccaccctccaggcagagagataggaaaatcggtttctgagtatatttctgttcagcctgtgagcc

SEQ ID NO: 61 - DNA, 101 bp, Homo sapiens
  source 1..101: mol_type = genomic DNA
catatgaaaatatctcttctttatataagagaaattactccagtcagaaggacttagaaacatgtttttttccttttaaacttttaagtcagtttttatga

SEQ ID NO: 62 - DNA, 101 bp, Homo sapiens
  source 1..101: mol_type = genomic DNA
cttttagtaattcagtacctgtttgagctagtctgtgctttatagtgtggagacaacttaactttccagggattctcagcagctgactggtagcttgccag

SEQ ID NO: 63 - DNA, 101 bp, Homo sapiens
  source 1..101: mol_type = genomic DNA
cttggtgatataactttgttttgtttacagagtacctgctcgggccaggtaaatgctattggatgtaatccagtagtgtgtaatataaattcaaaccatat

SEQ ID NO: 64 - DNA, 101 bp, Homo sapiens
  source 1..101: mol_type = genomic DNA
gtgtggccaccacaccagctttttttttttttttttcgtatttttgtagagacatggtttcactatgttgggcaggctggtctcgaactcctgacctgaag

SEQ ID NO: 65 - DNA, 101 bp, Homo sapiens
  source 1..101: mol_type = genomic DNA
taaatagcattttttaaaaaacagaatgaacttcaaaattaaagttgatttttaacttccatattagcaaatactcttcactactgaaagacagtactatt

SEQ ID NO: 66 - DNA, 101 bp, Homo sapiens
  source 1..101: mol_type = genomic DNA
caaggtacaactattcttcagtatgcacagacctctgatggacagcagatacttgtgcccagcaatcaggtggtcgtacaaagtaagtatgctttctgtct

SEQ ID NO: 67 - DNA, 592 bp, Homo sapiens
  source 1..592: mol_type = other DNA
gacgaggaggaggagccagaggaggaggaggaggagtggggcgacgttccgctgtcccctggagctcccttggagcggcccgccgaagaagaggaggacgaagaggacggccaggagcctgggggactcgccttgccgccgccgcctcctcccccgcctccgcccccgccccgcagccgggagcagcggcggaactaccagccgcggtggcggggcgagtacctgatggactacgacggcagccggcgcggcctggtgtgtatggtgtgcgggggcgcgctggccacgctcaaggtgagcaccatcaagcgccacatcctgcaggtgcaccccttctccatggacttcacgcctgaggagcgccagactatcctggaggcctacgaggaggcggcgctgcgctgcgacctggaggcgctcttcaacgccgtcatgaaccccaagacggccaacgtgccccagaccgtgcccatgaggctccggaagctgcccgactccttcttcaagccgccggagcccaaatcccactcccgacaggccagttgtatagtctcctgtcggagaccaaagggttttggaactcagaaaaaat

SEQ ID NO: 68 - DNA, 496 bp, Homo sapiens
  source 1..496: mol_type = other DNA
ccgggagcagcggcggaactaccagccgcggtggcggggcgagtacctgatggactacgacggcagccggcgcggcctggtgtgtatggtgtgcgggggcgcgctggccacgctcaaggtgagcaccatcaagcgccacatcctgcaggtgcaccccttctccatggacttcacgcctgaggagcgccagactatcctggaggcctacgaggaggcggcaggagctggcaaacacaccaaggccaccgccactgctgccaccactacagcccctccaccgccccctgctgcccctcctgcggcctcccaagcagcagcaacagcagccccaccgcccccaccagactatcaccatcaccaccagcagcacctgctgaacagtagcaataatggtggcagtggtgggataaacggagagcagcagccgcccgcttcaaccccaggggaccagaggaactcagccctgattgcggatattccttaactgataagaagc

SEQ ID NO: 69 - DNA, 507 bp, Homo sapiens
  source 1..507: mol_type = other DNA
gatcgactccagcaccgtagagaggattgaagacagccatagcccgggcgtggccgtgatacagttcgccgtcggggagcaccgagcccaggtcagcgttgaagttttggtagagtatcctttttttgtgtttggacagggctggtcatcctgctgtccggagagaaccagccagctctttgatttgccgtgttccaaactctcagttggggatgtctgcatctcgcttaccctcaagaacctgaagaacggctctgttaaaaagggccagcccgtggatcccagcaaggccggccccaaggccccgactgcctgcctgccaccacccaggccccagaggccagtgaccaaggcccgccggccaccaccccggccccaccggcgagcagagaccaaggcccgcctgccaccacccaggccccagagaccagcagagaccaaggtccctgaggagatccccccagaagtggtgcaggagtatgtggacatcatggaggagctgctagg

SEQ ID NO: 70 - DNA, 521 bp, Homo sapiens
  source 1..521: mol_type = other DNA
cctacaaaggatatatttgtaaaagaccaaaaattattgatgctaaacctactcatgaattacttacaacaaaagctgacacaaggaagatggacccttctaaaccgtcttccaacgtggccggagtagtcatcattgtgatcctcctgattttaacgggtgctggccttgccgcctatttcttttataagaaaagacgtgtgcacctacctcaagagggcgcctttgaaaacactctgtattttgagtctgtgagctctgacggccatgagtacatctacgtggaccccatgcagctgccctatgactccacgtgggagctgccgcgggaccagcttgtgctgggacgcaccctcggctctggggcctttgggcaggtggtggaggccacggctcatggcctgagccattctcaggccacgatgaaagtggccgtcaagatgcttaaatccacagcccgcagcagtgagaagcaagcccttatgtcggagctgaagatcatgagtcaccttgggccccac

SEQ ID NO: 71 - DNA, 385 bp, Homo sapiens
  source 1..385: mol_type = other DNA
gtgcgctctccccagcctgtcccttctccacggccacagtcccagcccccccactccagtccttccccaaggatgcagcctcagccttctccacaccacgtttccccacagacaagttccccacatcctggactggtagctgcccaggccaaccccatggaacaagggcattttgccagcccggaccagaattcaatgctttctcagcttgctagcaatccaggcatggcaaacctccatggtgcaagcgccacggacctgggactcagcaccgatttatgtctacccgctgcttactgtgagcgtgcaatgatgcgcttctcagagttggagatgaaagaaagagaaggtggccacccagcaaccaaagactccgagatgtgcc

SEQ ID NO: 72 - DNA, 423 bp, Homo sapiens
  source 1..423: mol_type = other DNA
tggaactgcgggcggaaagccagtgagacgtgcagcggctgcaacgcggcacgctactgcgggtccttctgccagcatcgggactgggagaagcatcaccacgtgtgtggccagagcctgcagggccccacagccgtggtggccgacccggtgcctggaccgcccgaagccgcccacagcctgggcccctccctgcctgtgggtgctgccagcctggtggatgacagccccacacctggctctccaggctccccgccctcaggcttcctgctgaactccaagttccccgagaaggtggagggacgcttttcagcagcccctctcgtggacctcagcctgtcaccaccatctgggctggactcccccaatggcagcagctcgctgtcccccgagcgccagggcaacggggacctgcctccagtg

SEQ ID NO: 73 - DNA, 709 bp, Homo sapiens
  source 1..709: mol_type = other DNA
ggcgcctggagaggaggctgaaggagtccctgcagaactggttccgggccgagtgtctcatggactatgacccgcgggggaaccggctggtgtgcatggcctgtggccgggcactgcccagcctgcacctggacgacatccgtgcccacgtgctggaggtgcaccctggctccctggggctcagcggcccccagcgcagtgccctgctgcaggcctgggggggccagcccgaggcgctgtctgagctcacccagtccccaccaggcgatgacctcgccccccaggacctgaccggaaagagccgggactcggcctccgctgctggagccccctcctctcaggatccctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaa

SEQ ID NO: 74 - DNA, 870 bp, Homo sapiens
  source 1..870: mol_type = other DNA
aaaaccttttgcggactctcaggtctcccagctctcccctgcagcagcaacaggtgcttagtatccttcacgccaacccccagctgttggctgcattcatcaagcagcgggctgccaagtatgccaactctaatccacaacccatccctgggcagcctggcatgccccaggggcagccagggctacagccacctaccatgccaggtcagcagggggtccactccaatccagccatgcagaacatgaatccaatgcaggcgggcgttcagagggctggcctgccccagcagcaaccacagcagcaactccagccacccatgggagggatgagcccccaggctcagcagatgaacatgaaccacaacaccatgccttcacaattccgagacatcttgagacctgtgttctccggctctccgcccatgaagagtctttcatccaccagtgcaggcggcaaaaagcaggctcagccaagctgcgcaccagcctccaggccgcctgccaaacagcagaaaattaaagaaaaccagaagacagatgtgctgtgtgcagacgaagaagaggattgccaggctgcctccctgctgcagaaatacaccgacaacagcgagaagccatccgggaagagactgtgcaaaaccaaacacttgatccctcaggagtccaggcggggattgccactgacaggggaatactacgtggagaatgccgatggcaaggtgactgtccggagattcagaaagcggccggagcccagttcggactatgatctgtcaccagccaagcaggagccaaagcccttcgaccgcttgcagcaactgctaccagcctcccagtccacacagctgccatgctcaagttcccctcagg

SEQ ID NO: 75 - DNA, 175 bp, Homo sapiens
  source 1..175: mol_type = other DNA
cgcgaggagctcaaccactgggcgcggcgctacagcgacgccgaggacacaaagaagggccccgctcccgccgcggcccggccccgcagcagctccgccggtcccgagtgcctctcgccagacctgcccctgcccaagcagctggtgtgtcgctgggccaagtgtaaccagctct

SEQ ID NO: 76 - DNA, 219 bp, Homo sapiens
  source 1..219: mol_type = other DNA
atttggaacagctcttggtgctggacaggcatctttgtttgggaacaaccaacctaagattggagggcctcttggtacaggagcctttggggcccctggatttaatactacgacagccactttgggctttggagccccccaggccccagtagaaaaggtagagcaactttttggagaaggaaaacagaagtccaaggagttaaagaaaatggacaaacc

SEQ ID NO: 77 - DNA, 455 bp, Homo sapiens
  source 1..455: mol_type = other DNA
ctgcggcgcaccctggaccagcgccgggccctggtcatgcagctctttcaggaccatggcttcttcccgtcagcccaggccacagccgccttccaggcccgctatgcagacatctttccctccaaggtttgtctgcagttgaagatccgtgaggtgcgccagaagatcatgcaggctgccactcccacggagcagccccctggagctgaggctcctctccctgtaccgccccccactggcaccgctgctgcccctgcccccactcccagccccgcagggggccctgaccccacctcacccagctcggactctggcacggcccaggctgccccgccactgcctccacccccagagtcggggcctggacagcctggctgggaggttaccggccccttacacacctacagcagctcccttttcagcccagcagaggggcccctgtcagcaggagaagg

SEQ ID NO: 78 - DNA, 101 bp, Homo sapiens; identical to SEQ ID NO: 8
  source 1..101: mol_type = genomic DNA

SEQ ID NO: 79 - DNA, 27 bp, Homo sapiens
  source 1..27: mol_type = genomic DNA
agggaccggagtcgggcacgcctgaga

SEQ ID NO: 80 - DNA, 101 bp, Homo sapiens; identical to SEQ ID NO: 10
  source 1..101: mol_type = genomic DNA

SEQ ID NO: 81 - DNA, 101 bp, Homo sapiens; identical to SEQ ID NO: 11
  source 1..101: mol_type = genomic DNA

SEQ ID NO: 82 - DNA, 25 bp, Homo sapiens
  source 1..25: mol_type = genomic DNA
ttggtcccctctccacctcgatcta

SEQ ID NO: 83 - DNA, 101 bp, Homo sapiens; identical to SEQ ID NO: 13
  source 1..101: mol_type = genomic DNA

SEQ ID NO: 84 - DNA, 53 bp, Homo sapiens
  source 1..53: mol_type = genomic DNA
ccgccaagaaaagaagttcccaaaaccactcctagtgagcccaagaaaaagca

SEQ ID NO: 85 - DNA, 48 bp, Homo sapiens
  source 1..48: mol_type = genomic DNA
aatacacagaaaaaaaattcctagaaaatagagattctttaataaata

SEQ ID NO: 86 - DNA, 115 bp, Homo sapiens
  source 1..115: mol_type = genomic DNA
accttcaagcagctggtggaggacctggaccgtgtccttaccgtggccagctcctcgaggagcggacctgtaaaactagaatttgatgtatctgatggcgccaccagcaaaaggg